(19) United States
(12) Patent Application Publication
(10) Pub. No.: US 2016/0067241 A1
(43) Pub. Date: Mar. 10, 2016

(54) COMPOSITIONS AND METHODS TO IMPROVE THE THERAPEUTIC BENEFIT OF SUBOPTIMALLY ADMINISTERED CHEMICAL COMPOUNDS INCLUDING SUBSTITUTED NAPHTHALIMIDES SUCH AS AMONAFIDE FOR THE TREATMENT OF IMMUNOLOGICAL, METABOLIC, INFECTIOUS, AND BENIGN OR NEOPLASTIC HYPERPROLIFERATIVE DISEASE CONDITIONS

(71) Applicant: Dennis M. BROWN, Menlo Park, CA (US)

(72) Inventor: Dennis M. BROWN, Menlo Park, CA (US)

(21) Appl. No.: 14/888,683

(22) PCT Filed: May 1, 2014
(86) PCT No.: PCT/US2014/036304

§ 371 Date: Nov. 2, 2015

Related U.S. Application Data
(60) Provisional application No. 61/818,098, filed on Jun. 13, 2013.

(51) Int. Cl. A61K 31/473

(52) U.S. Cl. CPC A61K 31/473

(57) ABSTRACT

The present invention describes methods and compositions for improving the therapeutic efficacy of therapeutic agents previously limited by suboptimal therapeutic performance by either improving efficacy as monotherapy or reducing side effects. Such methods and compositions are particularly applicable to naphthalimides such as amonafide or analogs, derivatives, or prodrugs thereof.

### CROSS-REFERENCE TO RELATED APPLICATION

This application claims the benefit of U.S. Provisional Patent Application Ser. No. 61/818,098 entitled “Compositions and Methods to Improve the Therapeutic Effect of Suboptimally Administered Compounds Including Substituted Naphthalimides Such as Amonafide for the Treatment of Immunological, Metabolic, Infectious, and Benign or Neoplastic Hyperproliferative Disease Conditions” by Dennis M. Brown, and filed on May 1, 2013, the contents of which are incorporated herein by this reference.

### TECHNICAL FIELD OF THE INVENTION

The present invention relates to the general field of treatment of immunological, metabolic, infectious, and benign or neoplastic hyperproliferative disease conditions, including oncology applications, with a focus on novel methods and compositions for the improved utility of chemical agents, compounds, dosage forms limited by suboptimal human therapeutic performance including substituted naphthalimides such as amonafide.

### BACKGROUND OF THE INVENTION

The search for and identification of cures for many life-threatening diseases that plague humans still remains an empirical and sometimes serendipitous process. While many advances have been made from basic scientific research to improvements in practical patient management, there still remains tremendous frustration in the rational and successful discovery of useful therapies particularly for life-threatening diseases such as cancer, inflammatory conditions, infectious diseases, conditions affecting the immune system, metabolic diseases and conditions, and other diseases and conditions.

Since the “War on Cancer” begun in the early 1970's by the United States National Cancer Institute (NCI) of the National Institutes of Health (NIH), a wide variety of strategies and programs have been created and implemented to prevent, diagnose, treat and cure cancer. One of the oldest and arguably most successful programs has been the synthesis and screening of small chemical entities (<1500 MW) for biological activity against cancer. This program was organized to improve and streamline the progression of events from chemical synthesis and biological screening to preclinical studies for the logical progression into human clinical trials with the hope of finding cures for the many types of life-threatening malignant tumors. The synthesis and screening of hundreds of thousands of chemical compounds from academic and industrial sources, in addition to the screening of natural products and extracts from prokaryotes, invertebrate animals, plant collections, and other sources from all over the world has been and continues to be a major approach for the identification of novel lead structures as potential new and useful medicines. This is in addition to other programs including biotherapeutics designed to stimulate the human immune system with vaccines, therapeutic antibodies, cytokines, lymphokines, inhibitors of tumor blood vessel development (angiogenesis) or gene and antisense therapies to alter the genetic make-up of cancer cells, as well as other clinical approaches.

The work supported by the NCI and other governmental agencies both domestic and foreign in academic or industrial research and development laboratories has resulted in an extraordinary body of biological, chemical and clinical information. In addition, large chemical libraries have been created, as well as highly characterized in vitro and in vivo biological screening systems that have been successfully used. However, from the tens of billions of dollars spent over the past thirty years supporting these programs both preclinically and clinically, only a small number of compounds have been identified or discovered that have resulted in the successful development of useful therapeutic products. Nevertheless, the biological systems both in vitro and in vivo and the “decision trees” used to warrant further animal studies leading to clinical studies have been validated. These programs, biological models, clinical trial protocols, and other studies remain critical for the discovery and development of any new therapeutic agent.

Unfortunately, many of the compounds that have successfully met the preclinical testing and federal regulatory requirements for clinical evaluation were either unsuccessful or disappointing in human clinical trials. Many compounds were found to have untoward or idiosyncratic side-effects that were discovered during human clinical Phase I dose-escalation studies used to determine the maximum tolerated dose (MTD) and side-effect profile. In some cases, these toxicities or the magnitude of their toxicity were not identified or predicted in preclinical toxicology studies. In other cases, chemical agents where in vitro and in vivo studies suggested a potentially unique activity against a particular tumor type, molecular target or biological pathway were not successful in human Phase II clinical trials where specific examination of particular cancer indications/types were evaluated in government sanctioned (e.g., U.S. FDA), IRB approved clinical trials. In addition, there are those cases where potential new agents were evaluated in randomized Phase III clinical trials where a significant clinical benefit could not be demonstrated have also been the cause of great frustration and disappointment. Finally, a number of compounds have reached commercialization but their ultimate clinical utility has been limited by poor efficacy as monotherapy (<25% response rates) and untoward dose-limiting side-effects (Grade III and IV) (e.g., myelosuppression, cardiotoxicity, gastrointestinal toxicities, or other significant toxicities).

In many cases, after the great time and expense of developing and moving an investigational compound into human clinical trials and where clinical failure has occurred, the tendency has been to return to the laboratory to create a better analog, look for agents with different structures but potentially related mechanisms of action, or undertake other research strategies. In some cases, efforts have been made to try additional Phase I or II clinical trials in an attempt to make some improvement with the side-effect profile or therapeutic effect in selected patients or cancer indications. In many of those cases, the results did not realize a significant enough improvement to warrant further clinical development toward product registration. Even for commercialized products, their ultimate use is still limited by suboptimal performance in many clinical contexts.

With so few therapeutics approved for cancer patients and the realization that cancer is a collection of diseases with a multitude of etiologies and that a patient's response and survival from therapeutic intervention is complex with many factors playing a role in the success or failure of treatment including disease indication, stage of invasion and metastatic spread, patient gender, age, health conditions, previous therapies or other illnesses, and genetic makeup of the patient, the opportunity for cures in the near term remains elusive. Moreover, the incidence of cancer continues to rise with an approximate 4% increase predicted for 2003 in the United States by the American Cancer Society such that over 1.3 million new cancer cases are estimated. In addition, with advances in diagnosis such as mammography for breast cancer and PSA tests for prostate cancer, more patients are being diagnosed at a younger age. For difficult to treat cancers, a patient's treatment options are often exhausted quickly resulting in a desperate need for additional treatment regimens. Even for the most limited of patient populations, any additional treatment opportunities would be of considerable value. This invention focuses on inventive compositions and methods for improving the therapeutic benefit of suboptimally administered chemical compounds including substituted naphthalimides such as amonafide.

Relevant literature includes Foye, W. O., “Cancer Chemotherapeutic Agents,” American Chemical Society, 1995, and Dorr, R. T., and Von Hoff, D. D., “Cancer Chemotherapy Handbook,” Appleton and Lange, 1994.

### SUMMARY OF THE INVENTION

This invention relates to novel compositions and methods to improve the utility of chemical agents with suboptimal performance in patients suffering with immunological disease, metabolic disease, infection, or hyperproliferative diseases including cancer. The invention describes novel improvements, pharmaceutical ingredients, dosage forms, excipients, solvents, diluents, drug delivery systems, preservatives, more accurate drug administration, improved dose determination and schedules, toxicity monitoring and ameliorization, techniques or agents to circumvent or reduce toxicity, techniques and tools to identify/predict those patients who might have a better outcome with a therapeutic agent by the use of phenotype or genotype determination through the use of diagnostic kits or pharmacokinetic or metabolism monitoring approaches. The invention also relates to the use of drug delivery systems, novel prodrugs, polymer conjugates, novel routes of administration, other agents to potentiate the activity of the compounds or inhibit the repair of suboptimal cellular effects or sublethal damage or to “push” the cell into more destructive cellular phases such as apoptosis. In some case, the use of these suboptimal therapeutics in conjunction with radiation or other conventional chemotherapeutic agents or biotherapeutic agents such as antibodies, vaccines, cytokines, lymphokines, gene and antisense therapies, or other biotherapeutic agents, would provide novel approaches and significant improvement.

In the inventive compositions and methods, the term suboptimal therapy includes agents where Phase I toxicity precluded further human clinical evaluation. It also includes those agents from Phase II trials where limited (<25% response rates) or no significant tumor responses were identified. Also, suboptimal therapy includes those agents, the subject of Phase III clinical trials the outcome of which was either medically or statistically not significant to warrant regulatory submission or approval by government agencies for commercialization or commercialized agents whose clinical performance (i.e. response rates) as a monotherapy are less than 25%, or whose side-effects are severe enough to limit wide utility. Agents with suboptimal clinical activity include but are not limited to the following: amonafide. More specifically, the inventive methods and compositions also focus on improvements for substituted naphthalimides including amonafide and derivatives or analogs thereof.

One aspect of the present invention is a method to improve the efficacy and/or reduce the side effects of suboptimally administered drug therapy comprising the steps of:

(1) identifying at least one factor or parameter associated with the efficacy and/or occurrence of side effects of the drug therapy; and

(2) modifying the factor or parameter to improve the efficacy and/or reduce the side effects of the drug therapy;

wherein the drug therapy comprises administration of amonafide or a derivative or analog thereof.

In one alternative, the drug therapy comprises administration of amonafide. In another alternative, the drug therapy comprises a derivative or analog of amonafide. The derivative or analog of amonafide can be selected from the group consisting of:

(1) a derivative of amonafide wherein the amino group attached to one of the six-membered aromatic rings has one or both of the hydrogens replaced with C₁-C₃ lower alkyl;

(2) a derivative of amonafide wherein the nitrogen connected to one of the six-membered rings through an ethylene linkage has one or both of the methyl groups bound thereto replaced with C₂-C₃ lower alkyl;

(3) a derivative of amonafide wherein the ethylene linkage is replaced with a propylene (C₃) or a butylene (C₄) linkage;

(4) a derivative of amonfide of Formula (II)

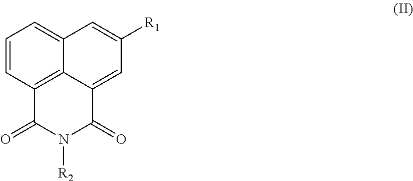

wherein: R₁ is selected from the group consisting of C₁-C₅ alkyl, amino, nitro, cyano, C₁-C₅ alkoxy, and hydrogen; and wherein R₂ is C₁-C₅ alkyl;

(5) a derivative of amonfide of Formula (III)

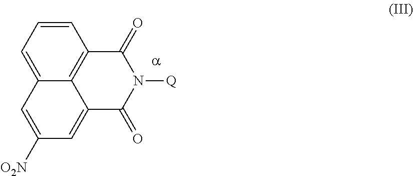

wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s)

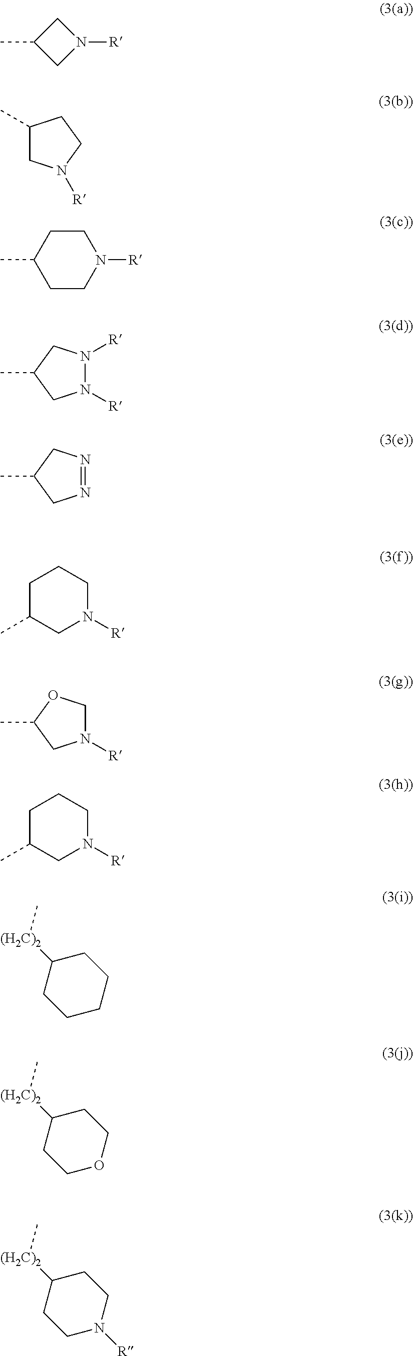

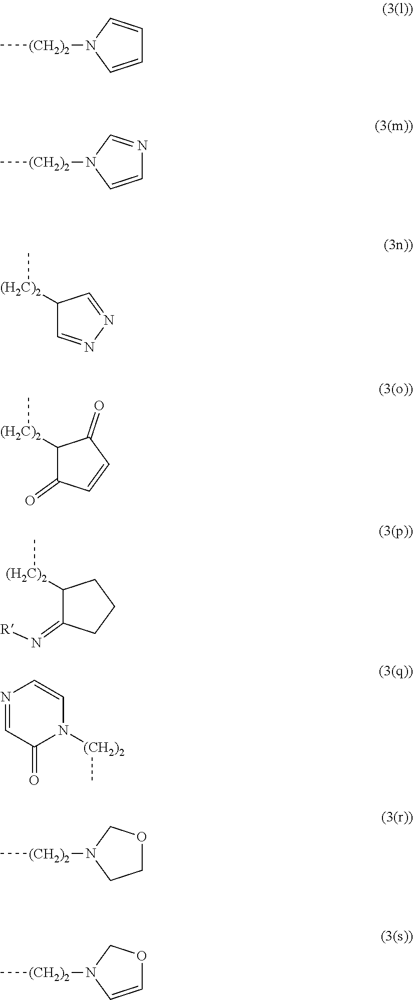

(6) a derivative of amonafide of Formula (III) wherein Q is selected from the group consisting of 1-R′-azetid-3-yl, 1-R′-pyrrolid-3-yl, 1-R′-piperid-4-yl, 1,2-diR′-1,2-diazolid-4-yl, 1,2-diazol-1-en-4-yl, 1-R′-piperid-4-yl, or 3-R′-oxazolid-5-yl, wherein R′ is selected from the group consisting of alkyl, alkenyl, acyl, alkoxy, aryl, amino, substituted amino, sulfo, sulfamoyl, carboxyl, carbamyl, and cyano;

(7) a derivative of amonafide of Formula (III) that is a naphthalimide wherein Q is —(CH₂)₂NR₂, where R is lower alkyl;

(8) a derivative of amonafide of Formula (III) that is a naphthalimide wherein Q is —(CH₂)₂NR₂, wherein NR₂ forms a heterocyclic group;

(9) a derivative of amonafide of Formula (III) that is a naphthalimide wherein Q is —(CH₂)₂NR₂ and wherein R₂ is —(CH₂)ₙ— or —(CH₂)ₘ—X—(CH₂)ₙ—, wherein m or n can be 0 to 5 and wherein X is NR″; wherein R″ is hydrogen, alkyl, alkenyl, acyl, alkoxy, aryl, amino, substituted amino, sulfo, sulfamoyl, carboxyl, carbamyl, cyano, or is not present; O; or S;

(10) a derivative of amonafide of Formula (III) wherein the tricyclic framework is derivatized so that it has one or more unsaturated bonds therein;

(11) a derivative of amonafide of Formula (III) wherein the tricyclic framework is derivativized so that it has at least one substituent selected from the group consisting of alkyl, aryl, and heteroaryl;

(12) a derivative of amonafide of Formula (III) wherein Q is selected from the group consisting of 1-pyrrolidyl, 3-R′-piperidyl, morpholino, 1-R′-piperazin-4-yl, 1-pyrrolyl, 1-imidazolyl, 1,3,5-triazol-1-yl, N-maleimido, 2-(R′-imino)pyrrolidyl, pyrazin-2-on-1-yl, 3-oxazolidyl, 3-oxazolyl, 2-pyrrolyl, 3-chloro-1-pyrrolidyl, 2-nitro-1-imidazolyl, 4-methoxy-1-imidazolyl, and 3-methyl-1-imidazolyl;

(13) a derivative of amonafide of Formula (III) wherein Q is selected from the group consisting of Subformulas 3(h), 3(i), 3(j), 3(k), 3(1), 3(m), 3(n), 3(o), 3(p), 3(q), 3(r), and 3(s), wherein R′ is selected from the group consisting of alkyl, alkenyl, acyl, alkoxy, aryl, amino, substituted amino, sulfo, sulfamoyl, carboxyl, carbamyl, and cyano;

(14) a derivative of amonafide of Formula (III) wherein the naphthalimide ring is modified to include one or more amino groups at positions other than position 3 of the naphthalimide ring;

(15) a derivative of amonafide of Formula (III) wherein the amino group at position 3 is replaced with an alternative substituent group selected from the group consisting of alkyl, aryl, nitro, amino, substituted amino, sulfamoyl, halo, carboxyl, carbamyl, and cyano;

(16) a derivative of amonafide of Formula (III) wherein an additional group is attached to the naphthalimide ring also comprising an amino group at position 3, the additional group being selected from the group consisting of alkyl, aryl, nitro, substituted amino, sulfamoyl, halo, carboxyl, carbamyl, and cyano;

(17) an analog of amonafide wherein the naphthalene ring is replaced with one bearing one or more nitrogen atoms in either or both rings;

(18) an analog of amonafide that is an isoquinoline analog of Formula (IV)

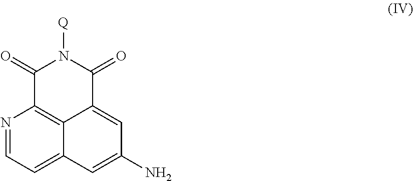

wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s);

(19) an analog of amonafide that is an isoquinoline analog of Formula (IV) wherein Q is —(CH₂)ₙ—N(CH₃)₂, wherein n is 1-12; and

(20) a derivative or analog of amonafide or of alternatives (1)-(19) including one or more optional substituents, provided that the optionally substituted amonafide derivative or analog possesses substantially equivalent pharmacological activity to amonafide as defined in terms of either or both topoisomerase II inhibition and DNA intercalation.

In general, therefore, derivatives or analogs of amonafide include compounds that can be described as derivatives of amonafide, derivatives of azonafide, derivatives of mitonafide, and derivatives of elinafide. Derivatives or analogs of amonafide also include heterocyclic-substituted bis-1,8-naphthalimide compounds, 1,8 naphthalimide imidazo {4,5,1-de}acridones, 2-substituted-1,2-dihydro-3H-dibenz[de,h]isoquinoline-1,3-diones, amino-substituted-[2′-(dimethylamino)ethyl]1,2-dihydro-3H-dibenz[de,h]isoquinoline-1,3-diones, tetrahydroazonafides, phenanthrene analogs of azonafide, and azaphenanthrenes.

The factor or parameter can be selected from the group consisting of:
(1) dose modification;
(2) route of administration;
(3) schedule of administration;
(4) indications for use;
(5) selection of disease stage;
(6) other indications;
(7) patient selection;
(8) patient/disease phenotype;
(9) patient/disease genotype;
(10) pre/post-treatment preparation
(11) toxicity management;
(12) pharmacokinetic/pharmacodynamic monitoring;
(13) drug combinations;
(14) chemosensitization;
(15) chemopotentiation;
(16) post-treatment patient management;
(17) alternative medicine/therapeutic support;
(18) bulk drug product improvements;
(19) diluent systems;
(20) solvent systems;
(21) excipients;
(22) dosage forms;
(23) dosage kits and packaging;
(24) drug delivery systems;
(25) drug conjugate forms;
(26) compound analogs;
(27) prodrugs;
(28) multiple drug systems;
(29) biotherapeutic enhancement;
(30) biotherapeutic resistance modulation;
(31) radiation therapy enhancement;
(32) novel mechanisms of action;
(33) selective target cell population therapeutics; and
(34) use with an agent to enhance its activity.

Another aspect of the invention is a composition to improve the efficacy and/or reduce the side effects of suboptimally administered drug therapy comprising an alternative selected from the group consisting of:

(1) a therapeutically effective quantity of a modified therapeutic agent or a derivative, analog, or prodrug of a therapeutic agent or modified therapeutic agent, wherein the modified therapeutic agent or the derivative, analog or prodrug of the therapeutic agent or modified therapeutic agent possesses increased therapeutic efficacy or reduced side effects as compared with an unmodified therapeutic agent;

(2) a composition comprising:
(a) a therapeutically effective quantity of a therapeutic agent, a modified therapeutic agent or a derivative, analog, or prodrug of a therapeutic agent or modified therapeutic agent; and
(b) at least one additional therapeutic agent, therapeutic agent subject to chemosensitization, therapeutic agent subject to chemopotentiation, diluent, excipient, solvent system, or drug delivery system, wherein the composition possesses increased therapeutic efficacy or reduced side effects as compared with an unmodified therapeutic agent;

(3) a therapeutically effective quantity of a therapeutic agent, a modified therapeutic agent, or a derivative, analog, or prodrug of a therapeutic agent or modified therapeutic agent that is incorporated into a dosage form, wherein the therapeutic agent, the modified therapeutic agent, or the derivative, analog, or prodrug of a therapeutic agent or modified therapeutic agent incorporated into the dosage form possesses increased therapeutic efficacy or reduced side effects as compared with an unmodified therapeutic agent;

(4) a therapeutically effective quantity of a therapeutic agent, a modified therapeutic agent, or a derivative, analog, or prodrug of a therapeutic agent or modified therapeutic agent that is incorporated into a dosage kit and packaging, wherein the therapeutic agent, the modified therapeutic agent, or the derivative, analog, or prodrug of a therapeutic agent or modified therapeutic agent incorporated into the dosage kit and packaging possesses increased therapeutic efficacy or reduced side effects as compared with an unmodified therapeutic agent; and

(5) a therapeutically effective quantity of a therapeutic agent, a modified therapeutic agent, or a derivative, analog, or prodrug of a therapeutic agent or modified therapeutic agent that is subjected to a bulk drug product improvement, wherein the therapeutic agent, the modified therapeutic agent, or the derivative, analog, or prodrug of a therapeutic agent or modified therapeutic agent subject to the bulk drug product improvement possesses increased therapeutic efficacy or reduced side effects as compared with an unmodified therapeutic agent; wherein the unmodified therapeutic agent is amonafide or a derivative or analog of amonafide, the modified therapeutic agent is a modification of amonafide or a derivative or analog of amonafide, and the derivative, analog, or prodrug is a derivative, analog, or prodrug of amonafide or of a derivative or analog of amonafide.

### BRIEF DESCRIPTION OF THE DRAWINGS

These and other features, aspects, and advantages of the present invention will become better understood with reference to the following description, appended claims, and accompanying drawings where:

FIG. 1 is a table detailing experiments on tumor inhibition by amonafide and other antineoplastic agents.

FIG. 2 is a table detailing further experiments on tumor inhibition by amonafide and other neoplastic agents.

### DETAILED DESCRIPTION OF THE INVENTION

This invention relates to novel compositions and methods to improve the utility of chemical agents including substituted naphthalimides such as amonafide with suboptimal performance for patients with cancer and with other diseases and conditions, including metabolic diseases, immunological diseases, and infectious diseases. The invention describes the novel development of improved pharmaceutical ingredients, dosage forms, excipients, solvents, diluents, drug delivery systems, preservatives, more accurate drug administrations, improved dose determination and schedules, toxicity monitoring and ameliorization, techniques or agents to circumvent or reduce toxicity, techniques and tools to identify/predict those patients who might have a better outcome with a therapeutic agent by the use of phenotype or genotype determination through the use of diagnostic kits or pharmacokinetic or metabolism monitoring approaches, the use of drug delivery systems, novel prodrugs, polymer conjugates, novel routes of administration, other agents to potentiate the activity of the compounds or inhibit the repair of suboptimal cellular effects or sub-lethal damage or to “push” the cell into more destructive cellular phases such as apoptosis. In some cases, the inventive examples include the use of these sub-optimal therapeutics in conjunction with radiation or other conventional chemotherapeutic agents or biotherapeutic agents such as antibodies, vaccines, cytokines, lymphokines, gene and antisense therapies, or other biotherapeutic agents.

By definition, the term “suboptimal therapy” includes agents where Phase I toxicity precluded further human clinical evaluation. It also includes those agents from Phase II trials where limited or no significant tumor responses were identified. In addition, it also includes those agents, the subject of Phase III clinical trials, whose outcome was either medically or statistically not significant to warrant submission or approval by regulatory agencies for commercialization or commercialized agents whose response rates as a monotherapy are less than 25% or whose side-effects are severe enough to limit wider utility. Agents with suboptimal activity include but are not limited to the following: amonafide. More specifically, the inventive methods and compositions also focus on improvements for substituted naphthalimides including amonafide; other substituted naphthalimides and analogs and derivatives thereof are described below.

Amonafide is 5-amino-2-[2-(dimethylamino)ethyl]-1H-benzo[de]isoquinoline-1,3(2H)-dione and has the structure shown below as Formula (I):

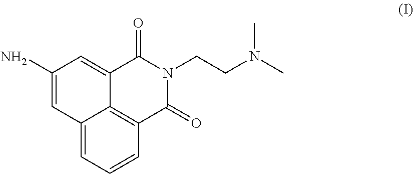 [SMILES: CN(C)CCN1C(=O)c2cccc3cc(N)cc(c23)C1=O]

The activity of amonafide has been described in U.S. Pat. No. 6,630,173 to Brown, U.S. Pat. No. 7,135,481 to Brown, United States Patent Application Publication No. 2004/0047918 by Brown, United States Patent Application Publication No. 2004/0082788 by Brown, United States Patent Application Publication No. 2005/0113579 by Brown, United States Patent Application Publication No. 2005/0142214 by Brown, United States Patent Application Publication No. 2005/0288310 by Brown, United States Patent Application Publication No. 2006/211648 by Brown, United States Patent Application Publication No. 2011/0003742 by Brown, and United States Patent Application Publication No. 2011/0262283 by Brown, all of which are incorporated herein by this reference. Additionally, the activity of other naphthalimides that can be considered analogs of amonafide against melanoma, colon cancer, and lymphocytic leukemia is described in U.S. Pat. No. 5,183,821 to Brana et al., incorporated herein by this reference. Also, the activity of other naphthalimides that can be considered analogs of amonafide, specifically azonafide and its derivatives, against cancers of the skin, lung, throat, stomach, salivary glands, colon, breast, prostate, pancreas, ovaries, uterus, endometrium, and against leukemia, melanoma, renal cell carcinoma, and multiple myeloma is described in U.S. Pat. No. 8,008,316 to Tarasova et al. Other types of cancer can also be treated. U.S. Pat. No. 7,541,463 to Qian et al., incorporated herein by this reference, describes the activity of sulfur-containing naphthalimide derivatives that can be considered analogs of amonafide against cancers including lung cancer, stomach cancer, liver cancer, leucocythemia, endometrial cancer, oophoroma, mammary cancer, colon cancer, prostatic cancer, and pituitary gland cancer. U.S. Pat. No. 7,741,337 to Van Quaquebeke et al., incorporated by this reference, describes the activity of a number of azonafide derivatives that can be considered analogs of amonafide against cancers including leukemia, non-small-cell lung cancer, small-cell lung cancer, CNS cancer, melanoma, ovarian cancer, renal cancer, prostate cancer, breast cancer, glioma, colon cancer, bladder cancer, sarcoma, pancreatic cancer, colorectal cancer, head and neck cancer, liver cancer, and lymphoma. United States Patent Application Publication No. 2011/0003742 by Brown, incorporated by this reference, describes the activity of amonafide against psoriasis, skin cancer, HPV-induced papilloma, HSV-induced shingles, colon cancer, bladder cancer, breast cancer, melanoma, ovarian cancer, prostatic carcinoma, and lung cancer.

In particular, regarding the activity of amonafide and its analogs, amonafide and its analogs have been shown to promote topoisomerase II-mediated DNA cleavage (Y.-W. Hsiang et al., “Topoisomerase II-Mediated DNA Cleavage by Amonafide and Its Structural Analogs,” Mol. Pharmacol. 36: 371-376 (1989)), incorporated herein by this reference.

In summary, amonafide and derivatives or analogs of amonafide can be expected to have antineoplastic activity against the following types of cancers: (1) melanoma; (2) colon cancer; (3) lymphocytic leukemia, including chronic lymphocytic leukemia; (4) skin cancer; (5) lung cancer, including small-cell lung cancer and non-small-cell lung cancer; (6) throat cancer; (7) stomach cancer; (8) salivary gland cancer; (9) breast cancer, including breast cancer characterized by the overexpression of Her2-neu and breast cancer characterized by resistance to topoisomerase II inhibitors; (10) prostate cancer; (11) pancreatic cancer; (12) ovarian cancer; (13) uterine cancer; (14) endometrial cancer; (15) other leukemias; (16) renal cell carcinoma; (17) multiple myeloma; (18) liver cancer; (19) pituitary gland cancer; (20) acute myeloid leukemia; (21) oophoroma; (22) glioma; (23) head and neck cancer; (23) colorectal cancer; (24) bladder cancer; (25) HPV-induced papilloma; (26) lymphoma, including both non-Hodgkin's lymphoma and Hodgkin's lymphoma; (27) myelodysplastic syndrome; (28) chronic myelocytic leukemia, including treatment of chronic myelocytic leukemia subsequent to the administration of homoharringtonine; (29) malignancies with overexpressed or mutated EGFR; (30) malignancies with overexpressed or mutated Her2/neu; (31) malignancies with overexpressed or mutated Braf; (32) malignancies with overexpressed or mutated BTK; (33) malignancies with overexpressed or mutated KRAS; (34) malignancies with overexpressed or mutated c-Myc; and (35) malignancies with overexpressed or mutated p53. Other indications for the use of amonafide and derivatives or analogs of amonafide are described below.

In particular, amonafide and derivatives or analogs of amonafide can be expected to have antineoplastic activity against the following types of cancers and related malignant conditions in which particular phenotypes or patterns of drug resistance exist: (1) triple-negative breast cancer; (2) acute leukemia, including, but not limited to, acute myeloid leukemia, acute erythroid leukemia, and acute lymphoblastic leukemia; (3) myelodysplastic syndrome; (4) chronic myelocytic leukemia, subsequent to or in combination with the administration of tyrosine kinase inhibitors or homoharringtonine; (5) chronic lymphocytic leukemia; (6) Hodgkin's lymphoma; (7) non-Hodgkin's lymphoma; (8) mycosis fungoides; (9) prostate cancer, particularly androgen-resistant prostate cancer; (10) lung small cell carcinoma, subsequent to or in combination with EGFR inhibitors such as erlotinib (Tarceva) or gefitinib (Iressa), wherein the lung small cell carcinoma is characterized by either wild-type or mutated EGFR; (11) lung non-small cell carcinoma, subsequent to or in combination with EGFR inhibitors such as erlotinib or gefitinib, wherein the lung non-small cell carcinoma is characterized by either wild-type or mutated EGFR; (12) breast cancer characterized by overexpressed Her-2-neu or overexpressed topoisomerase II (herceptin resistant); (13) glioblastoma that is resistant to one or both of the following therapeutic agents: temozolomide (Temodar) or bevacizumab (Avastin), or is characterized by EGFR variant III, either alone or in combination with other therapeutic agents; (14) malignancies characterized by overexpressed topoisomerase II; (16) malignancies characterized by overexpressed and/or mutated EGFR; and (17) prostate cancer.

Additionally, amonafide and derivatives and analogs thereof are expected to have activity against a number of non-malignant proliferative diseases, including psoriasis and HSV-induced shingles.

U.S. Pat. No. 8,014,957 to Redich et al., incorporated herein by this reference, describes amonafide as an inhibitor of Type II topoisomerase and a DNA intercalator.

Naphthalimides and derivatives thereof, including amonafide, are useful for treating cellular proliferative diseases such as tumors, e.g., a solid tumor. Solid tumors that are particularly amenable to treatment by administration of naphthalimides or derivatives thereof, including amonafide, include carcinomas and sarcomas. Carcinomas include malignant neoplasms derived from epithelial cells that tend to infiltrate or invade surrounding tissues and thus give rise to metastases. Adenocarcinomas are carcinomas derived from glandular tissue or in which the tumor cells form recognizable glandular structures. Sarcomas broadly include tumors whose cells are embedded in a fibrillar or homogeneous substance such as embryonic connective tissue. Proliferative diseases that can be treated by naphthalimides and derivatives thereof, including amonafide, include, but are not limited to, psoriasis, skin cancer, viral-induced hyperproliferative HPV-associated papilloma, HSV-associated shingles, colon cancer, bladder cancer, melanoma, ovarian carcinoma, prostatic carcinoma, and lung cancer.

Derivatives of amonafide include, but are not limited to, derivatives of amonafide in which: (i) the amino group attached to one of the six-membered aromatic rings has one or both of the hydrogens replaced with C₁-C₃ lower alkyl; (ii) the nitrogen connected to one of the six-membered rings through an ethylene linkage has one or both of the methyl groups bound thereto replaced with C₂-C₃ lower alkyl; or (iii) the ethylene linkage is replaced with a propylene (C₃) or a butylene (C₄) linkage.

Other substituted naphthalimide derivatives are described in U.S. Pat. No. 7,135,481 to Brown, incorporated herein by this reference, and are considered derivatives of amonfide as defined herein. These derivatives include derivatives of Formula (II), below:

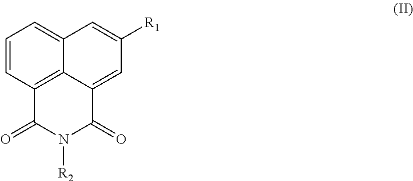

wherein: R₁ is selected from the group consisting of C₁-C₅ alkyl, amino, nitro, cyano, C₁-C₅ alkoxy, and hydrogen; and wherein R₂ is C₁-C₅ alkyl.

Still other naphthalimide derivatives defined as amonafide derivatives within the scope of the present invention are disclosed in United States Patent Application Publication No. 2004/0082788 by Brown, incorporated herein by this reference. These derivatives include the derivatives of Formula (III), below:

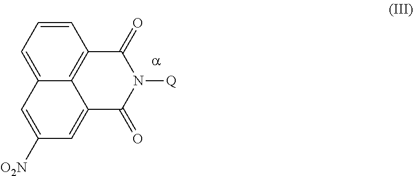

wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s), below:

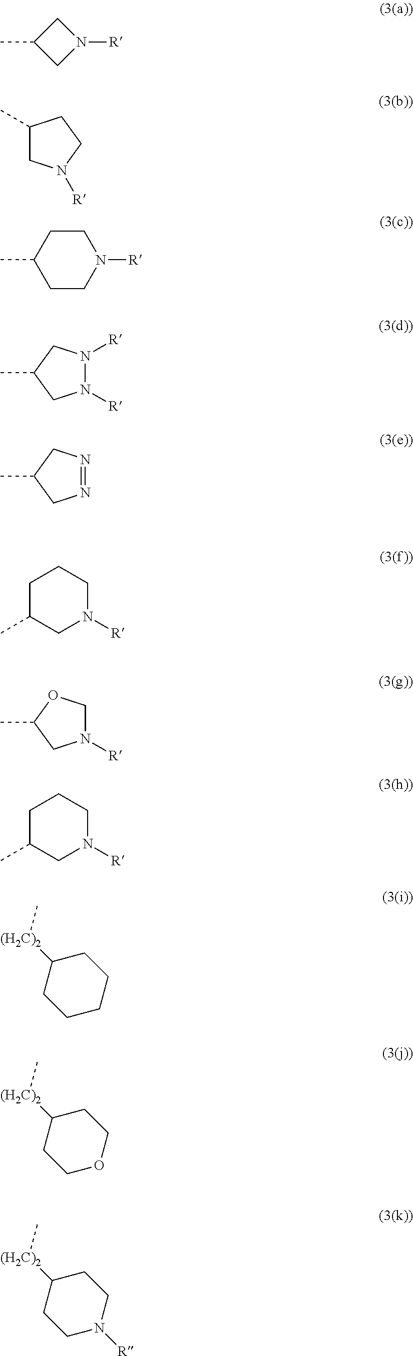

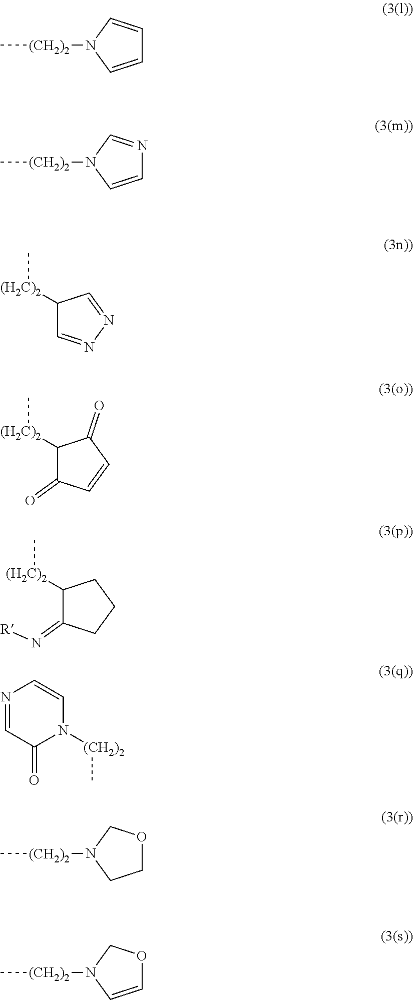

For example, in this class of analogs and derivatives, Q can be, but is not limited to, 1-R′-azetid-3-yl, 1-R′-pyrrolid-3-yl, 1-R′-piperid-4-yl, 1,2-diR′-1,2-diazolid-4-yl, 1,2-diazol-1-en-4-yl, 1-R′-piperid-4-yl, or 3-R′-oxazolid-5-yl, wherein R′ is selected from the group consisting of alkyl, alkenyl, acyl, alkoxy, aryl, amino, substituted amino, sulfo, sulfamoyl, carboxyl, carbamoyl, and cyano.

Additionally, in this class of analogs and derivatives, the structure of Formula (III) can represent a naphthalimide wherein Q is —(CH₂)₂NR₂, where R is lower alkyl such as methyl, ethyl, propyl, or butyl; alternatively, NR₂ in this representation can form a heterocyclic group.

Additionally, in this class of naphthalimide analogs and derivatives, R₂ can be —(CH₂)ₙ— or —(CH₂)ₘ—X—(CH₂)ₙ—, wherein m or n can be 0 to 5, and X can be NR″; wherein R″ can be hydrogen, alkyl, alkenyl, acyl, alkoxy, aryl, amino, substituted amino, sulfo, sulfamoyl, carboxyl, carbamoyl, cyano, or is not present; O; or S. Furthermore, the tricyclic framework of Formula (III) can be derivativized so that it has one or more unsaturated bonds therein. Additionally, the tricyclic framework of Formula (III) can be derivativized so that it has at least one substituent selected from the group consisting of alkyl, aryl, and heteroaryl.

In this class of analogs and derivatives, Q can alternatively be 1-pyrrolidyl, 3-R′-piperidyl, morpholino, 1-R′-piperazin-4-yl, 1-pyrrolyl, 1-imidazolyl, 1,3,5-triazol-1-yl, N-maleimido, 2-(R′-imino)pyrrolidyl, pyrazin-2-on-1-yl, 3-oxazolidyl, 3-oxazolyl, and groups or moieties known in the art, including, but not limited to, 2-pyrrolyl, 3-chloro-1-pyrrolidyl, 2-nitro-1-imidazolyl, 4-methoxy-1-imidazolyl, and 3-methyl-1-imidazolyl. In the structures of Subformulas (3(h)) through (3(s)), R′ can be alkyl, alkenyl, acyl, alkoxy, aryl, amino, substituted amino, sulfo, sulfamoyl, carboxyl, carbamoyl, cyano, or other functional groups known to those skilled in the art.

Additional compounds in this class of analogs and derivatives are naphthalimides having an amino group attached to other positions in the naphthalimide ring. For example, in one embodiment, the naphthalimide ring is modified to include one or more amino groups at positions other than position 3 of the naphthalimide ring. In yet another embodiment, the naphthalimide ring is modified to include one or more amino groups at other positions in addition to the amino group at position 3 of the naphthalimide ring. In yet another embodiment, the amino group at position 3 is replaced with an alternative substituent group. Examples of such alternative substituents include alkyl, aryl, nitro, amino, substituted amino, sulfamoyl, halo, carboxyl, carbamoyl, cyano, and other functional groups known to those skilled in the art. In yet another embodiment, an additional group is attached to the naphthalimide ring also comprising an amino group at position 3. Examples of such additional groups include alkyl, aryl, nitro, substituted amino, sulfamoyl, halo, carboxyl, carbamoyl, cyano, and other functional groups known to those skilled in the art.

In yet another alternative, the naphthalene ring can be replaced with one bearing one or more nitrogen atoms in either or both rings. An example would be isoquinoline analogs such as the isoquinoline analog of Formula (IV), below:

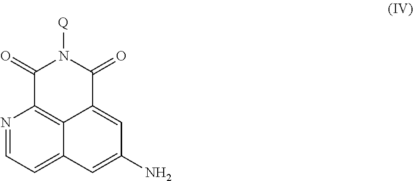

wherein Q is as previously defined. A preferred isoquinoline analog of amonafide is where Q is —(CH₂)ₙ—N(CH₃)₂, wherein n is 1-12 or more; in a more preferred embodiment, n is 1 to 6.

Additional derivatives and analogs of amonafide include azonafide and derivatives thereof of Formulas (V) and (VI), below,

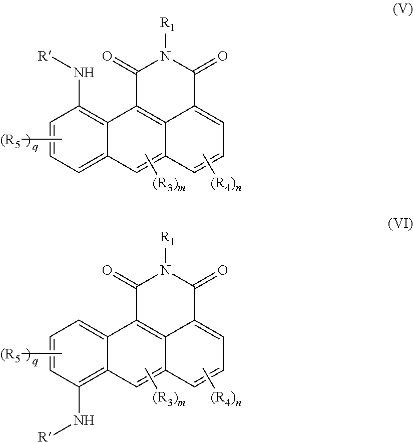

wherein: R₁ is monoalkylaminoalkyl or dialkylaminoalkyl; each of the substituents R₃, R₄, and R₅ is independently selected from the group consisting of hydrogen, halogen, C₁₋₇ alkyl, C₁₋₇ alkoxy, C₁₋₇ alkylthio, nitro, cyano, protected amino and halo-C₁₋₇ alkyl; m is the number of substituents R₃ and ranges from 0 to 1; n is the number of substituents R₄ and ranges from 0 to 3; q is the number of substituents R₅ and ranges from 0 to 3; R′ is a radical selected from the group consisting of C₁₋₁₂ alkyl, C₂₋₁₂ alkenyl, C₂₋₁₂ alkynyl, arylalkyl, Het¹alkyl, Het²alkyl, C₂₋₇ alkylsulfonyl, alkenylsulfonyl, alkynylsulfonyl, arylsulfonyl, aryloxyalkylsulfonyl, cycloalkylsulfonyl, arylalkylsulfonyl, Het¹sulfonyl, Het¹alkylsulfonyl, C₂₋₁₁ alkylcarbonyl, alkenylcarbonyl, alkynylcarbonyl, arylcarbonyl, aminocarbonyl, alkyloxycarbonyl, alkenyloxycarbonyl, aryloxycarbonyl, aryloxyalkylcarbonyl, cycloalkylcarbonyl, arylalkylcarbonyl, Het¹carbonyl, Het¹alkylcarbonyl, Het¹oxycarbonyl, Het¹alkyloxycarbonyl, alkylthiocarbonyl, alkenylthiocarbonyl, alkynylthiocarbonyl, arylthiocarbonyl, arylalkylthiocarbonyl, al kyloxythiocarbonyl, aryloxythiocarbonyl, alkyloxyalkylthiocarbonyl, aryloxyalkylthiocarbonyl, Het¹alkylthiocarbonyl, Het¹ oxythiocarbonyl, Het¹ alkyloxythiocarbonyl, alkylaminocarbonyl, alkenylaminocarbonyl, alkynylaminocarbonyl, arylaminocarbonyl, alkyloxyalkylaminocarbonyl, aryloxyalkylaminocarbonyl, cycloalkylaminocarbonyl, arylalkylaminocarbonyl, Het¹aminocarbonyl, Het¹alkylaminocarbonyl, Het¹ oxyalkylaminocarbonyl, Het¹alkyloxyaminocarbonyl, alkylaminothiocarbonyl, alkenylthioaminocarbonyl, alkynylaminothiocarbonyl, arylaminothiocarbonyl, arylalkylthioaminocarbonyl, alkyloxyalkylaminothiocarbonyl, aryloxyalkylaminothiocarbonyl, Het¹alkylaminothiocarbonyl, Het¹aminothiocarbonyl, and Het¹alkyloxyalkylthioaminocarbonyl, wherein one or more carbon atoms of the radical is (are) optionally substituted by one or more substituents independently selected from the group consisting of oxo, alkyl, cycloalkyl, alkyloxycarbonyl, carboxyl, aminocarbonyl, mono- or di-alkylaminocarbonyl, aminosulfonyl, alkyl-S(═O), hydroxy, cyano, halogen, haloalkyl, alkoxy, haloalkoxy, nitro, amino, monoalkylamino, and dialkylamino; and Het¹ and Het² are as shown in Formulas (VII) and (VIII), below:

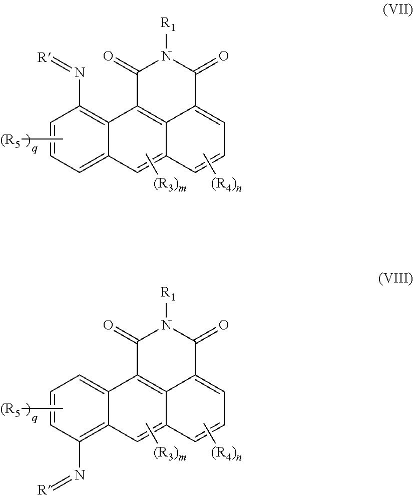

wherein: m, n, q, R₁, R₃, R₄, and R₅ are as defined with respect to Formulas (V) and (VI), and R′ is a radical selected from the group consisting of alkylidene, alkenylidene, alkynylidene, cycloalkenylidene, arylalkenylidene, arylalkynylidene, cycloalkylidene, arylalkylidene, Het¹alkylidene and Het²alkylidene, wherein one or more carbon atoms of the radical are optionally substituted by one or more substituents independently selected from the group consisting of alkyl, hydroxy, cyano, halogen, amino, and dialkylamino; as well as salts or solvates thereof. These analogs are described in U.S. Pat. No. 7,741,337 to Van Quaquebeke et al., incorporated herein by this reference.

Still additional analogs are the sulfur-containing analogs disclosed in U.S. Pat. No. 7,541,463 to Qian et al., incorporated herein by this reference. These analogs are of Formula (IX), below

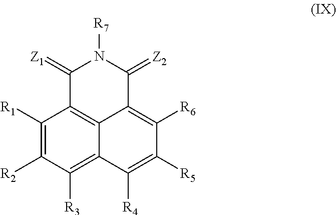

wherein: R₁, R₂, and R₆ are each independently selected from the group consisting of hydrogen, C₁₋₆ alkyl, C₁₋₆ haloalkyl, C₁₋₃ alkoxy, halogen, hydroxy, amino, and cyano; R₃, R₄, and R₅ are each independently selected from the group consisting of hydrogen, C₁₋₆ alkyl, C₁₋₆ haloalkyl, C₁₋₃ alkoxy, halogen, hydroxy, amino, and cyano, or, alternatively, R₃ and R₄ or R₄ and R₅ together form a 5-6 membered heterocyclic ring or an aryl-fused 5-6 membered heterocyclic ring; the heterocyclic ring has 1-3 heteroatoms each selected from S, N, and O and is optionally substituted with 1-3 substituents each selected from the group consisting of aryl, heteroaryl, C₁₋₆ alkyl, C₁₋₆ haloalkyl, C₁₋₆ alkoxy, C₁₋₆ haloalkoxy, halogen, amino, C₁₋₃ amino substituted with alkyl, nitro, hydroxy, cyano, an acyl group of 1-3 carbon atoms, and sulfonic acid; the aryl or heteroaryl group is optionally substituted with 1-3 substituents each selected from the group consisting of C₁₋₆ alkyl, C₁₋₆ haloalkyl, C₁₋₆ alkoxy, C₁₋₆ haloalkoxy, halogen, amino, nitro, hydroxy, cyano, an acyl group containing 1-3 carbon atoms, and sulfonic acid; Z₁ and Z₂ are O or S, with the proviso that at least one of Z₁ and Z₂ is S when R₃, R₄, and R₅ do not contain S; R₇ is C₁₋₆ alkyl, C₂₋₈ alkenyl, C₁₋₆ alkylamino, (C₁₋₆ alkyl)-N—(C₁₋₆ alkyl)₂, C₁₋₆ alkylpiperazinyl, arylacyloxy, or heterocyclic acyloxy; wherein the heterocyclic ring has 1-3 heteroatoms selected from S, N, and O, and is optionally substituted with 1-3 substituents selected from the group consisting of C₁₋₆ alkyl, C₁₋₆ haloalkyl, C₁₋₆ alkoxy, C₁₋₆ haloalkoxy, halogen, amino, nitro, hydroxy, and cyano; the heterocyclic ring is optionally substituted with 1-3 substituents selected from the group consisting of C₁₋₆ alkyl, C₁₋₆ haloalkyl, C₁₋₆ alkoxy, C₁₋₆ haloalkoxy, halogen, amino, nitro, hydroxy, cyano, and sulfonic acid; with the proviso that R₇ is not CH₂CH₂N(CH₃)₂ when R₄ and R₅ form a thienyl group. These compounds include N—(N′,N′)dimethylaminoethyl)benzo[k,l]thioxanthene-3,4-dicarboximide, N-(2′-piperazinylethyl)benzo[k,l]thioxanthene-3,4-dicarboximide, include N—(N′,N′)dimethylaminoethyl)-4H,6H-9-m-nitrophenyl-benzo[de]thiazo[5,4-g]isoquinoline-4,6-diketone, N—(N′,N′)dimethylaminoethyl)-4H,6H-9-phenyl-benzo[de]thiazo[5,4-g]isoquinoline-4,6-diketone, N—(N′,N′)dimethylaminoethyl)-4H,6H-9-p-methylphenyl-benzo[de]thiazo[5,4-g]isoquinoline-4,6-diketone, N—(N′,N′)dimethylaminoethyl)-4H,6H-9-p-methoxyphenyl-benzo[de]thiazo[5,4-g]isoquinoline-4,6-diketone, N—(N′,N′)dimethylaminoethyl)-4H,6H-9-o-chlorophenyl-benzo[de]thiazo[5,4-g]isoquinoline-4,6-diketone, N—(N′,N′)dimethylaminoethyl)-4H,6H-9-o-hydroxyphenyl-benzo[de]thiazo[5,4-g]isoquinoline-4,6-diketone, 5-(N′,N′-diethylaminoethyl)-4H,6H-benzo[de]-1,2,3-thiadiazol[5,4-g]isoquinoline-4,6-diketone, 5-butyl-4H,6H-benzo[de]-1,2,3-thiadiazol[5,4-g]isoquinoline-4,6-diketone, 5-(2′-piperazinylethyl)-4H,6H-benzo[de]-1,2,3-thiadiazol[5,4-g]isoquinoline-4,6-diketone, N—(N′,N′)-diethylaminoethyl)benzo[b]thieno[2,1-c]naphthalimide, N—(N′,N′)-diethylaminopropyl)benzo[b]thieno[2,1-c]naphthalimide, N-(2′-piperazinylethyl)benzo[b]thieno[2,1-c]naphthalimide, and N-butylbenzo[b]thieno[2,1-c]naphthalimide.

Additional compounds include salts of amonafide disclosed in U.S. Pat. No. 6,989,390 to Ajami et al., U.S. Pat. No. 6,693,198 to Ajami et al., and U.S. Pat. No. 5,420,137 to Brana et al., all of which are incorporated herein by this reference, including amonafide hydrochloride, amonafide methanesulfonate, amonafide malate, amonafide glycolate, amonafide succinate, amonafide maleate, amonafide fumarate, amonafide citrate, amonafide L-tartrate, amonafide L-aspartate, amonafide pyruvate, and amonafide 2-oxoglutarate.

Still other analogs of amonafide are the azonafide derivatives described in U.S. Pat. No. 8,008,316 to Tarasova et al., incorporated herein by this reference. These derivatives are substituted with peptides that may act as cell receptor-targeting ligands. An example of these derivatives is 2-{2-[(2-aminoethyl)methylamino]ethyl}-6-methoxy-1,2-dihydro-3H-dibenzo[de,h]isoquinoline-1,3-dione, which can be covalently conjugated to a peptide through reaction of the free amino group to the C-terminal carboxyl group of the peptide. Another example of these derivatives is 4-(2,5-dioxo-2,5-dihydropyrrol-1-yl)-N-{[2-(6-methoxy-1,3-dioxo-1H,3H-dibenzo[de,h]isoquinolin-2-yl-ethyl]-methylamino}-ethyl)-butyramide. The maleimido group can be used for reaction with a peptide. Yet another example of these derivatives is 7-(2-{[2-(6-methoxy-1,3-dioxo-1H,3H-dibenzo[de,h]isoquinolin-2-yl)-ethyl]-methylamino}-ethylcarbamoyl)-heptanoic acid 2,5-dioxo-pyrrolidin-1-yl ester. This derivative includes an activated N-hydroxysuccinimide ester that can be used for reaction with a peptide.

Still other analogs of amonafide are the heterocyclic-substituted bis-1,8-naphthalimide compounds described in U.S. Pat. No. 7,947,839 to Gazzard et al., incorporated herein by this reference. These compounds can be conjugated to therapeutically active agents, such as the monoclonal antibody trastuzumab. These compounds include N,N′-(bis-aminoethyl-1,3-propanediamine)-bis-(4-N-imidazolyl)-1,8 naphthalimide; N,N′-(bis-aminoethyl-1,3-propanediamine)-4-(N-imidazolyl)-4-hydroxyl-1,8 naphthalimide; N¹,N² bis methyl, N,N′-(bis-aminoethyl-1,3-propanediamine)-4-bromo, 4-N-imidazolyl 1,8 naphthalimide; N¹,N² bis methyl, N,N′-(bis-aminoethyl-1,3-propanediamine)-4-N-imidazolyl, 4-piperazinyl 1,8 naphthalimide; N¹—H, N²-methyl, N,N′-(bis-aminoethyl-1,3-propanediamine)-bis-(4-N-imidazolyl)-1,8 naphthalimide; N¹—H, N²-(methoxyethoxyethoxyacetamide)-(N,N′-(bis-aminoethyl-1,3-propanediamine)-bis 4-N-imidazolyl-1,8 naphthalimide); N-(tert-butylglutaramide), bis-aminoethyl-1,3-propanediamineybis-4-N-imidazolyl-1,8 naphthalimide; N,N′—(N-cyclopropylmethyl, bis-aminoethyl-1,3-propanediamine)-bis-4-N-imidazolyl-1,8 naphthalimide; N′-methyl, N²—(N-methylglycyl)-N,N′-(bis-aminoethyl-1,3-propanediamine)-bis 4-N-imidazolyl-1,8 naphthalimide; N¹,N² bis methyl, N,N′-(bis-aminoethyl-1,3-propanediamine)-4-N-imidazolyl, 4-(4-mercaptopropylpiperazinyl)-1,8 naphthalimide; N′-methyl,N²-(tert-butylglutaramide)-N,N′-(bis-aminoethyl-1,3-propanediamine)-bis-4-N-imidazolyl-1,8-naphthalimide; N′-methyl, N²-(2-(2-(2-aminoethoxy)ethoxy)acetamido)-N,N′-(bis-aminoethyl-1,3-propanediamine)-bis 4-N-imidazolyl-1,8 naphthalimide; N′-methyl, N²—(N-methylvaline)-N,N′-(bis-aminoethyl-1,3-propanediamine)-bis 4-N-imidazolyl-1,8 naphthalimide; N′-methyl, N²—(N methyl, N-tertbuytyloxyvaline)-N,N′-(bis-aminoethyl-1,3-propanediamine)-bis 4-N-imidazolyl-1,8 naphthalimide; N′-methyl, N²-tertbuytyloxycarbonyl)-N,N′-(bis-aminoethyl-1,3-propanediamine)-bis 4-N-imidazolyl-1,8 naphthalimide; N′-methyl, N²-glutaramide)-N,N′-(bis-aminoethyl-1,3-propanediamine)-bis 4-N-imidazolyl-1,8 naphthalimide; N,N′-(bis-aminoethyl-1,3-propanediamine)-4-dimethylamino, 4-N-imidazolyl-1,8 naphthalimide; N′-t-butyloxycarbonyl, N²-(2-(2-(2-(N-Fmoc)aminoethoxy)ethoxy)acetamido)-N,N′-(bis-aminoethyl-1,3-propanediamine)-bis 4-N-imidazolyl-1,8 naphthalimide; N′-t-butyloxycarbonyl, N²-(2-(2-(2-aminoethoxy)ethoxy)acetamido)-N,N′-(bis-aminoethyl-1,3-propanediamine)-bis-4-N-imidazolyl-1,8 naphthalimide; N¹,N² bis methyl, N,N′-(bis-aminoethyl-1,3-propanediamine)-4-N-imidazolyl, 4-(3-aminopropyl)amino)-1,8 naphthalimide; N¹,N² bis methyl, N,N′-(bis-aminoethyl-1,3-propanediamine)-4-N-imidazolyl, 4-(6-aminohexyl)amino)-1,8 naphthalimide; N¹,N² bis methyl, N,N′-(bis-aminoethyl-1,3-propanediamine)-4-N-imidazolyl, 4-N-(2-(N-9-fluorenylmethoxycarbonyl)aminoethoxy-tetraethoxy)-1,8 naphthalimide; N¹,N² bis methyl, N,N′-(bis-aminoethyl-1,3-propanediamine)-4-N-imidazolyl, 4-N-(3-t-butylpropionatetetraethoxy)-1,8 naphthalimide; N¹,N² bis-methyl, N,N′-(bis-aminoethyl-1,3-propanediamine)-4-thiol, 4-N-imidazolyl-1,8 naphthalimide; N¹,N² bis methyl, N,N′-(bis-aminoethyl-1,3-propanediamine)-4-dithio-(2-pyridyl), 4-N-imidazolyl-1,8 naphthalimide; N¹,N² bis methyl, N,N′-(bis-aminoethyl-1,3-propanediamine)-4-dithio-(3-propionic acid), 4-N-imidazolyl-1,8 naphthalimide; N-t-butyloxycarbonyl, N²-(2-(2-(2-aminoethoxy)triethoxy)propionamido)-N,N′-(bis-aminoethyl-1,3-propanediamine)-bis 4-N-imidazolyl-1,8 naphthalimide; N¹—H, N²-glycyl, N,N′-(bis-aminoethyl-1,3-propanediamine)-bis 4-N-imidazolyl-1,8 naphthalimide; N¹—H, N²—(N-methyl)glycyl, N,N′-(bis-aminoethyl-1,3-propanediamine)-bis 4-N-imidazolyl-1,8naphthalimide; N¹—H, N²—(N-methyl)alanyl, N,N′-(bis-aminoethyl-1,3-propanediamine)-bis 4-N-imidazolyl-1,8 naphthalimide; N′, N² bis glycyl, N,N′-(bis-aminoethyl-1,3-propanediamine)-bis 4-N-imidazolyl-1,8 naphthalimide; N′,N² bis(N-methyl glycyl), N,N′-(bis-aminoethyl-1,3-propanediamine)-bis 4-N-imidazolyl-1,8 naphthalimide; and N¹,N² bis(N-methyl alanyl), N,N′-(bis-aminoethyl-1,3-propanediamine)-bis 4-N-imidazolyl-1,8 naphthalimide. U.S. Pat. No. 7,947,839 to Gazzard et al. also discloses the analog of amonafide elinafide, which is unsubstituted 1,8-bis naphthalimide.

Still other analogs of amonafide are those described in U.S. Pat. No. 6,664,263 to Cholody et al., incorporated herein by this reference. These include 1,8 naphthalimide imidazo{4,5,1-de}acridones, such as 2-{3-{methyl[3-(6-oxo-6H-imidazo[4,5,1-de]acridin-5-yl)aminopropyl]amino}propyl}-5-nitro-1H-benz[de]isoquinoline-1,3(2H)-dione; 2-{3-{methyl[3-(8-fluoro-6-oxo-6H-imidazo[4,5,1-de]acridin-5-yl)aminopropyl]-amino}propyl}-5-nitro-1Hbenz[de]isoquinoline-1,3(2H)-dione; 2-{3-{methyl[3-(8-hydroxy-6-oxo-6H-imidazo[4,5,1-de]acridin-5-yl)aminopropyl]-amino}propyl}-5-nitro-1Hbenz[de]isoquinoline-1,3(2H)-dione; 2-{3-{methyl[3-(8-trifluoromethyl-6-oxo-6H-imidazo[4,5,1-de]acridin-5-yl)aminopropyl]amino}propyl}-5-nitro-1H-benz[de]isoquinoline-1,3(2H)-dione; 2-{3-{methyl[3-(6-oxo-6H-imidazo[4,5,1-de]acridin-5-yl)aminopropyl]amino}propyl}-5-amino-1H-benz[de]isoquinoline-1,3(2H)-dione; 2-{3-{methyl[3-(8-fluoro-6-oxo-6H-imidazo[4,5,1-de]acridin-5-yl)aminopropyl]-amino}propyl}-5-amino-1H-benz[de]isoquinoline-1,3(2H)-dione; 2-{3-{methyl[3-(8-hydroxy-6-oxo-6H-imidazo[4,5,1-de]acridin-5-yl)aminopropyl]-amino}propyl}-5-amino1H-benz[de]isoquinoline-1,3(2H)-dione; 2-{3-{4-[3-(6-oxo-6H-imidazo[4,5,1-de]acridin-5-yl)aminopropyl]piperazin-1-yl}propyl}-5-nitro-1H-benz[de]isoquinoline-1,3(2H)-dione; 2-{3-{4-[3-(6-oxo-6H-imidazo[4,5,1-de]acridin-5-yl)aminopropyl]piperazin-1-yl}propyl}-5-nitro-1H-benz[de]isoquinoline-1,3(2H)-dione; 2-{3-{4-[3-(8-fluoro-6-oxo-6H-imidazo[4,5,1-de]acridin-5-yl)aminopropyl]piperazin-1-yl}propyl}-5-nitro-1H-benz[de]isoquinoline-1,3(2H)-dione; 2-{3-{4-[3-(8-hydroxy-6-oxo-6H-imidazo[4,5,1-de]acridin-5-yl)aminopropyl]-piperazin-1-yl}propyl}-5-nitro-1H-benz[de]isoquinoline-1,3(2H)-dione; 2-{3-{4-[3-(8-trifluoromethyl-6-oxo-6H-imidazo[4,5,1-de]acridin-5-yl)aminopropyl]piperazin-1-yl}propyl}-5-nitro-1H-benz[de]isoquinoline-1,3(2H)-dione; 2-{3-{4-[3-(6-oxo-6H-imidazo[4,5,1-de]acridin-5-yl)aminopropyl]piperazin-1-yl}propyl}-5-amino-1H-benz[de]isoquinoline-1,3(2H)-dione; 2-{3-{4-[3-(8-fluoro-6-oxo-6H-imidazo[4,5,1-de]acridin-5-yl)aminopropyl]piperazin-1-yl}propyl}-5-amino-1H-benz[de]isoquinoline-1,3(2H)-dione; 2-{3-{4-[3-(8-hydroxy-6-oxo-6H-imidazo[4,5,1-de]acridin-5-yl)aminopropyl]piperazin-1-yl}propyl}-5-amino-1H-benz[de]isoquinoline-1,3(2H)-dione; and 2-{3-{4-[3-(8-trifluoromethyl-6-oxo-6H-imidazo[4,5,1-de]acridin-5-yl)aminopropyl]piperazin-1-yl}propyl}-5-amino-1H-benz[de]isoquinoline-1,3(2H)-dione.

Still other derivatives of amonafide are described in U.S. Pat. No. 5,854,006 to Hanigan et al., including γ-glutamylamonafide, shown as Formula (X):

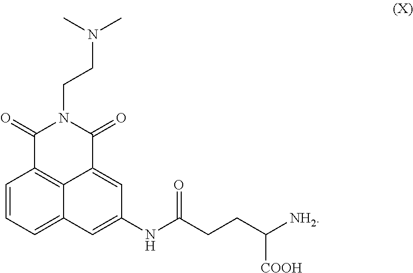 [SMILES: CN(C)CCN1C(=O)c2cccc3cc(NC(=O)CCC(N)C(=O)O)cc(c23)C1=O]

Still other amonafide derivatives and analogs include elinafide and additional derivatives and analogs described in M. F. Braña et al., “New Analogues of Amonafide and Elinafide, Containing Aromatic Heterocycles: Synthesis, Antitumor Activity, Molecular Modeling, and DNA Binding Properties,” J. Med. Chem. 47: 1391-1399 (2004), incorporated herein by this reference. Elinafide is shown as Formula (XI):

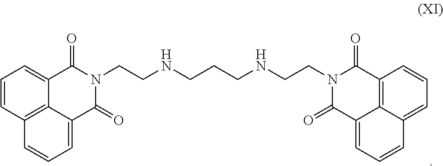 [SMILES: O=C1c2cccc3cccc(c23)C(=O)N1CCNCCCNCCN1C(=O)c2cccc3cccc(c23)C1=O]

Additional amonafide analogs are those of Formula (XII), below

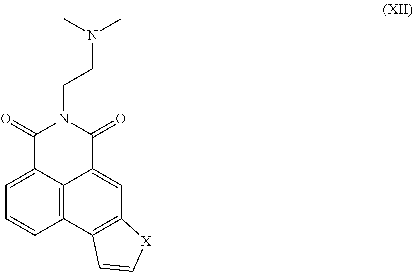

wherein X is selected from the group consisting of O and S, and those of Formula (XIII), below

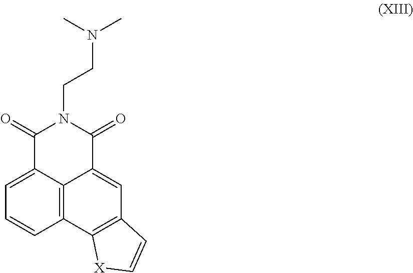

wherein X is selected from the group consisting of O and S.

Still more additional amonafide analogs described in M. F. Braña et al. (2004), supra, are compounds based on elinafide such as those of Formula (XIV), below

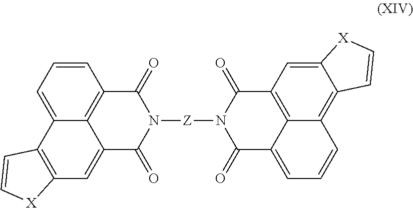

wherein the compound is selected from compounds having the following alternatives for X and Z: (i) X is O and Z is (CH₂)₂NH(CH₂)₃NH(CH₂)₂; (ii) X is O and Z is (CH₂)₂NCH₃(CH₂)₃NCH₃(CH₂)₂; (iii) X is S and Z is (CH₂)₂NH(CH₂)₃NH(CH₂)₂; and (iv) X is S and Z is CH₂)₂NCH₃(CH₂)₃NCH₃(CH₂)₂.

Still more additional amonafide analogs described in M. F. Braña et al. (2004), supra, are compounds based on elinafide such as those of Formula (XV), below

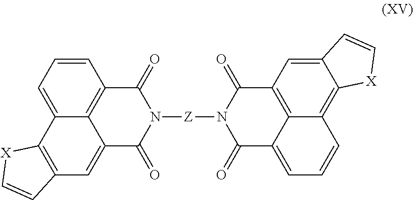

wherein the compound is selected from compounds having the following alternatives for X and Z: (i) X is O and Z is (CH₂)₂NH(CH₂)₃NH(CH₂)₂; (ii) X is O and Z is (CH₂)₂NCH₃(CH₂)₃NCH₃(CH₂)₂; (iii) X is S and Z is (CH₂)₂NH(CH₂)₃NH(CH₂)₂; and (iv) X is S and Z is CH₂)₂NCH₃(CH₂)₃NCH₃(CH₂)₂.

Still more additional analogs of amonafide are those described in Hsiang et al. (1989), supra, and shown below as Formulas (XVI) and (XVII)

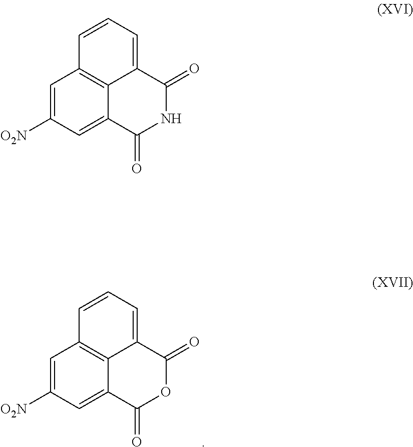 [SMILES: O=C1NC(=O)c2cc([N+](=O)[O-])cc3cccc1c23 ; O=C1OC(=O)c2cc([N+](=O)[O-])cc3cccc1c23]

Additional amonafide derivatives or analogs are described in S. M. Sami et al., “2-[2′-(Dimethylaminoethyl]-1,2-dihydro-3H-dibenz[de,h]isoquinoline-1,3-diones with Substituents at Positions 4, 8, 9, 10, and 11. Synthesis, Antitumor Activity, and Quantitative Structure-Activity Relationships,” J. Med. Chem. 39: 4978-4987 (1996), incorporated herein by this reference. These compounds include 10-chloro-2-[2′-(diethylamino)ethyl]-1,2-dihydro-3H-dibenz[de,h]isoquinoline-1,3-dione, 2-[2′-(diethylamino)ethyl]-1,2-dihydro-10-iodo-3H-dibenz[de,h]isoquinoline-1,3-dione, 2-[2′-(diethylamino)ethyl]-1,2-dihydro-10-fluoro-3H-dibenz[de,h]isoquinoline-1,3-dione, 2-[2′-(diethylamino)ethyl]-1,2-dihydro-4-methyl-3H-dibenz[de,h]isoquinoline-1,3-dione, 2-[2′-(diethylamino)ethyl]-1,2-dihydro-10-methyl-3H-dibenz[de,h]isoquinoline-1,3-dione, 2-[2′-(diethylamino)ethyl]-1,2-dihydro-4-hydroxy-3H-dibenz[de,h]isoquinoline 1,3-dione, 2-[2′-(diethylamino)ethyl]-1,2-dihydro-4-methoxy-3H-dibenz[de,h]isoquinoline 1,3-dione, and 2-[2′-(diethylamino)ethyl]-1,2-dihydro-4[[2′-(dimethylamino)ethyl]amino]-3H-dibenz[de,h]isoquinoline 1,3-dione.

Further amonafide derivatives or analogs are described in S. M. Sami et al., “Analogues of Amonafide with Novel Ring Systems,” J. Med. Chem. 43: 3067-3073 (2000), incorporated herein by this reference. These compounds include tetrahydroazonafides, which have the naphthalene chromophore of amonafide within the anthracene nucleus of azonafide; phenanthrene analogs, in which the linear anthracene nucleus is replaced by the bent phenanthrene nucleus; and azaphenanthrenes. In particular, these compounds include: 4-acetylamino-2-[2′-(dimethylamino)ethyl]1,2,8,9,10,11-hexahydro-3H-dibenz[de,h]isoquinoline-1,3-dione, 2-[2′-(dimethylamino)ethyl]1,2,8,9,10,11-hexahydro-4-(trimethylacetyl)amino-3H-dibenz[de,h]isoquinoline-1,3-dione, 2-[2′-(dimethylamino)ethyl]1,2,8,9,10,11-hexahydro-5-(trimethylacetyl)amino-3H-dibenz[de,h]isoquinoline-1,3-dione, 4-amino-2-[2′-(dimethylamino)ethyl]1,2,8,9,10,11-hexahydro-3H-dibenz[de,h]isoquinoline-1,3-dione, 5-amino-2-[2′-(dimethylamino)ethyl]1,2,8,9,10,11-hexahydro-3H-dibenz[de,h]isoquinoline-1,3-dione, 2-[2′-(dimethylamino)ethyl]1,2,8,9,10,11-hexahydro-3H-dibenz[de,h]isoquinoline-1,3-dione, 5-[2′-(dimethylamino)ethyl]-5,6-dihydro-4H-dibenz[de,h]isoquinoline-4,6-dione, 5-[2′-(dimethylamino)ethyl]-5,6-dihydro-8-nitro-4H-dibenz[de,h]isoquinoline-4,6-dione, 8-amino-5-[2′-(dimethylamino)ethyl]-5,6-dihydro-4H-dibenz[de,h]isoquinoline-4,6-dione, 5-[2′-(dimethylamino)ethyl]-5,6-dihydro-11-nitro-4H-dibenz[de,h]isoquinoline-4,6-dione, 11-amino-5-[2′-(dimethylamino)ethyl]-5,6-dihydro-4H-dibenz[de,g]isoquinoline-4,6-dione, 8-chloro-5-[2′-(dimethylamino)ethyl]-5,6-dihydro-4H-dibenz[de,g]isoquinoline-4,6-dione, 5-[2′-(dimethylamino)ethyl]-5,6-dihydro-8-hydroxy-4H-dibenz[de,h]isoquinoline-4,6-dione, 5-[2′-(dimethylamino)ethyl]-5,6-dihydro-8-methoxy-4H-dibenz[de,h]isoquinoline-4,6-dione, 11-chloro-5-[2′-(dimethylamino)ethyl]-5,6-dihydro-4H-dibenz[de,h]isoquinoline-4,6-dione, 5-[2′-(dimethylamino)ethyl]-5,6-dihydro-11-hydroxy-4H-dibenz[de,h]isoquinoline-4,6-dione, 5-[2′-(dimethylamino)ethyl]-5,6-dihydro-4H-quinolino[6,7,8-de]isoquinoline-4,6-dione, 5-[2′-(dimethylamino)ethyl]-5,6-dihydro-4H-quinolino[7,6,5-de]isoquinoline-4,6-dione, 3-acetylamino-5-[2′-(dimethylamino)ethyl]-5,6-dihydro-4H-quinolino[6,7,8-de]isoquinoline-4,6-dione, and 3-amino-5-[2′-(dimethylamino)ethyl]-5,6-dihydro-4H-quinolino[6,7,8-de]isoquinoline-4,6-dione.

Further amonafide derivatives and analogs are described in U.S. Pat. No. 5,635,506 to Alberts et al., incorporated herein by this reference.

In general, these amonafide derivatives or analogs have the structure of Formula (XVIII), below:

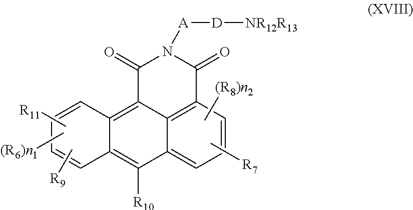

wherein: R₈, R₁₀, and R₆ are each independently selected from the group consisting of hydrogen, lower alkyl, aryl, lower alkanoyl, formyl, halogen, heterocyclic-lower alkyl, lower alkylsulfonyl, hydrazino, NR₂R₃, OR₁, amino-lower alkyleneoxy, mono-lower alkylamino-lower alkyleneoxy, di-lower alkylamino-lower alkyleneoxy, lower alkanoylamino, cyano, CO₂H, CONR₁R₂, SO₂NR₁R₂, SR₁, and a moiety of subformula (18(a));

—N═N—NR₁₄R₁₅ (18(a))

R₁ is selected from the group consisting of hydrogen, lower alkyl, aryl-lower alkyl, aryl, formyl, and lower alkanoyl; R₂ and R₃ are each independently selected from the group consisting of hydrogen, lower alkyl, aryl-lower alkyl, aryl, formyl, lower alkanoyl, mono-alkylamino-lower alkylene, di-alkylamino-lower alkylene, and hydroxyl-lower alkyl; R₉, R₁₁, and R₇ are each independently selected from the group consisting of hydrogen and lower alkyl; or R₉ and R₁₁, R₉ and R₁₀, or R₇ and R₁₀, together with the carbon atoms to which they are attached, form a benzene ring; A is (CR₄R₅)n₃, lower cycloalkyl, aryl, or a chemical bond; each R₄ and R₅ is independently hydrogen or lower alkyl, R₁₂ and R₁₃ are independently selected from the group consisting of hydrogen and lower alkyl, wherein the lower alkyl is unsubstituted or substituted with hydroxy, mercapto, lower alkoxy, lower alkylcarbonyloxy, carboxy, or carbo-lower alkoxy; or, in the alternative, R₁₂ and R₁₃ taken together with the nitrogen atom to which they are attached form a 3- to 6-membered heterocyclic ring; R₁₄ and R₁₅ are independently hydrogen or lower alkyl; D is a chemical bond, or, taken together with NR₁₂, forms a 5- or 6-membered heterocyclic ring; n₁ and n₂ are each independently 0, 1, or 2; and n₃ is 0, 1, 2, 3, 4, or 5.

Additional amonafide derivatives and analogs are described in S. M. Sami et al., “2-Substituted 1,2-Dihydro-3H-dibenz[de,h]isoquinoline-1,3-diones. A New Class of Antitumor Agent,” J. Med. Chem. 36: 765-770 (1993), incorporated herein by this reference. These compounds are of Formula (XIX)

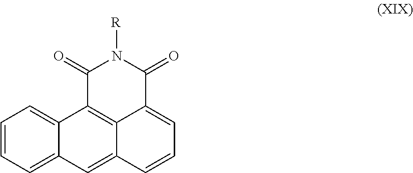

wherein R is selected from the group consisting of Subformulas (19(a)), (19(b)), (19(c)), (19(d)), (19(e)), (19(f)), (19(g)), (19(h)), (19(i)), (19(j)), (19(k)), (19(1)), (19(m)), (19(n)), (19(o)), (19(p)), (19(q)), (19(r)), and (19(s))

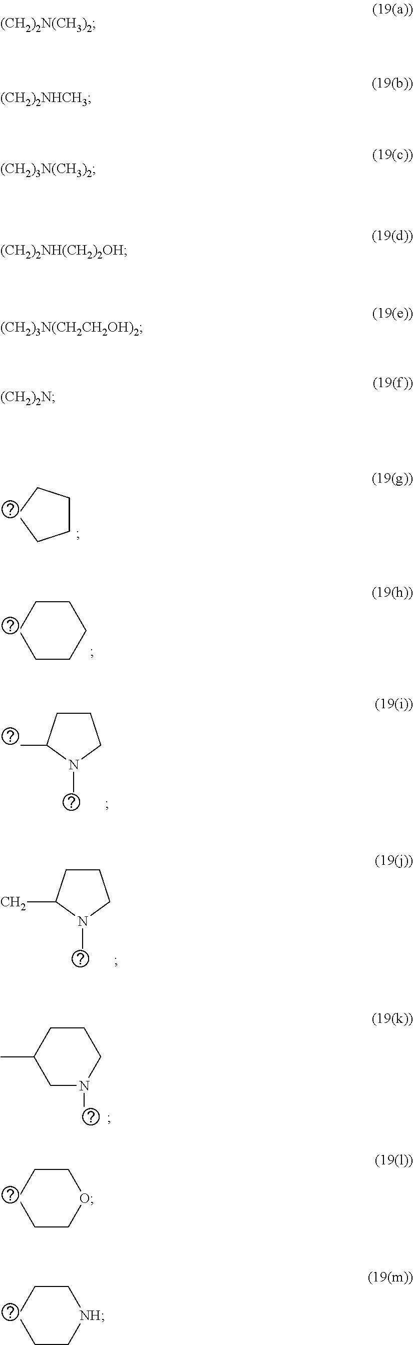

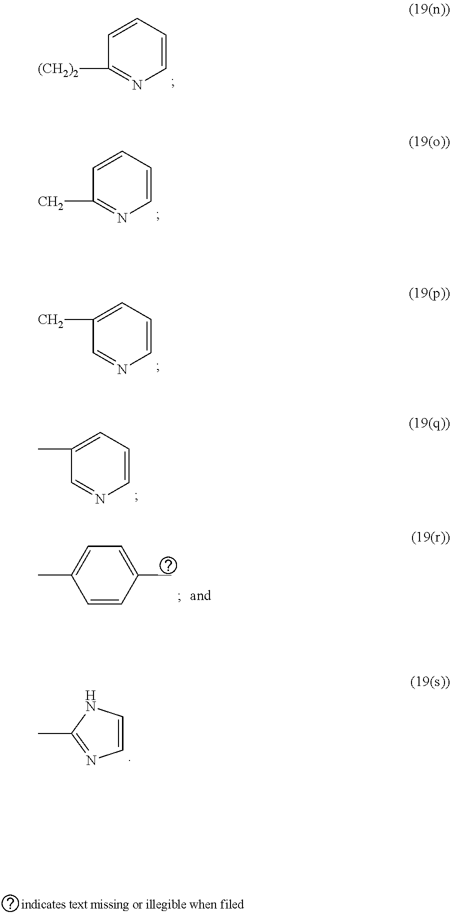

Additional derivatives and analogs of amonafide are described in S. M. Sami et al., “Amino-Substituted 2-[2′-(Dimethylamino)ethyl]1,2-dihydro-3H-dibenz[de,h]isoquinoline-1,3-diones. Synthesis, Antitumor Activity, and Quantitative Structure-Activity Relationship,” J. Med. Chem. 38: 983-993 (1995), incorporated herein by this reference. These compounds include: 2-[2′-(dimethylamino)ethyl]-1,2-dihydro-11-nitro-3H-dibenz[de,h]isoquinoline-1,3-dione; 11-amino-2-[2′-(dimethylamino)ethyl]-1,2-dihydro-3H-dibenz[de,h]isoquinoline-1,3-dione; 11-(acetylamino)-2-[2′-(dimethylamino)ethyl]-1,2-dihydro-3H-dibenz[de,h]isoquinoline-1,3-dione; 2-[2′-(dimethylamino)ethyl]-1,2-dihydro-8-nitro-3H-dibenz[de,h]isoquinoline-1,3-dione; 8-amino-2-[2′-(dimethylamino)ethyl]-1,2-dihydro-3H-dibenz[de,h]isoquinoline-1,3-dione; 4-(acetylamino)-2-[2′-(dimethylamino)ethyl]-1,2-dihydro-3H-dibenz[de,h]isoquinoline-1,3-dione; 4-amino-2-[2′-(dimethylamino)ethyl]-1,2-dihydro-3H-dibenz[de,h]isoquinoline-1,3-dione; 9-(acetylamino)-2-[2′-(dimethylamino)ethyl]-1,2-dihydro-3H-dibenz[de,h]isoquinoline-1,3-dione; 9-amino-2-[2′-(dimethylamino)ethyl]-1,2-dihydro-3H-dibenz[de,h]isoquinoline-1,3-dione; 10-amino-2-[2′-(dimethylamino)ethyl]-1,2-dihydro-3H-dibenz[de,h]isoquinoline-1,3-dione; 2-[2′-(dimethylamino)ethyl]-10-[(trimethylacetyl)amino]-1,2-dihydro-3H-dibenz[de,h]isoquinoline-1,3-dione; 8-(acetylamino)-2-[2′-(dimethylamino)ethyl]-1,2-dihydro-3H-dibenz[de,h]isoquinoline-1,3-dione; 6-(acetylamino)-2-[2′-(dimethylamino)ethyl]-1,2-dihydro-3H-dibenz[de,h]isoquinoline-1,3-dione; 7-(acetylamino)-2-[2′-(dimethylamino)ethyl]-1,2-dihydro-3H-dibenz[de,h]isoquinoline-1,3-dione; 7-amino-2-[2′-(dimethylamino)ethyl]-1,2-dihydro-3H-dibenz[de,h]isoquinoline-1,3-dione; 6-amino-2-[2′-(dimethylamino)ethyl]-1,2-dihydro-3H-dibenz[de,h]isoquinoline-1,3-dione; 2-[2′-(dimethylamino)ethyl]-4-[(trimethylacetyl)amino]-1,2-dihydro-3H-dibenz[de,h]isoquinoline-1,3-dione; 2-[2′-(dimethylamino)ethyl]-5-[(trimethylacetyl)amino]-1,2-dihydro-3H-dibenz[de,h]isoquinoline-1,3-dione; 5-amino-2-[2′-(dimethylamino)ethyl]-1,2-dihydro-3H-dibenz[de,h]isoquinoline-1,3-dione; and 5-(acetylamino)-2-[2′-(dimethylamino)ethyl]-1,2-dihydro-3H-dibenz[de,h]isoquinoline-1,3-dione.

Additional amonafide derivatives or analogs are described in U.S. Patent Application Publication Ser. No. 2010/0303719 by Huang et al., incorporated herein by this reference. These compounds include: (i) compounds of Formula (XX)

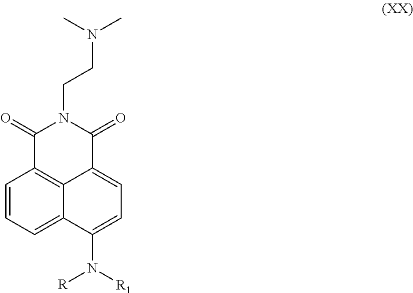

wherein: (a) R and R₁ are both H; (b) R is H and R₁ is propyl; (c) R is H and R₁ is allyl; (d) R is H and R₁ is (CH₂)₂OCH₃; (e) R is H and R₁ is hexyl; (f) R is H and R₁ is cyclohexyl; (g) R and R₁ form a pyridyl moiety with a 6-membered ring including one nitrogen atom in which the nitrogen atom is bonded to the remainder of the structure; and (h) R is ethyl and R₁ is ethyl; and (ii) the compound of Formula (XXI), below

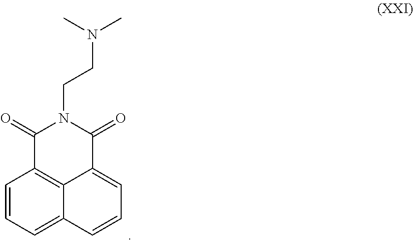 [SMILES: CN(C)CCN1C(=O)c2cccc3cccc(c23)C1=O]

Additional analogs of amonafide are disclosed in United States Patent Application Publication No. 2010/0303719 by Huang et al. and in United States Patent Application Publication No. 2013/0225634 by Huang et al., both incorporated herein by this reference. These compounds are stated not to be metabolized by NAT2 and thus to be more metabolically stable than amonafide. These include, but are not limited to, the specific compounds of Formula (XXII) to Formula (XXX), below:

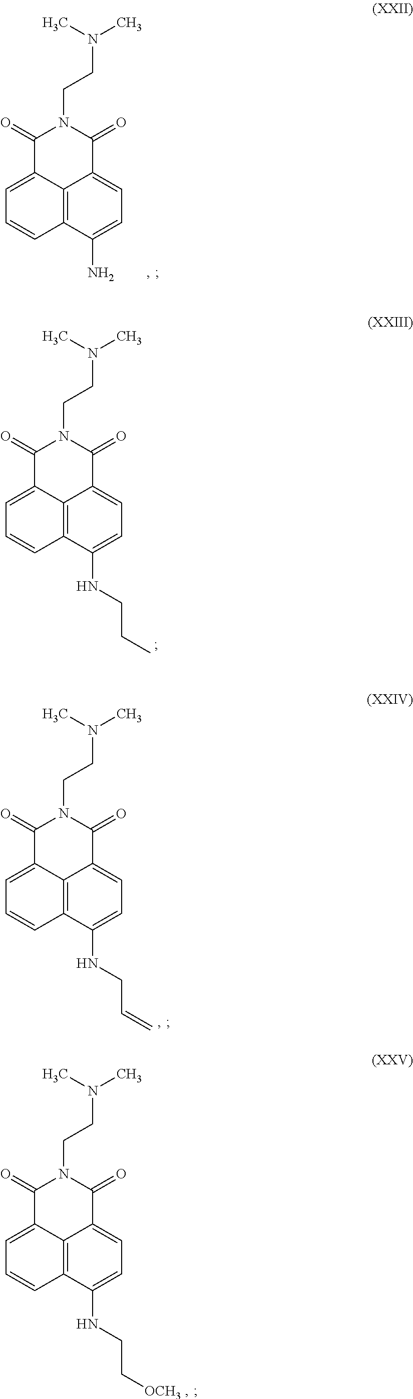 [SMILES: CN(C)CCN1C(=O)c2cccc3c(N)ccc(c23)C1=O ; CCCNc1ccc2c3c(cccc13)C(=O)N(CCN(C)C)C2=O ; C=CCNc1ccc2c3c(cccc13)C(=O)N(CCN(C)C)C2=O ; COCCNc1ccc2c3c(cccc13)C(=O)N(CCN(C)C)C2=O]

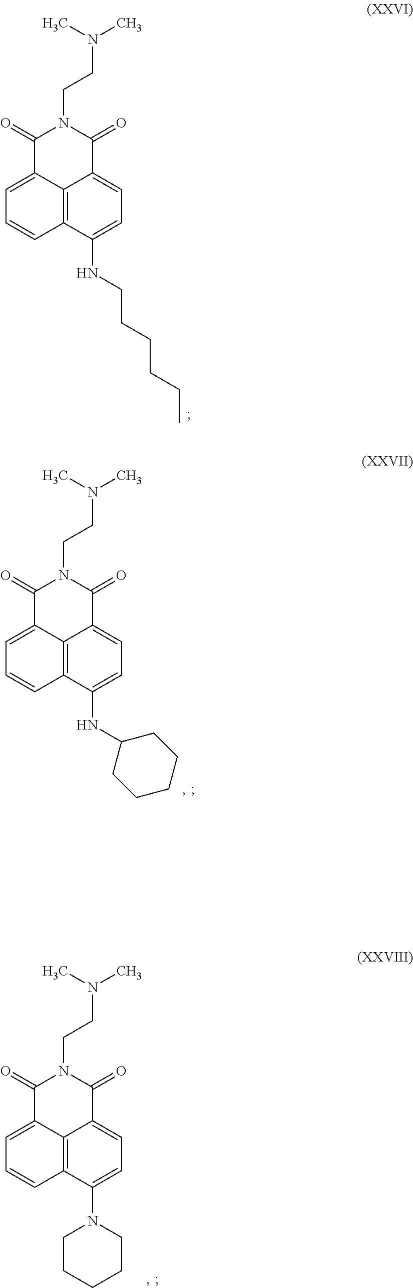 [SMILES: CCCCCCNc1ccc2c3c(cccc13)C(=O)N(CCN(C)C)C2=O ; CN(C)CCN1C(=O)c2cccc3c(NC4CCCCC4)ccc(c23)C1=O ; CN(C)CCN1C(=O)c2cccc3c(N4CCCCC4)ccc(c23)C1=O]

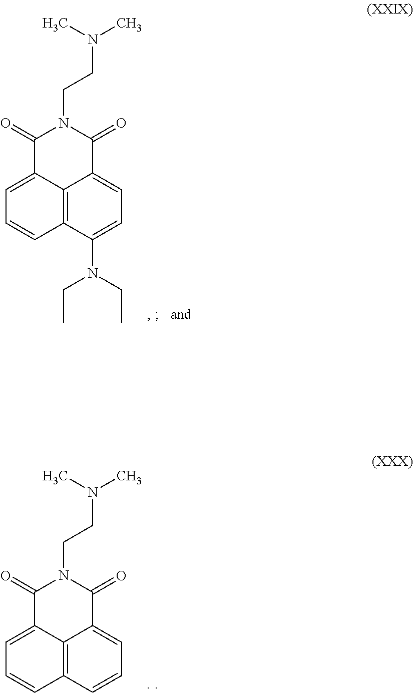 [SMILES: CCN(CC)c1ccc2c3c(cccc13)C(=O)N(CCN(C)C)C2=O ; CN(C)CCN1C(=O)c2cccc3cccc(c23)C1=O]

In general, therefore, derivatives or analogs of amonafide include compounds that can be described as derivatives of amonafide, derivatives of azonafide, derivatives of mitonafide, and derivatives of elinafide. Derivatives or analogs of amonafide also include heterocyclic-substituted bis-1,8-naphthalimide compounds, 1,8 naphthalimide imidazo{4,5,1-de}acridones, 2-substituted-1,2-dihydro-3H-dibenz[de,h]isoquinoline-1,3-diones, amino-substituted-[2′-(dimethylamino)ethyl]1,2-dihydro-3H-dibenz[de,h]isoquinoline-1,3-diones, tetrahydroazonafides, phenanthrene analogs of azonafide, and azaphenanthrenes.

As described above, and as detailed more generally below, derivatives and analogs of amonafide can be optionally substituted with one or more groups that do not substantially affect the pharmacological activity of the derivative or analog. These groups are generally known in the art. Definitions for a number of common groups that can be used as optional substituents are provided below; however, the omission of any group from these definitions cannot be taken to mean that such a group cannot be used as an optional substituent as long as the chemical and pharmacological requirements for an optional substituent are satisfied.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring. As used herein, the term “alkenyl” refers to an unbranched, branched or cyclic hydrocarbyl residue having one or more carbon-carbon double bonds. As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included. As used herein, the term “aryl” refers to a monocyclic or fused bicyclic moiety having the well-known characteristics of aromaticity; examples include phenyl and naphthyl, which can be optionally substituted. As used herein, the term “hydroxyaryl” refers to an aryl group including one or more hydroxyl groups as substituents; as further detailed below, further substituents can be optionally included. As used herein, the term “heteroaryl” refers to monocyclic or fused bicylic ring systems that have the characteristics of aromaticity and include one or more heteroatoms selected from O, S, and N. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valiences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents.

Substituent groups useful for substituting saturated carbon atoms in the specified group, moiety, or radical include, but are not limited to, —Z^{a}, ═O, —OZ^{b}, —SZ^{b}, ═S⁻, —NZ^{c}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z^{c}'s may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N— pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O⁻, —S(O₂)OZ^{b}, —OS(O₂)OZ^{b}, —OS(O₂)O⁻, —P(O)(O)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

Similarly, substituent groups useful for substituting nitrogen atoms in heteroalkyl and cycloheteroalkyl groups include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other stereoisomeric forms) as well as mixtures of stereoisomers in varying degrees of chiral purity or percentage of E and Z, including racemic mixtures, mixtures of diastereomers, and mixtures of E and Z isomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted olefin isomer. When the chemical name does not specify the isomeric form of the compound, it denotes any one of the possible isomeric forms or mixtures of those isomeric forms of the compound.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium. For example, ketone and enol are two tautomeric forms of one compound.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is a “hydrate.” Examples of hydrates include, but are not limited to, hemihydrate, monohydrate, dihydrate, trihydrate, hexahydrate, and other hydrated forms. It should be understood by one of ordinary skill in the art that the pharmaceutically acceptable salt and/or prodrug of the present compound may also exist in a solvate form. The solvate is typically formed via hydration which is either part of the preparation of the present compound or through natural absorption of moisture by the anhydrous compound of the present invention.

As used herein, the term “ester” means any ester of a present compound in which any of the —COON functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolyzable esters of the present compounds are the compounds whose carboxyls are present in the form of hydrolyzable ester groups. That is, these esters are pharmaceutically acceptable and can be hydrolyzed to the corresponding carboxyl acid in vivo.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. The general term “alkylene” encompasses more specific examples such as “ethylene,” wherein n is 2, “propylene,” wherein n is 3, and “butylene,” wherein n is 4. The hydrocarbyl groups of the alkylene can be optionally substituted as described above.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocyclyl can be fully saturated or partially saturated, but non-aromatic. For example, the general term “carbocyclyl” encompasses cycloalkyl. The carbocyclic and heterocyclic structures encompass compounds having monocyclic, bicyclic or multiple ring systems; and such systems may mix aromatic, heterocyclic, and carbocyclic rings. Mixed ring systems are described according to the ring that is attached to the rest of the compound being described.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk₁-N(Alk₂)(Alk3), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

Other combinations of substituents are known in the art and, are described, for example, in U.S. Pat. No. 8,344,162 to Jung et al., incorporated herein by this reference. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

Accordingly, methods and compositions according to the present invention encompass amonafide derivatives and analogs including one or more optional substituents as defined above, provided that the optionally substituted amonafide derivative or analog possesses substantially equivalent pharmacological activity to amonafide as defined in terms of either or both topoisomerase II inhibition and DNA intercalation. Methods for determination of topoisomerase II inhibition are known in the art and are described, for example, in A. Constantinou et al., “Novobiocin- and Phorbol-12-Myristate-13-Acetate-Induced Differentiation of Human Leukemia Cells Associates with a Reduction in Topoisomerase II Activity,” Cancer Res. 49: 1110-1117 (1989), incorporated herein by this reference. Methods for determination of DNA intercalation are known in the art and are described, for example, in H. Zipper et al., “Investigations on DNA Intercalation and Surface Binding by SYBR Green I, Its Structure Determination and Methodological Implications,” Nucl. Acids. Res. 32(12): e103 (2004), incorporated herein by this reference.

(I) Suboptimal Therapeutics

In general, examples of compounds with suboptimal therapeutic activity may include antimetabolites, DNA/nucleic acid binding/reactive agents, topoisomerase inhibitors, anti-tubulin agents, signal transduction inhibitors, protein synthesis inhibitors, inhibitors of DNA transcribing enzymes, DNA/RNA intercalating agents, DNA minor groove binders, drugs that block steroid hormone action, photochemically active agents, immune modifying agents, hypoxia selective cytotoxins, chemical radiation sensitizers and protectors, antisense nucleic acids, oligonucleotide and polynucleotide therapeutic agents, immune modifying agents, antitumor antibiotics, and other classes of therapeutic agents having antineoplastic, antiproliferative, or immune-system-modulating activity. Specific examples include: fluoropyrimidines, thiopurines, inhibitors of nucleoside diphosphate reductase, 2′-deoxyribonucleoside analogs, nucleosides, folic acid analogs, methotrexate, 6-diazo-5-oxo-norleucine, L-asparaginase, N-(phosphoacetyl)-L-aspartic acid, nitrogen mustard, mechlorethamine, chlorambucil, melphalan, cyclophosphamide, estramustine, platinum complexes, nitrosoureas, BCNU, CCNU, streptozotocin, alkyl sulfonates, busulfan, clomesone, triazenylimidazoles and related triazenes, mitozolomide, temozolomide, aziridines, tris(1-aziridinyl)phosphine sulfide, aziridinylphosphines, 3,6,-diaziridinyl-2,5-bis(carboethoxyamino)-1,4-benzoquinone (diaziquone) (AZQ), AZQ analogs, procarbazine, hexamethylamine, topoisomerase I inhibitors, camptothecin, camptothecin analogs, topoisomerase II inhibitors, anthracyclines, doxorubicin, epirubicin, etoposide, DNA intercalating agents, amsacrine, CI-921, 1′-carbamate analogs of amsacrine, 9-aminoacridine-4-carboxamides, acridine carboxamide, tricyclic carboxamides, 1-nitroacridine, acridine derivatives, diacridines, triacridines, podophyllotoxins, ellipticine, merbarone, benzisoquinolinediones, etoposide, teniposide, aminoanthraquinones, inhibitors of DNA-transcribing enzymes, transcription inhibitors, replication inhibitors, RNA replication inhibitors, polymerase inhibitors, rifamycins, actinomycins, DNA minor groove binding compounds, Hoechst 33258, mitomycins, CC-1065, mithramycins, chloromycins, olivomycins, phthalanilides, anthramycins, antimitotic agents, vinca alkaloids, vinblastine and analogs, vincristine and analogs, navelbine, colchicine and analogs, bleomycin and analogs, estramustine, aromatase inhibitors, tamoxifen, LHRH antagonists and analogs, porfimer, hematoporphyrins, electron-affinic oxygen mimetics, nitoaromatics, nitroheterocyclics, nitroimidizaoles, tirapazamine, mitomycins, menadione and analogs, napthoquinones, aziridoquinones, amine oxides, N-oxides, bioreductive agents, bioreductive alkylating agents, metal complexes, radiation sensitizers, radiation protectors, antisense agents, antigene agents, transcription factor inhibitors, ODN complexes, ribozymes, double stranded RNA, antitumor antibiotics, acivicin, aclararubicin, acodazole, acronycine, adozelesin, alanosine, allopurinol, altretamine, aminoglutethimide, amonafide, amsacrine, androgens, anguidine, aphidicolin glycinate, asaley, 5-azacitidine, azathioprine, Baker's Antifol, β-2′-deoxythioguanosine, bisantrene HCl, bleomycin sulfate, busulfan, buthionine sulfoximine (BSO), BWA 773U82, BW 502U83 HCl, BW 7U85 mesylate, caracemide, carbetimer, carboplatin, carmustine, chlorambucil, chloroquinoxaline sulfonamide, chlorozotocin, chromomycin A3, cisplatin, cladribine, carboplatin, oxaliplatin, rhodamine compounds, corticosteroids, CPT-11, cristanol cyclocytidine, cyclophosphamide, cytarabine, cytembena, dabis maleate, dacarbazine, dactinomycin, daunorubicin HCl, deazauridine, dexrazoxane, dianhydrogalactitol (DAG), dibromodulcitol, didemnin B, diethyldithiocarbamate, diglycoaldehyde, dihydro-5-azacytidine, doxorubicin, echinomycin, edatrexate, edelfosine, eflornithine, elsamitrucin, epirubicin, esorubicin, estramustine phosphate, estrogens, etanidazole, ethiofos, etoposide, fadrazole, fazarabine, fenretinide, finasteride, flavone acetic acid, floxuridine, fludarabine phosphate, 5-fluorouracil, flutamide, gallium nitrate, gemcitabine, goserelin acetate, hepsulfam, hexamethylene bisacetamide, amonafide, hydrazine sulfate, 4-hydroxyandrostenedione, hydroxyurea, idarubicin HCl, ifosfamide, 4-ipomeanol, iproplatin, isotretinoin, leuproloide acetate, levamisole, liposomal daunorubicin, liposomal doxorubicin, lomustine, lonidamine, maytansine, mechloethamine hydrochloride, melphalan, menogaril, 6-mercaptopurine, mesna, methotrexate, N-methylformamide, mifepristone, mitoguazone, mitomycin C, mitotane, mitoxantrone hydrochloride, nabilone, nafoxidine, neocarzinostatin, octreotide acetate, ormaplatin, oxaliplatin, paclitaxel, pala, pentostatin, piperazinedione, pipobroman, pirarubicin, piritrexim, piroxantrone hydrochloride, plicamycin, porfimer sodium, predimustine, procarbazine, progestins, pyrazofurin, razoxane, sargramostim, semustine, spirogermanium, streptonigrin, streptozocin, sulofenur, suramin sodium, tamoxifen, taxotere, tegafur, teniposide, terephthalamidine, teroxirone, thioguanine, thiotepa, thymidine, tiazofurin, topotecan, tormifene, treinoin, trifluoroperazine hydrochloride, trifluridine, trimetrexate, uracil mustard, vinblastine sulfate, vincristine sulfate, vindesine, vinorelbine, vinzolidine, Yoshi 864, zorubicin, 2-Cl-2′-deoxyadenosine, 3-deazauridine, 4-nitroestrone, 6-methylmercaptopurine riboside, 9-aminocamptothecin, nitrocamptothecin, irinotecan, CPT-11, acivicin, acodazole HCl, ADR-529, ICRF-187, amasacrine, aminothiadiazole, ADTA, antibiotic FR901228, aphidicolin glycinate, azacytidine, AZT, bizelesin, brefeldins, wortmannins, canthardins, bromodeoxyuridines, bryostatin, BSO, CAI, caracemide, carboplatin, chlorosulfaquinoxaline, sulfonamide, cisplatin, clomesone, cyclocytidine HCl, cyclodisone, cyclopentenylcytosine, deoxyspergualin, DHAC, didemnin B, dideoxy-β-fluorouracil, dideoxyadenosine, dideoxyinosine, dihydrotriazine benzene sulfonyl fluoride, dolastatin 10, ecteinascidin 743, etanidazole, ethiofos (WR-2721), fazarabine, flavopiridol, fludarabine phosphate, fostriecin, gallium nitrate, genistein, hepsulfam, HMBA, hydrazine sulfate, iododeoxyuridine, ipomeanol, KNI-272, leucovorin calcium, levamisole, melphalan, menogaril, merbarone, methotrexate, misonidazole, mitoguazone, mitoxantrone HCl, mitozolomide, N-methylformamide, O6-benzylguanine, PALA, pancratistatin, penclomedine, pentamethylmelamine HCl, pentamidine isethionate, pentostatin, perillyl alcohol, phyllanthoside, pibenzimole HCl, piroxantrone, pyrazine diazohydroxide, pyrazoloacridine, quinocarmycins, rebeccamycins, rhizoxin, semustine (methyl CCNU), Taxol, terephthalamidine, teroxirone, thioguanine, thymidine, tiazofurin, TMCA, 5-fluorouracil, methotrexate, cyclophosphamide, ras inhibitors, farnesylation inhibitors, bromodeoxyuridine, tetracycline compounds, arsenic trioxide, combretastatins, 2-methoxyestradiol, thalidomide and analogs, cephalotaxine derivatives, gleevec, stributyrin, triciribine phosphate, trimetrexate, UCN-01, 7-hydroxystaurosporine, uridine, lycurium, ritrosulfan, artemisinin, artesunate, lonidamine, mesna, bromomannitol, hydrazine sulfate, pipobroman, phenesterin, pyrazine diazohydroxide, cytembena, spirogermanium, terephthalamidine, bufalin, dibromodulcitol, gemcitabine, FMDC, colchicine, thiocolchicine, colchicine analogs, LHRH analogs, paclitaxel, MGBG, meisoindigo, indarubin analogs, metformin, phlorizin, and other compounds, including homoharringtonine (HHT).

In particular, the present invention is directed to amonafide and analogs and derivatives thereof. Amonafide is 5-amino-2-[2-(dimethylamino)ethyl]-1H-benzo[de]isoquinoline-1,3(2H)-dione. A derivative of amonafide is defined herein as a compound in which one or more groups or moieties present in amonafide is replaced with another group or moiety. An analog of amonafide is defined herein as a compound in which the benzo[de]isoquinoline ring structure of amonafide is replaced with another ring structure, such as, but not limited to, isoquinoline. A number of derivatives and analogs of amonafide are described below; others are known in the art.

Accordingly, within the scope of the present invention are derivatives or analogs of amonafide as follows:

(1) a derivative of amonafide wherein the amino group attached to one of the six-membered aromatic rings has one or both of the hydrogens replaced with C₁-C₃ lower alkyl;

(2) a derivative of amonafide wherein the nitrogen connected to one of the six-membered rings through an ethylene linkage has one or both of the methyl groups bound thereto replaced with C₂-C₃ lower alkyl;

(3) a derivative of amonafide wherein the ethylene linkage is replaced with a propylene (C₃) or a butylene (C₄) linkage;

(4) a derivative of amonfide of Formula (II) wherein: R₁ is selected from the group consisting of C₁-C₅ alkyl, amino, nitro, cyano, C₁-C₅ alkoxy, and hydrogen; and wherein R₂ is C₁-C₅ alkyl;

(5) a derivative of amonfide of Formula (III) wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s);

(6) a derivative of amonafide of Formula (III) wherein Q is selected from the group consisting of 1-R′-azetid-3-yl, 1-R′-pyrrolid-3-yl, 1-R′-piperid-4-yl, 1,2-diR′-1,2-diazolid-4-yl, 1,2-diazol-1-en-4-yl, 1-R′-piperid-4-yl, or 3-R′-oxazolid-5-yl, wherein R′ is selected from the group consisting of alkyl, alkenyl, acyl, alkoxy, aryl, amino, substituted amino, sulfo, sulfamoyl, carboxyl, carbamyl, and cyano;

(7) a derivative of amonafide of Formula (III) that is a naphthalimide wherein Q is —(CH₂)₂NR₂, where R is lower alkyl;

(8) a derivative of amonafide of Formula (III) that is a naphthalimide wherein Q is —(CH₂)₂NR₂, wherein NR₂ forms a heterocyclic group;

(9) a derivative of amonafide of Formula (III) that is a naphthalimide wherein Q is —(CH₂)₂NR₂ and wherein R₂ is —(CH₂)ₙ— or —(CH₂)ₘ—X—(CH₂)ₙ—, wherein m or n can be 0 to 5 and wherein X is NR″; wherein R″ is hydrogen, alkyl, alkenyl, acyl, alkoxy, aryl, amino, substituted amino, sulfo, sulfamoyl, carboxyl, carbamyl, cyano, or is not present; O; or S;

(10) a derivative of amonafide of Formula (III) wherein the tricyclic framework is derivatized so that it has one or more unsaturated bonds therein;

(11) a derivative of amonafide of Formula (III) wherein the tricyclic framework is derivativized so that it has at least one substituent selected from the group consisting of alkyl, aryl, and heteroaryl;

(12) a derivative of amonafide of Formula (III) wherein Q is selected from the group consisting of 1-pyrrolidyl, 3-R′-piperidyl, morpholino, 1-R′-piperazin-4-yl, 1-pyrrolyl, 1-imidazolyl, 1,3,5-triazol-1-yl, N-maleimido, 2-(R′-imino)pyrrolidyl, pyrazin-2-on-1-yl, 3-oxazolidyl, 3-oxazolyl, 2-pyrrolyl, 3-chloro-1-pyrrolidyl, 2-nitro-1-imidazolyl, 4-methoxy-1-imidazolyl, and 3-methyl-1-imidazolyl.

(13) a derivative of amonafide of Formula (III) wherein Q is selected from the group consisting of Subformulas 3(h), 3(i), 3(j), 3(k), 3(1), 3(m), 3(n), 3(o), 3(p), 3(q), 3(r), and 3(s), wherein R′ is selected from the group consisting of alkyl, alkenyl, acyl, alkoxy, aryl, amino, substituted amino, sulfo, sulfamoyl, carboxyl, carbamyl, and cyano;

(14) a derivative of amonafide of Formula (III) wherein the naphthalimide ring is modified to include one or more amino groups at positions other than position 3 of the naphthalimide ring;

(15) a derivative of amonafide of Formula (III) wherein the amino group at position 3 is replaced with an alternative substituent group selected from the group consisting of alkyl, aryl, nitro, amino, substituted amino, sulfamoyl, halo, carboxyl, carbamyl, and cyano;

(16) a derivative of amonafide of Formula (III) wherein an additional group is attached to the naphthalimide ring also comprising an amino group at position 3, the additional group being selected from the group consisting of alkyl, aryl, nitro, substituted amino, sulfamoyl, halo, carboxyl, carbamyl, and cyano;

(17) an analog of amonafide wherein the naphthalene ring is replaced with one bearing one or more nitrogen atoms in either or both rings;

(18) an analog of amonafide that is an isoquinoline analog of Formula (IV) wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s);

(19) an analog of amonafide that is an isoquinoline analog of Formula (IV) wherein Q is —(CH₂)ₙ—N(CH₃)₂, wherein n is 1-12; and

(20) a derivative or analog of amonafide or of alternatives (1)-(19) including one or more optional substituents, provided that the optionally substituted amonafide derivative or analog possesses substantially equivalent pharmacological activity to amonafide as defined in terms of either or both topoisomerase II inhibition and DNA intercalation.

In general, therefore, derivatives or analogs of amonafide include compounds that can be described as derivatives of amonafide, derivatives of azonafide, derivatives of mitonafide, and derivatives of elinafide. Derivatives or analogs of amonafide also include heterocyclic-substituted bis-1,8-naphthalimide compounds, 1,8 naphthalimide imidazo{4,5,1-de}acridones, 2-substituted-1,2-dihydro-3H-dibenz[de,h]isoquinoline-1,3-diones, amino-substituted-[2′-(dimethylamino)ethyl]1,2-dihydro-3H-dibenz[de,h]isoquinoline-1,3-diones, tetrahydroazonafides, phenanthrene analogs of azonafide, and azaphenanthrenes. Other derivatives or analogs of amonafide are described above.

(II) Dose Modification

Improvements for suboptimal chemotherapeutics including substituted naphthalimides such as amonafide and derivatives and analogs of amonafide are made by alterations to the time that the compound is administered, the use of dose-modifying agents that control the rate of metabolism of the compound, normal tissue protective agents, and other alterations. General examples include: variations of infusion schedules (e.g., bolus i.v. versus continuous infusion), the use of lymphokines (e.g., G-CSF, GM-CSF, EPO) to increase leukocyte count for improved immune response or for preventing anemia caused by myelosuppressive agents, or the use of rescue agents such as leucovorin for 5-FU or thiosulfate for cisplatin treatment. Specific inventive examples for substituted naphthalimides such as amonafide and derivatives and analogs of amonafide include: continuous i.v. infusion for hours to days; biweekly administration; doses greater than 5 mg/m²/day; progressive escalation of dosing from 1 mg/m²/day based on patient tolerance; doses less than 1 mg/m² for greater than 14 days; use of caffeine to modulate metabolism; use of isoniazid to modulate metabolism; selected and intermittent boost dose administrations; bolus single and multiple doses of 1-5 mg/m²; oral dosing including multiple daily dosing; micro dosing, immediate release dosing; slow release dosing; or controlled release dosing.

(III) Route of Administration

Improvements for suboptimal chemotherapeutics including substituted naphthalimides such as amonafide and derivatives and analogs of amonafide are made by alterations in the route by which the compound is administered. General examples include: changing route from oral to intravenous administration and vice versa; or the use of specialized routes such as subcutaneous, intramuscular, intraarterial, intraperitoneal, intralesional, intralymphatic, intratumoral, intrathecal, intravesicular, intracranial. Specific inventive examples for substituted naphthalimides such as amonafide and derivatives and analogs of amonafide include: topical administration; intravesicular administration for bladder cancer; oral administration; slow release oral delivery; intrathecal administration; intraarterial administration; continuous infusion; or intermittent infusion.

(IV) Schedule of Administration

Improvements for suboptimal chemotherapeutics including substituted naphthalimides such as amonafide and derivatives and analogs of amonafide are made by alterations to the time that the compound is administered. General examples include: changing from a monthly administration to a weekly or daily dosing or variations of the schedule. Specific inventive examples for substituted naphthalimides such as amonafide and derivatives and analogs of amonafide include: daily administration; weekly administration for three weeks; weekly administration for two weeks; biweekly administration; biweekly administration for three weeks with a 1-2 week rest period; intermittent boost dose administration; or administration daily for one week then once per week for multiple weeks.

(V) Indications for Use

Improvements for suboptimal chemotherapeutics including substituted naphthalimides such as amonafide and derivatives and analogs of amonafide are made by alterations in the types of disease or the clinical stage of disease for which the compound is administered. General examples include: the use of solid tumor agents for leukemias and vice versa, the use of antitumor agents for the treatment of benign hyperproliferative disease such as psoriasis or benign prostate hypertrophy, metabolic diseases, immunological diseases or infection. Specific inventive examples for substituted naphthalimides such as amonafide and derivatives and analogs of amonafide include: use for the treatment of triple-negative breast cancer; use for the treatment of acute leukemias; use for treatment of myelodysplastic syndrome; use for treatment of chronic myelocytic leukemia (CML), either subsequent to or in combination with the administration of tyrosine kinase inhibitors or homoharringtonine; use for treatment of chronic lymphocytic leukemia; use for treatment of Hodgkin's lymphoma; use for treatment of non-Hodgkin's lymphoma; use for treatment of mycosis fungoides; use for treatment of androgen-resistant prostate cancer; use for treatment of lung small-cell carcinoma, either subsequent to or in combination with the administration of EGFR inhibitors such as erlotinib (Tarceva) or gefitinib (Iressa), wherein the lung small cell carcinoma is characterized by either wild-type or mutated EGFR; use for treatment of lung non-small cell carcinoma, subsequent to or in combination with EGFR inhibitors such as erlotinib or gefitinib, wherein the lung non-small cell carcinoma is characterized by either wild-type or mutated EGFR; use for treatment of breast cancer characterized by overexpressed Her-2-neu; use for treatment of glioblastoma that is resistant to one or both of the following therapeutic agents: temozolomide (Temodar) or bevacizumab (Avastin), or is characterized by EGFR variant III, either alone or in combination with other therapeutic agents; use for treatment of malignancies characterized by overexpressed topoisomerase II; use for treatment of malignancies characterized by overexpressed and/or mutated EGFR; use for treatment of prostate cancer; use for treatment of malignancies characterized by overexpressed and/or mutated Her2/neu; use for treatment of malignancies characterized by overexpressed and/or mutated Braf; use for treatment of malignancies characterized by overexpressed and/or mutated BTK; use for treatment of malignancies characterized by overexpressed and/or mutated KRAS; use for treatment of malignancies characterized by overexpressed and/or mutated c-Myc; use for treatment of malignancies characterized by overexpressed and/or mutated p53; use for treatment of angiogenic diseases; use for treatment of benign prostate hypertrophy; use for treatment of psoriasis; use for treatment of gout; use for treatment of autoimmune conditions; use for prevention of transplantation rejection; use for restenosis prevention in cardiovascular disease; use in bone marrow transplantation; use as an anti-infective; or use in treatment for AIDS.

(VI) Disease Stages

Improvements for suboptimal chemotherapeutics including substituted naphthalimides such as amonafide and derivatives and analogs of amonafide are made by alterations in the stage of disease at diagnosis/progression that the compound is administered. General examples include: the use of chemotherapy for non-resectable local disease, prophylactic use to prevent metastatic spread or inhibit disease progression or conversion to more malignant stages. Specific inventive examples for substituted naphthalimides such as amonafide and derivatives and analogs of amonafide include: use for the treatment of localized polyp stage colon cancer; use for the treatment of leukoplakia in the oral cavity; use to induce angiogenesis inhibition to prevent or limit metastatic spread; or use against HIV with AZT, DDI, or reverse transcriptase inhibitors.

(VII) Other Indications

Improvements for suboptimal chemotherapeutics including substituted naphthalimides such as amonafide and derivatives and analogs of amonafide are made by using the compound for non-malignant diseases and conditions. General examples include: premalignant conditions, benign hyperproliferative conditions, treatment of infections, treatment of parasitic infections, usage to relieve pain, use for control of pleural effusions. Specific inventive examples for substituted naphthalimides such as amonafide and derivatives and analogs of amonafide include: use as an anti-infective agent; use as an antiviral agent; use as an antibacterial agent; use for control of pleural effusions; use as an antifungal agent; use as an antiparasitic agent; use for treatment of eczema; use for treatment of shingles; use for treatment of condylomata; use for treatment of human papilloma virus (HPV); or use for treatment of herpes simplex virus (HSV).

(VIII) Patient Selection

Improvements for suboptimal chemotherapeutics including substituted naphthalimides such as amonafide and derivatives and analogs of amonafide are made by alterations to the type of patient that would best tolerate or benefit from the use of the compound. General examples include: use of pediatric doses for elderly patients, altered doses for obese patients; exploitation of co-morbid disease conditions such as diabetes, cirrhosis, or other conditions that may uniquely exploit a feature of the compound. Specific inventive examples for substituted naphthalimides such as amonafide and derivatives and analogs of amonafide include: patients with disease conditions with high levels of metabolic enzymes such as histone deacetylase, protein kinases, ornithine decarboxylase; patients with disease conditions with low levels of metabolic enzymes such as histone deacetylase, protein kinases, or ornithine decarboxylase; patients with low or high susceptibility to thrombocytopenia or neutropenia; patients intolerant of GI toxicities; patients characterized by over- or under-expression of jun, GPCRs, signal transduction proteins, VEGF, prostate specific genes, protein kinases, or telomerase.

(IX) Patient/Disease Phenotype

Improvements for suboptimal chemotherapeutics including substituted naphthalimides such as amonafide and derivatives and analogs of amonafide are made by more precise identification of a patient's ability to tolerate, metabolize and exploit the use of the compound. General examples include: use of diagnostic tools and kits to better characterize a patient's ability to process/metabolize a chemotherapeutic agent or the patient's susceptibility to toxicity caused by potential specialized cellular, metabolic, or organ system phenotypes. Specific inventive examples for substituted naphthalimides such as amonafide and derivatives and analogs of amonafide include: diagnostic tools, techniques, kits and assays to confirm a patient's particular phenotype and for the measurement of metabolism enzymes and metabolites, histone deacetylase, protein kinases, ornithine decarboxylase, VEGF, a protein that is a gene product of a prostate specific gene, protein kinases, telomerase, a protein that is a gene product of jun, GPCR's, surrogate compound dosing or low dose drug pre-testing for enzymatic status.

One specific phenotypic indication potentially related to the toxicity of amonafide is the acetylator phenotype, as described in M. J. Ratain et al., “Paradoxical Relationship Between Acetylator Phenotype and Amonafide Toxicity,” Clin. Pharmacol. Ther. 50: 573-579, incorporated herein by this reference, and in F. Innocenti et al., “Pharmacogenetics of Cancer Agents: Lessons from Amonafide and Irinotecan,” Drug Metab. Dispos. 29: 596-600 (2001), incorporated herein by this reference.

(X) Patient/Disease Genotype

Improvements for suboptimal chemotherapeutics including substituted naphthalimides such as amonafide and derivatives and analogs of amonafide are made by testing and analyzing a patient's genotype for unique features that may be of value to predict efficacy, toxicity, metabolism, or other parameters relevant to therapeutic use of the suboptimal therapeutic. General examples include: biopsy samples of tumors or normal tissues (e.g., white blood cells) may be taken and analyzed to specifically tailor or monitor the use of a particular drug against a gene target; analysis of unique tumor gene expression pattern, SNP's (single nucleotide polymorphisms), to enhance efficacy or to avoid particular drug-sensitive normal tissue toxicities. Specific inventive examples for substituted naphthalimides such as amonafide and derivatives and analogs of amonafide include: diagnostic tools, techniques, kits and assays to confirm a patient's particular genotype; gene/protein expression chips and analysis; Single Nucleotide Polymorphisms (SNP's) assessment; SNP's for histone deacetylase, ornithine decarboxylase, GPCR's, protein kinases, telomerase, jun; identification and measurement of metabolism enzymes and metabolites; or use of a method to determine the genotype for N-acetyltransferase activity.

(XI) Pre/Post-Treatment Preparation

Improvements for suboptimal chemotherapeutics including substituted naphthalimides such as amonafide and derivatives and analogs of amonafide are made by specialized preparation of a patient prior to or after the use of a chemotherapeutic agent. General examples include: induction or inhibition of metabolizing enzymes, specific protection of sensitive normal tissues or organ systems. Specific inventive examples for substituted naphthalimides such as amonafide and derivatives and analogs of amonafide and derivatives and analogs of amonafide include: the use of colchicine or analogs; use of diuretics; use of uricosuric agents such as probenecid; use of uricase; non-oral use of nicotinamide; use of sustained release forms of nicotinamide; use of inhibitors of polyADP ribose polymerase; use of caffeine; leucovorin rescue; infection control; use of antihypertensives.

(XII) Toxicity Management

Improvements for suboptimal chemotherapeutics including substituted naphthalimides such as amonafide and derivatives and analogs of amonafide are made by use of additional drugs or procedures to prevent or reduce potential side-effects or toxicities. General examples include: the use of anti-emetics, anti-nausea agents, hematological support agents to limit or prevent neutropenia, anemia, thrombocytopenia, vitamins, antidepressants, treatments for sexual dysfunction, or use of other agents or methods to reduce potential side effects or toxicities. Specific inventive examples for substituted naphthalimides such as amonafide and derivatives and analogs of amonafide include: the use of colchicine or analogs; the use of uricosurics such as probenecid; the use of diuretics; the use of uricase; non-oral use of nicotinamide; use of sustained release forms of nicotinamide; use of inhibitors of polyADP-ribose polymerase; the use of caffeine; leucovorin rescue; the use of sustained release allopurinol; non-oral use of allopurinol; administration of bone marrow transplant stimulants, blood, platelet infusions, Neupogen, G-CSF; or GM-CSF; pain management; administration of anti-inflammatories; administration of fluids; administration of corticosteroids; administration of insulin control medications; administration of antipyretics; administration of anti-nausea treatments; administration of anti-diarrhea treatments; administration of N-acetylcysteine, administration of antihistamines; administration of agents for reduction of gastric toxicity.

(XIII) Pharmacokinetic/Pharmacodynamic Monitoring

Improvements for suboptimal chemotherapeutics including substituted naphthalimides such as amonafide and derivatives and analogs of amonafide are made by the use of monitoring drug levels after dosing in an effort to maximize a patient's drug plasma level, to monitor the generation of toxic metabolites, or to monitor of ancillary medicines that could be beneficial or harmful in terms of drug-drug interactions. General examples include: the monitoring of drug plasma protein binding, the monitoring of specific metabolites or breakdown products, or other products of biotransformation. Specific inventive examples for substituted naphthalimides such as amonafide and derivatives and analogs of amonafide include: multiple determinations of drug plasma levels; multiple determinations of metabolites in the blood or urine.

One method potentially useful for the monitoring of metabolism of amonafide or a derivative or analog of amonafide is an ELISA assay for the rapid determination of N-acetyltransferase (NAT2 phenotypes), described in U.S. Pat. No. 5,830,672 to Wainer et al., incorporated herein by this reference. Amonafide is converted to an active metabolite by way of the N-acetyltransferase NAT2, and it has been reported that there is a direct correlation between the acetylator phenotype and the degree of toxicity induced by amonafide, with patients possessing a phenotype for rapid acetylation at greater risk to problems associated with severe toxicity. In general, this ELISA assay measures the concentration of two metabolites of caffeine. The first of these metabolites is 5-acetamino-6-amino-1-methyluracil (AAMU); the second of these metabolites is either 5-acetamino-6-formylamino-1-methyluracil (AFMU) or 1-methylxanthine (1X).

(XIV) Drug Combinations

Improvements for suboptimal chemotherapeutics including substituted napthalamides such as amonafide are made by exploiting unique drug combinations that may provide a more than additive or synergistic improvement in efficacy or side-effect management. General examples include: alkylating agents with anti-metabolites, topoisomerase inhibitors with antitubulin agents. Specific inventive examples for substituted naphthalimides such as amonafide and derivatives and analogs of amonafide include: use with fraudulent nucleosides; use with fraudulent nucleotides; use with thymidylate synthetase inhibitors; use with signal transduction inhibitors; use with cisplatin or platinum analogs; use with alkylating agents; use with anti-tubulin agents; use with antimetabolites; use with berberine; use with apigenin; use with colchicine and analogs; use with genistein; use with etoposide; use with cytarabine; use with camptothecins; use with vinca alkaloids, including vinblastine; use with topoisomerase inhibitors; use with 5-fluorouracil; use with curcumin; use with NF-κB inhibitors; use with rosmarinic acid; use with mitoguazone and analogs; use with meisoindigo; use with imatinib; use with dasatinib; use with nilotinib; use with epigenetic modulators; use with transcription factor inhibitors; use with taxol; use with homoharringtonine; use with pyridoxal; use with spirogermanium; use with caffeine; use with nicotinamide; use with methylglyoxalbisguanylhydrazone; use with epidermal growth factor receptor (EGFR) inhibitors; use with poly-ADP ribose polymerase (PARP) inhibitors; use with Bruton's tyrosine kinase (BTK) inhibitors; use with c-Myc inhibitors; use with PTEN inhibitors; use with IDH inhibitors; use with polyamine analogs; use with thalidomide and analogs; use with homoharringtonine and analogs; use with bruceantin and analogs; use with bisantrene, amsacrine, or analogs of bisantrene or amsacrine; use with mitoxantrone; use with vosaroxin; use with dianhydrogalactitol or dibromodulcitol; use with 5-azacytidine; use with decitabine; use with anti-VEGF agents such as avastin; use with anti-CD20 agents such as rituximab; use with anti-EGFR vaccines; use with T-cell stimulants; use with dendritic cell vaccines; and use with PD inhibitors. Other drug combinations intended to modulate or affect specific targets or cellular processes are described below. When drug combinations are employed, more than one additional drug can be used (in addition to the amonafide or the derivative or analog of amonafide). The selection of one or more additional drugs is within the scope of one of ordinary skill in the art and can be made by analyzing the targets or pathways affected or modulated by each drug.

The use of amonafide or a derivative or analog of amonafide together with homoharringtonine or another cephalotaxine is described in U.S. Pat. No. 7,683,050 to Brown, incorporated herein by this reference.

Taxol is (2α,4α,5β,7β,10β,13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate and is used to treat lung cancer, ovarian cancer, breast cancer, head and neck cancer, and Kaposi's sarcoma. Spirogermanium is (2α,4α,5β,7β,10β,13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate and has antineoplastic activity.

(XV) Chemosensitization

Improvements for suboptimal chemotherapeutics including substituted naphthalimides such as amonafide and derivatives and analogs of amonafide are made by exploiting them as chemosensitizers where no measureable activity is observed when used alone but in combination with other therapeutics a more than additive or synergistic improvement in efficacy is observed. General examples include: misonidazole with alkylating agents, tirapazamine with cisplatin. Specific inventive examples for substituted naphthalimides such as amonafide and derivatives and analogs of amonafide include: as a chemosensitizer in combination with topoisomerase inhibitors; as a chemosensitizer in combination with fraudulent nucleosides; as a chemosensitizer in combination with fraudulent nucleotides; as a chemosensitizer in combination with thymidylate synthetase inhibitors; as a chemosensitizer in combination with signal transduction inhibitors; as a chemosensitizer in combination with cisplatin or platinum analogs; as a chemosensitizer in combination with alkylating agents; as a chemosensitizer in combination with anti-tubulin agents; as a chemosensitizer in combination with antimetabolites; as a chemosensitizer in combination with berberine; as a chemosensitizer in combination with apigenin; as a chemosensitizer in combination with colchicine or analogs of colchicine; as a chemosensitizer in combination with genistein; as a chemosensitizer in combination with etoposide; as a chemosensitizer in combination with cytarabine; as a chemosensitizer in combination with camptothecins; as a chemosensitizer in combination with vinca alkaloids; as a chemosensitizer in combination with 5-fluorouracil; as a chemosensitizer in combination with curcumin; as a chemosensitizer in combination with NF-κB inhibitors; as a chemosensitizer in combination with rosmarinic acid; or as a chemosensitizer in combination with mitoguazone.

(XVI) Chemopotentiation

Improvements for suboptimal chemotherapeutics including substituted naphthalimides such as amonafide are made by exploiting them as chemopotentiators where minimal therapeutic activity is observed alone but in combination with other therapeutics a more than additive or synergistic improvement in efficacy is observed. General examples include: dibromodulcitol with fraudulent nucleosides or fraudulent nucleotides. Specific inventive examples for substituted naphthalimides such as amonafide include: as a chemopotentiator in combination with fraudulent nucleosides; as a chemopotentiator in combination with fraudulent nucleotides; as a chemopotentiator in combination with thymidylate synthetase inhibitors; as a chemopotentiator in combination with signal transduction inhibitors; as a chemopotentiator in combination with cisplatin or platinum analogs; as a chemopotentiator in combination with alkylating agents; as a chemopotentiator in combination with anti-tubulin agents; as a chemopotentiator in combination with antimetabolites; as a chemopotentiator in combination with berberine; as a chemopotentiator in combination with apigenin; as a chemopotentiator in combination with colchicine or analogs of colchicine; as a chemopotentiator in combination with genistein; as a chemopotentiator in combination with etoposide; as a chemopotentiator in combination with cytarabine; as a chemopotentiator in combination with camptothecins; as a chemopotentiator in combination with vinca alkaloids; as a chemopotentiator in combination with topoisomerase inhibitors; as a chemopotentiator in combination with 5-fluorouracil; as a chemopotentiator in combination with curcumin; as a chemopotentiator in combination with NF-κB inhibitors; as a chemopotentiator in combination with rosmarinic acid; or as a chemopotentiator in combination with mitoguazone.

(XVII) Post-Treatment Patient Management

Improvements for suboptimal chemotherapeutics including substituted naphthalimides such as amonafide and derivatives and analogs of amonafide are made by drugs, treatments and or diagnostics to allow for the maximum benefit to patients treated with a compound. General examples include: pain management, nutritional support, anti-emetics, anti-nausea therapies, anti-anemia therapy, anti-inflammatories. Specific inventive examples for substituted naphthalimides such as amonafide and derivatives and analogs of amonafide include: use with therapies associated with pain management; nutritional support; anti-emetics; anti-nausea therapies; anti-anemia therapy; anti-inflammatories: antipyretics; immune stimulants.

(XVIII) Alternative Medicine/Therapeutic Support

Improvements for suboptimal chemotherapeutics including substituted naphthalimides such as amonafide and derivatives and analogs of amonafide are made by the use of unapproved/non-conventional therapeutics or methods to enhance effectiveness or reduce side effects. General examples include: hypnosis, acupuncture, meditation, herbal medications and extracts, applied kinesiology, prayer. Specific inventive examples for substituted naphthalimides such as amonafide and derivatives and analogs of amonafide include: hypnosis; acupuncture; meditation; herbal medications created either synthetically or through extraction including NF-κB inhibitors (such as parthenolide, curcumin, or rosmarinic acid); natural anti-inflammatories (including rhein or parthenolide); immunostimulants (such as those found in Echinacea); antimicrobials (such as berberine); flavonoids, isoflavones, and flavones (such as apigenenin, genistein, genistin, 6″-O-malonylgenistin, 6″-O-acetylgenistin, daidzein, daidzin, 6″-O-malonyldaidzin, 6″-O-acetylgenistin, glycitein, glycitin, 6″-O-malonylglycitin, and 6-O-acetylglycitin); applied kinesiology.

(XIX) Bulk Drug Product Improvements

Improvements for suboptimal chemotherapeutics including substituted naphthalimides such as amonafide and derivatives and analogs of amonafide are made by alterations in the pharmaceutical bulk substance. General examples include: salt formation, homogeneous crystalline structure, pure isomers. Specific inventive examples for substituted naphthalimides such as amonafide and derivatives and analogs of amonafide include: free base form; salt formation; homogeneous crystalline structure; amorphous structure; pure isomers; increased purity; lower residual solvents and heavy metals.

(XX) Diluent Systems

Improvements for suboptimal chemotherapeutics including substituted naphthalimides such as amonafide and derivatives and analogs of amonafide are made by alterations in the diluents used to solubilize and deliver/present the compound for administration. General examples include: Cremophor-EL, cyclodextrins for poorly water soluble compounds. Specific inventive examples for substituted naphthalimides such as amonafide and derivatives and analogs of amonafide include: use of emulsions; dimethylsulfoxide (DMSO); N-methylformamide (NMF); dimethylformamide (DMF); dimethylacetamide (DMA); ethanol; benzyl alcohol; dextrose-containing water for injection; Cremophor; cyclodextrins; PEG.

(XXI) Solvent Systems

Improvements for suboptimal chemotherapeutics including substituted naphthalimides such as amonafide and derivatives and analogs of amonafide are made by alterations in the solvents used or required to solubilize a compound for administration or for further dilution. General examples include: ethanol, dimethylacetamide (DMA). Specific inventive examples for substituted naphthalimides such as amonafide and derivatives and analogs of amonafide include: the use of emulsions; DMSO; NMF; DMF; DMA; ethanol; benzyl alcohol; dextrose-containing water for injection; Cremophor; PEG; salt systems.

(XXII) Excipients

Improvements for suboptimal chemotherapeutics including substituted naphthalimides such as amonafide and derivatives and analogs of amonafide are made by alterations in the materials/excipients, buffering agents, or preservatives required to stabilize and present a chemical compound for proper administration. General examples include: mannitol, albumin, EDTA, sodium bisulfite, benzyl alcohol. Specific inventive examples for substituted naphthalimides such as amonafide and derivatives and analogs of amonafide include: the use of mannitol; the use of albumin; the use of EDTA; the use of sodium bisulfite; the use of benzyl alcohol; the use of carbonate buffers; the use of phosphate buffers; the use of polyethylene glycol (PEG); the use of vitamin A; the use of vitamin D; the use of vitamin E; the use of esterase inhibitors; the use of cytochrome P450 inhibitors; the use of multi-drug resistance (MDR) inhibitors; the use of organic resins; or the use of detergents.

(XXIII) Dosage Forms

Improvements for suboptimal chemotherapeutics including substituted naphthalimides such as amonafide and derivatives and analogs of amonafide are made by alterations in the potential dosage forms of the compound dependent on the route of administration, duration of effect, plasma levels required, exposure to normal tissues potentially resulting in side effects, and exposure to metabolizing enzymes. General examples include: tablets, capsules, topical gels, creams, patches, suppositories. Specific inventive examples for substituted naphthalimides such as amonafide and derivatives and analogs of amonafide include: the use of tablets; the use of capsules; the use of topical gels; the use of topical creams; the use of patches; the use of suppositories; the use of lyophilized dosage fills; the use of immediate-release formulations; the use of slow-release formulations; the use of controlled-release formulations; or the use of liquid in capsules.

(XXIV) Dosage Kits and Packaging

Improvements for suboptimal chemotherapeutics including substituted naphthalimides such as amonafide and derivatives and analogs of amonafide are made by alterations in the dosage forms, container/closure systems, accuracy of mixing and dosage preparation and presentation. General examples include: amber vials to protect from light, stoppers with specialized coatings. Specific inventive examples for substituted naphthalimides such as amonafide and derivatives and analogs of amonafide include: the use of amber vials to protect from light; and stoppers with specialized coatings to improve shelf-life stability.

(XXV) Drug Delivery Systems

Improvements for suboptimal chemotherapeutics including substituted napthalamides such as amonafide and derivatives and analogs of amonafide are made by the use of delivery systems to improve the potential attributes of a pharmaceutical product such as convenience, duration of effect, or reduction of toxicities. General examples include: nanocrystals, bioerodible polymers, liposomes, slow release injectable gels, microspheres. Specific inventive examples for substituted naphthalimides such as amonafide and derivatives and analogs of amonafide include: the use of oral dosage forms; the use of nanocrystals; the use of nanoparticles; the use of cosolvents; the use of slurries; the use of syrups; the use of bioerodible polymers; the use of liposomes; the use of slow release injectable gels; or the use of microspheres.

(XXVI) Drug Conjugate Forms

Improvements for suboptimal chemotherapeutics including substituted naphthalimides such as amonafide and derivatives and analogs of amonafide are made by alterations to the parent molecule with covalent, ionic, or hydrogen bonded moieties to alter the efficacy, toxicity, pharmacokinetics, metabolism, or route of administration. General examples include: polymer systems such as polyethylene glycols, polylactides, polyglycolides, amino acids, peptides, multivalent linkers. Specific inventive examples for substituted naphthalimides such as amonafide and derivatives and analogs of amonafide include: the use of polymer systems such as polyethylene glycols; the use of polylactides; the use of polyglycolides; the use of amino acids; the use of peptides; the use of multivalent linkers; or the use of conjugates with fatty amines.

(XXVII) Compound Analogs

Improvements for suboptimal chemotherapeutics including substituted naphthalimides such as amonafide and derivatives and analogs of amonafide are made by alterations to the parent structure of a molecule with additional chemical functionalities that may alter efficacy, reduce toxicity, improve pharmacological performance, be compatible with a particular route of administration, or alter the metabolism of the therapeutic agent. General examples include: alteration of side chains to increase or decrease lipophilicity; additional chemical functionalities to alter reactivity, electron affinity, or binding capacity; salt forms. Specific inventive examples for substituted naphthalimides such as amonafide and derivatives and analogs of amonafide include: alteration of side chains to increase or decrease lipophilicity; additional chemical functionalities to alter reactivity, electron affinity, or binding capacity; salt forms.

(XXVIII) Prodrugs

Improvements for suboptimal chemotherapeutics including substituted napthalamides such as amonafide and derivatives and analogs of amonafide are made by alterations to the molecule such that improved pharmaceutical performance is gained with a variant of the active molecule in that after introduction into the body a portion of the molecule is cleaved to reveal the preferred active molecule. General examples include: enzyme sensitive esters, dimers, Schiff bases. Specific inventive examples for substituted naphthalimides such as amonafide and derivatives and analogs of amonafide include: the use of enzyme sensitive esters; the use of dimers; the use of Schiff bases; the use of pyridoxal complexes; the use of caffeine complexes; the use of plasmin-activated prodrugs; or the use of a drug targeting complex comprising a targeting carrier molecule that is selectively distributed to a specific cell type or tissue containing the specific cell type; a linker which is acted upon by a molecule that is present at an effective concentration in the environs of the specific cell type; and a therapeutically active agent to be delivered to the specific cell type.

(XXIX) Multiple Drug Systems

Improvements for suboptimal chemotherapeutics including substituted napthalamides such as amonafide and derivatives and analogs of amonafide are made by the use of additional compounds, such as therapeutic or biological agents that when administered in the proper fashion, a unique and beneficial effect can be realized. General examples include: inhibitors of multi-drug resistance, specific drug resistance inhibitors, specific inhibitors of selective enzymes, signal transduction inhibitors, repair inhibition. Specific inventive examples for substituted naphthalamides such as amonafide include the use of amonafide and derivatives and analogs of amonafide with: the use of inhibitors of multi-drug resistance; the use of specific drug resistance inhibitors; the use of specific inhibitors of selective enzymes; the use of signal transduction inhibitors; the use of meisoindigo; the use of imatinib; the use of hydroxyurea; the use of dasatinib; the use of capecitabine; the use of nilotinib; the use of repair inhibition; the use of topoisomerase inhibitors with non-overlapping side effects; PARP inhibitors; or EGFR inhibitors.

(XXX) Biotherapeutic Enhancement

Improvements for suboptimal chemotherapeutics including substituted naphthalimides such as amonafide and derivatives and analogs of amonafide are made by its use in combination as sensitizers/potentiators with biological response modifiers. General examples include: use in combination as sensitizers/potentiators with biological response modifiers, cytokines, lymphokines, therapeutic antibodies, antisense therapies, gene therapies. Specific inventive examples for substituted naphthalimides such as amonafide and derivatives and analogs of amonafide include: use in combination as sensitizers/potentiators with biological response modifiers; use in combination as sensitizers/potentiators with cytokines; use in combination as sensitizers/potentiators with lymphokines; use in combination as sensitizers/potentiators with therapeutic antibodies; use in combination as sensitizers/potentiators with antisense therapies; use in combination as sensitizers/potentiators with gene therapies; use in combination as sensitizers/potentiators with ribozymes; use in combination as sensitizers/potentiators with RNA interference; use in combination with vaccines (cellular or non-cellular); or use in combination with stem cells.

(XXXI) Biotherapeutic Resistance Modulation

Improvements for suboptimal chemotherapeutics including substituted naphthalimides such as amonafide and derivatives and analogs of amonafide are made by exploiting their selective use to overcome developing or complete resistance to the efficient use of biotherapeutics. General examples include: tumors resistant to the effects of biological response modifiers, cytokines, lymphokines, therapeutic antibodies, antisense therapies, gene therapies. Specific inventive examples for substituted naphthalimides such as amonafide and derivatives and analogs of amonafide include: use against tumors resistant to the effects of biological response modifiers; use against tumors resistant to the effects of cytokines; use against tumors resistant to the effects of lymphokines; use against tumors resistant to the effects of therapeutic antibodies; use against tumors resistant to the effects of antisense therapies; use against tumors resistant to the effects of gene therapies; use against tumors resistant to the effects of ribozymes; or use against tumors resistant to the effects of RNA interference.

(XXXII) Radiation Therapy Enhancement

Improvements for suboptimal chemotherapeutics including substituted naphthalimides such as amonafide and derivatives and analogs of amonafide are made by exploiting their use in combination with ionizing radiation, phototherapies, heat therapies, radio-frequency generated therapies. General examples include: hypoxic cell sensitizers, radiation sensitizers/protectors, photosensitizers, radiation repair inhibitors. Specific inventive examples for substituted naphthalimides such as amonafide include: use with hypoxic cell sensitizers; use with radiation sensitizers/protectors; use with photosensitizers; use with radiation repair inhibitors; use with thiol depletion; use with vaso-targeted agents; use with radioactive seeds; use with radionuclides; use with radiolabeled antibodies; use with brachytherapy; or use with bioreductive alkylating agents.

(XXXIII) Novel Mechanisms of Action

Improvements for suboptimal chemotherapeutics including substituted naphthalimides such as amonafide and derivatives and analogs of amonafide are made by optimizing their utility by determining the various mechanisms of actions or biological targets of a compound for greater understanding and precision to better exploit the utility of the molecule. General examples include: imatinib (Gleevec) for chronic myelocytic leukemia (CML), arsenic trioxide for acute promyelocytic leukemia (APL), retinoic acid for APL. Specific inventive examples for substituted naphthalimides such as amonafide and derivatives and analogs of amonafide include: use with inhibitors of poly-ADP ribose polymerase; use with agents that affect vasculature; use with agents that promote vasodilation; use with oncogenic targeted agents; use with signal transduction inhibitors; use with agents inducing EGFR inhibition; use with agents inducing Protein Kinase C inhibition; use with agents inducing Phospholipase C downregulation; use with agents including jun downregulation; use with agents modulating expression of histone genes; use with agents modulating expression of VEGF; use with agents modulating expression of ornithine decarboxylase; use with agents modulating expression of jun D; use with agents modulating expression of v-jun; use with agents modulating expression of GPCRs; use with agents modulating expression of protein kinase A; use with agents modulating expression of protein kinases other than protein kinase A; use with agents modulating expression of telomerase; use with agents modulating expression of prostate specific genes; use with agents modulating expression of histone deacetylase; or use with agents modulating expression of CHK2 checkpoint kinase.

(XXXIV) Selective Target Cell Population Therapeutics

Improvements for suboptimal chemotherapeutics including substituted naphthalimides such as amonafide and derivatives and analogs of amonafide are made by more precise identification and exposure of the compound to those select cell populations where the compounds effect can be maximally exploited. General examples include: tirapazamine and mitomycin c for hypoxic cells, vinca alkaloids for cells entering mitosis. Specific inventive examples for substituted naphthalimides such as amonafide and derivatives and analogs of amonafide include: use against radiation sensitive cells; use against radiation resistant cells; use against energy depleted cells; use against endothelial cells.

(XXXV) Use with Agents to Enhance Activity

Improvements for suboptimal chemotherapeutics including substituted naphthalimides such as amonafide and derivatives and analogs of amonafide are made by use of agents to enhance activity of the amonafide or the derivative or analog of amonafide. General examples include: use with nicotinamide, caffeine, tetandrine, or berberine. Specific inventive examples for substituted naphthalimides such as amonafide and derivatives and analogs of amonafide include: use with nicotinamide; use with caffeine; use with tetandrine; or use with berberine.

Accordingly, one aspect of the present invention is a method to improve the efficacy and/or reduce the side effects of suboptimally administered drug therapy comprising the steps of:

(1) identifying at least one factor or parameter associated with the efficacy and/or occurrence of side effects of the drug therapy; and

(2) modifying the factor or parameter to improve the efficacy and/or reduce the side effects of the drug therapy.

Typically, the factor or parameter is selected from the group consisting of:

(1) dose modification;

(2) route of administration;

(3) schedule of administration;

(4) indications for use;

(5) selection of disease stage;

(6) other indications;

(7) patient selection;

(8) patient/disease phenotype;

(9) patient/disease genotype;

(10) pre/post-treatment preparation

(11) toxicity management;

(12) pharmacokinetic/pharmacodynamic monitoring;

(13) drug combinations;

(14) chemosensitization;

(15) chemopotentiation;

(16) post-treatment patient management;

(17) alternative medicine/therapeutic support;

(18) bulk drug product improvements;

(19) diluent systems;

(20) solvent systems;

(21) excipients;

(22) dosage forms;

(23) dosage kits and packaging;

(24) drug delivery systems;

(25) drug conjugate forms;

(26) compound analogs;

(27) prodrugs;

(28) multiple drug systems;

(29) biotherapeutic enhancement;

(30) biotherapeutic resistance modulation;

(31) radiation therapy enhancement;

(32) novel mechanisms of action;

(33) selective target cell population therapeutics; and

(34) use with an agent enhancing its activity.

In one alternative, the suboptimally administered drug therapy is administration of amonafide.

In another alternative, the suboptimally administered drug therapy is administration of a derivative or analog of amonafide. Typically, the derivative or analog of amonafide is selected from the group consisting of:

(1) a derivative of amonafide wherein the amino group attached to one of the six-membered aromatic rings has one or both of the hydrogens replaced with C₁-C₃ lower alkyl;

(2) a derivative of amonafide wherein the nitrogen connected to one of the six-membered rings through an ethylene linkage has one or both of the methyl groups bound thereto replaced with C₂-C₃ lower alkyl;

(3) a derivative of amonafide wherein the ethylene linkage is replaced with a propylene (C₃) or a butylene (C₄) linkage;

(4) a derivative of amonfide of Formula (II) wherein: R₁ is selected from the group consisting of C₁-C₅ alkyl, amino, nitro, cyano, C₁-C₅ alkoxy, and hydrogen; and wherein R₂ is C₁-C₅ alkyl;

(5) a derivative of amonfide of Formula (III) wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s);

(6) a derivative of amonafide of Formula (III) wherein Q is selected from the group consisting of 1-R′-azetid-3-yl, 1-R′-pyrrolid-3-yl, 1-R′-piperid-4-yl, 1,2-diR′-1,2-diazolid-4-yl, 1,2-diazol-1-en-4-yl, 1-R′-piperid-4-yl, or 3-R′-oxazolid-5-yl, wherein R′ is selected from the group consisting of alkyl, alkenyl, acyl, alkoxy, aryl, amino, substituted amino, sulfo, sulfamoyl, carboxyl, carbamyl, and cyano;

(7) a derivative of amonafide of Formula (III) that is a naphthalimide wherein Q is —(CH₂)₂NR₂, where R is lower alkyl;

(8) a derivative of amonafide of Formula (III) that is a naphthalimide wherein Q is —(CH₂)₂NR₂, wherein NR₂ forms a heterocyclic group;

(9) a derivative of amonafide of Formula (III) that is a naphthalimide wherein Q is —(CH₂)₂NR₂ and wherein R₂ is —(CH₂)ₙ— or —(CH₂)ₘ—X—(CH₂)ₙ—, wherein m or n can be 0 to 5 and wherein X is NR″; wherein R″ is hydrogen, alkyl, alkenyl, acyl, alkoxy, aryl, amino, substituted amino, sulfo, sulfamoyl, carboxyl, carbamyl, cyano, or is not present; O; or S;

(10) a derivative of amonafide of Formula (III) wherein the tricyclic framework is derivatized so that it has one or more unsaturated bonds therein;

(11) a derivative of amonafide of Formula (III) wherein the tricyclic framework is derivativized so that it has at least one substituent selected from the group consisting of alkyl, aryl, and heteroaryl;

(12) a derivative of amonafide of Formula (III) wherein Q is selected from the group consisting of 1-pyrrolidyl, 3-R′-piperidyl, morpholino, 1-R′-piperazin-4-yl, 1-pyrrolyl, 1-imidazolyl, 1,3,5-triazol-1-yl, N-maleimido, 2-(R′-imino)pyrrolidyl, pyrazin-2-on-1-yl, 3-oxazolidyl, 3-oxazolyl, 2-pyrrolyl, 3-chloro-1-pyrrolidyl, 2-nitro-1-imidazolyl, 4-methoxy-1-imidazolyl, and 3-methyl-1-imidazolyl.

(13) a derivative of amonafide of Formula (III) wherein Q is selected from the group consisting of Subformulas 3(h), 3(i), 3(j), 3(k), 3(1), 3(m), 3(n), 3(o), 3(p), 3(q), 3(r), and 3(s), wherein R′ is selected from the group consisting of alkyl, alkenyl, acyl, alkoxy, aryl, amino, substituted amino, sulfo, sulfamoyl, carboxyl, carbamyl, and cyano;

(14) a derivative of amonafide of Formula (III) wherein the naphthalimide ring is modified to include one or more amino groups at positions other than position 3 of the naphthalimide ring;

(15) a derivative of amonafide of Formula (III) wherein the amino group at position 3 is replaced with an alternative substituent group selected from the group consisting of alkyl, aryl, nitro, amino, substituted amino, sulfamoyl, halo, carboxyl, carbamyl, and cyano;

(16) a derivative of amonafide of Formula (III) wherein an additional group is attached to the naphthalimide ring also comprising an amino group at position 3, the additional group being selected from the group consisting of alkyl, aryl, nitro, substituted amino, sulfamoyl, halo, carboxyl, carbamyl, and cyano;

(17) an analog of amonafide wherein the naphthalene ring is replaced with one bearing one or more nitrogen atoms in either or both rings;

(18) an analog of amonafide that is an isoquinoline analog of Formula (IV) wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s);

(19) an analog of amonafide that is an isoquinoline analog of Formula (IV) wherein Q is —(CH₂)ₙ—N(CH₃)₂, wherein n is 1-12; and

(20) a derivative or analog of amonafide or of alternatives (1)-(19) including one or more optional substituents, provided that the optionally substituted amonafide derivative or analog possesses substantially equivalent pharmacological activity to amonafide as defined in terms of either or both topoisomerase II inhibition and DNA intercalation.

In another alternative, the derivative or analog of amonafide is selected from the group consisting of derivatives of amonafide, derivatives of azonafide, derivatives of mitonafide, and derivatives of elinafide.

In yet another alternative, the derivative or analog of amonafide is selected from the group consisting of heterocyclic-substituted bis-1,8-naphthalimide compounds, 1,8 naphthalimide imidazo{4,5,1-de}acridones, 2-substituted-1,2-dihydro-3H-dibenz[de,h]isoquinoline-1,3-diones, amino-substituted-[2′-(dimethylamino)ethyl]1,2-dihydro-3H-dibenz[de,h]isoquinoline-1,3-diones, tetrahydroazonafides, phenanthrene analogs of azonafide, and azaphenanthrenes.

Typically, when the suboptimally administered drug therapy is used to treat a hyperproliferative disease, the hyperproliferative disease is cancer. Methods according to the present invention and compositions according to the present invention suitable for use in those methods are applicable to many forms of cancer, including, but not limited to: (A) breast cancer, including: (1) ductal carcinoma, including ductal carcinoma in situ (DCIS) (comedocarcinoma, cribriform, papillary, micropapillary), infiltrating ductal carcinoma (IDC), tubular carcinoma, mucinous (colloid) carcinoma, papillary carcinoma, metaplastic carcinoma, and inflammatory carcinoma; (2) lobular carcinoma, including lobular carcinoma in situ (LCIS) and invasive lobular carcinoma; and (3) Paget's disease of the nipple; (B) cancers of the female reproductive system, including: (1) cancers of the cervix uteri, including cervical intraepithelial neoplasia (Grade I), cervical intraepithelial neoplasia (Grade II), cervical intraepithelial neoplasia (Grade III) (squamous cell carcinoma in situ), keratinizing squamous cell carcinoma, nonkeratinizing squamous cell carcinoma, verrucous carcinoma, adenocarcinoma in situ, adenocarcinoma in situ, endocervical type, endometrioid adenocarcinoma, clear cell adenocarcinoma, adenosquamous carcinoma, adenoid cystic carcinoma, small cell carcinoma, and undifferentiated carcinoma; (2) cancers of the corpus uteri, including endometrioid carcinoma, adenocarcinoma, adenocanthoma (adenocarcinoma with squamous metaplasia), adenosquamous carcinoma (mixed adenocarcinoma and squamous cell carcinoma, mucinous adenocarcinoma, serous adenocarcinoma, clear cell adenocarcinoma, squamous cell adenocarcinoma, and undifferentiated adenocarcinoma; (3) cancers of the ovary, including serous cystadenoma, serous cystadenocarcinoma, mucinous cystadenoma, mucinous cystadenocarcinoma, endometrioid tumor, endometrioid adenocarcinoma, clear cell tumor, clear cell cystadenocarcinoma, and unclassified tumor; (4) cancers of the vagina, including squamous cell carcinoma and adenocarcinoma; and (5) cancers of the vulva, including vulvar intraepithelial neoplasia (Grade I), vulvar intraepithelial neoplasia (Grade II), vulvar intraepithelial neoplasia (Grade III) (squamous cell carcinoma in situ); squamous cell carcinoma, verrucous carcinoma, Paget's disease of the vulva, adenocarcinoma (NOS), basal cell carcinoma (NOS), and Bartholin's gland carcinoma; (C) cancers of the male reproductive system, including: (1) cancers of the penis, including squamous cell carcinoma; (2) cancers of the prostate, including adenocarcinoma, sarcoma, and transitional cell carcinoma of the prostate; (3) cancers of the testis, including seminomatous tumor, nonseminomatous tumor, teratoma, embryonal carcinoma, yolk sac tumor, and Choriocarcinoma; (D) cancers of the cardiac system, including sarcoma (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma, and squamous cell carcinoma of the pharynx; (F) cancers of the lung, including squamous cell carcinoma (epidermoid carcinoma), variants of squamous cell carcinoma, spindle cell carcinoma, small cell carcinoma, carcinoma of other cells, carcinoma of intermediate cell type, combined oat cell carcinoma, adenocarcinoma, acinar adenocarcinoma, papillary adenocarcinoma, bronchiolo-alveolar carcinoma, solid carcinoma with mucus formation, large cell carcinoma, giant cell carcinoma, clear cell carcinoma, and sarcoma; (G) cancers of the gastrointestinal tract, including: (1) cancers of the ampulla of Vater, including primary adenocarcinoma, carcinoid tumor, and lymphoma; (2) cancers of the anal canal, including adenocarcinoma, squamous cell carcinoma, and melanoma; (3) cancers of the extrahepatic bile ducts, including carcinoma in situ, adenocarcinoma, papillary adenocarcinoma, adenocarcinoma, intestinal type, mucinous adenocarcinoma, clear cell adenocarcinoma, signet-ring cell carcinoma, adenosquamous carcinoma, squamous cell carcinoma, small cell (oat) carcinoma, undifferentiated carcinoma, carcinoma (NOS), sarcoma, and carcinoid tumor; (4) cancers of the colon and rectum, including adenocarcinoma in situ, adenocarcinoma, mucinous adenocarcinoma (colloid type; greater than 50% mucinous carcinoma), signet ring cell carcinoma (greater than 50% signet ring cell), squamous cell (epidermoid) carcinoma, adenosquamous carcinoma, small cell (oat cell) carcinoma, undifferentiated carcinoma, carcinoma (NOS), sarcoma, lymphoma, and carcinoid tumor; (5) cancers of the esophagus, including squamous cell carcinoma, adenocarcinoma, leiomyosarcoma, and lymphoma; (6) cancers of the gallbladder, including adenocarcinoma, adenocarcinoma, intestinal type, adenosquamous carcinoma, carcinoma in situ, carcinoma (NOS), clear cell adenocarcinoma, mucinous adenocarcinoma, papillary adenocarcinoma, signet-ring cell carcinoma, small cell (oat cell) carcinoma, squamous cell carcinoma, and undifferentiated carcinoma; (7) cancers of the lip and oral cavity, including squamous cell carcinoma; (8) cancers of the liver, including hepatoma (hepatocellular carcinoma), cholangiocarcinoma, hepatoblastoma, angiosarcoma, hepatocellular adenoma, and hemangioma; (9) cancers of the exocrine pancreas, including duct cell carcinoma, pleomorphic giant cell carcinoma, giant cell carcinoma, osteoclastoid type, adenocarcinoma, adenosquamous carcinoma, mucinous (colloid) carcinoma, cystadenocarcinoma, acinar cell carcinoma, papillary carcinoma, small cell (oat cell) carcinoma, mixed cell typed, carcinoma (NOS), undifferentiated carcinoma, endocrine cell tumors arising in the islets of Langerhans, and carcinoid; (10) cancers of the salivary glands, including acinic (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well differentiated or low grade), and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenosquamous carcinoma, squamous cell carcinoma, small cell carcinoma, undifferentiated carcinoma, lymphoma, sarcoma, and carcinoid tumor; and (12) cancers of the small intestine, including adenocarcinoma, lymphoma, carcinoid tumors, Kaposi's sarcoma, leiomyoma, hemangioma, lipoma, neurofibroma, and fibroma; (H) cancers of the urinary system, including: (1) cancers of the kidney, including renal cell carcinoma, carcinoma of Bellini's collecting ducts, adenocarcinoma, papillary carcinoma, tubular carcinoma, granular cell carcinoma, clear cell carcinoma (hypernephroma), sarcoma of the kidney, and nephroblastoma; (2) cancers of the renal pelvis and ureter, including transitional cell carcinoma, papillary transitional cell carcinoma, squamous cell carcinoma, and adenocarcinoma; (3) cancers of the urethra, including transitional cell carcinoma, squamous cell carcinoma, and adenocarcinoma; and (4) cancers of the urinary bladder, including carcinoma in situ, transitional urothelial cell carcinoma, papillary transitional cell carcinoma, squamous cell carcinoma, adenocarcinoma, undifferentiated; (I) cancers of muscle, bone, and soft tissue, including: (1) cancers of bone, including: (a) bone-forming: osteosarcoma; (b) cartilage-forming: chondrosarcoma and mesenchymal chondrosarcoma; (c) giant cell tumor, malignant; (d) Ewing's sarcoma; (e) vascular tumors: hemangioendothelioma, hemangiopericytoma, and angiosarcoma; (f) connective tissue tumors: fibrosarcoma, liposarcoma, malignant mesenchymoma, and undifferentiated sarcoma; and (g) other tumors: chordoma and adamantinoma of long bones; (2) cancers of soft tissues, including: alveolar soft-part sarcoma, angiosarcoma, epithelioid sarcoma, extraskeletal chondrosarcoma, fibrosarcoma, leiomyosarcoma, liposarcoma, malignant fibrous histiocytoma, malignant hemangiopericytoma, malignant mesenchymoma, malignant schwannoma, rhabdomyosarcoma, synovial sarcoma, and sarcoma (NOS); (3) cancers of the nervous system, including cancers of the skull (osteoma, hemangioma, granuloma, xanthoma, osteitis deformans), cancers of the meninges (meningioma, meningiosarcoma, gliomatosis), cancers of the brain (astrocytoma, medulloblastoma, glioma, ependymoma, germinoma (pilealoma), glioblastoma multiforme, oligodendroglioma, schwannoma, retinoblastoma, congenital tumors), and cancers of the spinal cord neurofibroma, meningioma, glioma, sarcoma); (4) hematologic cancers, including myeloid leukemia (acute and chronic), acute lymphoblastic leukemia, chronic lymphocytic leukemia, myeloproliferative diseases, multiple myeloma; myelodysplastic syndrome), Hodgkin's disease, and non-Hodgkin's lymphoma (malignant lymphoma); (5) cancers of the endocrine system, including: (a) cancers of the thyroid gland, including papillary carcinoma (including those with follicular foci), follicular carcinoma, medullary carcinoma, and undifferentiated (anaplastic) carcinoma; and (b) neuroblastomas, including sympathicoblastoma, sympathicogonioma, malignant ganglioneuroma, gangliosympathicoblastoma, and ganglioneuroma; (6) cancers of the skin, including squamous cell carcinoma, spindle cell variant of squamous cell carcinoma, basal cell carcinoma, adenocarcinoma developing from sweat or sebaceous gland, and malignant melanoma; (7) cancers of the eye, including: (a) cancers of the conjunctiva, including carcinoma of the conjunctiva; (b) cancers of the eyelid, including basal cell carcinoma, squamous cell carcinoma, melanoma of the eyelid, and sebaceous cell carcinoma; (c) cancers of the lacrimal gland, including adenocarcinoma, adenoid cystic carcinoma, carcinoma in pleomorphic adenoma, mucoepidermoid carcinoma, and squamous cell carcinoma; (d) cancers of the uvea, including spindle cell melanoma, mixed cell melanoma, and epithelioid cell melanoma; (e) cancers of the orbit, including sarcoma of the orbit, soft tissue tumor, and sarcoma of bone; and (f) retinoblastoma. In particular, methods according to the present invention and compositions according to the present invention are particularly suitable for the treatment of the following types of cancers: (1) melanoma; (2) colon cancer; (3) chronic lymphocytic leukemia; (4) skin cancer; (5) lung cancer, including small-cell lung cancer and non-small-cell lung cancer; (6) throat cancer; (7) stomach cancer; (8) salivary gland cancer; (9) breast cancer, including triple-negative breast cancer and breast cancer characterized by overexpression of Her-2/neu; (10) prostate cancer, including androgen-resistant prostate cancer; (11) pancreatic cancer; (12) ovarian cancer; (13) uterine cancer; (14) endometrial cancer; (15) other leukemias; (16) renal cell carcinoma; (17) multiple myeloma; (18) liver cancer; (19) pituitary gland cancer; (20) acute myeloid leukemia; (21) oophoroma; (22) glioma; (23) head and neck cancer; (23) colorectal cancer; (24) bladder cancer; (25) HPV-induced papilloma; (26) lymphoma, including both non-Hodgkin's lymphoma and Hodgkin's lymphoma; (27) myelodysplastic syndrome; (28) chronic myelocytic leukemia, including treatment of chronic myelocytic leukemia subsequent to the administration of homoharringtonine; (29) malignancies with overexpressed or mutated EGFR; (30) malignancies with overexpressed or mutated Her2/neu; (31) malignancies with overexpressed or mutated Braf; (32) malignancies with overexpressed or mutated BTK; (33) malignancies with overexpressed or mutated KRAS; (34) malignancies with overexpressed or mutated c-Myc; and (35) malignancies with overexpressed or mutated p53. In addition, methods according to the present invention and compositions according to the present invention are also particularly suitable for treatment of several non-malignant proliferative conditions, including psoriasis and HSV-induced shingles.

The following improvements all apply either to amonafide itself or derivatives or analogs of amonafide as indicated with respect to the specific improvement indicated below, unless either amonafide or derivatives or analogs of amonafide are specifically indicated.

When the improvement is made by dose modification, the dose modification can be, but is not limited to, at least one dose modification selected from the group consisting of:
(a) continuous i.v. infusion for hours to days;
(b) biweekly administration;
(c) doses greater than 5 mg/m²/day;
(d) progressive escalation of dosing from 1 mg/m²/day based on patient tolerance;
(e) doses less than 1 mg/m² for greater than 14 days;
(f) use of caffeine to modulate metabolism;
(g) use of isoniazid to modulate metabolism;
(h) selected and intermittent boost dose administrations;
(i) bolus single and multiple doses of 1-5 mg/m²;
(j) oral dosing including multiple daily dosing;
(k) micro-dosing;
(l) immediate release dosing;
(m) slow release dosing; and
(n) controlled release dosing.

When the improvement is made by route of administration, the route of administration can be, but is not limited to, a route of administration selected from the group consisting of:
(a) topical administration;
(b) intravesicular administration for bladder cancer;
(c) oral administration;
(d) slow release oral delivery;
(e) intrathecal administration;
(f) intraarterial administration;
(g) continuous infusion; and
(h) intermittent infusion.

When the improvement is made by schedule of administration, the schedule of administration can be, but is not limited to, a schedule of administration selected from the group consisting of:
(a) daily administration;
(b) weekly administration for three weeks;
(c) weekly administration for two weeks;
(d) biweekly administration;
(e) biweekly administration for three weeks with a 1-2 week rest period;
(f) intermittent boost dose administration; and
(g) administration daily for one week then once per week for multiple weeks.

When the improvement is made by an indication for use, the indication for use can be, but is not limited to, an indication for use selected from the group consisting of:
(a) use for treatment of triple-negative breast cancer;
(b) use for treatment of acute leukemias;
(c) use for treatment of chronic myelocytic leukemia (CML), either subsequent to or in combination with the administration of tyrosine kinase inhibitors or homoharringtonine;
(d) use for treatment of chronic lymphocytic leukemia;
(e) use for treatment of Hodgkin's lymphoma;
(f) use for treatment of non-Hodgkin's lymphoma;
(g) use for treatment of mycosis fungoides;
(h) use for treatment of prostate cancer, especially androgen-resistant prostate cancer;
(i) use for treatment of lung small-cell carcinoma, either subsequent to or in combination with the administration of EGFR inhibitors such as erlotinib (Tarceva) or gefitinib (Iressa), wherein the lung small-cell carcinoma is characterized by either wild-type or mutated EGFR;
(j) use for treatment of lung non-small cell carcinoma, subsequent to or in combination with EGFR inhibitors such as erlotinib or gefitinib, wherein the lung non-small cell carcinoma is characterized by either wild-type or mutated EGFR;
(k) use for treatment of breast cancer characterized by overexpressed Her-2-neu;
(l) use for treatment of glioblastoma that is resistant to one or both of the following therapeutic agents: temozolomide (Temodar) or bevacizumab (Avastin), or is characterized by EGFR variant III, either alone or in combination with other therapeutic agents;
(m) use for treatment of malignancies characterized by overexpressed topoisomerase II;
(n) use for treatment of malignancies characterized by overexpressed and/or mutated EGFR;
(o) use for treatment of prostate cancer;
(p) use for treatment of malignancies characterized by overexpressed and/or mutated Her2/neu;
(q) use for treatment of malignancies characterized by overexpressed and/or mutated Braf;
(r) use for treatment of malignancies characterized by overexpressed and/or mutated BTK;
(s) use for treatment of malignancies characterized by overexpressed and/or mutated KRAS;
(t) use for treatment of malignancies characterized by overexpressed and/or mutated c-Myc;
(u) use for treatment of malignancies characterized by overexpressed and/or mutated p53;
(v) use for treatment of myelodysplastic syndrome;
(w) use for treatment of angiogenic diseases;
(x) use for treatment of benign prostate hypertrophy;
(y) use for treatment of psoriasis;
(z) use for treatment of gout;
(aa) use for treatment of autoimmune conditions;
(ab) use for prevention of transplantation rejection;
(ac) use for restenosis prevention in cardiovascular disease;
(ad) use in bone marrow transplantation;
(ae) use as an anti-infective; and
(af) use in treatment for AIDS.

Triple-negative breast cancer is a form of breast cancer that is characterized by tumors that do not express estrogen receptor (ER), progesterone receptor (PR), or HER-2 genes. This form of breast cancer represents an important clinical challenge because these cancers do not respond to endocrine therapy or a number of targeted agents. Current treatment strategies for triple-negative breast cancer include many chemotherapy agents, such as the anthracyclines, taxanes, ixabepilone, and platinum agents, as well as selected biologic agents and possibly anti-EGFR drugs.

Tyrosine kinase inhibitors used for treatment of chronic myelocytic leukemia (CML) include, but are not limited to, imatinib, bosutinib, nilotinib, dasatinib, erlotinib, afatinib, and dacomitinib. Additional tyrosine kinase inhibitors are known in the art. For example, the use of tyrosine kinase inhibitors is described in United States Patent Application Publication No. 2011/0206661 by Zhang et al., which is directed to trimethoxyphenyl inhibitors of tyrosine kinase, and in United States Patent Application Publication No. 2011/0195066, which is directed to quinoline inhibitors of tyrosine kinase, both of which are incorporated herein by this reference. The use of tyrosine kinase inhibitors is also described in United States Patent Application Publication No. 2011/053968 by Zhang et al., incorporated herein by this reference, which is directed to aminopyridine inhibitors of tyrosine kinase. The use of tyrosine kinase inhibitors is also described in United States Patent Application Publication No. 2010/0291025, incorporated herein by this reference, which is directed to indazole inhibitors of tyrosine kinase. The use of tyrosine kinase inhibitors is also described in United States Patent Application Publication No. 2010/0190749 by Ren et al., incorporated herein by this reference; these tyrosine kinase inhibitors are benzoxazole compounds; compounds of this class can also inhibit mTOR and lipid kinases such as phosphoinositide 3-kinases. The use of tyrosine kinase inhibitors is also described in U.S. Pat. No. 8,242,270 by Lajeunesse et al., incorporated herein by this reference; these tyrosine kinase inhibitors are 2-aminothiazole-5-aromatic carboxamides. Still other tyrosine kinase inhibitors are known in the art or are under development, and are described in B. J. Druker & N. B. Lydon, “Lessons Learned from the Development of an Abl Tyrosine Kinase Inhibitor for Chronic Myelogenous Leukemia,” J. Clin. Invest. 105: 3-7 (2000), incorporated herein by this reference.

Homoharringtonine (omacetaxine mepesuccinate) has the structure shown below:

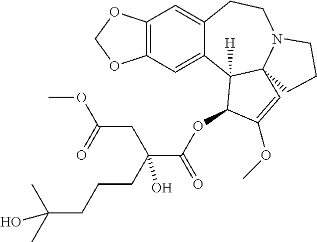

and is a protein translation inhibitor. Homoharringtonine inhibits protein translation by preventing the initial elongation step of protein synthesis. It interacts with the ribosomal A-site and prevents the correct positioning of amino acid side chains of incoming aminoacyl-tRNAs.

Androgen-resistant prostate cancer, also known as castration-resistant prostate cancer, is characterized by reactivation of androgen-regulated processes and is detectable by an increase in prostate-specific antigen (PSA) despite the administration of androgen deprivation therapy; it has been suggested that sufficient androgens remain available even subsequent to the administration of androgen deprivation therapy through reactions employing progesterone as a starting material for the synthesis of dihydrotestosterone (J. A. Locke et al., “Androgen Levels Increase by Intratumoral De Novo Steroidogenesis During Progression of Castration-Resistant Prostate Cancer,” Cancer Res. 68: 6407-6415 (2008), incorporated herein by this reference).

EGFR inhibitors include, but are not limited to, erlotinib (Tarceva) and gefitinib (Iressa). These EGFR inhibitors specifically inhibit the EGFR tyrosine kinase. Mutations in the EGFR gene may affect the sensitivity of EGFR to EGFR inhibitors such as erlotinib and gefitinib. At least some of these mutations may increase sensitivity to EGFR inhibitors (J. G. Paez et al., “EGFR Mutations in Lung Cancer: Correlation with Clinical Response to Gefitinib Therapy,” Science 304: 1497-1500 (2004), incorporated herein by this reference; R. Sordella et al., “Gefitinib-Sensitizing EGFR Mutations in Lung Cancer Activate Anti-Apoptotic Pathways,” Science 305: 1163-1167 (2005), incorporated herein by this reference). However, relapses are frequent; at least some relapses are associated with a mutation at amino acid 790 of EGFR in which threonine is changed to methione (T790M) (S. Kobayashi et al., “EGFR Mutation and Resistance of Non-Small-Cell Lung Cancer to Gefitinib,” New Engl. J. Med. 352: 786-792 (2005), incorporated herein by this reference).

Other EGFR inhibitors are known in the art. EGFR inhibitors include, but are not limited to, erlotinib, gefitinib, lapatinib, lapatinib ditosylate, afatinib, canertinib, neratinib, (E)-2-methoxy-N-(3-(4-(3-methyl-4-(6-methylpyridin-3-yloxy)phenylamino)quinazolin-6-yl)allyl)acetamide (CP-724,714), 2-[(3,4-dihydroxyphenyl)methylene]-propanedinitrile (AG 18), 2-bromo-4-[(6,7-dimethoxy-4-quinazolinyl)amino]-phenol (WHI-P154), N-(2-(4-(3-chloro-4-(3-(trifluoromethyl)phenoxy)phenylamino)-5H-pyrrolo[3,2-d]pyrimidin-5-yl)ethyl)-3-hydroxy-3-methylbutanamide (TAK-285), N-[4-[[3-chloro-4-[(3-fluorophenyl)methoxy]phenyl]amino]-6-quinazolinyl]-2-propenamide 4-methylbenzenesulfonate (AST-1306), (R)—N4-(3-chloro-4-(thiazol-2-ylmethoxy)phenyl)-N6-(4-methyl-4,5-dihydrooxazol-2-yl)quinazoline-4,6-diamine (ARRY334543), icotinib, N-(3-chlorophenyl)-6,7-dimethoxyquinazolin-4-amine (AG-1478), 2-[[3,5-bis(1,1-dimethylethyl)-4-hydroxyphenyl]methylene]-propanedinitrile (SF 6847), dacomitinib, desmethyl erlotinib, 2-(4-(3-ethynylphenylamino)-7-(2-methoxyethoxy)quinazolin-6-yloxy)ethanol hydrochloride (OSI-420), N-(3-(5-chloro-2-(4-(4-methylpiperazin-1-yl)phenylamino)pyrimidin-4-ylthio)phenyl)acrylamide (WZ-8040), N-(3-(5-chloro-2-(2-methoxy-4-(4-methylpiperazin-1-yl)phenylamino)pyrimidin-4-yloxy)phenyl)acrylamide (WZ4002), N-(3-(5-chloro-2-(4-(4-methylpiperazin-1-yl)phenylamino)pyrimidin-4-yloxy)phenyl)acrylamide (WZ3146), (E)-N-benzyl-2-cyano-3-(3,4-dihydroxyphenyl)acrylamide (AG-490), N-(3,4-dichloro-2-fluorophenyl)-6-methoxy-7-(((3aR,5r,6aS)-2-methyl-octahydrocyclopenta[c]pyrrol-5-yl)methoxy)quinazolin-4-amine (XL647), N-(3-bromophenyl)-6,7-dimethoxyquinazolin-4-amine hydrochloride (PD153035), and (S)-morpholin-3-ylmethyl 4-(1-(3-fluorobenzyl)-1H-indazol-5-ylamino)-5-methylpyrrolo[1,2-f][1,2,4]triazin-6-ylcarbamate (BMS-599626). Still other EGFR inhibitors are known in the art, including monoclonal antibodies and derivatives thereof. Such monoclonal antibodies and derivatives thereof include cetuximab, panitumumab, matuzumab, nimotuzumab, trastuzumab, zalutumumab, and zatuximab. In addition, such monoclonal antibodies and derivatives thereof can be conjugated to therapeutic agents such as toxins or radionuclides. The conjugation of monoclonal antibodies to radionuclides is described in K. K. Bhargava & S. A. Acharya, “Labeling of Monoclonal Antibodies with Radionuclides,” Semin. Nucl. Med. 19: 187-201 (1989), incorporated herein by this reference. The conjugation of monoclonal antibodies to non-radionucleotide therapeutic agents is described in P. Chames et al., “Therapeutic Antibodies: Successes, Limitations, and Hopes for the Future,” Br. J. Pharmacol. 157: 220-233 (2009), incorporated herein by this reference. The non-radionuclide therapeutic agents can include, a fragment of Pseudomonas exotoxin, diphtheria toxin, the A chain of ricin, Staphylococcus aureus enterotoxin, mertansine, a calicheamicin cytotoxic agent, interleukin-2, and other agents known in the art. Monoclonal antibodies can also be fused to effector proteins and membrane proteins. As used herein in this context, the term “monoclonal antibodies” includes, but is not limited to, chimeric antibodies, humanized antibodies, antibody fragments such as scFv fragments, diabodies, heavy chain antibodies (HcAbs), and single-domain antibodies (sdAbs). Suh monoclonal antibodies are not necessarily produced as the result of cell fusion between B cells and myeloma cells, and can be produced in other eukaryotic cells or even bacterial cells according to methods known in the art.

Additional EGFR inhibitors, including derivatives of erlotinib and salts thereof, are described in United States Patent Application Publication No. 2013/0012528 by Cheng, incorporated herein by this reference.

Overexpression of Her-2/neu, particularly in breast cancer, is associated in some cases with advanced disease and relative resistance to conventional chemotherapy. In such cases, the use of cisplatin plus a recombinant humanized anti-p185HER2 monoclonal antibody has been suggested (M. D. Pegram et al., “Phase II Study of Receptor-Enhanced Chemosensitivity Using Recombinant Humanized Anti-p185HER2/neu Monoclonal Antibody Plus Cisplatin in Patients with HER2/neu Overexpressing Metastatic Breast Cancer Refractory to Chemotherapy Treatment,” J. Clin. Oncol. 16: 2659-2671 (1998), incorporated herein by this reference). The overexpression of Her-2/neu is also associated with changes in the regulation of a number of genes, including proline 4-hydroxylase, galectin 1, galectin 3, fibronectin 1, p-cadherin, which are genes involved in cell-matrix interactions, and genes involved with cell proliferation and transformation. A number of genes associated with MYC signaling were also differentially expressed (A. Mackay et al., “cDNA Microarray Analysis of Genes Associated with ERBB2 (HER2/neu) Overexpression in Human Mammary Luminal Epithelial Cells,” Oncogene 22: 2680-2688 (2003), incorporated herein by this reference).

EGFR variant III is a variant of EGFR that does not respond to gefitinib; cells possessing the variant do not show reduction of phosphorylation subsequent to treatment with gefitinib. Additionally, although such cells may show a degree of reduction of phosphorylation of EGFR after more extended treatment with gefitinib, these cells continue to be resistant to the antineoplastic effects of gefitinib, possibly because the phosphorylation of Akt is unaffected in cells with variant III while being inhibited in EGFR-expressing cells after treatment with gefitinib (C. A. Learn, “Resistance to Tyrosine Kinase Inhibition by Mutant Epidermal Growth Factor Receptor Variant III Contributes to the Neoplastic Phenotype of Glioblastoma Multiforme,” Clin. Cancer Res. 10: 3216-3224 (2004), incorporated herein by this reference). Conventional treatments for glioblastoma include temozolomide, frequently administered with radiotherapy, bevacizumab (Avastin), and the protein therapeutic APG 101.

Braf, more specifically serine/threonine-protein kinase B-Raf, is a signal-transducing kinase that is mutated in some human cancers (H. Davis et al., “Mutations of the BRAF Gene in Human Cancers,” Nature 417: 949-954 (2002), incorporated herein by this reference). One particular mutation, V600E, substitutes glutamic acid for valine at position 600 of the protein. Other mutations are known to exist. One drug useful for treating cancers with the V600E mutation is vemurafenib.

Bruton's tyrosine kinase (BTK) is a tyrosine kinase that plays a key role in B-cell maturation. Ibrutinib is a selective BTK inhibitor.

KRAS is a GTPase that acts as a molecular on-off switch; it can be activated in various malignancies (O. Kranenburg, “The KRAS Oncogene: Past, Present, and Future,” Biochim. Biophys. Acta 1756: 81-82 (2005), incorporated herein by this reference). The KRAS gene may be amplified in colorectal cancer, among other types of malignancies.

The gene c-Myc is a regulator gene that codes for a transcription factor and is frequently mutated in malignancies, including carcinoma of the cervix, colon, breast, lung, and stomach. It also may be amplified in malignancies, including ovarian cancer. Its activity is described in R. Cotterman et al., “N-Myc Regulates a Widespread Euchromatic Program in the Human Genome Partially Independent of Its Role as a Classical Transcription Factor,” Cancer Res. 68: 9654-9662 (2008), incorporated herein by this reference.

Tumor protein p53 is a protein encoded by the TP53 gene in humans. Mutations or deletions of p53 are frequently associated with malignancies. The role of p53 is described in K. M. Leung et al., “The Candidate Tumor Suppressor ING1 b Can Stabilize p53 by Disrupting the Regulation of p53 by MDM2,” Cancer Res. 68: 4890-4893 (2002)

When the improvement is made by selection of disease stage, the selection of disease stage can be, but is not limited to, at least one selection of disease stage selected from the group consisting of:
(a) use for the treatment of localized polyp stage colon cancer;
(b) use for the treatment of leukoplakia in the oral cavity;
(c) use to induce angiogenesis inhibition to prevent or limit metastatic spread; and
(d) use against HIV with AZT, DDI, or reverse transcriptase inhibitors.

When the improvement is made by other indications, the other indications can be, but are not limited to, at least one other indication selected from the group consisting of:
(a) use as an anti-infective agent;
(b) use as an antiviral agent;
(c) use as an antibacterial agent;
(d) use for control of pleural effusions;
(e) use as an antifungal agent;
(f) use as an antiparasitic agent;
(g) use for treatment of eczema;
(h) use for treatment of shingles;
(i) use for treatment of condylomata;
(j) use for treatment of human papilloma virus (HPV); and
(k) use for treatment of herpes simplex virus (HSV).

When the improvement is made by patient selection, the patient selection can be, but is not limited to, a patient selection carried out by a criterion selected from the group consisting of:
(a) selecting patients with a disease condition characterized by a high level of a metabolic enzyme selected from the group consisting of histone deacetylase, protein kinases, and ornithine decarboxylase;
(b) selecting patients with a disease condition characterized by a low level of a metabolic enzyme selected from the group consisting of histone deacetylase, protein kinases, and ornithine decarboxylase;
(c) selecting patients with a low or high susceptibility to a condition selected from the group consisting of thrombocytopenia and neutropenia;
(d) selecting patients intolerant of GI toxicities; and
(e) selecting patients characterized by over- or under-expression of a gene selected from the group consisting of jun, GPCRs, signal transduction proteins, VEGF, prostate specific genes, protein kinases, and telomerase.

The cellular proto-oncogene c-Jun encodes a protein that, in combination with c-Fos, forms the AP-1 early response transcription factor. This proto-oncogene plays a key role in transcription and interacts with a large number of proteins affecting transcription and gene expression. It is also involved in proliferation and apoptosis of cells that form part of a number of tissues, including cells of the endometrium and glandular epithelial cells. G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994), incorporated herein by this reference. GPCR receptors include, but are not limited to, acetylcholine receptors, β-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors.

When the improvement is made by analysis of patient or disease phenotype, the analysis of patient or disease phenotype can be, but is not limited to, a method of analysis of patient or disease phenotype carried out by a method selected from the group consisting of:
(a) use of a diagnostic tool, a diagnostic technique, a diagnostic kit, or a diagnostic assay to confirm a patient's particular phenotype;
(b) use of a method for measurement of a marker selected from the group consisting of histone deacetylase, ornithine decarboxylase, VEGF, a protein that is a gene product of a prostate specific gene, a protein that is a gene product of jun, and a protein kinase;
(c) surrogate compound dosing;
(d) low dose pre-testing for enzymatic status; and
(e) use of a method to determine the phenotype for N-acetyltransferase activity.

When the improvement is made by analysis of patient or disease genotype, the analysis of patient or disease genotype can be, but is not limited to, a method of analysis of patient or disease genotype carried out by a method selected from the group consisting of:
(a) use of a diagnostic tool, a diagnostic technique, a diagnostic kit, or a diagnostic assay to confirm a patient's particular genotype;
(b) use of a gene chip;
(c) use of gene expression analysis;
(d) use of single nucleotide polymorphism (SNP) analysis;
(e) measurement of the level of a metabolite or a metabolic enzyme; and
(f) use of a method to determine the genotype for N-acetyltransferase activity.

The use of gene chips is described in A. J. Lee & S. Ramaswamy, “DNA Microarrays in Biological Discovery and Patient Care” in Essentials of Genomic and Personalized Medicine (G. S. Ginsburg & H. F. Willard, eds., Academic Press, Amsterdam, 2010), ch. 7, pp. 73-88, incorporated herein by this reference).

When the method is the use of single nucleotide polymorphism (SNP) analysis, the SNP analysis can be carried out on a gene selected from the group consisting of histone deacetylase, ornithine decarboxylase, VEGF, a prostate specific gene, c-Jun, and a protein kinase. The use of SNP analysis is described in S. Levy and Y.-H. Rogers, “DNA Sequencing for the Detection of Human Genome Variation” in Essentials of Genomic and Personalized Medicine (G. S. Ginsburg & H. F. Willard, eds., Academic Press, Amsterdam, 2010), ch. 3, pp. 27-37, incorporated herein by this reference.

Still other genomic techniques such as copy number variation analysis and analysis of DNA methylation can be employed. Copy number variation analysis is described in C. Lee et al., “Copy Number Variation and Human Health” in Essentials of Genomic and Personalized Medicine (G. S. Ginsburg & H. F. Willard, eds., Academic Press, Amsterdam, 2010), ch. 5, pp. 46-59, incorporated herein by this reference. DNA methylation analysis is described in S. Cottrell et al., “DNA Methylation Analysis: Providing New Insight into Human Disease” in Essentials of Genomic and Personalized Medicine (G. S. Ginsburg & H. F. Willard, eds., Academic Press, Amsterdam, 2010), ch. 6, pp. 60-72, incorporated herein by this reference.

The use of N-acetyl transferase genotyping to determine effective and nontoxic dosing for naphthalimides, including amonafide, is described in United States Patent Publication No. 2011/0003742 to Brown, incorporated herein by this reference. In particular, this genotyping is intended to reduce the potential occurrence of leukocytopenia. Naphthalimides, such as amonafide, are metabolically processed. The first step of metabolism is to acylate the naphthalimide by way of N-acetyl transferase (NAT). In one aspect, the invention utilizes an assay to genotype a patient to determine whether he falls within one of two phenotypes: (1) slow acylators of naphthalimide or (2) fast acylators which include either the rapid (R) homozygous or intermediate (I) genotype. In most cases, the fast phenotype includes heterozygous genotypes of rapid and intermediate NAT-2 genes. See D. W. Hein et al., “Molecular Genetics and Epidemiology of the NAT1 and NAT2 Acetylation Polymorphisms,” Cancer Epidemiology, Biomarkers & Prevention Vol. 9, 29-42, January 2000, incorporated herein by this reference; the intermediate type is heterozygous, while the rapid type is homozygous. Acetyl naphthalimide, in general, has significant anti-tumor activity. However, the acylated naphthalimide, e.g., acetyl amonafide, has a profound impact upon the white blood cell count (WBC) of the patient. In particular, acetyl amonafide has been shown to induce leukocytopenia and, in particular, granulocytopenia. By genotyping a patient prior to treatment, it is possible to determine the dosage levels and intervals of naphthalimide administration so as to minimize leukocytopenia, thereby controlling a significant toxic side effect. For example, a slow acylator will have lower levels of acylated naphthalimide and a lower ratio of acylated naphthalimide to naphthalimide as compared to a fast acylator. Such patients are least likely to present a severe leukocytopenia. Accordingly, such patients can tolerate an increase in the normal dosage of the naphthalimide for treatment. On the other hand, a fast acylator will have a higher level of acylated naphthalimide and a higher ratio of acylated naphthalimide as compared to naphthalimide. Such patients are more likely to present a significant leukocytopenia upon treatment with naphthalimide. In such cases, the dose of the naphthalimide can be decreased based on the genotype prior to administration so as to reduce the likelihood of severe leukocytopenia. For example, the dosage of a naphthalimide such as amonafide, for a slow acylator would be in the range of 300-1000 mg/m², more preferably between 400 and 600 mg/m², and most preferably between 450 and 550 mg/m². In the case of the fast acylator, naphthalimide dosages would be reduced to between 50 and 450 mg/m², more preferably between 150 and 450 mg/m², and most preferably between 350 and 450 mg/m². In the case of the fast acylator, these dosages can be increased when used in conjunction with GCSF and may be as high as the dosage for the slow acylator. In addition to the foregoing, the fast or slow acylator genotype of the patient may also be used to dose the patient with anti-leukocytopenia agents such as granulocyte colony stimulating factor (GCSF) also referred to as Neupogen® from Amgen, Thousand Oaks, Calif. Accordingly, higher doses of GCSF are called for in case of fast acylators that are treated with naphthalimide. Alternatively, in the case of slow acylators, the dosage of GCSF can be reduced or eliminated entirely in the naphthalimide treatment regime. The use of genotyping patients prospectively to identify fast and slow acylator phenotypes provides the opportunity to selectively employ GCSF, to boost neutrophil counts for patients at greater risk for neutropenia (e.g., rapid acylators can be dosed above 300 mg/m²/week). In these cases, the potential for increased naphthalimide doses may be boosted if the GCSF maintains relatively normal leukocyte levels. In addition, for slow acylators, the opportunity to increase naphthalimide doses above, for example, 600 mg/m²/week may also exist if GCSF can be used. With the identification of a patient's NAT-2 genotype, GCSF therapy may be initiated prior to the initiation of naphthalimide in an effort to increase leukocyte count to prevent the myelosuppressive effects of the naphthalimide. The GCSF, for example, administered either intravenously or subcutaneously at doses ranging from 3-10 μg/kg given daily could boost the leucocyte count such that vulnerable rapid acylators could safely receive the established doses for that phenotype but may allow for the opportunity to increase naphthalimide dosages and/or the frequency of dosing (e.g., daily, two times per week, etc.). The same opportunity may also exist for slow acylators where ultra high dosing (e.g., >650 mg/m²) may be achieved with GCSF supportive therapy.

When the improvement is made by pre/post-treatment preparation, the pre/post-treatment preparation can be, but is not limited to, a method of pre/post treatment preparation selected from the group consisting of:
(a) the use of colchicine or an analog thereof;
(b) the use of a uricosuric;
(c) the use of uricase;
(d) the non-oral use of nicotinamide;
(e) the use of a sustained-release form of nicotinamide;
(f) the use of an inhibitor of poly-ADP ribose polymerase;
(g) the use of caffeine;
(h) the use of leucovorin rescue;
(i) infection control; and
(j) the use of an anti-hypertensive agent.

Uricosurics include, but are not limited to, probenecid, benzbromarone, and sulfinpyrazone. A particularly preferred uricosuric is probenecid. Uricosurics, including probenecid, may also have diuretic activity.

Poly-ADP ribose polymerase inhibitors are described in G. J. Southan & C. Szabó, “Poly(ADP-Ribose) Inhibitors,” Curr. Med. Chem. 10: 321-240 (2003), incorporated herein by this reference, and include nicotinamide, 3-aminobenzamide, substituted 3,4-dihydroisoquinolin-1(2H)-ones and isoquinolin-1(2H)-ones, benzimidazoles, indoles, phthalazin-1(2H)-ones, quinazolinones, isoindolinones, phenanthridinones, and other compounds.

Leucovorin rescue comprises administration of folinic acid (leucovorin) to patients in which methotrexate has been administered. Leucovorin is a reduced form of folic acid that bypasses dihydrofolate reductase and restores hematopoietic function. Leucovorin can be administered either intravenously or orally.

In one alternative, wherein the pre/post treatment is the use of a uricosuric, the uricosuric is probenecid or an analog thereof.

When the improvement is made by toxicity management, the toxicity management can be, but is not limited to, a method of toxicity management selected from the group consisting of:
(a) the use of colchicine or an analog thereof;
(b) the use of a uricosuric;
(c) the use of uricase;
(d) the non-oral use of nicotinamide;
(e) the use of a sustained-release form of nicotinamide;
(f) the use of an inhibitor of polyADP-ribose polymerase;
(g) the use of caffeine;
(h) the use of leucovorin rescue;
(i) the use of sustained-release allopurinol;
(j) the non-oral use of allopurinol;
(k) the administration of bone marrow transplant stimulants, blood, platelet infusions, Neupogen, G-CSF; or GM-CSF;
(l) pain management;
(m) the administration of anti-inflammatories;
(n) the administration of fluids;
(o) the administration of corticosteroids;
(p) the administration of insulin control medications;
(q) the administration of antipyretics;
(r) the administration of anti-nausea treatments;
(s) the administration of anti-diarrhea treatments;
(t) the administration of N-acetylcysteine;
(u) the administration of antihistamines; and
(v) the administration of agents for reduction of gastric toxicity.

Filgrastim is a granulocytic colony-stimulating factor (G-CSF) analog produced by recombinant DNA technology that is used to stimulate the proliferation and differentiation of granulocytes and is used to treat neutropenia; G-CSF can be used in a similar manner. GM-CSF is granulocyte macrophage colony-stimulating factor and stimulates stem cells to produce granulocytes (eosinophils, neutrophils, and basophils) and monocytes; its administration is useful to prevent or treat infection.

Anti-inflammatory agents are well known in the art and include corticosteroids and non-steroidal anti-inflammatory agents (NSAIDs). Corticosteroids with anti-inflammatory activity include, but are not limited to, cortisone, beclomethasone dipropionate, betamethasone, dexamethasone, prednisone, methylprednisolone, triamcinolone, fluocinolone acetonide, and fludrocortisone. Non-steroidal anti-inflammatory agents include, but are not limited to, acetylsalicylic acid (aspirin), sodium salicylate, choline magnesium trisalicylate, salsalate, diflunisal, sulfasalazine, olsalazine, acetaminophen, indomethacin, sulindac, tolmetin, diclofenac, ketorolac, ibuprofen, naproxen, flurbiprofen, ketoprofen, fenoprofin, oxaprozin, mefenamic acid, meclofenamic acid, piroxicam, meloxicam, nabumetone, rofecoxib, celecoxib, etodolac, nimesulide, aceclofenac, alclofenac, alminoprofen, amfenac, ampiroxicam, apazone, araprofen, azapropazone, bendazac, benoxaprofen, benzydamine, bermoprofen, benzpiperylon, bromfenac, bucloxic acid, bumadizone, butibufen, carprofen, cimicoxib, cinmetacin, cinnoxicam, clidanac, clofezone, clonixin, clopirac, darbufelone, deracoxib, droxicam, eltenac, enfenamic acid, epirizole, esflurbiprofen, ethenzamide, etofenamate, etoricoxib, felbinac, fenbufen, fenclofenac, fenclozic acid, fenclozine, fendosal, fentiazac, feprazone, filenadol, flobufen, florifenine, flosulide, flubichin methanesulfonate, flufenamic acid, flufenisal, flunixin, flunoxaprofen, fluprofen, fluproquazone, furofenac, ibufenac, imrecoxib, indoprofen, isofezolac, isoxepac, isoxicam, licofelone, lobuprofen, lomoxicam, lonazolac, loxaprofen, lumaricoxib, mabuprofen, miroprofen, mofebutazone, mofezolac, morazone, nepafanac, niflumic acid, nitrofenac, nitroflurbiprofen, nitronaproxen, orpanoxin, oxaceprol, oxindanac, oxpinac, oxyphenbutazone, pamicogrel, parcetasal, parecoxib, parsalmide, pelubiprofen, pemedolac, phenylbutazone, pirazolac, pirprofen, pranoprofen, salicin, salicylamide, salicylsalicylic acid, satigrel, sudoxicam, suprofen, talmetacin, talniflumate, tazofelone, tebufelone, tenidap, tenoxicam, tepoxalin, tiaprofenic acid, tiaramide, tilmacoxib, tinoridine, tiopinac, tioxaprofen, tolfenamic acid, triflusal, tropesin, ursolic acid, valdecoxib, ximoprofen, zaltoprofen, zidometacin, and zomepirac, and the salts, solvates, analogues, congeners, bioisosteres, hydrolysis products, metabolites, precursors, and prodrugs thereof.

The clinical use of corticosteroids is described in B. P. Schimmer & K. L. Parker, “Adrenocorticotropic Hormone; Adrenocortical Steroids and Their Synthetic Analogs; Inhibitors of the Synthesis and Actions of Adrenocortical Hormones” in Goodman & Gilman's The Pharmacological Basis of Therapeutics (L. L. Brunton, ed., 11^{th} ed., McGraw-Hill, New York, 2006), ch. 59, pp. 1587-1612, incorporated herein by this reference.

Anti-nausea treatments include, but are not limited to, ondansetron, metoclopramide, promethazine, cyclizine, hyoscine, dronabinol, dimenhydrinate, diphenhydramine, hydroxyzine, medizine, dolasetron, granisetron, palonosetron, ramosetron, domperidone, haloperidol, chlorpromazine, fluphenazine, perphenazine, prochlorperazine, betamethasone, dexamethasone, lorazepam, and thiethylperazine.

Anti-diarrheal treatments include, but are not limited to, diphenoxylate, difenoxin, loperamide, codeine, racecadotril, octreoside, and berberine.

N-acetylcysteine is an antioxidant and mucolytic that also provides biologically accessible sulfur.

Agents for reduction of gastric toxicity include, but are not limited to, ferruginol (C. Areche et al., “Gastroprotective Activity of Ferruginol in Mice and Rats: Effects on Gastric Secretion, Endogenous Prostaglandins and Non-Protein Sulfhydryls,” J. Pharm. Pharmacol. 60: 245-251 (2008)), incorporated herein by this reference.

When the improvement is made by pharmacokinetic/pharmacodynamic monitoring, the pharmacokinetic/pharmacodynamic monitoring can be, but is not limited to a method selected from the group consisting of:
(a) multiple determinations of blood plasma levels; and
(b) multiple determinations of at least one metabolite in blood or urine.

Typically, determination of blood plasma levels or determination of at least one metabolite in blood or urine is carried out by immunoassays. Methods for performing immunoassays are well known in the art, and include radioimmunoassay, ELISA (enzyme-linked immunosorbent assay), competitive immunoassay, immunoassay employing lateral flow test strips, and other assay methods.

One method potentially useful for the monitoring of metabolism of amonafide or a derivative or analog of amonafide is an ELISA assay for the rapid determination of N-acetyltransferase (NAT2 phenotypes), described in U.S. Pat. No. 5,830,672 to Wainer et al., incorporated herein by this reference. Amonafide is converted to an active metabolite by way of the N-acetyltransferase NAT2, and it has been reported that there is a direct correlation between the acetylator phenotype and the degree of toxicity induced by amonafide, with patients possessing a phenotype for rapid acetylation at greater risk to problems associated with severe toxicity. In general, this ELISA assay measures the concentration of two metabolites of caffeine. The first of these metabolites is 5-acetamino-6-amino-1-methyluracil (AAMU); the second of these metabolites is either 5-acetamino-6-formylamino-1-methyluracil (AFMU) or 1-methylxanthine (1X).

When the improvement is made by drug combination, the drug combination can be, but is not limited to, a drug combination selected from the group consisting of:
(a) use with fraudulent nucleosides;
(b) use with fraudulent nucleotides;
(c) use with thymidylate synthetase inhibitors;
(d) use with signal transduction inhibitors;
(e) use with cisplatin or platinum analogs;
(f) use with alkylating agents;
(g) use with anti-tubulin agents;
(h) use with antimetabolites;
(i) use with berberine;
(j) use with apigenin;
(k) use with colchicine or an analog thereof;
(l) use with genistein;
(m) use with etoposide;
(n) use with cytarabine;
(o) use with camptothecins;
(p) use with vinca alkaloids;
(q) use with topoisomerase inhibitors;
(r) use with 5-fluorouracil;
(s) use with curcumin;
(t) use with NF-κB inhibitors;
(u) use with rosmarinic acid;
(v) use with mitoguazone;
(w) use with meisoindigo;
(x) use with imatinib;
(y) use with dasatinib;
(z) use with nilotinib;
(aa) use with epigenetic modulators;
(ab) use with transcription factor inhibitors;
(ac) use with taxol;
(ad) use with homoharringtonine;
(ae) use with pyridoxal;
(af) use with spirogermanium;
(ag) use with caffeine;
(ah) use with nicotinamide;
(ai) use with methylglyoxalbisguanylhydrazone;
(aj) use with poly-ADP ribose polymerase (PARP) inhibitors;
(ak) use with EGFR inhibitors;
(al) use with Bruton's tyrosine kinase (BTK) inhibitors;
(am) use with c-Myc inhibitors;
(an) use with PTEN inhibitors;
(ao) use with IDH inhibitors;
(ap) use with polyamine analogs;
(aq) use with thalidomide and analogs;
(ar) use with homoharringtonine and analogs;
(as) use with bruceantin and analogs;
(at) use with bisantrene, amsacrine, or analogs of bisantrene or amsacrine;
(au) use with mitoxantrone;
(av) use with vosaroxin;
(aw) use with dianhydrogalactitol or dibromodulcitol;
(ax) use with 5-azacytidine;
(ay) use with decitabine;
(az) use with anti-VEGF agents such as bevacizumab;
(ba) use with anti-CD20 agents such as rituximab;
(bb) use with anti-EGFR vaccines;
(bc) use with T-cell stimulants;
(bd) use with dendritic cell vaccines; and
(be) use with PD inhibitors.

Topoisomerase inhibitors include, but are not limited to, irinotecan, topotecan, camptothecin, lamellarin D, amsacrine, etoposide, etoposide phosphate, teniposide, doxorubicin, and 4-[2-(3,5-dioxo-1-piperazinyl)-1-methylpropyl]piperazine-2,6-dione (ICRF-193).

Fraudulent nucleosides include, but are not limited to, cytosine arabinoside, gemcitabine, and fludarabine; other fraudulent nucleosides are known in the art.

Fraudulent nucleotides include, but are not limited to, tenofovir disoproxil fumarate and adefovir dipivoxil; other fraudulent nucleotides are known in the art.

Thymidylate synthetase inhibitors include, but are not limited to, raltitrexed, pemetrexed, nolatrexed, ZD9331, GS7094L, fluorouracil, and BGC 945.

Signal transduction inhibitors are described in A. V. Lee et al., “New Mechanisms of Signal Transduction Inhibitor Action: Receptor Tyrosine Kinase Down-Regulation and Blockade of Signal Transactivation,” Clin. Cancer Res. 9: 516s (2003), incorporated herein in its entirety by this reference.

Alkylating agents include, but are not limited to, Shionogi 254-S, aldo-phosphamide analogues, altretamine, anaxirone, Boehringer Mannheim BBR-2207, bendamustine, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)₂, dianhydrogalactitol, dibromodulcitol, other substituted hexitols, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsulfam, ifosfamide, iproplatin, lomustine, mafosfamide, melphalan, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromustine, Tanabe Seiyaku TA-077, tauromustine, temozolomide, teroxirone, tetraplatin and trimelamol, uramustine, as described in U.S. Pat. No. 7,446,122 by Chao et al., incorporated herein by this reference.

Anti-tubulin agents include, but are not limited to, vinca alkaloids, taxanes, podophyllotoxin, halichondrin B, and homohalichondrin B.

Antimetabolites include, but are not limited to: methotrexate, pemetrexed, 5-fluorouracil, capecitabine, cytarabine, gemcitabine, 6-mercaptopurine, and pentostatin, alanosine, AG2037 (Pfizer), 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrill-Dow DDFC, deazaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, piritrexim, plicamycin, Asahi Chemical PL-AC, Takeda TAC-788, thioguanine, tiazofurin, Erbamont TIF, trimetrexate, tyrosine kinase inhibitors, tyrosine protein kinase inhibitors, Taiho UFT and uricytin.

Berberine has antibiotic activity and prevents and suppresses the expression of pro-inflammatory cytokines and E-selectin, as well as increasing adiponectin expression.

Apigenin is a flavone that can reverse the adverse effects of cyclosporine and has chemoprotective activity, either alone or derivatized with a sugar.

Colchicine is a tricyclic alkaloid that exerts its activity by binding to the protein tubulin. Analogs of colchicine include, but are not limited to, cholchiceinamide, N-desacetylthiocolchicine, demecolcine, N-acetyliodocolchinol, trimethylcolchicinie acid (TMCA) methyl ether, N-acetylcolchinol, TMCA ethyl ether, isocolchicine, isocolchiceinamide, iso-TMCA methyl ether, colchiceine, TMCA, N-benzoyl TMCA, colchicosamide, colchicoside, colchinol and colchinoic acid (M. H. Zweig & C. F. Chignell, “Interaction of Some Colchicine Analogs, Vinblastine and Podophyllotoxin with Rat Brain Microtubule Protein,” Biochem. Pharmacol. 22: 2141-2150 (1973) and B. Yang et al., “Syntheses and Biological Evaluation of Ring C-Modified Colchicine Analogs,” Bioorg. Med. Chem. Lett. 20: 3831-3833 (2010)), both of which are incorporated herein by this reference.

Genistein is an isoflavone with the systemic name 5,7-dihydroxy-3-(4-hydroxyphenyl)chromen-4-one. Genistein has a number of biological activities, including activation of PPARs, inhibition of several tyrosine kinases, inhibition of topoisomerase, antioxidative activity, activation of Nrf2 antioxidative response, activation of estrogen receptor beta, and inhibition of the mammalian hexose transporter GLUT2.

Etoposide is an anticancer agent that acts primarily as a topoisomerase II inhibitor. Etoposide forms a ternary complex with DNA and the topoisomerase II enzyme, prevents re-ligation of the DNA strands and thus induces DNA strand breakage and promotes apoptosis of the cancer cells.

Cytarabine is a nucleoside analog replacing the ribose with arabinose. It can be incorporated into DNA and also inhibits both DNA and RNA polymerases and nucleotide reductase. It is particularly useful in the treatment of acute myeloid leukemia and acute lymphocytic leukemia,

Camptothecins include camptothecin, homocamptothecin, topotecan, irinotecan, DB 67, BNP 1350, exatecan, lurtotecan, ST 1481, and CKD 602. These compounds act as topoisomerase I inhibitors and block DNA synthesis in cancer cells.

Vinca alkaloids include vinblastine, vincristine, vindesine, and vinorelbine.

Topoisomerase inhibitors include topoisomerase I inhibitors and topoisomerase II inhibitors. Topoisomerase I inhibitors include the camptothecins and lamellarin D. Topoisomerase II inhibitors include, in addition to amonafide and derivatives and analogs thereof, etoposide, teniposide, doxorubicin, daunorubicin, mitoxantrone, amsacrine, ellipticines, and aurintricarboxylic acid. A number of plant-derived naturally-occurring phenolic compounds, such as genistein, quercetin, and resveratrol, exhibit inhibitory activity toward both topoisomerase I and topoisomerase II.

5-fluorouracil is a base analog that acts as a thymidylate synthase inhibitor and thereby inhibits DNA synthesis. When deprived of a sufficient supply of thymidine, rapidly dividing cancer cells die by a process known as thymineless death.

Curcumin is believed to have anti-neoplastic, anti-inflammatory, antioxidant, anti-ischemic, anti-arthritic, and anti-amyloid properties and also has hepatoprotective activity.

NF-κB inhibitors include, but are not limited to bortezomib.

Rosmarinic acid is a naturally-occurring phenolic antioxidant that also has anti-inflammatory activity.

Mitoguazone is an inhibitor of polyamine biosynthesis through competitive inhibition of S-adenosylmethionine decarboxylase.

Meisoindigo is active via several, possibly novel mechanisms of action. It has cell cycle specific effects, including arrest in G(O)/G1 for AML cell lines and G2/M arrest for HT-29 colorectal cell lines. It also stimulates apoptosis through a number of mechanisms, including the upregulation of p21 and p27 and the downregulation of Bcl-2 in primary AML cells, as well as upregulation of Bak and Bax in AML cells (DKO insensitive to chemotherapy), and a novel caspase-dependent pathway in K562 cells. Meisoindigo also has effects on mitochondria, but with no change in Bcl-2, Bax, and Bid protein expression. Meisoindigo also stimulates the cleavage of pro-caspase 3, 8, 9 and PARP in HL-60 myeloid cells. Meisoindigo also is directed to multiple cellular targets, which are possibly synergistic and complementary. For example, it promotes differentiation of human myeloblastic leukemic cells, accompanied by downregulation of c-myb gene expression. It also promotes inhibition of DNA and RNA synthesis in W256 cells, microtubule assembly, glycogen synthase kinase-3β (GSK-3β) (at 5-50 nM), CDK1/cyclin B, and CDK5/p25 (tau microtubule protein phosphorylation). Additionally, meisoindigo decreases 8-catenin and c-myc (HL-60 cells, but not in K562), affects the Wnt pathway through inhibiting GSK-3β and downregulating β-catenin and c-myc protein expression. Meisoindigo also promotes upregulation of CD11b, promoting myeloid differentiation, and upregulation of Ahi-1 in Jurkat cells (inducing phosphorylation of c-Myb). Furthermore, meisoindigo exhibits antiangiogenic effects, including decreased VEGF protection, VCAM-1, tubule formulation in HUVEC, and ECV304 apoptosis.

Imatinib is an inhibitor of the receptor tyrosine kinase enzyme ABL and is used to treat chronic myelogenous leukemia, gastrointestinal stromal tumors, and other hyperproliferative disorders.

Dasatinib is an inhibitor of BCR/ABL and Src family tyrosine kinases and is used to treat chronic myelogenous leukemia and acute lymphoblastic leukemia.

Nilotinib is another tyrosine kinase inhibitor approved for the treatment of chronic myelogenous leukemia; it inhibits the kinases BCR/ABL, KIT, LCK, EPHA3, and a number of other kinases.

Epigenetic modulators include polyamine-based epigenetic modulators, such as the polyamine-based epigenetic modulators described in S. K. Sharma et al., “Polyamine-Based Small Molecule Epigenetic Modulators,” Med. Chem. Commun. 3: 14-21 (2012), and L. G. Wang & J. W. Chiao, “Prostate Cancer Chemopreventive Activity of Phenethyl Isothiocyanate Through Epigenetic Regulation (Review), Int. J. Oncol. 37: 533-539 (2010), both incorporated herein by this reference.

Transcription factor inhibitors include 1-(4-hexaphenyl)-2-propane-1-one, 3-fluoro-4-[[2-hydroxy-2-(5,5,8,8-tetramethyl-5,6,7,8,-tetrahydro-2-naphthalenyl)acetyl]amino]-benzoic acid (BMS 961), 4-[5-[8-(1-Methylethyl)-4-phenyl-2-quinolinyl]-1H-pyrrolo-2-benzoic acid (ER-50891), 7-Ethenyl-2-(3-fluoro-4-hydroxyphenyl)-5-benzoxazolol (ERB 041), and other compounds. Transcription factor inhibitors are described in T. Berg, “Inhibition of Transcription Factors with Small Organic Molecules,” Curr. Opin. Chem. Biol. 12: 464-471 (2008), incorporated herein by this reference.

Tetrandrine has the chemical structure 6,6′,7,12-tetramethoxy-2,2′-dimethyl-1β-berbaman and is a calcium channel blocker that has anti-inflammatory, immunologic, and antiallergenic effects, as well as an anti-arrhythmic effect similar to that of quinidine. It has been isolated from Stephania tetranda and other Asian herbs.

VEGF inhibitors include bevacizumab (Avastin), which is a monoclonal antibody against VEGF, itraconazole, and suramin, as well as batimastat and marimastat, which are matrix metalloproteinase inhibitors, and cannabinoids and derivatives thereof.

Cancer vaccines are being developed. Typically, cancer vaccines are based on an immune response to a protein or proteins occurring in cancer cells that does not occur in normal cells. Cancer vaccines include Provenge for metastatic hormone-refractory prostate cancer, Oncophage for kidney cancer, CimaVax-EGF for lung cancer, MOBILAN, Neuvenge for Her2/neu expressing cancers such as breast cancer, colon cancer, bladder cancer, and ovarian cancer, Stimuvax for breast cancer, and others. Cancer vaccines are described in S. Pejawar-Gaddy & O. Finn, “Cancer Vaccines: Accomplishments and Challenges,” Crit. Rev. Oncol. Hematol. 67: 93-102 (2008), incorporated herein by this reference.

The use of methylglyoxalbisguanylhydrazone in cancer therapy has been described in D. D. Von Hoff, “MGBG: Teaching an Old Drug New Tricks,” Ann. Oncol. 5: 487-493 (1994), incorporated herein by this reference.

Poly-ADP ribose polymerase inhibitors are described in G. J. Southan & C. Szabó, “Poly(ADP-Ribose) Inhibitors,” Curr. Med. Chem. 10: 321-240 (2003), incorporated herein by this reference, and include nicotinamide, 3-aminobenzamide, substituted 3,4-dihydroisoquinolin-1(2H)-ones and isoquinolin-1(2H)-ones, benzimidazoles, indoles, phthalazin-1(2H)-ones, quinazolinones, isoindolinones, phenanthridinones, and other compounds. Poly-ADP ribose polymerase (PARP) inhibitors include, but are not limited to: (1) derivatives of tetracycline as described in U.S. Pat. No. 8,338,477 to Duncan et al.; (2) 3,4-dihydro-5-methyl-1(2H)-isoquinoline, 3-aminobenzamide, 6-aminonicotinamide, and 8-hydroxy-2-methyl-4(3H)-quinazolinone, as described in U.S. Pat. No. 8,324,282 by Gerson et al.; (3) 6-(5H)-phenanthridinone and 1,5-isoquinolinediol, as described in U.S. Pat. No. 8,324,262 by Yuan et al.; (4) (R)-3-[2-(2-hydroxymethylpyrrolidin-1-yl)ethyl]-5-methyl-2H-isoquinolin-1-one, as described in U.S. Pat. No. 8,309,573 to Fujio et al.; (5) 6-alkenyl-substituted 2-quinolinones, 6-phenylalkyl-substituted quinolinones, 6-alkenyl-substituted 2-quinoxalinones, 6-phenylalkyl-substituted 2-quinoxalinones, substituted 6-cyclohexylalkyl substituted 2-quinolinones, 6-cyclohexylalkyl substituted 2-quinoxalinones, substituted pyridones, quinazolinone derivatives, phthalazine derivatives, quinazolinedione derivatives, and substituted 2-alkyl quinazolinone derivatives, as described in U.S. Pat. No. 8,299,256 to Vialard et al.; (6) 5-bromoisoquinoline, as described in U.S. Pat. No. 8,299,088 to Mateucci et al.; (7) 5-bis-(2-chloroethyl)amino]-1-methyl-2-benzimidazolebutyric acid, 4-iodo-3-nitrobenzamide, 8-fluoro-5-(4-((methylamino)methyl)phenyl)-3,4-dihydro-2H-azepino[5,4,3-cd]indol-1(6H)-one phosphoric acid, and N-[3-(3,4-dihydro-4-oxo-1-phthalazinyl)phenyl]-4-morpholinebutanamide methanesulfonate, as described in U.S. Pat. No. 8,227,807 to Gallagher et al.; (8) pyridazinone derivatives, as described in U.S. Pat. No. 8,268,827 to Branca et al.; (9) 4-[3-(4-cyclopropanecarbonyl-piperazine-1-carbonyl)-4-fluorobenzyl]-2H-phthalazin-1-one, as described in U.S. Pat. No. 8,247,416 to Menear et al.; (10) tetraaza phenalen-3-one compounds, as described in U.S. Pat. No. 8,236,802 to Xu et al.; (11) 2-substituted-1H-benzimidazole-4-carboxamides, as described in U.S. Pat. No. 8,217,070 to Zhu et al.; (12) substituted 2-alkyl quinazolinones, as described in U.S. Pat. No. 8,188,103 to Van der Aa et al.; (13) 1H-benzimidazole-4-carboxamides, as described in U.S. Pat. No. 8,183,250 to Penning et al.; (13) indenoisoquinolinone analogs, as described in U.S. Pat. No. 8,119,654 to Jagtap et al.; (14) benzoxazole carboxamides, described in U.S. Pat. No. 8,088,760 to Chu et al; (15) diazabenzo[de]anthracen-3-one compounds, described in U.S. Pat. No. 8,058,075 to Xu et al.; (16) dihydropyridophthalazinones, described in U.S. Pat. No. 8,012,976 to Wang et al., (17) substituted azaindoles, described in U.S. Pat. No. 8,008,491 to Jiang et al.; (18) fused tricyclic compounds, described in U.S. Pat. No. 7,956,064 to Chua et al.; (19) substituted 6a,7,8,9-tetrahydropyrido[3,2-e]pyrrolo[1,2-a]pyrazin-6(5H)-ones, described in U.S. Pat. No. 7,928,105 to Gangloff et al.; and (20) thieno[2,3-c]isoquinolines, described in U.S. Pat. No. 7,825,129. Other PARP inhibitors are known in the art.

EGFR inhibitors, including both small molecules and monoclonal antibodies, are described above. Other EGFR inhibitors are known in the art.

Bruton's tyrosine kinase (BTK) is a kinase enzyme that plays a key role in the maturation of B cells and in mast cell activation through the high-affinity IgE receptor. Deficiencies in BTK activity are associated with the primary immunodeficiency disease X-linked agammaglobulinemia. The Btk gene is located on the X-chromosome. BTK contains a PH domain that binds phosphatidyl inositol (3,4,5)-triphosphate (PIP3). PIP3 induces BTK to phosphorylate phospholipase C, which in turn hydrolyzes phosphatidyl inositol diphosphate into two second messengers, inositol triphosphate and diacylglycerol, which in turn modulate the activity of downstream proteins in B cells. BTK inhibitors include, but are not limited to: LFM-A13 (α-cyano-β-hydroxy-β-methyl-N-(2,5-dibromophenyl)propenamide; terreic acid ((1R,6S)-3-Hydroxy-4-methyl-7-oxabicyclo[4.1.0]hept-3-ene-2,5-dione); ibrutinib; pyrazolo[3,4-d]pyrimidine and pyrrolo[2,3-d]pyrimidine compounds as disclosed in U.S. Pat. No. 8,377,946 to Chen et al., incorporated herein by this reference; 2,4-disubstituted pyrimidines as disclosed in U.S. Pat. No. 8,338,439 to Singh et al., incorporated herein by this reference; 6-phenyl-imidazo[1,2-a]pyridine and 6-phenyl-imidazo[1,2-b]pyridazine derivatives, as disclosed in U.S. Pat. No. 8,324,211 to Dewdney et al., incorporated herein by this reference; 5-phenyl-1H-pyridin-2-one, 6-phenyl-2H-pyridazin-3-one, and 5-phenyl-1H-pyrazin-2-one derivatives, as disclosed in U.S. Pat. No. 8,318,719 to Dewdney et al. and U.S. Pat. No. 8,297,077, incorporated herein by this reference; 1-(3-(4-amino-3-(4-phenoxyphenyl)-1H-pyrazolo[3,4-d]pyrimidin-1-yl)piperidin-1yl)prop-2-en-1-one, (E)-1-(3-(4-amino-3-(4-phenoxyphenyl)-1H-pyrazolo[3,4-d]pyrimidin-1-yl)piperidin-1-yl)but-2-en-1-one, 1-(3-(4-amino-3-(4-phenoxyphenyl)-1H-pyrazolo[3,4-d]pyrimidin-1-yl)piperidin-1-yl)sulfonylethene, 1-(3-(4-amino-3-(4-phenoxyphenyl)-1H-pyrazolo[3,4-d]pyrimidin-1-yl)piperidin-1-yl)prop-2-yn-1-one, 1-(4-(4-amino-3-(4-phenoxyphenyl)-1H-pyrazolo[3,4-d]pyrimidin-1-yl)piperidin-1-yl)prop-2-en-1-one, N-((1s,4s)-4-(4-amino-3-(4-phenoxyphenyl)-1H-pyrazolo[3,4-d]pyrimidin-1-yl)cyclohexyl)acrylamide, 14(R)-3-(4-amino-3-(4-phenoxyphenyl)-1H-pyrazolo[3,4-d]pyrimidin-1-yl)pyrrolidin-1-yl)prop-2-en-1-one, 1-((S)-3-(4-amino-3-(4-phenoxyphenyl)-1H-pyrazolo[3,4-d]pyrimidin-1-yl)pyrrolidin-1-yl)prop-2-en-1-one, 14(R)-3-(4-amino-3-(4-phenoxyphenyl)-1H-pyrazolo[3,4-d]pyrimidin-1-yl)piperidin-1-yl)prop-2-en-1-one, 14(S)-3-(4-amino-3-(4-phenoxyphenyl)-1H-pyrazolo[3,4-d]pyrimidin-1-yl)piperidin-1-yl)prop-2-en-1-one, and (E)-1-(3-(4-amino-3-(4-phenoxyphenyl)-1H-pyrazolo[3,4-d]pyrimidin-1-yl)piperidin-1-yl)-4-(dimethylamino)but-2-en-1-one, as disclosed in U.S. Pat. No. 8,236,812 to Honigberg et al., incorporated herein by this reference; pyrazolo[3,4-d]pyrimidines, as disclosed in U.S. Pat. No. 8,232,280, incorporated herein by this reference; 2-(4-fluoro-2-methylphenylamino)-1,6-dimethyl-7-[3-(4-methyl-4-oxo-4λ⁵[1,4]azaphosphinan-1-yl)-propenyl]-1,8-dihydro-imidazo[4,5-h]isoquinolin-9-one, 2-(2,6-dichloro-phenylamino)-1,6-dimethyl-7-[3-(4-methyl-4-oxo-4λ⁵[1,4]azaphosphinan-1-yl)-propenyl]-1,8-dihydro-imidazo[4,5-h]isoquinolin-9-one, 2-(4-fluoro-2-methylphenylamino)-1,6-dimethyl-7-[3-(4-oxo-4-phenyl-4λ⁵-[1,4]azaphosphinan-1-yl)-propenyl]-1,8-dihydro-imidazo[4,5-h]isoquinolin-9-one, 2-(3-fluoro-6-methylphenylamino)-1,6-dimethyl-7-[3-(4-oxo-4-phenyl-4λ⁵-[1,4]azaphosphinan-1-yl)-propenyl]-1,8-dihydro-imidazo[4,5-h]isoquinolin-9-one, 2-(2,6-dichlorophenylamino)-1,6-dimethyl-7-[3-(4-oxo-4-phenyl-4λ⁵-[1,4]azaphosphinan-1-yl)-propenyl]-1,8-dihydro-imidazo[4,5-h]isoquinolin-9-one, 2-(2,4-dichloro-6-methylphenylamino)-1,6-dimethyl-7-[3-(4-oxo-4-phenyl-4λ⁵-[1,4]azaphosphinan-1-yl)-propenyl]-1,8-dihydro-imidazo[4,5-h]isoquinolin-9-one, 2-(3-fluoro-6-methylphenylamino)-1,6-dimethyl-7-{2-[(4-oxo-4-phenyl-4λ⁵-[1,4]azaphosphinan-1-yl)-propenyl]-1,8-dihydro-imidazo[4,5-h]isoquinolin-9-one, 2-(2,4-dichloro-6-methylphenylamino)-1,6-dimethyl-7-[3-(4-oxo-4-phenyl-4λ⁵-[1,4]azaphosphinan-1-yl)-propyl]-1,8-dihydro-imidazo[4,5-h]isoquinolin-9-one, 2-(4-fluoro-2-methylphenylamino)-1,6-dimethyl-7-[3-(4-oxo-4-phenyl-4λ⁵-[1,4]azaphosphinan-1-yl)-propyl]-1,8-dihydro-imidazo[4,5-h]isoquinolin-9-one, 2-(4-fluoro-2-methylphenylamino)-1,6-dimethyl-7-[3-(4-oxo-4-(4-fluorophenyl)-4λ⁵-[1,4]azaphosphinan-1-yl)-propenyl]-1,8-dihydro-imidazo[4,5-h]isoquinolin-9-one, 2-(3-fluoro-6-methylphenylamino)-1,6-dimethyl-7-[3-(4-oxo-4-(4-methoxyphenyl)-4λ⁵-[1,4]azaphosphinan-1-yl)-propenyl]-1,8-dihydro-imidazo[4,5-h]isoquinolin-9-one, 2-(4-fluoro-2-methylphenylamino)-1,6-dimethyl-7-{3-[4-(4-fluorophenylmethyl)-4-oxo-4λ⁵-[1,4]azaphosphinan-1-yl]-propenyl}-1,8-dihydro-imidazo[4,5-h]isoquinolin-9-one, 2-(4-fluoro-2-methylphenylamino)-1,6-dimethyl-7-{3-[4-(cyclopropylmethyl)-4-oxo-4λ⁵-[1,4]azaphosphinan-1-yl]-propenyl}-1,8-dihydro-imidazo[4,5-h]isoquinolin-9-one, 2-(4-fluoro-2-methylphenylamino)-1,6-dimethyl-7-{3-[4-(cyclopropyl)-4-oxo-4λ⁵-[1,4]azaphosphinan-1-yl]-propenyl}-1,8-dihydro-imidazo[4,5-h]isoquinolin-9-one, 2-(4-fluoro-2-methylphenylamino)-1,6-dimethyl-7-{3-[(1-oxo-1-methyl-1λ⁵-phosphinan-4-yl)-carbonylamino]propenyl}-1,8-dihydro-imidazo[4,5-h]isoquinolin-9-one, 2-(4-fluoro-2-methylphenylamino)-1,6-dimethyl-7-{3-[(1-oxo-1-trans-phenyl-1λ⁵-phosphinan-4-yl)-carbonylamino]propenyl}-1,8-dihydro-imidazo[4,5-h]isoquinolin-9-one, 2-(4-fluoro-2-methyl-phenylamino)-1,6-dimethyl-7-{3-[(1-oxo-1-cis-phenyl-1λ⁵-phosphinan-4-yl)-carbonylamino]propenyl}-1,8-dihydro-imidazo[4,5-h]isoquinolin-9-one, 2-(2,6-dichlorophenylamino)-1,6-dimethyl-7-[3-(N-phenylpiperazin-1-yl)-propenyl]-1,8-dihydro-imidazo[4,5-h]isoquinolin-9-one, 2-(4-fluoro-2-methylphenylamino)-1,6-dimethyl-7-[3-(N-phenylpiperazin-1-yl)-propenyl]-1,8-dihydro-imidazo[4,5-h]isoquinolin-9-one, 2-(4-fluoro-2-methylphenylamino)-1,6-dimethyl-7-[3-(N-(4-chlorophenyl)-piperazin-1-yl)-propenyl]-1,8-dihydro-imidazo[4,5-h]isoquinolin-9-one, 2-(4-fluoro-2-methylphenylamino)-1,6-dimethyl-7-[3-(N-methylcarbonylpiperazin-1-yl)-propenyl]-1,8-dihydro-imidazo[4,5-h]isoquinolin-9-one, 2-(4-fluoro-2-methylphenylamino)-1,6-dimethyl-7-[3-(N-phenylcarbonylpiperazin-1-yl)-propenyl]-1,8-dihydro-imidazo[4,5-h]isoquinolin-9-one, 2-(4-fluoro-2-methylphenylamino)-1,6-dimethyl-7-[3-(N-methylsulfonylpiperazin-1-yl)-propenyl]-1,8-dihydro-imidazo[4,5-h]isoquinolin-9-one, 2-(3-fluoro-2-methylphenylamino)-1,6-dimethyl-7-[3-(N-methylsulfonylpiperazin-1-yl)-propenyl]-1,8-dihydro-imidazo[4,5-h]isoquinolin-9-one, 2-(4-fluoro-2-methylphenylamino)-1,6-dimethyl-7-[3-(N-(2,6-dichlorophenylmethyl)-piperazin-1-yl)-propenyl]-1,8-dihydro-imidazo[4,5-h]isoquinolin-9-one, 2-(4-fluoro-2-methylphenylamino)-1,6-dimethyl-7-[3-(N-phenylsulfonylpiperazin-1-yl)-propenyl]-1,8-dihydro-imidazo[4,5-h]isoquinolin-9-one, 2-(4-fluoro-2-methylphenylamino)-1,6-dimethyl-7-[3-(N-(4-fluorophenyl)-piperazin-1-yl)-propenyl]-1,8-dihydro-imidazo[4,5-h]isoquinolin-9-one, 2-(4-fluoro-2-methylphenylamino)-1,6-dimethyl-7-[3-(N-tert-butyloxycarbonylpiperazin-1-yl)-propenyl]-1,8-dihydro-imidazo[4,5-h]isoquinolin-9-one, 2-(4-fluoro-2-methylphenylamino)-1,6-dimethyl-7-[3-(N—(N,N-dimethylaminosulfonyl)-piperazin-1-yl)-propenyl]-1,8-dihydro-imidazo[4,5-h]isoquinolin-9-one, 2-(4-fluoro-2-methylphenylamino)-1,6-dimethyl-7-[3-(N-ethylcarbonylpiperazin-1-yl)-propenyl]-1,8-dihydro-imidazo[4,5-h]isoquinolin-9-one, 2-(4-fluoro-2-methylphenylamino)-1,6-dimethyl-7-[3-(N-Osopropylsulfonylypiperazin-1-yl)-propenyl]-1,8-dihydro-imidazo[4,5-h]isoquinolin-9-one, 2-(4-fluoro-2-methylphenylamino)-1,6-dimethyl-7-[3-(N-(ethylsulfonyl)-piperazin-1-yl)-propenyl]-1,8-dihydro-imidazo[4,5-h]isoquinolin-9-one, and 2-(4-fluoro-2-methylphenylamino)-1,6-dimethyl-7-[3-(N-isopropylcarbonylpiperazin-1-yl)-propenyl]-1,8-dihydro-imidazo[4,5-h]isoquinolin-9-one, as disclosed in U.S. Pat. No. 8,067,395 to Jankowski et al., incorporated herein by this reference; 4-tert-butyl-N-(2-methyl-3-{1-methyl-5-[5-(morpholine-4-carbonyl)-pyridin-2-ylamino]-6-oxo-1,6-dihydro-pyridazin-3-yl}-phenyly benzamide, 4-tert-butyl-N-(2-methyl-3-{1-methyl-5-[5-(4-methyl-piperazine-1-carbonyl)pyridin-2-ylamino]-6-oxo-1,6-dihydro-pyridazin-3-yl}-phenyl)-benzamide, 4-tert-butyl-N-{2-methyl-3-[1-methyl-6-oxo-5-(pyridin-2-ylamino)-1,6-dihydropyridazin-3-yl]-phenyl)-benzamide, 4-tert-butyl-N-{2-methyl-3-[1-methyl-6-oxo-5-(pyrimidin-4-ylamino)-1,6-dihydropyridazin-3-yl]-phenyl)-benzamide, 4-(1-hydroxy-1-methyl-ethyl)-N-(2-methyl-3-1-methyl-5-[5-(4-methyl-piperazine-1-carbonyl)-pyridin-2-ylamino]-6-oxo-1,6-dihydro-pyridazin-3-yl}-phenyl)benzamide, 4-tert-butyl-piperazine-1-carboxylic acid (2-methyl-3-{1-methyl-5-(morpholine-4-carbonyl)-pyridin-2-ylamino]-6-oxo-1,6-dihydro-pyridazin-3-yl}-phenyl)-amide, 4-tert-butyl-2-methoxy-N-(2-methyl-3-{1-methyl-5-[5-(morpholine-4-carbonyl)pyridin-2-ylamino]-6-oxo-1,6-dihydro-pyridazin-3-yl}-phenyl)-benzamide, 7-tert-butyl-3-(2-methyl-3-1-methyl-5-[5-(morpholine-4-carbonyl)-pyridin-2-ylamino]-6-oxo-1,6-dihydro-pyridazin-3-yl}-phenyl)-3H-quinazolin-4-one, 6-{6-[3-(4-tert-butyl-benzoylamino)-2-methyl-phenyl]-2-methyl-3-oxo-2,3-dihydro-pyridazin-4-ylamino}-nicotinic acid methyl ester, 3-tert-butoxy-azetidine-1-carboxylic acid (2-methyl-3-{1-methyl-5-[5-(morpholine-4-carbonyl)-pyridin-2-ylamino]-6-oxo-1,6-dihydro-pyridazin-3-yl}-phenyl)-amide, 4-tert-butyl-N-(2-methyl-3-{1-methyl-5-[2-(4-methyl-piperazin-1-yl)-pyrimidin-4-ylamino]-6-oxo-1,6-dihydro-pyridazin-3-yl}-phenyl)-benzamide, 4-tert-butyl-N-{2-methyl-3-[1-methyl-5-(2-methylsulfanyl-pyrimidin-4-ylamino)-6-oxo-1,6-dihydro-pyridazin-3-yl]-phenyl}-benzamide, 4-tert-butyl-N-{3-[5-(2-methanesulfonyl-pyrimidin-4-ylamino)-1-methyl-6-oxo-1,6-dihydropyridazin-3-yl]-2-methyl-phenyl}-benzamide, 4-tert-butyl-N-{2-methyl-3-[1-methyl-5-(2-morpholin-4-yl-pyrimidin-4-ylamino)-6-oxo-1,6-dihydro-pyridazin-3-yl]-phenyl}-benzamide, 4-(1-hydroxy-1-methyl-ethyl)-N-(2-methyl-3-{1-methyl-5-[5-(morpholine-4-carbonyl)pyridin-2-ylamino]-6-oxo-1,6-dihydro-pyridazin-3-yl}-phenyl)-benzamide, 4-(1-hydroxy-1-methyl-ethyl)-N-(3-{1-methyl-5-[5-(morpholine-4-carbonyl)pyridin-2-ylamino]-6-oxo-1,6-dihydro-pyridazin-3-yl}-phenyl)-benzamide, 4-tert-butyl-N-{3-[5-(2-methoxy-pyrimidin-4-ylamino)-1-methyl-6-oxo-1,6-dihydropyridazin-3-yl]-2-methyl-phenyl}-benzamide; 4-tert-butyl-N-(3-{5-[2-(2-dimethylamino-ethoxy)-pyrimidin-4-ylamino]-1-methyl-6-oxo-1,6-dihydro-pyridazin-3-yl}-2-methyl-phenyl)-benzamide, 4-tert-butyl-N-(2-methyl-3-{1-methyl-6-oxo-5-[2-(pyrrolidin-3-ylmethoxy)-pyrimidin-4-ylamino]-1,6-dihydro-pyridazin-3-yl}-phenyl)-benzamide, 4-tert-butyl-N-(3-{5-[2-(3-hydroxymethyl-pyrrolidin-1-yl)-pyrimidin-4-ylamino]-1-methyl-6-oxo-1,6-dihydro-pyridazin-3-yl}-2-methyl-phenyl)-benzamide, 4-tert-butyl-N-{2-methyl-3-[1-methyl-6-oxo-5-(2-pyrrolidin-1-yl-pyrimidin-4-ylamino)-1,6-dihydro-pyridazin-3-yl]-phenyl}-benzamide, 4-tert-butyl-N-(3-{5-[2-(3-hydroxy-pyrrolidin-1-yl)-pyrimidin-4-ylamino]-1-methyl-6-oxo-1,6-dihydro-pyridazin-3-yl}-2-methyl-phenyl)-benzamide, 4-tert-butyl-N-{2-methyl-3-[1-methyl-5-(3-methyl-ureido)-6-oxo-1,6-dihydro-pyridazin-3-yl]-phenyl}-benzamide, 4-tert-butyl-N-(2-methyl-3-{1-methyl-5-[4-(morpholine-4-carbonyl)-phenylamino]-6-oxo-1,6-dihydro-pyridazin-3-yl}-phenyl)-benzamide, 4-tert-butyl-N-(3-{1-methyl-5-[5-(morpholine-4-carbonyl)-pyridin-2-ylamino]-6-oxo-1,6-dihydro-pyridazin-3-yl}-phenyl)-benzamide, 4-tert-butyl-N-(3-{5-[4-(4-hydroxy-piperidine-1-carbonyl)-phenylamino]-1-methyl-6-oxo-1,6-dihydro-pyridazin-3-yl}-2-methyl-phenyl)-benzamide, 4-tert-butyl-N-{3-[5-ethyl-ureido)-1-methyl-6-oxo-1,6-dihydro-pyridazin-3-2-methylphenyl}-benzamide; 4-dimethylamino-N-(3-{5-[5-(4-hydroxy-piperidine-1-carbonyl)-pyridin-2-ylamino]-1-methyl-6-oxo-1,6-dihydro-pyridazin-3-yl]-2-methyl-phenyl)-benzamide, N-(3-hydroxy-4,4-dimethyl-pentyl)-2-(3-{1-methyl-5-[5-(morpholine-4-carbonyl)-pyridin-2-ylamino]-6-oxo-1,6-dihydro-pyridazin-3-yl}-phenyl)-acetamide, 4-tert-butyl-2-hydroxy-N-(2-methyl-3-{1-methyl-5-[5-(morpholine-4-carbonyl)-pyridin-2-ylamino]-6-oxo-1,6-dihydro-pyridazin-3-yl}-phenyl)-benzamide, 6-{3-[2-(1,3-dihydro-isoindol-2-yl)-2-oxo-ethyl]-phenyl}-4-[5-(4-hydroxypiperidine-1-carbonyl)-pyridin-2-ylamino]-2-methyl-2H-pyridazin-3-one, 7-tert-butyl-3-(2-methyl-3-{1-methyl-5-[5-(morpholine-4-carbonyl)-pyridin-2-ylamino]-6-oxo-1,6-dihydro-pyridazin-3-yl}-phenyl)-2,3-dihydro-1H-quinazolin-4-one, 6-{3-[2-(2-isopropoxy-azetidin-1-yl)-2-oxo-ethyl]-phenyl}-2-methyl-4-[5-(morpholine-4-carbonyl)-pyridin-2-ylamino]-2H-pyridazin-3-one, 6-{3-[2-(4-tert-butyl-phenyl)-2-oxo-ethyl]-phenyl}-2-methyl-4-[5-(morpholine-4-carbonyl)pyridin-2-ylamino]-2H-pyridazin-3-one, 6-{3-[2-(4-tert-butyl-piperazin-1-yl)-2-oxo-ethyl]-phenyl}-2-methyl-4-[5-(morpholine-4-carbonyl)-pyridin-2-ylamino]-2H-pyridazin-3-one, 6-{3-[2-(3-tert-butoxy-azetidin-1-yl)-2-oxo-ethyl]-phenyl}-2-methyl-4-[5-(morpholine-4-carbonyl)-pyridin-2-ylamino]-2H-pyridazin-3-one, 6-{3-[2-(1,3-dihydro-isoindol-2-yl)-2-oxo-ethyl]-phenyl}-2-methyl-4-[5-(morpholine-4-carbonyl)-pyridin-2-ylamino]-2H-pyridazin-3-one, 6-{3-[2-(4-isopropyl-piperazin-1-yl)-2-oxo-ethyl]-phenyl}-2-methyl-4-[5-(morpholine-4-carbonyl)-pyridin-2-ylamino]-2H-pyridazin-3-one, 6-{3-[2-(4-tert-butyl-piperidin-1-yl)-2-oxo-ethyl]-phenyl}-2-methyl-4-[5-(morpholine-4-carbonyl)-pyridin-2-ylamino]-2H-pyridazin-3-one, N-(3,3-dimethyl-butyl)-2-(3-{1-methyl-5-[5-(morpholine-4-carbonyl)-pyridin-2-ylamino]-6-oxo-1,6-dihydro-pyridazin-3-yl}-phenyl)-acetamide, 6-{3-[2-(4-acetyl-piperazin-1-yl)-2-oxo-ethyl]-phenyl}-2-methyl-4-[5-(morpholine-4-carbonyl)-pyridin-2-ylamino]-2H-pyridazin-3-one, 4-cyclopropyl-N-{2-hydroxymethyl-3-[1-methyl-5-(1-methyl-1H-pyrazol-3-ylamino)-6-oxo-1,6-dihydro-pyridazin-3-yl]-phenyl)-benzamide, and 4-cyclopropyl-N-(2-hydroxymethyl-3-1-methyl-5-[5-(morpholine-4-carbonyl)pyridin-2-ylamino]-6-oxo-1,6-dihydro-pyridazin-3-yl}-phenyl)-benzamide, as disclosed in U.S. Pat. No. 7,943,618 to Dewdney et al., incorporated herein by this reference; 6-dimethylamino-2-(3-{1-methyl-5-[5-(morpholine-4-carbonyl)-pyridin-2-ylamino]-6-oxo-1,6-dihydro-pyridin-3-yl}-phenyl)-2H-isoquinolin-1-one, 6-dimethylamino-2-(2-methyl-3-{1-methyl-5-[5-(morpholine-4-carbonyl)-pyridin-2-ylamino]-6-oxo-1,6-dihydro-pyridin-3-yl}-phenyl)-2H-isoquinolin-1-one, 6-dimethylamino-2-(2-hydroxymethyl-3-{1-methyl-5-[5-(morpholine-4-carbonyl)-pyridin-2-ylamino]-6-oxo-1,6-dihydro-pyridin-3-yl}-phenyl)-2H-isoquinolin-1-one, 6-dimethylamino-2-{2-hydroxymethyl-3-[1-methyl-5-(1-methyl-1H-pyrazol-3-ylamino)-6-oxo-1,6-dihydro-pyridin-3-yl]-phenyl}-2H-isoquinolin-1-one, 1-{5-[3-(6-dimethylamino-1-oxo-1H-isoquinolin-2-yl)-2-hydroxymethyl-phenyl]-1-methyl-2-oxo-1,2-dihydro-pyridin-3-yl}-3-ethyl-urea, 2-(2-hydroxymethyl-3-{1-methyl-5-[5-(morpholine-4-carbonyl)-pyridin-2-ylamino]-6-oxo-1,6-dihydropyridin-3-yl}-phenyl)-6-(1-methyl-cyclopropyl)-2H-isoquinolin-1-one, 2-{2-hydroxymethyl-3-[1-methyl-5-(5-morpholin-4-ylmethyl-pyridin-2-ylamino)-6-oxo-1,6-dihydro-pyridin-3-yl]-phenyl}-6-(1-methyl-cyclopropyl)-2H-isoquinolin-1-one, 2-(2-hydroxymethyl-3-{1-methyl-5-[5-(4-methyl-piperazine-1-carbonyl)-pyridin-2-ylamino]-6-oxo-1,6-dihydro-pyridin-3-yl}-phenyl)-6-(1-methyl-cyclopropyl)-2H-isoquinolin-1-one, 6-cyclopropyl-2-{2-hydroxymethyl-3-[1-methyl-5-(5-morpholin-4-ylmethyl-pyridin-2-ylamino)-6-oxo-1,6-dihydro-pyridin-3-yl]-phenyl}-2H-isoquinolin-1-one, 6-cyclopropyl-2-(2-hydroxymethyl-3-{1-methyl-5-[5-(4-methyl-piperazine-1-carbonyl)-pyridin-2-ylamino]-6-oxo-1,6-dihydro-pyridin-3-yl}-phenyl)-2H-isoquinolin-1-one, 6-cyclopropyl-2-(2-hydroxymethyl-3-{1-methyl-5-[5-(morpholine-4-carbonyl)-pyridin-2-ylamino]-6-oxo-1,6-dihydro-pyridin-3-yl}-phenyl)-2H-isoquinolin-1-one, 6-cyclopropyl-2-{2-hydroxymethyl-3-[1-methyl-5-(5-morpholin-4-yl-pyridin-2-ylamino)-6-oxo-1,6-dihydro-pyridin-3-yl]-phenyl}-2H-isoquinolin-1-one, 6-cyclopropyl-2-(2-hydroxymethyl-3-{1-methyl-5-[5-(morpholine-4-carbonyl)-pyridin-2-ylamino]-6-oxo-1,6-dihydro-pyridin-3-yl}-phenyl)-3-methyl-2H-isoquinolin-1-one, 2-{2-hydroxymethyl-3-[1-methyl-5-(5-morpholin-4-yl-pyridin-2-ylamino)-6-oxo-1,6-dihydro-pyridin-3-yl]-phenyl}-6-(1-methyl-cyclopropyl)-2H-isoquinolin-1-one, 6-dimethylamino-2-{2-hydroxymethyl-3-[1-methyl-5-(5-morpholin-4-yl-pyridin-2-ylamino)-6-oxo-1,6-dihydro-pyridin-3-yl]-phenyl}-2H-isoquinolin-1-one, 6-tert-butyl-2-(2-hydroxymethyl-3-{1-methyl-5-[5-(morpholine-4-carbonyl)-pyridin-2-ylamino]-6-oxo-1,6-dihydro-pyridin-3-yl}-phenyl)-2H-isoquinolin-1-one, 6-cyclopropyl-3-hydroxymethyl-2-(2-hydroxymethyl-3-{1-methyl-5-[5-(morpholine-4-carbonyl)-pyridin-2-ylamino]-6-oxo-1,6-dihydro-pyridin-3-yl}-phenyl)-2H-isoquinolin-1-one, 6-cyclopropyl-3-hydroxymethyl-2-{2-hydroxymethyl-3-[1-methyl-5-(5-morpholin-4-yl-pyridin-2-ylamino)-6-oxo-1,6-dihydro-pyridin-3-yl]-phenyl}-2H-isoquinolin-1-one, 6-cyclopropyl-3-dimethylaminomethyl-2-{2-hydroxymethyl-3-[1-methyl-5-(5-morpholin-4-yl-pyridin-2-ylamino)-6-oxo-1,6-dihydro-pyridin-3-yl]-phenyl}-2H-isoquinolin-1-one, 3-tert-butoxymethyl-6-cyclopropyl-2-{3-[5-(6-fluoro-pyridin-2-ylamino)-1-methyl-6-oxo-1,6-dihydro-pyridin-3-yl]-2-hydroxymethyl-phenyl}-2H-isoquinolin-1-one, 6-dimethylamino-2-{2-hydroxymethyl-3-[1-methyl-5-(6-methylamino-pyridin-2-ylamino)-6-oxo-1,6-dihydro-pyridin-3-yl]-phenyl}-2H-isoquinolin-1-one, 6-dimethylamino-2-(2-hydroxymethyl-3-{1-methyl-5-[6-(4-methyl-piperazin-1-yl)-pyridin-2-ylamino]-6-oxo-1,6-dihydro-pyridin-3-yl}-phenyl)-2H-isoquinolin-1-one, 2-{3-[5-(6-amino-pyridin-2-ylamino)-1-methyl-6-oxo-1,6-dihydro-pyridin-3-yl]-2-hydroxymethyl-phenyl}-6-dimethylamino-2H-isoquinolin-1-one, 2-(6-{5-[3-(6-dimethylamino-1-oxo-1H-isoquinolin-2-yl)-2-hydroxymethyl-phenyl]-1-methyl-2-oxo-1,2-dihydro-pyridin-3-ylamino}-pyridin-3-yloxy)-N-methyl-acetamide; 2-{3-[5-(5,6-dimethoxy-pyridin-2-ylamino)-1-methyl-6-oxo-1,6-dihydro-pyridin-3-yl]-2-hydroxymethyl-phenyl}-6-dimethylamino-2H-isoquinolin-1-one, 6-dimethylamino-2-(2-hydroxymethyl-3-{5-[5-methoxy-6-(2-methoxy-ethoxy)-pyridin-2-ylamino]-1-methyl-6-oxo-1,6-dihydro-pyridin-3-yl}-phenyl)-2H-isoquinolin-1-one, 6-dimethylamino-2-(2-hydroxymethyl-3-{5-[6-methoxy-5-(2-methoxy-ethoxy)-pyridin-2-ylamino]-1-methyl-6-oxo-1,6-dihydro-pyridin-3-yl}-phenyl)-2H-isoquinolin-1-one, 2-(3-{5-[5,6-bis-(2-methoxy-ethoxy)-pyridin-2-ylamino]-1-methyl-6-oxo-1,6-dihydro-pyridin-3-yl}-2-hydroxymethyl-phenyl)-6-dimethylamino-2H-isoquinolin-1-one, 6-dimethylamino-2-{2-hydroxymethyl-3-[1-methyl-5-(2-morpholin-4-yl-pyrimidin-4-ylamino)-6-oxo-1,6-dihydro-pyridin-3-yl]-phenyl}-2H-isoquinolin-1-one, 6-dimethylamino-2-(2-hydroxymethyl-3-{1-methyl-5-[2-(4-methyl-piperazin-1-yl)-pyrimidin-4-ylamino]-6-oxo-1,6-dihydro-pyridin-3-yl}-phenyl)-2H-isoquinolin-1-one; 2-[4-(6-{5-[3-(6-dimethylamino-1-oxo-1H-isoquinolin-2-yl)-2-hydroxymethyl-phenyl]-1-methyl-2-oxo-1,2-dihydropyridin-3-ylamino}-pyridin-3-yl)-piperazin-1-yl]-isobutyramide, 2-(3-{5-[6-(4-acetylpiperazin-1-yl)-pyridin-2-ylamino]-1-methyl-6-oxo-1,6dihydropyridin-3-yl}-2-hydroxymethyl-phenyl)-6-dimethylamino-2H-isoquinolin-1-one, 6-dimethylamino-2-{3-[5-(5-ethyl-1H-pyrazol-3-ylamino)-1-methyl-6-oxo-1,6-dihydro-pyridin-3-yl]-2-hydroxymethyl-phenyl}-2H-isoquinolin-1-one, 6-dimethylamino-2-(3-{5-[5-(2-hydroxy-ethoxy)-6-(2-methoxy-ethoxy)-pyridin-2-ylamino]-1-methyl-6-oxo-1,6-dihydro-pyridin-3-yl}-2-hydroxymethyl-phenyl)-2H-isoquinolin-1-one, 6-cyclopropyl-2-(2-hydroxymethyl-3-{1-methyl-5-[5-(morpholine-4-carbonyl)-pyridin-2-ylamino]-6-oxo-1,6-dihydro-pyridazin-3-yl}-phenyl)-2H-isoquinolin-1-one, and 2-(2-hydroxymethyl-3-{1-methyl-5-[5-(morpholine-4-carbonyl)-pyridin-2-ylamino]-6-oxo-1,6-dihydro-pyridazin-3-yl}-phenyl)-6-(1-methyl-cyclopropyl)-2H-isoquinolin-1-one, as disclosed in U.S. Pat. No. 7,906,509 to Kennedy-Smith et al., incorporated herein by this reference; 6-dimethylamino-2-(2-methyl-3-{1-methyl-5-[5-(morpholine-4-carbonyl)-pyridin-2-ylamino]-6-oxo-1,6-dihydro-pyridin-3-yl}-phenyl)-2H-phthalazin-1-one, 6-dimethylamino-2-(2-hydroxymethyl-3-{1-methyl-5-[5-(morpholine-4-carbonyl)-pyridin-2-ylamino]-6-oxo-1,6-dihydro-pyridin-3-yl}-phenyl)-2H-phthalazin-1-one, 6-dimethylamino-2-{2-hydroxymethyl-3-[1-methyl-5-(5-morpholin-4-yl-pyridin-2-ylamino)-6-oxo-1,6-dihydro-pyridin-3-yl]-phenyl}-2H-phthalazin-1-one, 6-tert-butyl-2-(2-hydroxymethyl-3-{1-methyl-5-[5-(morpholine-4-carbonyl)-pyridin-2-ylamino]-6-oxo-1,6-dihydro-pyridin-3-yl}-phenyl)-2H-phthalazin-1-one, 6-tert-butyl-2-{2-hydroxymethyl-3-[1-methyl-5-(5-morpholin-4-yl-pyridin-2-ylamino)-6-oxo-1,6-dihydro-pyridin-3-yl]-phenyl}-2H-phthalazin-1-one, 6-tert-butyl-2-[2-hydroxymethyl-3-(5-{5-[4-(2-methoxy-ethyl)-piperazin-1-yl]-pyridin-2-ylamino}-1-methyl-6-oxo-1,6-dihydro-pyridin-3-yl)-phenyl]-2H-phthalazin-1-one, 6-dimethylamino-2-[2-hydroxymethyl-3-(5-{5-[4-(2-methoxy-ethyl)-piperazin-1-yl]-pyridin-2-ylamino}-1-methyl-6-oxo-1,6-dihydro-pyridin-3-yl)-phenyl]-2H-phthalazin-1-one, 6-dimethylamino-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-phthalazin-1-one, 2-(3-{5-[5-(4-acetyl-piperazin-1-yl)-pyridin-2-ylamino]-1-methyl-6-oxo-1,6-dihydro-pyridin-3-yl}-2-hydroxymethyl-phenyl)-6-tert-butyl-2H-phthalazin-1-one, 2-(3-{5-[5-(4-acetyl-piperazin-1-yl)-pyridin-2-ylamino]-1-methyl-6-oxo-1,6-dihydro-pyridin-3-yl}-2-hydroxymethyl-phenyl)-6-dimethylamino-2H-phthalazin-1-one, 6-tert-butyl-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-phthalazin-1-one, 6-tert-butyl-2-(3-{5-[5-(4-ethyl-piperazin-1-yl)-pyridin-2-ylamino]-1-methyl-6-oxo-1,6-dihydro-pyridin-3-yl}-2-hydroxymethyl-phenyl)-2H-phthalazin-1-one, 6-tert-butyl-2-{2-hydroxymethyl-3-[1-methyl-6-oxo-5-(5-piperazin-1-yl-pyridin-2-ylamino)-1,6-dihydro-pyridin-3-yl]-phenyl}-2H-phthalazin-1-one, and 4-(6-{5-[3-(6-tert-butyl-1-oxo-1H-phthalazin-2-yl)-2-hydroxymethyl-phenyl]-1-methyl-2-oxo-1,2-dihydro-pyridin-3-ylamino}-pyridin-3-yl)-piperazine-1-carboxylic acid tert-butyl ester, as disclosed in U.S. Pat. No. 7,902,194 to Dewdney et al., incorporated herein by this reference; pyrazolopyrimidines, as disclosed in U.S. Pat. No. 7,741,330 to Chen et al., incorporated herein by this reference; imidazo[1,5-f][1,2,4]triazines, as disclosed in U.S. Pat. No. 7,732,454 to Verner, incorporated herein by this reference; 1-(3-(4-amino-3-(4-phenoxyphenyl)-1H-pyrazolo[3,4-d]pyrimidin-1-yl)piperidin-1-yl)prop-2-en-1-one, (E)-1-(3-(4-amino-3-(4-phenoxyphenyl)-1H-pyrazolo[3,4-d]pyrimidin-1-yl)piperidin-1-yl)but-2-en-1-one, 1-(3-(4-amino-3-(4-phenoxyphenyl)-1H-pyrazolo[3,4-d]pyrimidin-1-yl)piperidin-1-yl)sulfonylethene, 1-(3-(4-amino-3-(4-phenoxyphenyl)-1H-pyrazolo[3,4-d]pyrimidin-1-yl)piperidin-1-yl)prop-2-yn-1-one, 1-(4-(4-amino-3-(4-phenoxyphenyl)-1H-pyrazolo[3,4-d]pyrimidin-1-yl)piperidin-1-yl)prop-2-en-1-one, N-((1S,4S)-4-(4-amino-3-(4-phenoxyphenyl)-1H-pyrazolo[3,4-d]pyrimidin-1-yl)cyclohexyl)acrylamide, 1-((R)-3-(4-amino-3-(4-phenoxyphenyl)-1H-pyrazolo[3,4-d]pyrimidin-1-yl)pyrrolidin-1-yl)prop-2-en-1-one, 14(S)-3-(4-amino-3-(4-phenoxyphenyl-1H-pyrazolo[3,4-d]pyrimidin-1-yl)pyrrolidin-1-yl)prop-2-en-1-one, 14(R)-3-(4-amino-3-(4-phenoxyphenyl)-1H-pyrazolo[3,4-d]pyrimidin-1-yl)piperidin-1-yl)prop-2-en-1-one, 1-((S)-3-(4-amino-3-(4-phenoxyphenyl)-1H-pyrazolo[3,4-d]pyrimidin-1-yl)piperidin-1-yl)prop-2-en-1-one, and (E)-1-(3-(4-amino-3-(4-phenoxyphenyl)-1H-pyrazolo[3,4-d]pyrimidin-1-yl)piperidin-1-yl)-4-(dimethylamino)but-2-en-1-one, disclosed in U.S. Pat. No. 7,514,444 to Honigberg et al., incorporated herein by this reference; imidazo[1,2-a]pyrazin-8-ylamines, disclosed in U.S. Pat. No. 7,405,295 to Currie et al., incorporated herein by this reference; α-cyano-β-hydroxy-β-methyl-N-(2,5-dibromophenyl)-propenamide, α-cyano-β-hydroxy-β-methyl-N-[4-(methylsulfonyl)phenyl]-propenamide, α-cyano-β-hydroxy-β-methyl-N-[3-methylsulfonyl)phenyl]-propenamide, α-cyano-β-hydroxy-β-methyl-N-[3-bromo-4-(trifluoromethoxy)-phenyl]propenamide, α-cyano-β-hydroxy-β-methyl-N-(2,4-dibromophenyl)-propenamide, α-cyano-β-hydroxy-β-methyl-N-(2,4-dichlorophenyl)-propenamide, α-cyano-β-hydroxy-β-methyl-N-(2,5-dichlorophenyl)-propenamide, α-cyano-β-hydroxy-β-methyl-N-(3,4-didichlorophenyl)-propenamide, or pharmaceutically aceptable salts thereof, as disclosed in U.S. Pat. No. 6,753,348 to Uckun et al., incorporated herein by this reference; and calanolides, as disclosed in U.S. Pat. No. 6,306,897 to Uckun et al., incorporated herein by this reference. Other inhibitors of BTK are known in the art.

EGFR inhibitors include both monoclonal antibodies and small molecule inhibitors. Monoclonal antibody EGFR inhibitors include, but are not limited to, cetuximab, panitumumab, zalututmumab, nimotuzumab, and matuzumab. Small molecule EGFR inhibitors include, but are not limited to, gefitinib, erlotinib, and lapatinib.

c-Myc inhibitors include, but are not limited to, (Z,E)-5-(4-ethylbenzylidine)-2-thioxothiazolidin-4-one

PTEN inhibitors include, but are not limited to, SF1670 (N-(9,10-dihydro-9,10-dioxo-2-phenanthrenyl)-2,2-dimethyl-propanamide).

IDH inhibitors include, but are not limited to, AGI-5198 and AGI-221.

Polyamine analogs are described in R. A. Casero, Jr. & P. M. Woster, “Recent Advances in the Development of Polyamine Analogs as Antitumor Agents,” J. Med. Chem. 52: 4551-4573 (2009), incorporated herein by this reference; these agents include, but are not limited to, α-difluoromethylornithine, tetraamine A, tetraamine B, tetraamine C, bis(ethyl)polyamines, and macrocyclic polyamines.

Thalidomide, and its analogs lenalidomide and pomalidomide, inhibit myeloma cells by a number of mechanisms, including inhibition of angiogenesis, inhibition of production of interleukin-6, activation of apoptotic pathways through caspase-8-mediated cell death, and other mechanisms. The antineoplastic activity of thalidomide is described in W. D. Figg et al., “A Randomized Phase II Trial of Thalidomide, an Angiogenesis Inhibitor, in Patients with Androgen-Independent Prostate Cancer,” Clin. Cancer Res. 7: 1888-1893 (2001), incorporated herein by this reference.

Bruceantin is a quassinoid obtained from Brucea sp. It acts as an inhibitor of protein synthesis via interference at the peptidyltransferase site; an analog is brusatol.

Bisantrene is an anthracene derivative possessing antineoplastic activity (H. W. Yap et al., “Bisantrene: An Active New Drug in the Treatment of Metastatic Breast Cancer,” Cancer Res. 43: 1402-1404 (1983). Amsacrine is a planar fused ring intercalating agent; its activity is described in M. A. Horstmann et al., “Amsacrine Combined with Etoposide and High-Dose Methylprednisolone as Salvage Therapy in Acute Lymphoblastic Leukemia in Children,” Haematologica 90: 1701-1703 (2005), incorporated herein by this reference.

Mitoxantrone is an anthracenedione antineoplastic agent that acts as a type II topoisomerase inhibitor that acts as an intercalating agent; an analog is pixantrone.

Vosaroxin is a quinolone derivative with the structure 74(35,45)-3-methoxy-4-(methylamino)pyrrolidin-1-yl)-4-oxo-1-(thiazol-2-yl)-1,4-dihydro-1,8-naphthyridine-3-carboxylic acid. Its use is described in J. E. Lancet et al., “A Phase Ib Study of Vosaroxin, an Anticancer Quinolone Derivative, in Patients with Relapsed or Refractory Acute Leukemia,” Leukemia 25: 1808-1814 (2011), incorporated herein by this reference.

The use of dianhydrogalactitol and analogs, including dibromodulcitol, as antineoplastic agents, is described in PCT Patent Application Publication No. WO 2012/024367 by Brown, incorporated herein by this reference.

The antineoplastic agent 5-azacytidine is a chemical analog of cytidine that inhibits DNA methyltransferase, causing hypomethylation of DNA. Decitabine, or 5-aza-2′-deoxycytidine, has a similar mechanism of action.

Bevacizumab is a monoclonal antibody that inhibits VEGF-A.

Rituximab is an anti-CD20 monoclonal antibody. Other anti-CD20 monoclonal antibodies include ocrelizumab, ofatumumab, and obinutuzumab.

Anti-EGFR vaccines are described in K. C. Foy et al., “Peptide Vaccines and Peptidomimetics of EGFR (HER-1) Ligand Binding Domain Inhibit Cancer Cell Growth in Vitro and in Vivo,” J. Immunol. 191: 217-227 (2013), incorporated herein by this reference.

T-cell stimulants include, but are not limited to, tetrachlorodecaoxide, imiquimod, and resiquimod.

The use of dendritic cell vaccines is described in J. Banchereau et al., “Immune and Clinical Responses in Patients with Metastatic Melanoma to CD34⁺ Progenitor-Derived Dendritic Cell Vaccine,” Cancer Res. 61: 6451-6458 (2001), incorporated herein by this reference.

PD inhibitors include, but are not limited to, nivolumab and lambrolizumab.

Other therapeutic agents can be used in combination with amonafide and analogs thereof. These therapeutic agents modulate or affect a large number of pathways, targets, or cellular processes. These therapeutic agents can also be incorporated in compositions according to the present invention, as described below, wherein the composition comprises a drug combination. These pathways, targets, and cellular processes include, but are not limited to:

(1) c-Met (U.S. Pat. No. 8,691,838 to Albrecht et al.; quinoline derivatives such as 5-phenyl-3-(quinolin-6-ylmethyl)-6,7-dihydro-3H-[1,2,3]triazolo[4,5-c]pyridin-4(5H)-one; U.S. Pat. No. 8,658,643 Schadt et al.; pyrimidinyl pyridazinones, including 6-(1-methyl-1H-pyrazol-4-yl)-2-{3-[5-(2-morpholin-4-ylethoxy)pyrimidin-2-yl]-benzyl}-2H-pyridazin-3-one; U.S. Pat. No. 8,637,518 to Stieber et al.; pyridazinone compounds including (25,35)-2-amino-3-methoxy-N-[2-(2-{3-[3-(1-methyl-1H-pyrazol-4-yl)-6-oxo-6H-pyridazin-1-ylmethyl]phenyl}pyrimidin-5-yloxy)ethyl]-butyramide; U.S. Pat. No. 8,623,870 to Dorsch et al.; pyridazinone derivatives, including 6-(3,5-difluorophenyl)-2-{3-[5-(1-piperidin-4-yl-1H-pyrazol-4-yl)thiazol-2-yl]benzyl}-2H-pyridazin-3-one; U.S. Pat. No. 8,586,599 to Becker et al.; 6-(1-methyl-1H-pyrazol-4-yl)-2-{3-[5-(2-morpholin-4-yl-ethoxy)-pyrimidin-2-yl]-benzyl}-2H-pyridazin-3-one dihydrogenphosphate; U.S. Pat. No. 8,563,561 to Schadt et al.; derivatives of 3-(3-pyrimidin-2ylbenzyl)-1,2,4-triazolo[4,3-b]pyrimidine; U.S. Pat. No. 8,557,813 to Dorsch et al.; 2-benzylpyridazinone compounds including 2-[3-(5-bromopyrimidin-2-yl)benzyl]-6-cyclopropyl-2H-pyridazin-3-one; U.S. Pat. No. 8,551,989 to Michels et al.; substituted 4-(indazolyl)-1,4-dihydropyridines; U.S. Pat. No. 8,524,900 to Albrecht et al.; fused heterocyclic derivatives; U.S. Pat. No. 8,518,938 Lauffer et al.; phenyl-substituted aminopyridine compounds; U.S. Pat. No. 8,481,524 to Lauffer et al.; substituted aminopyridine compounds; U.S. Pat. No. 8,445,489 to Stieber et al.; aryl ether pyridazinone derivatives; U.S. Pat. No. 8,435,986 to Stieber et al.; bicyclic triazole derivatives including 6-bromo-1-{3-[5-(2-morpholin-4-ylethoxy)pyrimidin-2-yl]-benzyl}-1H-1,2,3-triazolo[4,5-b]pyrazine; U.S. Pat. No. 8,435,981 to Dorsch et al.; 2-heterocyclylbenzyl)pyridazinone derivatives, including 6-(3,5difluorophenyl)-2-[3-(5-methyl-1,2,4-oxadiazol-3-yl)benzyl]-2H-pyridazin-3-one; United States Patent Application Publication No. 2013/0184261 by Dorsch et al.; pyridazinones including 3-(1-{3-[5-(1-methylpiperidin-4-ylmethoxy)pyrimidin-2-yl]benzyl}-6-oxo-1,6-dihydropyridazin-3-yl)benzonitrile; United States Patent Application Publication No. 2013/0184269 by Dorsch et al.; pyridazinones including 4-{3-[3-(3,5-difluorophenyl)-6-oxo-6H-pyridazin-1-ylmethyl]phenyl}morpholin-3-one; United States Patent Application Publication No. 2013/0131055 by Michels et al.; 4-(furo[3,2-c]pyridin-2-yl)-1,4-dihydropyridine derivatives; United States Patent Application Publication No. 2013/0131037 by Dorsch et al.; 2-(heterocyclylbenzyl)pyridazinone derivatives including 6-(3,5-difluorophenyl)-2-[3-(5-methyl-1,2,4-oxadiazol-3-yl)benzyl]-2H-pyridazin-3-one; U.S. Pat. No. 8,431,572 to Schadt et al.; 2-oxo-3-benzylbenzoxazol-2-one derivatives including 3-(4-methylpiperazin-1-yl)propyl[3-(5-methoxy-2-oxobenzoxazol-3-ylmethyl)phenyl]carbamate; U.S. Pat. No. 8,426,397 Dorsch et al.; 3-(3-pyrimidin-2-ylbenzyl)-1,2,4-triazolo[4,3-b]pyridazine derivatives including methyl 3-[3-(5-bromopyrimidin-2-yl)benzyl]-1,2,4-triazolo[4,3-b]pyridazine-6-carboxylate; U.S. Pat. No. 8,598,184 to Zhang; 3H-[1,2,3]triazolo[4,5-d]pyrimidine derivatives including 6-((5-chloro-3H-[1,2,3]triazolo[4,5-d]pyrimidin-3-yl)methyl)quinoline; United States Patent Application Publication No. 2013/0116231 Wilson et al.; 1,4-dihydropyridazinone derivatives including 1-(1-methyl-1H-pyrazol-4-yl)-3-[(quinolin-6-yloxy)methyl]pyridazin-4(1H)-one); c-Met is a proto-oncogene that encodes a protein known as hepatocyte growth factor receptor (HG FR);

(2) Aurora kinase (U.S. Pat. No. 8,691,810 to Michaelides et al.; pyrrolopyridine and pyrrolopyrimidine derivatives; U.S. Pat. No. 8,436,179 to Michaelides et al.; 4-amino-N-[3-(diethylamino)propyl]-3-(4-{[(3-fluorophenyl)carbamoyl]amino}phenyl)thieno[3,2-c]pyridine-7-carboxamide; U.S. Pat. No. 8,426,408 to Curtin et al.; pyrimidines including (1S,2S,3R,4R)-3-({5-fluoro-2-[(1-methyl-1H-pyrazol-4-yl)amino]pyrimidin-4-yl}amino)bicyclo[2.2.1]hept-5-ene-2-carboxamide; United States Patent Application Publication No. 2014/0018405 by Zahn et al.; aniline derivatives including 3-[3-[[4-(methylaminomethyl)anilino]-phenylmethylidene]-2-oxo-1H-indol-6-yl]-N-ethylprop-2-ynamide; United States Patent Application Publication No. 2013/0303529 by Albrecht et al.; fused heterocyclic derivatives; PCT Patent Application Publication No. WO 2013/090666 by Shi et al.; N-(4-{4-amino-7-[1-(2-hydroxyethyl)-1H-pyrazol-4-yl]thieno[3,2-c]pyridin-3-yl}phenyl)-N′-(3-fluorophenyl)urea); Aurora kinases are serine/threonine kinases that control chromatid segregation in cell division;

(3) Anti-apoptotic proteins and/or apoptosis promoters (U.S. Pat. No. 8,686,136 to Bruncko et al.; benzenesulfonamide derivatives including -(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-(trifluoromethyl)benzenesulfonamide; U.S. Pat. No. 8,614,318 to Bruncko et al.; benzenesulfonamide derivatives including N-(6-(4-((4′-chloro(1,1′-biphenyl)-2-yl)methyl)piperazin-1-yl)-1,2-benzisoxazol-3-yl)-3-nitro-4-((2-(phenylsulfanyl)ethyl)amino)benzenesulfonamide; U.S. Pat. No. 8,604,036 to Dorsch et al.; pyridazinone derivatives including 6-(3,5-difluorophenyl)-2-(5′-methyl-2,2′-bipyridinyl-6-ylmethyl)-2H-pyridazin-3-one; U.S. Pat. No. 8,604,028 to Michels et al.; 4-(furo[3,2-c]pyridin-2-yl)-1,4-dihydropyridine derivatives; U.S. Pat. No. 8,586,754 to Bruncko et al.; piperazinylpiperidine compounds including 4-(4-{[2-(4-chlorophenyl)-4,4-dimethylcyclohex-1-en-1-yl]methyl}piperazin-1-yl)-2-(1H-indol-5-yloxy)-N-({4-[(1-methylpiperidin-4-yl)amino]-3-nitrophenyl}sulfonyl)benzamide; U.S. Pat. No. 8,580,794 to Doherty et al.; piperazinylpyrrole compounds including methyl trans-4-{[(4-{[4-(4-{[2-(4-chlorophenyl)-4,4-dimethylcyclohex-1-en-1-yl]methyl}piperazin-1-yl)-2-(1H-pyrrolo[2,3-b]pyridin-5-yloxy)benzoyl]sulfamoyl}-2-nitrophenyl)amino]methyl}cyclohexanecarboxylate; U.S. Pat. No. 8,580,781 to Dorsch et al.; pyridazinone derivatives, including 3-(1-{3-[5-(1-methyl-piperidin-4-ylmethoxy)pyrimidin-2-yl]benzyl}-6-oxo-1-,6-dihydropyridazin-3-yl)benzonitrile; U.S. Pat. No. 8,563,735 Bruncko et al.; piperazinyl derivatives including 4-(4-{[2-(4-chlorophenyl)-4,4-dimethylcyclohex-1-en-1-yl]methyl}piperazin-1-yl)-N-{[5-chloro-6-(tetrahydro-2H-pyran-4-ylmethoxy)pyridin-3-yl]sulfonyl}-2-(1H-indazol-4-yloxy)benzamide; U.S. Pat. No. 8,557,983 Doherty et al.; substituted piperazine derivatives including N-({3-chloro-4-[(4-fluorotetrahydro-2H-pyran-4-yl)methoxy]phenyl}sulfonyl)-4-(4-{[2-(4-chlorophenyl)-4,4-dimethylcyclohex-1-en-1-yl]methyl}piperazin-1-yl)-2-[(6-fluoro-1H-indol-5-yl)oxy]benzamide; U.S. Pat. No. 8,546,399 to Bruncko et al.; pyrrolopyrimidine derivatives including trans-4-(4-{[2-(4-chlorophenyl)-4,4-dimethylcyclohex-1-en-1-yl]methyl}piperazin-1-yl)-N-({4-[(4-morpholin-4-ylcyclohexyl)amino]-3-nitrophenyl}sulfonyl)-2-(1H-pyrrolo[2,3-b]pyridin-5-yloxy)benzamide; U.S. Pat. No. 8,518,970 to Baell et al.; tetrahydroquinolinyl derivatives including 2-(1-(benzo[d]thiazol-2-ylcarbamoyl)-1,2,3,4-tetrahydroquinolin-7-yl)thiazole-4-carboxylic acid; U.S. Pat. No. 8,426,422 to Hexamer et al.; piperazinyl derivatives including -(4-{[2-(4-chlorophenyl)-4,4-dimethylcyclohex-1-en-1-yl]methyl}piperazin-1-yl)-2-(1H-indol-5-yloxy)-N-[(3-nitrophenyl)sulfonyl]benzamide; United States Patent Application Publication No. 2014/0073640 by Judd et al.; tetrahydroisoquinoline derivatives including N-(1,3-benzothiazol-2-yl)-2-(4-{[(4-{[2R)-4-(dimethylamino)-1-(phenylsulfanyl)butan-2-yl]amino}-3-nitrophenyl)sulfonyl]carbamoyl}-1,3-thiazol-2-yl)-1,2,3,4-tetrahydroisoquinoline-8-carboxamide; United States Patent Application Publication No. 2014/0066621 by Bruncko et al.; piperazinyl derivatives including 2-(3-amino-5-chlorophenoxy)-4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)benzamide; United States Patent Application Publication No. 2014/0005190 by Baell et al.; tetrahydroquinoline derivatives including -(1-(benzo[d]thiazol-2-ylcarbamoyl)-1,2,3,4-tetrahydroquinolin-7-yl)-5-(4-fluorophenoxy)thiazole-4-carboxamide; United States Patent Application Publication No. 2013/0267534 by Bruncko et al.; piperazine derivatives including 4-(4-{[2-(4-chlorophenyl)-4,4-dimethylcyclohex-1-en-1-yl]methyl}piperazin-1-yl)-N-[(4-{[(trans-4-hydroxy-4-methylcyclohexyl)methyl]amino}-3-nitrophenyl)sulfonyl]-2-(1H-pyrrolo[2,3-b]pyridin-5-yloxy)benzamide; United States Patent Application Publication No. 2013/0184278 by Kunzer et al.; piperidyl derivatives including 4-[4-(cyclohexylmethyl)-4-methoxypiperidin-1-yl]-N-{[5-({(1R)-3-(dimethylamino)-1-[(phenylthio)methyl]propyl}amino)-4-nitrothien-2-yl]sulfonyl}benzamide; PCT Patent Application Publication No. WO 2013/055897 by Wang et al; 8-carbamoyl-2-(2,3-disubstituted pyrid-6-yl)-1,2,3,4-tetrahydroisoquinoline derivatives);

(4) HGF (U.S. Pat. No. 8,685,983 to Kim et al.; substituted amide derivatives including N-(3-fluoro-4-(6-(pyrrolidine-1-carboxamido)pyrimidin-4-yloxy)phenyl)-1-(2-hydroxy-2-methylpropyl)-5-methyl-3-oxo-2-phenyl-2,3-dihydro-1H-pyrazole-4-carboxamide); HGF is hepatocyte growth factor and is a cellular growth and mobility factor;

(5) VEGF (U.S. Pat. No. 8,685,983 to Kim et al.; substituted amide derivatives including N-(3-fluoro-4-(6-(pyrrolidine-1-carboxamido)pyrimidin-4-yloxy)phenyl)-1-(2-hydroxy-2-methylpropyl)-5-methyl-3-oxo-2-phenyl-2,3-dihydro-1H-pyrazole-4-carboxamide; U.S. Pat. No. 8,642,624 to Chen et al.; substituted alkylamine compounds including N-(3,3-dimethylindolin-6-yl)-{2-[(4-pyridylmethyl)amino](3-pyridyl)}carboxamide; U.S. Pat. No. 8,592,589 to Zhang et al.; N-(4-{4-amino-7-[1-(2-hydroxyethyl)-1H-pyrazol-4-yl]thieno[3,2-c]pyridin-3-yl}phenyl)-N′-(3-fluorophenyl)urea); VEGF is vascular endothelial growth factor;

(6) ALK and/or EML-4-ALK fusion protein (U.S. Pat. No. 8,680,111 to Bailey et al.; pyrazole derivatives including including 10(R)-7-amino-12-fluoro-2,10,16-trimethyl-15-oxo-10,15,16,17-tetrahydro-2-H-8,4-(metheno)pyrazolo[4,3-h][2,5,11]benzoxadiazacyclotetradecine-3-carbonitrile; United States Patent Application Publication No. 2013/0252961 by Bailey et al.; macrocyclic compounds; United States Patent Application Publication No. 2013/0217668 by Boezio et al.; benzimidazole and azabenzimidazole compounds; United States Patent Application Publication No. 2013/0158019 Bryan et al.; pyrimidine compounds); ALK is anaplastic lymphoma kinase and frequently exists as a fusion protein with EML4 as ELM4-ALK in malignancies;

(7) IAP BIR domains (U.S. Pat. No. 8,648,094 to Laurent et al.; heterocyclic compounds containing naphthalenesulfonamide and pyrrolidine moieties; U.S. Pat. No. 8,575,113 to Jarvis et al.; pyrrolidine compounds); IAP (inhibitors of apoptosis) is a family of anti-apoptotic proteins which possess a BIR (baculovirus) IAP repeat, a domain of about 70 amino acid residues;

(8) phosphoinositide-3 kinase, including phosphoinositide-3 kinase-α (U.S. Pat. No. 8,633,204 Cheng et al.; 4-methylpyridopyrimidinone compounds including 2-amino-8-[trans-4-(2-hydroxyethoxy)cyclohexyl]-6-(6-methoxypyridin-3-yl)-4-methylpyrido[2,3-d]pyrimidin-7(8H)-one; U.S. Pat. No. 8,598,157 to Wunberg et al.; 5-alkynylpyridines including N-[2-methoxy-5-(4-methyl-6-morpholin-4-yl-pyrimidin-5-ylethynyl)-pyridin-3-yl]benzenesulfonamide); these enzymes act as signal transducers and phosphorylate the hydroxyl group at the 3-position of phosphatidylinositol;

(9) CDK 4/6 and/or FLT3 (U.S. Pat. No. 8,623,885 to Fakhoury et al.; fused tricyclic compounds; U.S. Pat. No. 8,623,885 to Chen et al.; fused tricyclic compounds);

(10) proteasome inhibitors (U.S. Pat. No. 8,597,904 to Bachmann et al.; syrbactin compounds); proteasomes are protein complex that degrade proteins by proteolysis;

(11) EGFR inhibitors, including antibodies (U.S. Pat. No. 8,580,263 to Adams et al.; anti-EGFR antibodies, including bispecific antibodies; U.S. Pat. No. 8,466,165 to Fakhoury et al.; 4-phenylamino-quinazolin-6-yl amides; United States Patent Application Publication No. 2013/0266563 by Gokaraju et al.; substituted 4-(selenophen-2(or-3)-ylaminopyrimidine compounds including 3-(6,7-dimethoxyquinazolin-4-ylamino)-5-tert-butylselenophene-2-carboxamide;) EGFR is epidermal growth factor receptor;

(12) PDK1 (U.S. Pat. No. 8,575,203 to Engelhardt et al.; quinoxaline, quinoline and quinazoline compounds, including N-[3-[4-[[(3R)-1-methylpyrrolidin-3-yl]methoxy]quinolin-6-yl]prop-2-ynyl]-2-oxo-1-[(3,4,5-trifluorophenyl)methyl]pyridine-3-carboxamide; United States Patent Application Publication No. 2013/0210832 by McConnell et al.; 1H-imidazo[4,5-c]quinolines including 1-[(3,4-difluorophenyl)methyl]-N-[3-(2-methyl-1-propan-2-ylimidazo[4,5-c]quinolin-8-yl)prop-2-ynyl]-6-oxopyrimidine-5-carboxamide; U.S. Pat. No. 8,664,236 to Heinrich et al.; 1H-pyrrolo[2,3-b]pyridine derivatives including N4-(3-fluorophenyl)-N4-methyl-6-[5-(1-methyl-1H-pyrazol-4-yl)-1H-pyrrolo[2,3-b]pyridin-3-yl]pyrimidine-2,4-diamine; U.S. Pat. No. 8,648,201 to Calderini et al.; aminopyridine derivatives including 6-{2-[2-amino-5-(1-methyl-1H-pyrazol-4-yl)pyridin-3-yl]-3H-benzimidazol-5-yl}-5-methyl-4,5-dihydro-2H-pyridazin-3-one; U.S. Pat. No. 8,575,163 to Wucherer-Plietker et al.; pyrrolopyridinylpyrimidin-2-ylamine derivatives including 2-amino-6-[5-(1-methyl-1H-pyrazol-4-yl)-1H-pyrrolo[2,3-b]pyridin-3-yl]pyrimidin-4-yl}-1-(3-fluoropyridin-4-yl)propan-1-ol; U.S. Pat. No. 8,546,390 to Dorsch et al.; 6-(pyrrolopyridinyl)pyrimidin-2-ylamine derivatives including 4-butyl-6-(1H-pyrrolo[2,3-b]pyridin-3-yl)pyrimidin-2-yl-amine; U.S. Pat. No. 8,541,584 to Dorsch et al.; 3-(1,2,3-triazol-4-yl)pyrrolo[2,3-b]pyridine including 3-(1-benzyl-1H-1,2,3-triazol-4-yl)-1H-pyrrolo[2,3-b]pyridine; U.S. Pat. No. 8,455,477 to Katz et al.; with pyrazolopyridines and imidazopyridines including 6-bromo-4-methoxypyrazolo[1,5-a]pyridine; United States Patent Application Publication No. 2013/0310391 by Dorsch et al.; 7-azaindole derivatives including 3-(1H-pyrrolo[2,3-b]pyridin-3-yl)quinolin-2-ylamine; United States Patent Application Publication No. 2013/0245355 by Buchstaller et al.; 3-heteroaryl-substituted pyrrolo[2,3-b]pyridine derivatives including 1-{3-[5-(1-methyl-1H-pyrazol-4-yl)-1H-pyrrolo[2,3-b]pyridin-3-yl]isoxazol-5-yl}-1-phenylethanol; United States Patent Application Publication No. 2013/0231519 by Heinrich et al; 1H-pyrrolo[2,3-b]pyridine derivatives including N4-(3-fluorophenyl)-N4-methyl-6-[5-(1-methyl-1H-pyrazol-4-yl)-1H-pyrrolo[2,3-b]pyridin-3-yl]pyrimidine-2,4-diamine); PDK1 is phosphoinositide-dependent kinase 1;

(13) Tie-2 (U.S. Pat. No. 8,557,996 Chaffee et al.; heteroaryl-substituted alkynes including 3-(3H-imidazo[4,5-b]pyridin-6-ylethynyl)-4-methyl-N-(3-(trifluoromethyl)phenyl)benzamide; U.S. Pat. No. 8,476,434 to Geuns-Meyer et al.; substituted heterocyclic compounds; United States Patent Application Publication No. 2013/0190290 by Su Meier et al.; 1-phenyl-1H-imidazo[1,2-b]pyrazole derivatives); Tie-2 is an angiopoietin receptor

(14) autotaxin (U.S. Pat. No. 8,557,824 Schiemann et al.; piperidinyl derivatives including 4-chlorobenzyl 4-[4-(3-amino-1H-indazol-6-ylcarbamoyl)thiazol-2-yl]piperidine-1-carboxylate; U.S. Pat. No. 8,530,650 to Schiemann et al.; 2,5-diamino-substituted [4,3-d]pyrimidines; U.S. Pat. No. 8,497,283 to Schultz et al. substituted benzotriazole compounds including 6-(1H-benzotriazole-5-carbonyl)-2-[3-(4-chloro-phenyl)ureido]-4,5,6,7-tetrahydro-thieno[2,3-c]pyridine-3-carboxylic acid amide; U.S. Pat. No. 8,557,824 to Schiemann et al.; thiazole derivatives including 4-chlorobenzyl 4-[4-(1H-benzotriazol-5-ylcarbamoyl)thiazo]-2-yl]piperazine-1-carboxylate; U.S. Pat. No. 8,552,001 to Schiemann et al.; sulfoxide derivatives); autotaxin is also known as ectonucleotide pyrophosphatase/phosphodiesterase 2 and is a secreted enzyme involved in the generation of the signaling molecule lysophosphatidic acid (LPA);

(15) IGF1R (U.S. Pat. No. 8,546,443 to Treu et al.; benzylic oxindole pyrimidines; U.S. Pat. No. 8,536,180 to Clark et al.; substituted pyrimidines, including 1-[3-({4-[2-(2-benzyl-1H-benzimidazol-5-yl)imidazo[1,2-a]pyridin-3-yl]pyrimidin-2-yl}amino)phenyl]pyrrolidin-2-one; U.S. Pat. No. 8,486,933 to Wang et al.; pyrimidines, including 5-bromo-N²-[4-(4-ethylpiperazin-1-yl)phenyl]-N⁴-[2-(trifluoromethyl)-1H-benzimidazol-5-yl]pyrimidine-2,4-diamine; United States Patent Application Publication No. 2014/0045832 by Balachandran et al.; phenoxy-substituted morpholinosulfonyl compounds including (S)-4-(2-carbamoyl-5-chloro-3-(2-(phenoxymethyl)morpholinosulfonyl)-1H-indol-7-ylamino)-4-oxobutanoic acid); IGFR1 is insulin-like growth factor receptor-1;

(16) Eg5 (U.S. Pat. No. 8,524,732 to Schiemann et al.; substituted tetrahydroquinolines); Eg5 is a member of the kinesin family of proteins and is involved in chromosome movement in mitosis;

(17) HER1 (EGFR, ErbB1), HER2 (neu, ErbB2), HER3 (ErbB3) and HER4 (ErbB4); (U.S. Pat. No. 8,524,722 Schirok et al.; substituted tricyclic compounds

(18) CHK-1 kinase (U.S. Pat. No. 8,518,952 to Braganza et al.; 6-substituted 2-heterocyclylaminopyrazine compounds); CHK-1 is a checkpoint kinase that coordinates the cellular DNA damage response and regulates progression through the cel cycle;

(19) mTor (U.S. Pat. No. 8,476,431 to Ren et al.; benzoxazole compounds; U.S. Pat. No. 8,476,282 to Ren et al.; benzoxazole compounds; United States Patent Application Publication No. 2013/0281474 by Meng et al.; fused tricyclic compounds; United States Patent Application Publication No. 2013/0150362 by Zhao et al.; pyrazolo[1,5-a]pyrimidine compounds including (1R,4R)-4-(7-amino-6-bromo-3-(6-phenylpyridin-3-yl)pyrazolo[1,5-a]pyrimidin-5-yl)-1-methylcyclohexanecarboxylic acid; U.S. Pat. No. 8,507,492 to Perrin-Ninkovic et al.; pyrazino[2,3-b]pyrazines including 7-(6-(1H-1,2,4-triazol-3-yl)pyridin-3-yl)-1-((cis-4-methoxycyclohexyl)methyl)-3,4-dihydropyrazino[2,3-b]pyrazin-2(1H)-one; U.S. Pat. No. 8,492,381 Perrin-Ninkovic et al.; pyrazino[2,3-b]pyrazines including 7-(6-(1H-1,2,4-triazol-3-yl)pyridin-3-yl)-1-(cis-4-hydroxycyclohexyl)-3,4-dihydropyrazino[2,3-b]pyrazin-2(1H)-one); mTor is the mammalian target of rapamycin and can control cell proliferation; it is dysregulated in certain malignancies;

(20) lipid kinases (U.S. Pat. No. 8,476,431 to Ren et al.; benzoxazole compounds; U.S. Pat. No. 8,476,282 to Ren et al.; benzoxazole compounds);

(21) SGK-1 (possibly also SGK-2 and SGK-3) (U.S. Pat. No. 8,466,170 to Klein et al.; 7-azaindole derivatives); SGK-1 is a serine/threonine kinase that regulates ion channels;

(22) matrix metalloproteinase 9 (U.S. Pat. No. 8,455,205 to Devy et al.; matrix metalloproteinase 9 binding proteins); this proteinase can promote malignancy, including invasion of tumor cells and metastasis;

(23) CDC7 (U.S. Pat. No. 8,450,320 to Zhu et al.; pyrrolopyrazinones, including 2-fluoro-5-{[1-oxo-7-(pyridin-4-yl)-1,2-dihydropyrrolo[1,2-a]pyrazin-4-yl]methyl}benzonitrile; U.S. Pat. No. 8,435,980 to Florjancic et al.; pyrrolopyridines including 6-chloro-N-cyclohexyl-4-(1H-pyrrolo[2,3-b]pyridin-3-yl)pyridin-2-amine); CDC-7 is a kinase involed in regulation of the cell cycle;

(24) MCL-1 U.S. Pat. No. 8,445,679 to Wang et al.; 7-substituted indoles, including 7-(3-((4-(4-acetylpiperazin-1-yl)phenoxy)methyl)-1,5-dimethyl-1H-pyrazol-4-yl)-3-(3-(1-naphthyloxy)propyl)-1-(pyridine-3-ylmethyl)-1H-indole-2-carboxylic acid; United States Patent Application Publication No. 2014/0051683 Wang et al.; 7-substituted indole derivatives including 3-(3-(1-naphthyloxy)propyl)-7-((E)-2-phenylvinyl)-1H-indole-2-carboxylic acid; PCT Patent Application Publication No. WO 2014/047427 by Lee et al.; substituted benzofuran, benzothiophene, or indole compounds); MCL-1 is induced myeloid leukemia cell differentiation protein 1 and may act by inhibiting apoptosis;

(25) FAK (U.S. Pat. No. 8,440,822 to Luzzio et al.; sulfonyl amide derivatives; United States Patent Application Publication No. 2013/0158005 by Heinrich et al.; pyrimidine compounds including N-(2-{2-[2-(4-methanesulfonylphenylamino)pyrimidin-4-yl]ethyl}phenyl)-N-methylmethanesulfonamide); FAK is focal adhesion kinase, which is involved in tumor metastasis;

(26) sphingosine kinases (U.S. Pat. No. 8,436,186 to Stieber et al.; thiazolyl piperidine derivatives, including 4-(2-methyl-3-{4-[4-(5,5,8,8-tetramethyl-5,6,7,8-tetrahydronaphthalen-2-yl)thiazol-2-yl]piperidin-1-yl}propyl)morpholine);

(27) PDGFR (U.S. Pat. No. 8,436,179 to Michaelides et al.; 4-amino-N-[3-(diethylamino)propyl]-3-(4-{[(3-fluorophenyl)carbamoyl]amino}phenyl)thieno[3,2-c]pyridine-7-carboxamide); PDGFR is platelet-derived growth factor receptor;

(28) hedgehog (U.S. Pat. No. 8,431,597 to Munchhof et al.; benzimidazole derivatives); the hedgehog pathway is a key signaling pathway involved in differention;

(29) Kdr (U.S. Pat. No. 8,586,566 to Wang et al.; unsaturated heterocyclic derivatives including (E)-N-(4-methyl-3-(2-(6-(methylamino)-9H-purin-9-yl)vinyl)phenyl)-3-(trifluoromethyl)benzamide (also inhibitors of Src); U.S. Pat. No. 8,470,851 to Zou et al.; substituted acetylenic imidazo[1,2-a]pyridines, including 3-(imidazo[1,2-a]pyridin-3-ylethynyl)-4-methyl-N-(3-(4-methyl-1H-imidazol-1-yl)-5-(trifluoromethyl)phenyl)benzamide (also inhibitors of Src); Kdr is vascular endothelial growth factor 2 and is involved in atherogenesis;

(30) Pim (United States Patent Application Publication No. 2014/0031360 by Wang et al.; bicyclic compounds including 5-(6-cyclopropyl-2-pyrazinyl)-3-(6-(4-piperidinyloxy)-2-pyrazinyl)-1H-indazole; PCT Patent Application No. WO 2013/0130660 by Cee et al.; heterocyclic amides including -(2-methyl-3-((1-methylcyclopropyl)amino)-5-quinoxalinyl)-1,5,6,7-tetrahydro-4H-pyrrolo[3,2-c]pyridin-4-one); Pim is a serine/threonine kinase involved in cellular signaling;

(31) TGF-β receptor (United States Patent Application Publication No. 2013/0345227 by Dorsch et al.; pyrido[2,3-b]pyrazine derivatives; United States Patent Application Publication No. 2013/0102608 by Hoelzemann et al.; bipyridyl derivatives);

(32) BET bromodomains (United States Patent Application Publication No. 2013/0331382 by Hubbard et al.; pyridinone and pyridazinone derivatives including 3-methyl-5-(2-phenoxyphenyl)pyridin-2(1H)-one; United States Patent Application Publication No. 2013/0281450 by Pratt et al.; isoindolone derivatives including 1-(1,3-benzodioxol-5-yl)-3-methyl-2,5,6,7-tetrahydro-4H-isoindol-4-one); bromodomains are acetyl-lysine recognition domains in several proteins that can regulate the activity of c-Met;

(33) NAMPT (United States Patent Application Publication No. 2013/0303511 by Clark et al.; imidazopyridine derivatives including 4-(1-benzoylpiperidin-4-yl)-N-(imidazo[1,2-a]pyridin-7-ylmethyl)benzamide; PCT Patent Application Publication No. WO 2013/170118 by Sweis et al.; thiazolecarboxamide; including 2-{(4-fluorobenzyl)[4-(pyridin-3-yl)benzyl]amino}-N-[3-(1H-imidazol-1-yl)propyl]-1,3-thiazole-5-carboxamide); NAMPT is nicotinamide phosphoribosyltransferase;

(34) wee-1 (United States Patent Application Publication No. 2013/0225589 by Woods et al.; pyridopyrimidinone compounds including ethyl 2-{[4-(4-methylpiperazin-1-yl)phenyl]amino}-5-oxo-6-(prop-2-en-1-yl)-5,6-dihydropyrido[4,3-d]pyrimidine-8-carboxylate); wee-1 is a nuclear serine/threonine kinase that regulates cell cycle progression;

(35) Smoothened (United States Patent Application No. 2013/0210800 by Nair et al.; pyridine-2 derivatives); Smoothened is a G-protein-coupled receptor that is part of the hedgehog pathway;

(36) B-Raf (United States Patent Application No. 2013/0225562 by Ettmayer et al.; pyridyltriazoles including 5-tert-butyl-furan-2-carboxylic acid (5-{4-[5-(4-isopropyl-piperazin-1-yl)-pyridin-3-yl]-[1,2,3]triazol-1-yl}-6-methyl-pyridin-3-yl)-amide; United States Patent Application No. 2013/0190286 by Steurer et al.; phenyltriazoles including N-(5-tert-butyl-1,2-oxazol-3-yl)-4-methyl-3-[4-[5-(4-methylpiperazin-1-yl)pyridin-3-yl]triazol-1-yl]benzamide);

(37) LPA receptor antagonists (United States Patent Application Publication No. 2013/0165478 by Schiemann et al.; pyrazolopyridinone derivatives including 3-(3-fluorophenyl)-4-hydroxy-4-trifluoromethyl-1,4,5,7-tetrahydropyrazolo[3,4b]pyridin-6-one);

(38) FGFR (United States Patent Application Publication No. 2013/0158000 by Brohm et al.; disubstituted 5-(1-benzothiophen-2-yl)pyrrolo[2,1-f][1,2,4]triazin-4-amine derivatives); FDFR is fibroblast growth factor receptor;

(39) HDAC6 (PCT Patent Application Publication No. WO 2013/078544 by Wang et al.; heterocyclic amides; United States Patent Application Publication No. 2013/0281484 by Kozikowski et al; fused tricyclic compounds including at least one phenyl moiety); HDAC6 is one of the isoforms of histone deacetylase;

(40) androgen receptor (reduction of expression) (PCT Patent Application Publication No. WO 2013/071177 by Stein et al.; 17-heteroarylsteroid derivatives; United States Patent Application Publication No. 2014/088120 by Dilly et al.; benzo(iso)oxazolepiperidines);

(41) BTK (PCT Patent Application Publication No. WO 2013/113097 by Wang et al.; azaspiro cyclic molecules including 1-(6-(4-amino-3-(4-phenoxyphenyl)-1H-pyrazolo[3,4-d]pyrimidin-1-yl)-2-azaspiro[3.3]heptan-2-yl)prop-2-en-1-one; U.S. Pat. No. 8,697,711 to Honigberg et al.; pyrazolo-pyrimidinyl-piperidine compounds including (R)-1-(3-(4-amino-3-(4-phenoxyphenyl)-1H-pyrazolo[3,4-d]pyrimidin-1-yl)piperi-din-1-yl)prop-2-en-1-one; U.S. Pat. No. 8,501,724 to Chen et al.; purinone derivatives; United States Patent Application Publication No. 2014/0080844 by Chen et al.; pyrazolo-pyrimidinyl-piperidine compounds including 1-(3-(4-amino-3-(4-(3,4-dihydroxyphenoxy)phenyl)-6-hydroxy-1H-pyrazolo[3,4-d]pyrimidin-1-yl)piperidin-1-yl)propan-1-one; United States Patent Application Publication No. 2014/0079690 by Buggy et al.; pyrazolo-pyrimidinyl-piperidine compounds including (E)-4-(N-(2-hydroxyethyl)-N-methylamino)-1-(3-(4-phenoxyphenyl)-1H-pyrazolo[3,4-d]pyrimidin-1-yl)piperidin-1-yl)but-2-en-1-one; United States Patent Application Publication No. 2013/0310402 Buggy et al.; pyrazolo-pyrimidinyl-piperidine compounds including (R)-1-(3-(4-amino-3-(4-phenoxyphenyl)-1H-pyrazolo[3,4-d]pyrimidin-1-yl)piperidin-1-yl)prop-2-en-1-one; United States Patent Application Publication No. 2013/0172314 by Chen et al.; pyrazolo[3,4-d]pyrimidine and pyrrolo[2,3-d]pyrimidine compounds);

(42) phosphodiesterase 9 (PCT Patent Application Publication No. WO 2013/142269 by Ripka et al.; imidazotriazinone compounds including (−)-2-((3,4-trans)-1-benzyl-4-methylpyrrolidin-3-yl)-7-(tetrahydro-2H-pyran-4-yl)imidazo[1,5-f][1,2,4]triazin-4(3H)-one;

(43) IGFR (PCT Patent Application Publication No. WO 2013/110585 by Treu; 5,8-dihydro-6H-pyrazolo[3,4-h]quinazolines; PCT Patent Application Publication No. WO 2013/148227 by Roychoudhury et al.; morpholinoindole derivatives including (S)-3-((2-(([1,1′-biphenyl]-3-yloxy)methyl)morpholino)sulfonyl)-5-chloro-1H-indole-2-carboxamide);

(44) LR (PCT Patent Application Publication No. WO 2013/110585 by Treu; 5,8-dihydro-6H-pyrazolo[3,4-h]quinazolines);

(45) EZH2 (PCT Patent Application Publication No. WO 2014/049488 by Edwards et al.; benzamide and heterobenzamide compounds); EZH2 is a histone lysine N-methyltransferase;

(46) tankyrase (PCT Patent Application Publication No. WO 2014/036022 by Bregman et al.; quinazolinones including trans-N-(2-(4-oxo-3,4-dihydro-2-quinazolinyl)ethyl)-4-(5-phenyl-1,3,4-oxadiazol-2-yl)cyclohexanecarboxamide; PCT Patent Application Publication No. WO 2013/134079 by Bregman et al.; oxazolidinone compounds; PCT Patent Application Publication No. WO 2014/023390 by Dorsch et al.; azaisoquinoline derivatives; PCT Patent Application Publication No. WO 2013/143663; bicyclic pyrazinone derivatives including 3-(4-benzyloxy-phenyl)-2H-pyrrolo[1,2-a]pyrazin-1-one); tankyrase is a telomeric poly(ADP-ribose) polymerase;

(47) fatty acid synthase (FAS) (PCT Patent Application Publication No. WO 2014/044356 by Staehle et al.; hydropyrrolopyrrole derivatives including 1-[5-(4-isoquinolin-6-yl-benzoyl)-hexahydro-pyrrolo[3,4-c]pyrrol-2-yl]-propan-1-one; U.S. Pat. No. 8,598,153 to Singh et al.; derivatives of 3-acetamido-2,4-dihydroxybenzoic acid);

(48) PARP (PCT Patent Application Publication No. WO 2014/048532 by Dorsch et al.; substituted quinazoline derivatives; PCT Patent Application Publication No. WO 2013/117288 by Buchstaller et al.; tetrahydroquinazolinone derivatives including 2-(4-tert-butyl-piperazin-1-yl)-5,6,7,8-tetrahydro-3H-quinazolin-4-one; PCT Patent Application Publication No. WO 2013/164061 by Dorsch et al.; pyrrolotriazinone derivatives, including 2-p-tolyl-3H-pyrrolo[2,1-f][1,2,4]triazin-4-one); PARP is poly(ADP-ribose polymerase);

(49) GCN2 (PCT Patent Application Publication No. WO 2013/110309 by Dorsch et al.; triazolo[4,5-d]pyrimidines including [3-(4-methoxy-phenyl)-3H-[1,2,3]triazolo[4,5-d]pyrimidin-5-yl]-(1-phenyl-1H-pyrazol-4-yl)-amine; PCT Patent Application Publication No. WO 2013/131609 by Schiemann et al.; triazolopyrazine derivatives including 4-methanesulfonyl-phenyl)-[5-(1-methyl-1H-indazol-5-yl)-[1,2,4]triazolo[1,5-a]pyrazin-2-yl]amine); GCN2 is a serine/threonine protein kinase that binds to uncharged transfer RNA;

(50) TBK1 (PCT Patent Application Publication No. WO 2013/0117285 by Eggenweiler et al.; with furo[3,2-b]pyridine and thieno[3,2-b] derivatives including 5-[2-(4-morpholin-4-yl-phenyl)-furo[3,2-b]pyridin-7-yl]-2-(tetrahydro-pyran-4-yloxy)-benzonitrile); TBK1 is a serine/threonine protein kinase that can mediate NF-κB activation;

(51) IKK (PCT Patent Application Publication No. WO 2013/0117285 by Eggenweiler et al.; with furo[3,2-b]pyridine and thieno[3,2-b] derivatives including 5-[2-(4-morpholin-4-yl-phenyl)-furo[3,2-b]pyridin-7-yl]-2-(tetrahydro-pyran-4-yloxy)-benzonitrile); IKK is IκB kinase and is involved in the inflammatory response;

(52) Syk (PCT Patent Application Publication No. WO 2014/023385 by Burgdorf et al.; pyridopyrimidine derivatives including N2-((cis)-2-amino-cyclohexyl)-8-(1-methyl-1H-pyrazol-4-yl)-pyrido[4,3-d]pyrimidine-2,5-diamine; PCT Patent Application Publication No. WO 2013/124025 by Burgdorf et al.; furopyrimidine derivatives including 3-{7-[5-((R)-1-amino-ethyl)-2-methoxy-phenyl]-furo[3,2-b]pyridin-2-yl}-4-methoxybenzamide; PCT Patent Application Publication No. WO 2013/124026 by Deutsch et al.; 8-substituted 2-amino-[1,2,4]triazolo[1,5-a]pyrazines including (8-biphenyl-2-yl-[1,2,4]triazolo[1,5-a]pyrazin-2-yl)-(4-methoxy-phenyl)amine; U.S. Pat. No. 8,551,984 to Altman et al.; aminopyridines including (1R,4S)-4-[5-(3-cyclopropyl-5-{[4-(trifluoromethyl)pyrimidin-2-yl]amino}phenyl)-1,3-thiazol-2-yl]-4-hydroxy-2,2-dimethylcyclohexanecarboxylic acid; United States Patent Application Publication No. 2014/0100250 Altman et al.; pyridylaminopurines; PCT Patent Application Publication No. WO 2014/031438 by Haidle et al; substituted phenyl compounds including 4-{[(3′-methyl-5′-{[4-(trifluoromethyl)pyrimidin-2-yl]amino}bipheny 1-3-yl)sulfonyl]amino}benzoic acid; PCT Patent Application Publication No. WO 2013/192088 by Haidle et al.; substituted diazine and triazine Syk inhibitors including N-[3-(2-aminopyrimidin-5-yl)-5-methylphenyl]-4-(trifluoromethyl)pyrimidin-2-amine; PCT Patent Application Publication No. WO 2014/048065 by Machacek et al.; triazolyl derivatives including 2-methyl-3-[4-(4-methyl-6-{[4-(trifluoromethyl)pyridin-2-yl]amino}pyridin-2-yl)-1H-1,2,3-triazol-1-yl]propane-1,2-diol; PCT Patent Application Publication No. WO 2013/192125 by Machacek et al.; pyrazolyl derivatives including N-(3-methyl-5-(pyrazolo[1,5-a]pyridin-3-yl)phenyl)-4-(trifluoromethyl)pyrimidin-2-amine); Syk is spleen tyrosine kinase, a non-receptor kinase that is involved in signal transduction;

(53) AKT (U.S. Pat. No. 8,691,825 to Chen et al.; imidazopyrimidine derivatives including 1-[4-(6-phenylimidazo[1,2-a]pyrimidin-7-yl)phenyl]cyclobutanamine; U.S. Pat. No. 8,614,221 to Fan et al.; substituted fused naphthyridines including trans-3-amino-1-methyl-3-[4-(2-methyl-7-phenylpyrazolo[1,5-a]pyrido[3,2-e]pyrimidin-8-yl)phenyl]cyclobutanol; U.S. Pat. No. 8,536,193 to Furuyama et al.; substituted [1,2,4]triazolo[4,3-a]-1,5-naphthyridine compounds including trans-3-amino-3-{4-[1-(difluoromethyl)-8-phenyl[1,2,4]triazolo[4,3-a]-1,5-naphthyridin-7-yl]phenyl}-1-methylcyclobutanol); AKT, also known as protein kinase B, is a serine/threonine protein kinase that plays a key role in cellular proliferation and migration;

(54) HDAC (U.S. Pat. No. 8,686,020 to Hamblett et al.; substituted spirocyclic compounds including N-(2-aminophenyl)-6-(7-benzyl-2,7-diazaspiro[4.4]non-2-yl)nicotinamide; U.S. Pat. No. 8,466,193 to Verner et al.; 1,2-disubstituted-1H-benzimidazole-6-carboxylic acid hydroxyamide compounds, 1,3-disubstituted-indole-6-carboxylic acid hydroxyamide compounds, 1,3-disubstituted-azaindole-6-carboxylic acid hydroxyamide compounds, or substituted-1H-pyrrole-2-yl-N-hydroxyacrylamide compounds; U.S. Pat. No. 8,461,189 to Heidebrecht, Jr. et al.; pyridyl derivatives including pyridin-3-ylmethyl{[4-({[3-amino-6-(2-thienyl)pyridin-2-yl]amino}carbonyl)phenyl]methyl}carbamate; United States Patent Application Publication No. 2013/0156727 by Buggy et al.; 3-disubstituted-1H-indole-6-carboxylic acid hydroxyamides; United States Patent Application Publication No. 2013/0142758 by Verner et al.; hydroxamates; United States Patent Application Publication No. 2013/0137690 by Harrington et al.; 4-carboxybenzylamino derivatives including pyridin-3-ylmethyl[(4-{[(4-aminobiphenyl-3-yl)amino]carbonyl}phenyl)methyl]carbamate; United States Patent Application Publication No. 2013/0131041 by Berk et al.; spirocyclic compounds including N-(2-aminophenyl)-6-(4-oxo-1-phenyl-1,3,8-triazaspiro[4.5]dec-8-yl)nicotinamide; U.S. Pat. No. 8,653,278 to Kozikowski et al.; substituted biphenyl compounds; United States Patent Application Publication No. 2013/0156727 Buggy et al.; indole derivatives; HDAC is a group of enzymes (histone deacetylases) that remove acetyl groups from the ε-N-acetyl lysines of histone molecules;

(55) Ras, Raf, mutant B-Raf, VEGFR2 (KDR, Flk-1), FGFR2/3, c-Kit, PDGFR-13, or CSF-1R (U.S. Pat. No. 8,592,459 Aikawa et al; substituted benzimidazoles, including {1-methyl-5-[2-(5-trifluoromethyl-1H-imidazol-2-yl)-pyridin-4-yloxy]-1H-benzoimidazol-2-yl}-(4-trifluoromethylphenyl)-amine);

(56) methionine aminopeptidase-2 (U.S. Pat. No. 8,546,406 to Heinrich et al.; triazole derivatives including 5-((R)-1-[1,2,4]triazolo[1,5-a]pyrimidin-7-ylpyrrolidin-2-yl-methoxy)quinoline; United States Patent Application Publication No. 2013/0296274 by Heinrich et al.; phenylpyrrolidines including (S)-3-amino-2-oxo-1-phenylpyrrolidine-3-carboxylic acid (3-chloro-5-fluorobenzyl)amide);

(57) IκB kinases, JAK1, JAK2, JAK3 and TYK2 (U.S. Pat. No. 8,518,964 to Truchon et al.; tricyclic compounds including 1-amino-7-(trifluoromethyl)-5H-pyrido[4,3-b]indole-4-carboxamide);

(58) ERK (United States Patent Application Publication No. 2013/0261125 by Shipps, Jr. et al.; indazole derivatives including N-[3-[(2-fluorophenyl)methyl]-3-hydroxycyclohexyl]-3-(2-methyl-4-pyridinyl)-1H-indazole-5-carboxamide; United States Patent Application Publication No. 2013/0237518 by Boga et al; heterocyclic compounds; PCT Patent Application Publication No. WO 2014/052563 by Wilson et al.; 1H-pyrazolo-[4,3-c]pyridine derivatives; PCT Patent Application Publication No. WO 2013/063214 by Lim et al; tricyclic compounds); ERK is a group of kinases known as extracellular-signal-related

(59) CDK5 (United States Patent Application Publication No. 2013/0225591 by Machacek et al.; imidothiazoles including 2-[4-(2-oxo-1,3-oxazolidin-3-yl)phenyl]-N-[3-(trifluoromethyl)imidazo[5,1-b][1,3]thiazol-7-yl]acetamide) is cyclin-dependent kinase-5 and is involved in malignancy, apparently by reducing the activity of the actin regulatory protein caldesmon;

(60) GSK3-13 (United States Patent Application Publication No. 2013/0225591 by Machacek et al.; imidothiazoles including 2-[4-(2-oxo-1,3-oxazolidin-3-yl)phenyl]-N-[3-(trifluoromethyl)imidazo[5,1-b][1,3]thiazol-7-yl]acetamide); GSK3 is glycogen synthase kinase and is involved in a large number of signaling pathways;

(61) a kinase selected from the group consisting of Abl, Abl (T3151), BCR-Abl, ALK, BLK, CDK5, CDK2, CDK3, CDK7, CDK8, CSF1R, EML4-ALK, FAK, FER, FLT1, FLT3, FLT4, HIPK4, JNK2, KDR, kit, LCK, p38, RET, RIPK1, SLK, TEL-ALK, TIE1, TNK1, TTK and Src (United States Patent Application Publication No. 2013/0184287 by Gray et al.; indazole derivatives);

(62) p53 (increased activity) (PCT Patent Application Publication No. WO 2013/062923 by Nair et al.; macrocycles including 1-[2-(2-methoxyethoxy)phenyl-4-[[(10aR-6,7,10,10a,11,12,13,14-octahydro-16-oxo-1-(trifluoromethyl)-11(S)-[[5-(trifluoromethyl)-3-thienyl]oxy]-16H-dipyrido[1,2-e; 2′,3′-b][1,5]oxaazacycloundecin-11-yl]carbonyl]piperazine); p53 is a cellular protein that regulates the cell cycle and suppresses malignancy;

(63) HDM2 (PCT Patent Application Publication No. WO 2013/096150 by Ma et al.; substituted piperidines including 6-chloro-2-{[(2R,3S)-2-(2-morpholin-4-ylethyl)-1-{[4-(trifluoromethyl)pyridin-3-yl]carbonyl}-3-{[5-(trifluoromethyl)thiophen-3-yl]oxy}piperidin-3-yl]carbonyl}1,2,3,4-tetrahydroisoquinoline); HDM2 is a negative regulator of p53 and may thus have malignancy-promoting activity;

(64) acid ceramidase (U.S. Pat. No. 8,697,379 to Bielawska et al.; lysosomotropic inhibitors of acid ceramidase);

(65) BMI-1 (protein expression modulators) (U.S. Pat. No. 8,680,113 to Moon et al.; imidazolylpyrimidine compounds including N-(2,6-dichloro-4-methoxyphenyl)-4-(2-methylimidazo[1,2-a]pyrimidin-3-yl)-thiazol-2-amine); BMI-1 is a polycomb ring finger oncogene;

(66) Hsp70 (inducers of expression) (U.S. Pat. No. 8,680,100 to Jiang et al.; sulfonylhydrazide compounds including N′-(3-fluorophenylcarbonothioyl)-2-(2-(3-fluorophenylcarbonothioyl)-2-methylhydrazinyl)-N′-methyl-2-oxoethanesulfonohydrazide; U.S. Pat. No. 8,609,720 to Chen et al.; substituted macrocyclic compounds; U.S. Pat. No. 8,581,004 to Kowalczyk-Przelowka et al.; substituted macrocyclic compounds; U.S. Pat. No. 8,461,199 to Masazumi et al.; transition metal complexes of a bis[thio-hydrazide amide] compound; United States Patent Application Publication No. 2014/0031429 by Koya et al.; substituted macrocyclic compounds); Hsp70 is a member of the heat shock family of proteins and are involved in protein folding;

(67) Hsp90 (inhibitors of activity) (U.S. Pat. No. 8,648,104 to Du et al.; mercaptotriazoles, including 3-(2,4-dihydroxy-5-ethyl-phenyl)-4-(1-isopropyl-7-methoxy-indol-4-yl)-5-mercapto-triazole; U.S. Pat. No. 8,648,071 to Burlison et al.; hydrazonamide compounds; U.S. Pat. No. 8,629,285 Ying et al.; imidazole compounds; U.S. Pat. No. 8,486,932 to Burlison et al.; triazole compounds; U.S. Pat. No. 8,450,500 to Chimmanamada et al.; pyrrole compounds; United States Patent Application Publication No. 2013/0345219 by Lee et al.; triazinone and diazinone derivatives; United States Patent Application Publication No. 2013/0296378 by Sun et al.; substituted triazole compounds including 2-ethyl-6-[5-mercapto-4-(1-methyl-1H-indol-5-yl)-4H-[1,2,4]triazol-3-yl]pyridine-3,5-diol); Hsp90 is another heat shock protein with a number of functions in protein folding but which may stabilize growth factor receptors and signaling molecules in malignant cells;

(68) tyrosine kinases including inhibitors of BLK, BMX, EGFR, HER2, HER4, ITK, TEC, BTK, and TXK (U.S. Pat. No. 8,673,925 to Goldstein; pyrazolopyrimidines including (S)-2-(2-((4-amino-3-(2-fluoro-4-phenoxyphenyl)-1H-pyrazolo[3,4-d]pyrimidin-1-yl)methyppyrrolidine-1-carbonyl)-4,4-dimethylpent-2-enenitrile, 2-(2-((4-amino-3-(4-(2,3-difluorophenoxy)-2-fluorophenyl)-1H-pyrazolo[3,4-d]-pyrimidin-1-yl)methyppyrrolidine-1-carbonyl)-4,4-dimethylpent-2-enenitrile, 2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile; United States Patent Application Publication No. 2014/0073626 Goldstein et al.; azaindole derivatives including 3-(3-(7H-pyrrolo[2,3-d]pyrimidin-4-yl)phenyl)-2-cyano-N,N-dimethylacrylamide);

(69) inhibitors of angiogenesis (U.S. Pat. No. 8,648,096 to Muller et al.; N-methylaminomethyl isoindole compounds; U.S. Pat. No. 8,642,624 to Chen et al.; substituted alkylamine derivatives; U.S. Pat. No. 8,614,328 to Ruchelman et al.; 4′-O substituted isoindoline compounds; U.S. Pat. No. 8,518,972 Man et al.; 4′-arylmethoxy isoindoline derivatives; U.S. Pat. No. 8,492,395 to Muller et al.; 7-substituted quinazolinone derivatives; U.S. Pat. No. 8,481,568 to Muller et al.; isoindole-imide compounds including 1-cyclohexyl-3-[2-(2,6-dioxo-piperidin-3-yl)-1-oxo-2,3-dihydro-1H-isoindol-4-ylmethyl]-urea);

(70) peptide deformylase (U.S. Pat. No. 8,614,237 to Djaballah et al.; benzofuran-4,5-diones);

(71) INK pathway (U.S. Pat. No. 8,603,527 to Bhat et al.; 4-((9-((3S)-tetrahydro-3-furanyl)-8-((2,4,6-trifluorophenyl)amino)-9H-purin-2-yl)amino)-trans-cyclohexanol);

(72) tubulin polymerization (U.S. Pat. No. 8,598,366 to Sun et al.; oxazole derivatives; U.S. Pat. No. 8,501,790 to Demko et al.; [1,2,3]triazole derivatives; United States Patent Application Publication No. 2014/0045795 by Sun et al.; isoxazole derivatives);

(73) Src (U.S. Pat. No. 8,461,167 to Wang et al.; acetylenic heteroaryl compounds, including 4-methyl-N-{4-[(4-methylpiperazin-1-yl)methyl]-3-(trifluoromethyl)phenyl}-3-(pyrimidin-5-ylethynyl)benzamide; U.S. Pat. No. 8,586,566 to Wang et al.; unsaturated heterocyclic derivatives including (E)-N-(4-methyl-3-(2-(6-(methylamino)-9H-purin-9-yl)vinyl)phenyl)-3-(trifluoromethyl)benzamide); Src is a tyrosine-specific non-receptor protein kinase that may promote signals associated with malignant cells;

(74) prevention of metastasis (U.S. Pat. No. 8,569,348 to Shalwitz et al.; phenylsulfamic acid compounds including 4-{(S)-2-[(S)-2-(tert-butoxycarbonylamino)-3-phenylpropanamido]-2-(4-ethylthiazol-2-yl)ethyl}phenylsulfamic acid);

(75) thromboxane receptor/thromboxane synthase (U.S. Pat. No. 8,551,489 to Moussa et al.);

(76) prolyl hydroxylase inhibitors that can stabilize hypoxia inducible factor 1α and hypoxia inducible factor 2 (U.S. Pat. No. 8,536,181 Gardner et al.; piperazine compounds including methyl 4-{[1-(4-chlorobenzyl)-3-hydroxy-2-oxo-1,2-dihydropyridin-4-yl]methyl}piperazine-1-carboxylate);

(77) TNF-α (United States Patent Application Publication No. 2014/0073669 by Ruchelman et al.; 4′-O-substituted isoindoline compounds; United States Patent Application Publication No. 2013/0324518 by Man et al.; 4′-arylmethoxy isoindoline compounds); TNF-α is tumor necrosis factor-α and activates a number of signaling pathways associated with inflammation and the acute phase reaction;

(78) TTK (United States Patent Application Publication No. 2014/0051679 by Pauls et al.; indazolylpyrrolidine compounds including N-(3-(3-(morpholinomethyl)phenyl)-1H-indazol-5-yl)-2-(pyrrolidin-1-yl)-2-(thiophen-3-yl)acetamide); TTK is a dual specificity protein kinase;

(79) inhibition of production of cytokines such as TNF-α, IL-113, IL-12, IL-18, GM-CSF, and/or IL-6 (United States Patent Application Publication No. 2013/0289274 by Muller et al.; 6-, 7-, or 8-substituted quinazolinone derivatives);

(80) PAK (United States Patent Application Publication No. 2013/0158043 by Campbell et al.; substituted pyrido[2,3-d]pyrimidin-7(8H)-one compounds); PAK is one of a number of p21-activated kinases that serve as targets for GTP binding proteins and may be involved in cell motion and migration;

(81) PI3K (United States Patent Application Publication No. 2014/0100214 by Castro et al.; fused heterocyclic compounds; United States Patent Application Publication No. 2013/0217670 by Klein et al.; quinoxaline derivatives including 1-methyl-1H-imidazole-4-sulfonic acid [3-(6-methoxybenzo-1,3-dioxol-4-ylamino)quinoxalin-2-yl]amide; United States Patent Application Publication No. 2013/021081 9 by Klein et al.; quinazoline derivatives including 4-(2,3-dihydroindol-1-yl)-6-(1H-pyrrolo[2,3-b]pyridin-5-yl)quinazoline); PI3K is phosphoinositide-3 kinase, and is involved in cellular functions such as cell growth, proliferation, differentiation, motility, survival and intracellular trafficking, which in turn are involved in cancer; and

(82) Smac mimetics (United States Patent Application Publication No. 2013/0225567 by Reiser et al.; 5-alkynylpyridines); SMAC is also known as second mitochondria-derived activator of caspases and can act to promote apoptosis.

Other agents that can be used together with amonafide or a derivative or analog of amonafide to affect or modulate various pathways or targets include: (i) annellated 4-(indazolyl)-1,4-dihydropyridine derivatives as kinase inhibitors (U.S. Pat. No. 8,642,595 to Michels et al.); (ii) fused tricyclic dual inhibitors of CDK 4/6 and FLT3 (U.S. Pat. No. 8,623,885 to Fakhoury et al.); (iii) immunotoxins comprising: (a) a ligand that binds to a protein on the cancer cell attached to; (b) a toxin that is cytotoxic to the cancer cell (U.S. Pat. No. 8,545,840 to Zangemeister-Wittke et al.; (iv) phosphorus derivatives as kinase inhibitors (United States Patent Application Publication No. 2014/0066406 by Wang et al.); (v) bicyclic heteroaryl kinase inhibitors (United States 2013/0231337 by Zou et al.); (vi) spinosyns (U.S. Pat. No. 8,697,661 to Kritikou; and (vii) securinine or securinine analogs (United States Patent Application Publication No. 2014/0018383 by Wald et al.)

United States Patent Application Publication No. 2010/0069458 by Atadja et al., incorporated herein by this reference discloses the use of the following additional therapeutic agents, which can be used together with an alkylating hexitol derivative as described above:

(1) ACE inhibitors, including, but not limited to, benazepril, enazepril, captopril, enalapril, fosinopril, lisinopril, moexipril, quinapril, ramipril, perindopril and trandolapril;

(2) adenosine kinase inhibitors, including, but not limited to, 5-iodotubericidin;

(3) adrenal cortex antagonists, including, but not limited to, mitotane;

(4) AKT pathway inhibitors (protein kinase B inhibitors) including, but not limited to, deguelin and 1,5-dihydro-5-methyl-1-β-D-ribofuranosyl-1,4,5,6,8-pentaazaacenaphthylen-3-amine;

(5) angiogenesis inhibitors, including, but not limited to, fumagillin, Shikonin, Tranilast, ursolic acid; suramin; thalidomide, lenalidomide; phthalazines, including, but not limited to, 1-(4-chloroanilino)-4-(4-pyridylmethyl)phthalazine, 1-(4-methylanilino)-4-(4-pyridylmethyl)phthalazine, 1-(3-chloroanilino)-4-(4-pyridylmethyl)phthalazine, 1-anilino-4-(4-pyridylmethyl)phthalazine, 1-benzylamino-4-(4-pyridylmethyl)phthalazine, 1-(4-methoxyanilino)-4-(4-pyridylmethyl)phthalazine, 1-(3-benzyloxyanilino)-4-(4-pyridylmethyl)phthalazine, 1-(3-methoxyanilino)-4-(4-pyridylmethyl)phthalazine, 1-(2-methoxyanilino}-4-(4-pyridylmethyl)phthalazine, 1-(4-trifluoromethylanilino)-4-(4-pyridylmethyl)phthalazine, 1-(4-fluoroanilino)-4-(4-pyridylmethyl)phthalazine, 1-(3-hydroxyanilino)-4-(4-pyridylmethyl)phthalazine, 1-(4-hydroxyanilino)-4-(4-pyridylmethyl)phthalazine, 1-(3-aminoanilino)-4-(4-pyridylmethyl)phthalazine, 1-(3,4-dichloroanilino)-4-(4-pyridylmethyl)phthalazine, 1-(4-bromoanilino)-4-(4-pyridylmethyl)phthalazine, 1-(3-chloro-4-methoxyanilino)-4-(4-pyridylmethyl)phthalazine, 1-(4-cyanoanilino)-4-(4-pyridylmethyl)phthalazine, 1-(3-chloro-4-fluoroanilino)-4-(4-pyridylmethyl)phthalazine, 1-(3-methylanilino)-4-(4-pyridylmethyl)phthalazine, and other phthalazines disclosed in PCT Patent Application Publication No. WO 98/035958 by Bold et al., incorporated herein in its entirety by this reference, isoquinolines disclosed in PCT Patent Application Publication No. WO 00/09495 by Altmann et al., incorporated herein in its entirety by this reference, including 1-(3,5-dimethylanilino)-4-(pyridin-4-ylmethyl)-isoquinoline; phthalazines disclosed in PCT Patent Application Publication No. WO 00/59509 by Bold et al., incorporated herein in its entirety by this reference, including E-1-(3-methylanilino)-4-[(2-(pyridin-3-yl)vinyl]phthalazine, Z-1-(3-methylanilino)-4-[(2-(pyridin-3-yl)vinyl]phthalazine, 1-(3-methylanilino)-4-[(2-(pyridin-3-yl)ethyl]phthalazine, 1-(3-methylanilino)-4-[{2-(pyridin-4-yl)vinyl]phthalazine, 1-(4-chloro-3-trifluoromethylanilino)-4-[(2-(pyridin-3-yl)ethyl]phthalazine, 1-(4-chloroanilino)-4-[(2-(pyridin-3-yl)ethyl]phthalazine, 1-(3-chlorobenzylamino)-4-[(2-(pyridin-3-yl)ethyl]phthalazine, 1-(4-chloro-3-trifluoromethylanilino)-4-[3-(pyridin-3-yl)propyl]phthalazine, 1-(4-chloroanilino)-4-[3-(pyridin-3-yl)propyl]phthalazine, 1-(3-chloro-5-trifluoromethylanilino)-4-[3-(pyridin-3-yl)propyl]phthalazine, and 1-(4-tert-butylanilino)-4-[3-(pyridin-3-yl)propyl]phthalazine; and monoclonal antibodies;

(6) angiostatic steroids, including, but not limited to, anecortave, triamcinolone, hydrocortisone, 11α-epihydrocotisol, cortexolone, 17α-hydroxyprogesterone, corticosterone, desoxycorticosterone, testosterone, estrone, and dexamethasone;

(7) anti-androgens, including, but not limited to, nilutamide and bicalutamide;

(8) anti-estrogens, including, but not limited to, toremifene, letrozole, testolactone, anastrozole, bicalutamide, flutamide, exemestane, tamoxifen, fulvestrant, and raloxifene;

(9) anti-hypercalcemia agents, including, but not limited to, gallium (III) nitrate hydrate and pamidronate disodium;

(10) apoptosis inducers, including, but not limited to, 2-[[3-(2,3-dichlorophenoxy)propyl]amino]-ethanol, gambogic acid, embellin, and arsenic trioxide;

(11) ATI receptor antagonists, including, but not limited to, valsartan;

(12) aurora kinase inhibitors, including, but not limited to, binucleine 2;

(13) aromatase inhibitors, including, but not limited to: (a) steroids, including, but not limited to, atamestane, exemestane, and formestane; and (b) non-steroids, including, but not limited to, aminoglutethimide, roglethimide, pyridoglutethimide, trilostane, testolactone, ketoconazole, vorozole, fadrozole, anastrozole, and letrozole;

(14) bisphosphonates, including, but not limited to, etidronic acid, clodronic acid, tiludronic acid, alendronic acid, ibandronic acid, risedronic acid, and zoledronic acid;

(15) Bruton's tyrosine kinase inhibitors, including, but not limited to, terreic acid;

(16) calcineurin inhibitors, including, but not limited to, cypermethrin, deltamethrin, fenvalerate, and tyrphostin 8;

(17) CaM kinase II inhibitors, including, but not limited to, the 5-isoquinolinesulfonic acid 4-[(2S)-2-[(5-isoquinolinylsulfonyl)methylamino]-3-oxo-3-(4-phenyl-1-piperazinyl)propyl]phenyl ester, and N-[2-[[[3-(4-chlorophenyl)-2-propenyl]methyl]amino]methyl]phenyl]-N-(2-hydroxyethyl)-4-methoxy-benzenesulfonamide;

(18) CD45 tyrosine phosphatase inhibitors, including, but not limited to, [[2-(4-bromophenoxy)-5-nitrophenyl]hydroxymethyl]-phosphonic acid;

(19) CDC25 phosphatase inhibitors, including, but not limited to, 2,3-bis[(2-hydroyethyl)thio]-1,4-naphthalenedione;

(20) CHK kinase inhibitors, including, but not limited to, debromohymenialdisine;

(21) compounds targeting/decreasing a protein or lipid kinase activity; or a protein or lipid phosphatase activity; or further anti-angiogenic compounds, including, but not limited to, protein tyrosine kinase and/or serine and/or threonine kinase inhibitors or lipid kinase inhibitors, including, but not limited to:
(a) compounds targeting, decreasing or inhibiting the activity of the vascular endothelial growth factor receptors (VEGFR) or of vascular endothelial growth factor (VEGF), including, but not limited to, 7H-pyrrolo[2,3-d]pyrimidine derivatives, including: [6-[4-(4-ethyl-piperazine-1-ylmethyl)-phenyl]-7H-pyrrolo[2,3-d]pyrimidinpyrimidin-4-yl]-(R)-1-phenyl-ethylyamine (known as AEE788), BAY 43-9006; and isoquinoline compounds disclosed in PCT Patent Application Publication No. WO 00/09495, such as (4-tert-butyl-phenyl)-94-pyridin-4-ylmethyl-isoquinolin-1-ylyamine;
(b) compounds targeting, decreasing or inhibiting the activity of the platelet-derived growth factor-receptor (PDGFR), including, but not limited to: N-phenyl-2-pyrimidine-amine derivatives, e.g., imatinib, SU101, SU6668 and GFB-111;
(c) compounds targeting, decreasing or inhibiting the activity of the fibroblast growth factor-receptor (FGFR);
(d) compounds targeting, decreasing or inhibiting the activity of the insulin-like growth factor receptor 1 (IGF-1R), including, but not limited to: the compounds disclosed in WO 02/092599 and derivatives thereof of 4-amino-5-phenyl-7-cyclobutyl-pyrrolo[2,3-d]pyrimidine derivatives;
(e) compounds targeting, decreasing or inhibiting the activity of the Trk receptor tyrosine kinase family;
(f) compounds targeting, decreasing or inhibiting the activity of the Axl receptor tyrosine kinase family;
(g) compounds targeting, decreasing or inhibiting the activity of the c-Met receptor;
(h) compounds targeting, decreasing or inhibiting the activity of the Ret receptor tyrosine kinase;
(i) compounds targeting, decreasing or inhibiting the activity of the Kit/SCFR receptor tyrosine kinase;
(j) compounds targeting, decreasing or inhibiting the activity of the C-kit receptor tyrosine kinases, including, but not limited to, imatinib;
(k) compounds targeting, decreasing or inhibiting the activity of members of the c-Abl family and their gene-fusion products, e.g., BCR-Abl kinase, such as N-phenyl-2-pyrimidine-amine derivatives, including, but not limited to: imatinib, 6-(2,6-dichlorophenyl)-2-[(4-fluoro-3-methylphenyl)amino]-8-methyl-pyrido[2,3-d]pyrimidin-7(8H)-one (PD180970), methyl-4-[N-(2′,5′-dihydroxybenzyl)amino]benzoate (Tyrphostin AG957), 4-[[(2,5-dihydroxyphenyl)methyl]amino]benzoic acid tricyclo[3.3.1.13,7]dec-1-yl ester (adaphostin or NSC 680410), 6-(2,6-dichlorophenyl)-8-methyl-2-(3-methylsulfanylanilino)pyrido[2,3-d]pyrimidin-7-one (PD173955), and desatinib;
(l) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of the cyclin-dependent kinase family (CDK) and are especially those staurosporine derivatives disclosed in U.S. Pat. No. 5,093,330, such as, but not limited to, midostaurin; examples of further compounds include, e.g., UCN-01; safingol, sorafenib, Bryostatin 1; Perifosine; Ilmofosine; 3-[3-[2,5-Dihydro-4-(1-methyl-1H-indol-3-yl)-2,5-dioxo-1H-pyrrol-3-yl]-1H-indol-1-yl]propyl carbamimidothioic acid ester (RO 318220), 3-[(8S)-8-[(dimethylamino)methyl]-6,7,8,9-tetrahydropyrido[1,2-a]indol-10-yl]-4-(1-methyl-1H-indol-3-yl)-1H-pyrrole-2,5-dione (RO 320432), 12-(2-cyanoethyl)-6,7,12,13-tetrahydro-13-methyl-5-oxo-5H-indolo[2,3-a]pyrrolo[3,4-c]carbazole (GO 6976); Isis 3521; (S)-13-[(dimethylamino)methyl]-10,11,14,15-tetrahydro-4,9:16, 21-dimetheno-1H,13H-dibenzo[e,k]pyrrolo[3,4-h][1,4,13]oxadiazacyl clohexadecene-1,3(2H)dione (LY333531), LY379196; isoquinoline compounds, such as those disclosed in PCT Patent Application Publication No. WO 00/09495; farnesyltransferase inhibitors, including, but not limited to, tipifarnib and lonafarnib; 2-(2-chloro-4-iodo-phenylamino)-N-cyclopropylmethoxy-3,4-difluoro-benzamide (PD184352); and QAN697, a PI3K inhibitor;
(m) compounds targeting, decreasing or inhibiting the activity of protein-tyrosine kinase, such as, but not limited to, imatinib mesylate, a tyrphostin, pyrymidylaminobenzamide and derivatives thereof; a tyrphostin is preferably a low molecular weight (Mᵣ<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810, Tyrphostin AG 99, Tyrphostin AG 213, Tyrphostin AG 1748, Tyrphostin AG 490, Tyrphostin B44, Tyrphostin B44 (+) enantiomer, Tyrphostin AG 555, AG 494, Tyrphostin AG 556; Tyrphostin AG957, and adaphostin (4-{[(2,5-dihydroxyphenyl)methyl]amino}-benzoic acid adamantyl ester or NSC 680410);
(n) compounds targeting, decreasing or inhibiting the activity of the epidermal growth factor family of receptor tyrosine kinases (EGFR, ErbB2, ErbB3, ErbB4 as homodimers or heterodimers), such as, but not limited to, those compounds, proteins or monoclonal antibodies generically and specifically disclosed in PCT Patent Application Publication No. WO 97/02266 by Traxler et al. such as (R)-6-(4-hydroxyphenyl)-4-[(1-phenylethyl)-amino]-7H-pyrrolo-[2,3-d]pyrimidine, or in European Patent Application Publication No. EP 0564409 by Zimmermann, PCT Patent Application Publication No. WO 99/03854 by Zimmermann et al., European Patent Application Publication No. EP 0520722 by Barker et al., European Patent Application Publication No. EP 0566226 by Barker et al., European Patent Application Publication EP 0787722 by Wissner et al., European Patent Application Publication EP 0837063 by Arnold et al., U.S. Pat. No. 5,747,498 by Schnur et al., PCT Patent Application Publication WO 98/10767 by McMahon et al., PCT Patent Application Publication WO 97/30034 by Barker, PCT Patent Application Publication WO 97/49688 by Schnur, PCT Patent Application Publication WO 97/38983 by Bridges et al., PCT Patent Application Publication WO 96/30347 by Schnur et al., including, but not limited to, N-(3-ethylnylphenyl)-6,7-bis(2-methoxyethoxy)-4-quinazolinamine (CP 358774 or erlotinib), PCT Patent Application Publication WO 96/33980 by Gibson et al., including, but not limited to, N-(3-chloro-4-fluoro-phenyl)-7-methoxy-6-(3-morpholin-4-ylpropoxy)quinazolin-4-amine (gefitinib); and PCT Patent Application Publication WO 95/03283 by Barker et al., including, but not limited to, compound 6-amino-4-(3-methylphenyl-amino)-quinazoline (ZM105180); monoclonal antibodies, including, but not limited to trastuzumab and cetuximab; and other small molecule inhibitors, including, but not limited to: canertinib, pelitinib, lapatinib, and 7H-pyrrolo-[2,3-d]pyrimidine derivatives which are disclosed in PCT Patent Application Publication WO 03/013541 by Bold et al.;

(22) compounds which target, decrease or inhibit the activity of a protein or lipid phosphatase, including, but not limited to, inhibitors of phosphatase 1, phosphatase 2A, PTEN or CDC25, such as, but not limited to okadaic acid or a derivative thereof;

(23) compounds which induce cell differentiation processes, including, but not limited to, retinoic acid, α-tocopherol, γ-tocopherol, δ-tocopherol, α-tocotrienol, γ-tocotrienol, and δ-tocotrienol;

(24) cRAF kinase inhibitors, including, but not limited to, 3-(3,5-dibromo-4-hydroxybenzylidene)-5-iodo-1,3-dihydroindol-2-one and 3-(dimethylamino)-N-[3-[(4-hydroxybenzoyl)amino]-4-methylphenyl]-benzamide;

(25) cyclin dependent kinase inhibitors, including, but not limited to, N9-isopropyl-olomoucine; olomoucine; purvalanol B, roascovitine, kenpaullone, and purvalanol A;

(26) cysteine protease inhibitors, including, but not limited to, N-[(1S)-3-fluoro-2-oxo-1-(2-phenyl]ethyl)propyl]amino]-2-oxo-1-(phenylmethyl)ethyl]-4-morpholinecarboxamide;

(27) DNA intercalators, including, but not limited to, plicamycin and dactinomycin;

(28) DNA strand breakers, including, but not limited to, bleomycin;

(29) E3 ligase inhibitors, including, but not limited to, N-((3,3,3-trifluoro-2-trifluoromethyl)propionyl)sulfanilamide;

(30) EDG binders, including, but not limited to, FTY720;

(31) endocrine hormones, including, but not limited to, leuprolide and megestrol acetate;

(32) farnesyltransferase inhibitors, including, but not limited to, α-hydroxyfarnesylphosphonic acid, 2-[[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-, 1-methylethyl butanoic acid ester (2S), and manumycin A;

(33) Flk-1 kinase inhibitors, including, but not limited to, 2-cyano-3-[4-hydroxy-3,5-bis(1-methylethyl)phenyl]-N-(3-phenylpropyl)-, (2-E)-2-propenamide;

(34) Flt-3 inhibitors, including, but not limited to, N-benzoyl-staurosporine, midostaurin, and N-(2-diethylaminoethyl)-5-[(Z)-(5-fluoro-2-oxo-1H-indol-3-ylidene)methyl]-2,4-dimethyl-1H-pyrrole-3-carboxamide (sunitinib);

(35) gonadorelin agonists, including, but not limited to, abarelix, goserelin, and goserelin acetate;

(36) heparanase inhibitors, including, but not limited to, phosphomannopentaose sulfate (P1-88);

(37) histone deacetylase (HDAC) inhibitors, including, but not limited to, compounds disclosed in PCT Patent Application Publication No. WO 02/22577 by Bair et al., including, but not limited to, N-hydroxy-3-[4-[[(2-hydroxyethyl)[2-(1H-indol-3-yl)ethyl]-amino]methyl]phenyl]-2E-2-propenamide, suberoylanilide hydroxamic acid, 4-(2-amino-phenylcarbamoyl)-benzyl]-carbamic acid pyridine-3-ylmethyl ester and derivatives thereof, butyric acid, pyroxamide, trichostatin A, oxamflatin, apicidin, depsipeptide, depudecin, trapoxin, HC toxin, and sodium phenylbutyrate;

(38) HSP90 inhibitors, including, but not limited to: 17-allylamino,17-demethoxygeldanamycin (17AAG); a geldanamycin derivative; other geldanamycin-related compounds; radicicol; and 5-(2,4-dihydroxy-5-isopropyl-phenyl)-4-(4-morpholin-4-ylmethyl-phenyl)-isoxazole-3-carboxylic acid ethylamide;

(39) IκBα inhibitors (IKKs), including, but not limited to, 3-[(4-methylphenyl)sulfonyl]-(2E)-2-propenenitrile;

(40) insulin receptor tyrosine kinase inhibitors, including, but not limited to, hydroxy-2-naphthalenylmethylphosphonic acid;

(41) c-Jun N-terminal kinase inhibitors, including, but not limited to, pyrazoleanthrone and epigallocatechin gallate;

(42) microtubule binding agents, including, but not limited to: vinblastine sulfate; vincristine sulfate; vindesine; vinorelbine; docetaxel; paclitaxel; discodermolides; colchicines; and epothilones and derivatives thereof, such as epothilone B or a derivative thereof;

(43) mitogen-activated protein (MAP) kinase inhibitors, including, but not limited to, N-[2-[[[3-(4-chlorophenyl)-2-propenyl]methyl]amino]methyl]phenyl]-N-(2-hydroxyethyl)-4-methoxy-benzenesulfonamide;

(44) MDM2 inhibitors, including, but not limited to, trans-4-iodo,4′-boranyl-chalcone;

(45) MEK inhibitors, including, but not limited to, bis[amino[2-aminophenyl)thio]methylene]-butanedinitrile;

(46) methionine aminopeptidase inhibitors, including, but not limited to, bengamide and derivatives thereof;

(47) MMP inhibitors, including, but not limited to: actinonin; epigallocatechin gallate; collagen peptidomimetic and non-peptidomimetic inhibitors; tetracycline derivatives such as hydroxamate, batimastat, marimastat, primomastat, TAA211, N-hydroxy-2(R)-[[(4-methoxyphenyl)sulfonyl](3-picolyl)amino]-3-methylbutanamide hydrochloride (MMI270B), and AAJ996;

(48) NGFR tyrosine kinase inhibitors, including, but not limited to, Tyrphostin AG 879;

(49) p38 MAP kinase inhibitors, including, but not limited to, 3-(dimethylamino)-N-[3-[(4-hydroxybenzoyl)amino]-4-methylphenyl]-benzamide;

(50) p56 tyrosine kinase inhibitors, including, but not limited to, 9,10-dihydro-3-hydroxy-1-methoxy-9,10-dioxo-2-anthracenecarboxaldehyde and Tyrphostin 46;

(51) PDGFR tyrosine kinase inhibitors, including, but not limited to, Tyrphostin AG 1296; Tyrphostin 9, 2-amino-4-(1H-indol-5-yl)-1,3-butadiene-1,1,3-tricarbonitrile, and imatinib;

(52) phosphatidylinositol 3-kinase inhibitors, including, but not limited to, wortmannin and quercetin dihydrate;

(53) phosphatase inhibitors, including, but not limited to, cantharidic acid, cantharidin, and (E)-N-[4-(2-carboxyethenyl)benzoyl]glycyl-L-α-glutamyl-L-leucinamide;

(54) platinum agents, including, but not limited to, carboplatin, cisplatin, oxaliplatin, satraplatin, and ZD0473;

(55) protein phosphatase inhibitors, including, but not limited to:
(a) PP1 and PP2A inhibitors, including, but not limited to, cantharidic acid and cantharidin;
(b) tyrosine phosphatase inhibitors, including, but not limited to, L-P-bromotetramisole oxalate, benzylphosphonic acid, and (5R)-4-hydroxy-5-(hydroxymethyl)-3-(1-oxohexadecyl)-2(5H)-furanone;

(56) PKC inhibitors, including, but not limited to, -[1-[3-(dimethylamino)propyl]-1H-indol-3-yl]-4-(1H-indol-3-yl)-1H-pyrrolo-2,5-dione, sphingosine, staurosporine, Tyrphostin 51, and hypericin;

(57) PKC delta kinase inhibitors, including, but not limited to, rottlerin;

(58) polyamine synthesis inhibitors, including, but not limited to, (RS)-2,5-diamino-2-(difluoromethyl)pentanoic acid (DMFO);

(59) proteasome inhibitors, including, but not limited to, aclacinomycin A, gliotoxin, and bortezomib;

(60) PTP1B inhibitors, including, but not limited to, (E)-N-[4-(2-carboxyethenyl)benzoyl]glycyl-L-α-glutamyl-L-leucinamide;

(61) protein tyrosine kinase inhibitors, including, but not limited to: Tyrphostin AG 126; Tyrphostin AG 1288; Tyrphostin AG 1295; geldanamycin; and genistein;

(62) SRC family tyrosine kinase inhibitors, including, but not limited to, 1-(1,1-dimethylethyl)-3-(1-naphthalenyl)-1H-pyrazolo[3,4-d]pyrimidin-4-amine, and 3-(4-chlorophenyl)-1-(1,1-dimethylethyl)-1H-pyrazolo[3,4-d]pyrimidin-4-amine;

(63) Syk tyrosine kinase inhibitors including, but not limited to, piceatannol;

(64) Janus (JAK-2 and/or JAK-3) tyrosine kinase inhibitors, including, but not limited to, Tyrphostin AG 490, and 2-naphthyl vinyl ketone;

(65) inhibitors of Ras oncogenic isoforms, including, but not limited to, (2S)-2-[[(2S)-2-[(2S,3S)-2-[(2R)-2-amino-3-mercaptopropyl]amino]-3-methylpentyl]oxy]-1-oxo-3-phenylpropyl]amino]-4-(methylsulfonyl)-butanoic acid 1-methylethyl ester (L-744832), DK8G557, and tipifarnib;

(66) retinoids, including, but not limited to, isotretinoin and tretinoin;

(67) ribonucleotide reductase inhibitors, including, but not limited to, hydroxyurea and 2-hydroxy-1H-isoindole-1,3-dione;

(68) RNA polymerase II elongation inhibitors, including, but not limited to, 5,6-dichloro-1-beta-D-ribofuranosylbenzimidazole;

(69) S-adenosylmethionine decarboxylase inhibitors, including, but not limited to, 5-amidino-1-tetralone-2′-amidinohydrazone and other compounds disclosed in U.S. Pat. No. 5,461,076 to Stanek et al., incorporated herein by this reference;

(70) serine/threonine kinase inhibitors, including, but not limited to, sorafenib and 2-aminopurine;

(71) compounds which target, decrease, or inhibit the activity or function of serine/threonine mTOR kinase, including, but not limited to, everolimus, temsirolimus, zotarolimus, rapamycin, derivatives and analogs of rapamycin, deforolimus, AP23841, sirolimus, and everolimus;

(72) somatostatin receptor antagonists, including, but not limited to, octreotide and pasireotide (SOM230);

(73) sterol biosynthesis inhibitors, including, but not limited to, terbinadine;

(74) telomerase inhibitors, including, but not limited to, telomestatin; and

(75) topoisomerase inhibitors, including, but not limited to:
(a) topoisomerase I inhibitors, including, but not limited to, topotecan, gimatecan, irinotecan, camptothecin and its analogues, 9-nitrocamptothecin and the macromolecular camptothecin conjugate PNU-16614, macromolecular camptothecin conjugates described in PCT Patent Application Publication No. WO 99/17804 by Angelucci et al., 10-hydroxycamptothecin acetate salt, etoposide idarubicin hydrochloride, teniposide, doxorubicin; epirubicin hydrochloride, mitoxantrone hydrochloride, and daunorubicin hydrochloride; and
(b) topoisomerase II inhibitors, including, but not limited to, anthracyclines, such as doxorubicin, including liposomal formulations thereof, daunorubicin, including liposomal formulations thereof, epirubicin, idarubicin, nemorubicin, mitoxantrone, losoxantrone, etoposide, and eniposide;

(76) VEGFR tyrosine kinase inhibitors, including, but not limited to, 3-(4-dimethylaminobenzylidenyl)-2-indolinone; and

(77) RANKL inhibitors, including, but not limited to, denosumab.

When the improvement is made by chemosensitization, the chemosensitization can comprise, but is not limited to, the use of amonafide or a derivative or analog thereof as a chemosensitizer in combination with an agent selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(i) berberine;
(k) apigenin;
(l) colchicine or an analog of colchicine;
(m) genistein;
(n) etoposide;
(o) cytarabine;
(p) camptothecin;
(q) vinca alkaloids;
(r) 5-fluorouracil;
(s) curcumin;
(t) NF-κB inhibitors;
(u) rosmarinic acid; and
(v) mitoguazone.

When the improvement is made by chemopotentiation, the chemopotentiation can comprise, but is not limited to, the use of amonafide or a derivative or analog thereof as a chemopotentiator in combination with an agent selected from the group consisting of:
(a) fraudulent nucleosides;
(b) fraudulent nucleotides;
(c) thymidylate synthetase inhibitors;
(d) signal transduction inhibitors;
(e) cisplatin or platinum analogs;
(f) alkylating agents;
(g) anti-tubulin agents;
(h) antimetabolites;
(i) berberine;
(j) apigenin;
(k) colchicine or analogs of colchicine;
(l) genistein;
(m) etoposide;
(n) cytarabine;
(o) camptothecins;
(p) vinca alkaloids;
(q) topoisomerase inhibitors;
(r) 5-fluorouracil;
(s) curcumin;
(t) NF-κB inhibitors;
(u) rosmarinic acid; and
(v) mitoguazone.

When the improvement is made by post-treatment management, the post-treatment management can be, but is not limited to, a method selected from the group consisting of:
(a) a therapy associated with pain management;
(b) nutritional support;
(c) administration of an anti-emetic;
(d) an anti-nausea therapy;
(e) administration of an anti-inflammatory agent;
(f) administration of an antipyretic agent; and
(g) administration of an immune stimulant.

When the improvement is made by alternative medicine/post-treatment support, the alternative medicine/post-treatment support can be, but is not limited to, a method selected from the group consisting of:
(a) hypnosis;
(b) acupuncture;
(c) meditation;
(d) administration of a herbal medication created either synthetically or through extraction; and
(e) applied kinesiology.

In one alternative, when the method is administration of a herbal medication created either synthetically or through extraction, the herbal medication created either synthetically or through extraction can be selected from the group consisting of:
(a) a NF-κB inhibitor;
(b) a natural anti-inflammatory;
(c) an immunostimulant;
(d) an antimicrobial; and
(v) a flavonoid, isoflavone, or flavone.

When the herbal medication created either synthetically or through extraction is a NF-κB inhibitor, the NF-κB inhibitor can be selected from the group consisting of parthenolide, curcumin, and rosmarinic acid. When the herbal medication created either synthetically or through extraction is a natural anti-inflammatory, the natural anti-inflammatory can be selected from the group consisting of rhein and parthenolide. When the herbal medication created either synthetically or through extraction is an immunostimulant, the immunostimulant can be a product found in or isolated from Echinacea. When the herbal medication created either synthetically or through extraction is an anti-microbial, the anti-microbial can be berberine. When the herbal medication created either synthetically or through extraction is a flavonoid or flavone, the flavonoid, isoflavone, or flavone can be selected from the group consisting of apigenin, genistein, apigenenin, genistein, genistin, 6″-O-malonylgenistin, 6″-O-acetylgenistin, daidzein, daidzin, 6″-O-malonyldaidzin, 6″-O-acetylgenistin, glycitein, glycitin, 6″-O-malonylglycitin, and 6-O-acetylglycitin.

When the improvement is made by a bulk drug product improvement, the bulk drug product can be, but is not limited to, a bulk drug product improvement selected from the group consisting of:
(a) preparation as a free base form;
(b) salt formation;
(c) preparation as a homogeneous crystalline structure;
(d) amorphous structure;
(e) preparation as a pure isomer;
(f) increased purity;
(g) preparation with lower residual solvent content; and
(h) preparation with lower residual heavy metal content.

When the improvement is made by use of a diluent, the diluent can be, but is not limited to, a diluent selected from the group consisting of:
(a) an emulsion;
(b) dimethylsulfoxide (DMSO);
(c) N-methylformamide (NMF)
(d) dimethylformamide (DMF)
(e) dimethylacetamide (DMA);
(f) ethanol;
(g) benzyl alcohol;
(h) dextrose-containing water for injection;
(i) Cremophor;
(j) cyclodextrins; and
(k) PEG.

When the improvement is made by use of a solvent system, the solvent system can be, but is not limited to, a solvent system selected from the group consisting of:
(a) an emulsion;
(b) DMSO;
(c) NMF;
(d) DMF;
(e) DMA;
(f) ethanol;
(g) benzyl alcohol;
(h) dextrose-containing water for injection;
(i) Cremophor;
(j) PEG; and
(k) salt systems.

When the improvement is made by use of an excipient, the excipient can be, but is not limited to, an excipient selected from the group consisting of:
(a) mannitol;
(b) albumin;
(c) EDTA;
(d) sodium bisulfite;
(e) benzyl alcohol;
(f) carbonate buffers;
(g) phosphate buffers;
(h) PEG;
(i) vitamin A;
(j) vitamin D;
(k) vitamin E;
(l) esterase inhibitors;
(m) cytochrome P450 inhibitors;
(n) multi-drug resistance (MDR) inhibitors;
(o) organic resins; and
(p) detergents.

Suitable esterase inhibitors include, but are not limited to, ebelactone A and ebelactone B.

Suitable cytochrome P450 inhibitors include, but are not limited to, 1-aminobenzotriazole, N-hydroxy-N′-(4-butyl-2-methylphenyl)formamidine, ketoconazole, methoxsalen, metyrapone, roquefortine C, proadifen, 2,3′,4,5′-tetramethylstilbene, and troleandomycin.

Suitable MDR inhibitors include, but are not limited to, 5′-methoxyhydnocarpin, INF 240, INF 271, INF 277, INF 392, INF 55, reserpine, and GG918. MDR inhibitors are described in M. Zloh & S. Gibbons, “Molecular Similarity of MDR9 Inhibitors,” Int. J. Mol. Sci. 5: 37-47 (2004), incorporated herein by this reference.

Suitable organic resins include, but are not limited to, a partially neutralized polyacrylic acid, as described in U.S. Pat. No. 8,158,616 to Rodgers et al., incorporated herein by this reference.

Suitable detergents include, but are not limited to, nonionic detergents such as a polysorbate or a poloxamer, and are described in PCT Patent Application Publication No. WO/1997/039768 by Bjorn et al., incorporated herein by this reference.

When the improvement is made by use of a dosage form, the dosage form can be, but is not limited to, a dosage form selected from the group consisting of:
(a) tablets;
(b) capsules;
(c) topical gels;
(d) topical creams;
(e) patches;
(f) suppositories;
(g) lyophilized dosage fills;
(h) immediate-release formulations;
(i) slow-release formulations;
(j) controlled-release formulations; and
(k) liquid in capsules.

Formulation of pharmaceutical compositions in tablets, capsules, and topical gels, topical creams or suppositories is well known in the art and is described, for example, in United States Patent Application Publication No. 2004/0023290 by Griffin et al., incorporated herein by this reference.

Formulation of pharmaceutical compositions as patches such as transdermal patches is well known in the art and is described, for example, in U.S. Pat. No. 7,728,042 to Eros et al., incorporated herein by this reference.

Lyophilized dosage fills are also well known in the art. One general method for the preparation of such lyophilized dosage fills, applicable to dibromodulcitol and derivatives thereof, comprises the following steps:

(1) Dissolve the drug in water for injection precooled to below 10° C. Dilute to final volume with cold water for injection to yield a 40 mg/mL solution.

(2) Filter the bulk solution through an 0.2-μm filter into a receiving container under aseptic conditions. The formulation and filtration should be completed in 1 hour.

(3) Fill nominal 1.0 mL filtered solution into sterilized glass vials in a controlled target range under aseptic conditions.

(4) After the filling, all vials are placed with rubber stoppers inserted in the “lyophilization position” and loaded in the prechilled lyophilizer. For the lyophilizer, shelf temperature is set at +5° C. and held for 1 hour; shelf temperature is then adjusted to −5° C. and held for one hour, and the condenser, set to −60° C., turned on.

(5) The vials are then frozen to 30° C. or below and held for no less than 3 hours, typically 4 hours.

(6) Vacuum is then turned on, the shelf temperature is adjusted to −5° C., and primary drying is performed for 8 hours; the shelf temperature is again adjusted to −5° C. and drying is carried out for at least 5 hours.

(7) Secondary drying is started after the condenser (set at −60° C.) and vacuum are turned on. In secondary drying, the shelf temperature is controlled at +5° C. for 1 to 3 hours, typically 1.5 hours, then at 25° C. for 1 to 3 hours, typically 1.5 hours, and finally at 35-40° C. for at least 5 hours, typically for 9 hours, or until the product is completely dried.

(8) Break the vacuum with filtered inert gas (e.g., nitrogen). Stopper the vials in the lyophilizer.

(9) Vials are removed from the lyophilizer chamber and sealed with aluminum flip-off seals. All vials are visually inspected and labeled with approved labels.

Immediate-release formulations are described in U.S. Pat. No. 8,148,393 to van Dalen et al., incorporated herein by this reference. Immediate-release formulations can include, for example, conventional film-coated tablets.

Slow-release formulations are described in U.S. Pat. No. 8,178,125 to Wen et al., incorporated herein by this reference. Slow-release formulations can include, for example, microemulsions or liquid crystals.

Controlled-release formulations are described in U.S. Pat. No. 8,231,898 to Oshlack et al., incorporated herein by this reference. Controlled-release formulations can include, for example, a matrix that includes a controlled-release material. Such a controlled-release material can include hydrophilic and/or hydrophobic materials, such as gums, cellulose ethers, acrylic resins, protein derived materials, waxes, shellac, and oils such as hydrogenated castor oil or hydrogenated vegetable oil. However, any pharmaceutically acceptable hydrophobic or hydrophilic controlled-release material which is capable of imparting controlled-release of the amonafide or derivative or analog thereof may be used in accordance with the present invention. Preferred controlled-release polymers include alkylcelluloses such as ethylcellulose, acrylic and methacrylic acid polymers and copolymers, and cellulose ethers, especially hydroxyalkylcelluloses (e.g., hydroxypropylmethylcellulose) and carboxyalkylcelluloses. Preferred acrylic and methacrylic acid polymers and copolymers include methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), and glycidyl methacrylate copolymers.

When the improvement is made by use of dosage kits and packaging, the dosage kits and packaging can be, but are not limited to, dosage kits and packaging selected from the group consisting of the use of amber vials to protect from light and the use of stoppers with specialized coatings to improve shelf-life stability.

When the improvement is made by use of a drug delivery system, the drug delivery system can be, but is not limited to, a drug delivery system selected from the group consisting of:
(a) oral dosage forms;
(b) nanocrystals;
(c) nanoparticles;
(d) cosolvents;
(e) slurries;
(f) syrups;
(g) bioerodible polymers;
(h) liposomes;
(i) slow-release injectable gels; and
(j) microspheres.

Nanocrystals are described in U.S. Pat. No. 7,101,576 to Hovey et al., incorporated herein by this reference.

Nanoparticles for drug delivery are described in U.S. Pat. No. 8,258,132 to Bosch et al., incorporated herein by this reference. Typically, such nanoparticles have an average particle size of the active ingredient of less than about 1000 nm, more preferably, less than about 400 nm, and most preferably, less than about 250 nm. The nanoparticles can be coated with a surface stabilizer, such as, but not limited to, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxes 3550® and 934® (Union Carbide)), polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose, hydroxypropylmethyl-cellulose phthalate, noncrystalline cellulose, magnesium aluminium silicate, triethanolamine, polyvinyl alcohol (PVA), polyvinylpyrrolidone (PVP), 4-(1,1,3,3-tetramethylbutylyphenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), dialkylesters of sodium sulfosuccinic acid (e.g., Aerosol OT®, which is a dioctyl ester of sodium sulfosuccinic acid (American Cyanamid)), dioctyl sodium sulfosuccinate (DOSS), docusate sodium (Ashland Chem. Co., Columbus, Ohio); Duponol P®, which is a sodium lauryl sulfate (DuPont); Triton X-200®, which is an alkyl aryl polyether sulfonate (Rohm and Haas); Crodestas F-110®, which is a mixture of sucrose stearate and sucrose distearate (Croda Inc.); p-isononylphenoxy-poly-(glycidol), also known as Olin-IOG® or Surfactant 10-G® (Olin Chemicals, Stamford, Conn.); Crodestas SL-40® (Croda, Inc.); and SA9OHCO, which is C₁₈H₃₇CH₂(CON(CH₃)—OCH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methyl-glucamide; n-heptyl-β-D-glucopyranoside; n-heptyl β-D-thioglucoside; n-hexyl β-D-glucopyranoside; nonanoyl-N-methylglucamide; n-nonanoyl β-D-glucopyranoside; octanoyl-N-methylglucamide; n-octyl β-D-glucopyranoside; and octyl β-D-thioglucopyranoside.

Pharmaceutically acceptable cosolvents are described in U.S. Pat. No. 8,207,195 to Navratil et al., incorporated herein by this reference, and include, but are not limited to, water, methanol, ethanol, 1-propanol, isopropanol, 1-butanol, isobutanol, t-butanol, acetone, methyl ethyl ketone, acetonitrile, ethyl acetate, benzene, toluene, xylene(s), ethylene glycol, dichloromethane, 1,2-dichloroethane, N-methylformamide, N,N-dimethylformamide, N-methylacetamide, pyridine, dioxane, and diethyl ether.

Slurries for use in pharmaceutical formulations are described in United States Patent Application Publication No. 2006/0229277 by Laxminarayan, incorporated herein by this reference.

Syrups for use in pharmaceutical formulations are described in U.S. Pat. No. 8,252,930 to Stoit et al., incorporated herein by this reference. Such syrups can include the active ingredient and a syrup-forming component such as sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents.

Bioerodible polymers are described in U.S. Pat. No. 7,318,931 to Okumu et al., incorporated herein by this reference. A bioerodible polymer decomposes when placed inside an organism, as measured by a decline in the molecular weight of the polymer over time. Polymer molecular weights can be determined by a variety of methods including size exclusion chromatography (SEC), and are generally expressed as weight averages or number averages. A polymer is bioerodible if, when in phosphate buffered saline (PBS) of pH 7.4 and a temperature of 37° C., its weight-average molecular weight is reduced by at least 25% over a period of 6 months as measured by SEC. Useful bioerodible polymers include polyesters, such as poly(caprolactone), poly(glycolic acid), poly(lactic acid), and poly(hydroxybutryate); polyanhydrides, such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

Liposomes are well known as drug delivery vehicles. Liposome preparation is described in European Patent Application Publication No. EP 1332755 by Weng et al., incorporated herein by this reference.

Slow release injectable gels are known in the art and are described, for example, in B. Jeong et al., “Drug Release from Biodegradable Injectable Thermosensitive Hydrogel of PEG-PLGA-PEG Triblock Copolymers,” J. Controlled Release 63: 155-163 (2000), incorporated herein by this reference.

The use of microspheres for drug delivery is known in the art and is described, for example, in H. Okada & H. Taguchi, “Biodegradable Microspheres in Drug Delivery,” Crit. Rev. Ther. Drug Carrier Sys. 12: 1-99 (1995), incorporated herein by this reference.

Another drug delivery system potentially usable with amonafide or a derivative or analog of amonafide is the amphiphilic block copolymer system described in U.S. Pat. No. 7,311,901 to Seo et al., incorporated herein by this reference. In general, the amphiphilic block copolymer comprises a hydrophilic block and a hydrophobic block with a terminal hydroxyl group, wherein the terminal hydroxyl group of the hydrophobic block is substituted with a tocopherol or cholesterol group. U.S. Pat. No. 7,311,901 to Seo et al. further describes polymeric compositions capable of forming stable micelles in an aqueous solution, comprising the amphiphilic block copolymer and a polylactic acid derivative wherein one or both ends of the polylactic acid derivative are covalently bound to at least one carboxyl group.

Yet another drug delivery system potentially useful with amonafide or a derivative or analog of amonafide is the emulsion vehicle described in U.S. Pat. No. 6,485,383 to Lambert et al., incorporated herein by this reference. In general, this emulsion vehicle comprises an emulsion of α-tocopherol stabilized by biocompatible surfactants. Also included in the emulsion is pegylated vitamin E. Pegylated α-tocopherol includes polyethylene glycol subunits attached by a succinic acid diester at the ring hydroxyl of vitamin E and serves as a primary surfactant and a stabilizer as well as a secondary solvent in emulsions of α-tocopherol.

Yet another drug delivery system potentially useful with amonafide or a derivative or analog of amonafide are the biodegradable polymer compositions described in U.S. Pat. No. 6,238,687 to Mao et al., incorporated herein by this reference. These polymers contain phosphorus and desaminotyrosyl L-tyrosine linkages in the polymer backbone.

Yet another drug delivery system potentially useful with amonafide or a derivative or analog of amonafide are the pharmaceutically acceptable substantially anhydrous injectable semi-solid compositions described in U.S. Pat. No. 5,573,781 to Brown et al., incorporated herein by this reference. The compositions comprise a water immiscible fatty acid matrix and a cytostatic agent, such as amonafide or a derivative or analog thereof. Typically, the matrix material will be fatty acid ester compositions, having the desired flowable and viscosity characteristics, either as a natural characteristic or as a result of additives. Suitable lipid compositions will comprise fatty acid esters, either a single fatty acid ester or a mixture of fatty acid esters, which are biodegradable in the host, by themselves or in combination with one or more physiologically accceptable thickening agents, particularly fatty acid salts or synthetic and/or longer chain fatty acid esters, e.g. waxy esters. Suitable fatty acid ester compositions will comprise a single or mixture of fatty acid esters, and may comprise two or more different fatty acid esters, usually not more than ten different fatty acid esters. Suitable fatty acid esters include mono-, di- and tri-glycerides, as well as mono- and dibasic acid esters, e.g. ethyl oleate, isopropyl myristate, or other such esters, where the carboxylic acid group will usually have at least 6, more usually at least 8 carbon atoms, preferably at least about 12 carbon atoms, and may be saturated or unsaturated, usually having not more than 3 sites of ethylenic unsaturation per acid moiety, and the fatty acid esters will have at least 8 carbon atoms and not more than about 60 carbon atoms, usually not more than about 50 carbon atoms. Of particular interest are glycerides having fatty acids of from about 12 to 24 carbon atoms, saturated or unsaturated, naturally occurring or synthetic. The alcohols will usually have from about 1 to 6, usually 1 to 5, more usually 1 to 3 hydroxyl groups and not more than two ether groups and will usually be from 2 to 6, more usually 2 to 3 carbon atoms. The fatty acid esters of the subject invention will not include esters which are modified with additional functional groups which increase the water solubility properties of the esters, e.g. such as polyoxyethylated castor oil or other alkyleneoxy modified fatty acid esters. The fatty acid esters may be added as partially pure fractions or complex mixtures such as saturated or partially saturated glycerides, e.g. oils and fats. Any carboxylic acid ester oil which is physiologically acceptable can be employed as the matrix component, where the oil may be a single or combination of oils, which may or may not be partially hydrogenated. Specific physiologically acceptable oils of interest include vegetable oils, such as sesame, peanut, soybean, cottonseed, corn, olive, persic, castor, and the like.

When the improvement is made by use of a drug conjugate form, the drug conjugate form can be, but is not limited to, a drug conjugate form selected from the group consisting of:
(a) a polymer system;
(b) polylactides;
(c) polyglycolides;
(d) amino acids;
(e) peptides;
(f) multivalent linkers; and
(g) conjugates with fatty amines.

Polylactide conjugates are well known in the art and are described, for example, in R. Tong & C. Cheng, “Controlled Synthesis of Camptothecin-Polylactide Conjugates and Nanoconjugates,” Bioconjugate Chem. 21: 111-121 (2010), incorporated by this reference.

Polyglycolide conjugates are also well known in the art and are described, for example, in PCT Patent Application Publication No. WO 2003/070823 by Elmaleh et al., incorporated herein by this reference.

Multivalent linkers are known in the art and are described, for example, in United States Patent Application Publication No. 2007/0207952 by Silva et al., incorporated herein by this reference. For example, multivalent linkers can contain a thiophilic group for reaction with a reactive cysteine, and multiple nucleophilic groups (such as NH or OH) or electrophilic groups (such as activated esters) that permit attachment of a plurality of biologically active moieties to the linker.

Conjugates with fatty amines are disclosed in U.S. Pat. No. 8,552,054 by Swindell et al., incorporated herein by this reference. Typically, the fatty acid portion of the fatty amine is selected from the group consisting of octanoic (caprylic); nonanoic (pelargonic); decanoic (capric); undecanoic (hendecanoic); dodecanoic (lauric); tridecanoic; tetradecanoic (myristic); pentadecanoic; hexadecanoic (pamitic); heptadecanoic (margaric); octadecanoic (stearic); 12-hydroxy stearic; nonadecanoic; eicosanoie (arachidic); heneicosanoic; docosanoic (behenic); tricosanoic; tetracosanoic (lignoceric); 10-undecenoic (hendecenoic); 11-dodecenoic; 12-tridecenoic; 9-tetradecenoic (myristoleic); 9-trans-tetradecenoic (myristelaidic); 10-pentadecenoic; 10-trans-pentadecenoic; 9-hexadecenoic (palmitoleic); 8-trans-hexadecenoic (palmitelaidic); 10-heptadecenoic; 10-trans-heptadecenoic; 6-octadecenoic (petroselinic); 6-trans-octadecenoic (petroselaidic); 8-octadecenoic (oleic); 9-11-octadecenoic (vaccenic); 11-trans-octadecenoic (transvaccenic); 9-cis-12 hydroxy-octadecenoic (ricinoleic); 9-trans-12-hydroxy-octadecenoic (ricinelaidic); 7-nonadecenoic; 7-trans-nonadecenoic; 10-nonadecenoic; 10-trans-nonadecenoic; 10-13-nonadecadienoic; 10-13-trans-nonadecadienoic; 8-12-octadecadienoic (linoleic); 9-trans-12-trans octadecadienoic (linoelaidic); octadecadienoic (conjugated); 9-12-15-octadecatrienoic (linolenic); 6-9-12-octadecatrienoic (gamma linolenic); 11-trans-eicosenoic; 8-eicosenoic; 11-eicosenoic; 5-eicosenoic; 11-14-eicosadienoic; 8-11-14-eicosatrienoic (homogamma linolenic); 11-14-17-eicosatrienoic; 5-8-11-14-eicosatetraenoic (arachidonic); 5-8-11-14-17-eicosapentaenoic; 7-10-13-16-19-docosapentaenoic; arachidonic; 13-docosenoic (erucic); 13-transdocosenoic (brassidic); 13-16-docosadienoic; 13-16-19-docosatrienoic; 7-10-13-16-docosatetraenoic-; 4-7-10-13-16-19-docosahexaenoic (DHA); 12-heneicosenoic; 12-15-heneicosadienoic; 14-tricosenoic; and 15-tetracosenoic (nervonic).

Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the ε-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the .epsilon.-amino group of the side chain of lysine. Many other electrophilic reagents are known that will react with the ε-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydiimidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques,” (Academic Press, San Diego, 1996), pp. 154-158, incorporated herein by this reference. In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail.

Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiimides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. Such cross-linking reagents and reactions are described in G. T. Hermanson, “Bioconjugate Techniques” (Academic Press, San Diego, 1996), incorporated herein by this reference.

When the improvement is made by use of a compound analog, the compound analog can be, but is not limited to, a compound analog selected from the group consisting of:
(a) alteration of side chains to increase or decrease lipophilicity;
(b) addition of an additional chemical functionality to alter a property selected from the group consisting of reactivity, electron affinity, and binding capacity; and
(c) alteration of salt form.

When the improvement is made by use of a prodrug system, the prodrug system can be, but is not limited to, a prodrug system selected from the group consisting of:
(a) the use of enzyme sensitive esters;
(b) the use of dimers;
(c) the use of Schiff bases;
(d) the use of pyridoxal complexes;
(e) the use of caffeine complexes;
(f) the use of plasmin-activated prodrugs; and
(g) the use of a drug targeting complex comprising a targeting carrier molecule that is selectively distributed to a specific cell type or tissue containing the specific cell type; a linker which is acted upon by a molecule that is present at an effective concentration in the environs of the specific cell type; and a therapeutically active agent to be delivered to the specific cell type.

The use of prodrug systems is described in T. Jarvinen et al., “Design and Pharmaceutical Applications of Prodrugs” in Drug Discovery Handbook (S. C. Gad, ed., Wiley-Interscience, Hoboken, N. J., 2005), ch. 17, pp. 733-796, incorporated herein by this reference. This publication describes the use of enzyme sensitive esters as prodrugs. The use of dimers as prodrugs is described in U.S. Pat. No. 7,879,896 to Allegretti et al., incorporated herein by this reference. The use of peptides in prodrugs is described in S. Prasad et al., “Delivering Multiple Anticancer Peptides as a Single Prodrug Using Lysyl-Lysine as a Facile Linker,” J. Peptide Sci. 13: 458-467 (2007), incorporated herein by this reference. The use of Schiff bases as prodrugs is described in U.S. Pat. No. 7,619,005 to Epstein et al., incorporated herein by this reference. The use of caffeine complexes as prodrugs is described in U.S. Pat. No. 6,443,898 to Unger et al., incorporated herein by this reference.

Another potential prodrug system for amonafide and derivatives or analogs of amonafide is the use of a plasmin-activated prodrug as described in U.S. Pat. No. 7,402,556 to Trouet et al., incorporated herein by this reference. In general, these prodrugs comprise: (1) the therapeutically active agent capable of entering a target cell, in this case, amonafide or a derivative or analog of amonafide as described above; (2) an oligopeptide having the formula X-Y, wherein X is a plasmin peptide substrate of 2-4 amino acids and Y is a peptide fragment comprising 1-2 amino acids having large side chains; (3) a stabilizing group; and (4) optionally, a linker group not cleavable by plasmin. In this prodrug arrangement, the oligopeptide is directly linked to the stabilizing group at a first attachment site of the oligopeptide and the oligopeptide is directly linked to the therapeutically active agent (i.e., amonafide or a derivative or analog of amonafide) or indirectly linked through the linker group to the therapeutically active agent at a second attachment site of the oligopeptide. The stabilizing group hinders cleavage of the oligopeptide by enzymes present in whole blood. The prodrug incorporating the therapeutically active agent is cleavable by plasmin.

Yet another potential prodrug system for amonafide and derivatives or analogs of amonafide is the use of the drug complex of U.S. Pat. No. 6,368,598 to D'Amico et al., incorporated herein by this reference. In general, such a drug complex comprises a targeting carrier molecule that is selectively distributed to a specific cell type or tissue containing the specific cell type; a linker which is acted upon by a molecule that is present at an effective concentration in the environs of the specific cell type; and a therapeutically active agent to be delivered to the specific cell type, such as, in this application, amonafide or a derivative or analog of amonafide. In one application, the cell type is cells of the prostate and the drug complex is cleaved by the activity of prostate specific antigen (PSA).

When the improvement is made by use of a multiple drug system, the multiple drug system can be, but is not limited to, a multiple drug system selected from the group consisting of the use of amonafide or a derivative or analog of amonafide with:
(a) inhibitors of multi-drug resistance;
(b) specific drug resistance inhibitors;
(c) specific inhibitors of selective enzymes;
(d) signal transduction inhibitors;
(e) meisoindigo;
(f) imatinib;
(g) hydroxyurea;
(h) dasatinib;
(i) capecitabine;
(j) nilotinib;
(k) repair inhibition agents;
(l) topoisomerase inhibitors with non-overlapping side effects;
(m) PARP inhibitors; and
(n) EGFR inhibitors.

Multi-drug resistance inhibitors are described in U.S. Pat. No. 6,011,069 to Inomata et al., incorporated herein by this reference.

Specific drug resistance inhibitors are described in T. Hideshima et al., “The Proteasome Inhibitor PS-341 Inhibits Growth, Induces Apoptosis, and Overcomes Drug Resistance in Human Multiple Myeloma Cells,” Cancer Res. 61: 3071-3076 (2001), incorporated herein by this reference.

Signal transduction inhibitors are described in A. V. Lee et al., “New Mechanisms of Signal Transduction Inhibitor Action: Receptor Tyrosine Kinase Down-Regulation and Blockade of Signal Transactivation,” Clin. Cancer Res. 9: 516s (2003), incorporated herein in its entirety by this reference. Signal transduction inhibitors can include, but are not limited to, BCL/ABL kinase inhibitors, epidermal growth factor (EGF) receptor inhibitors, her-2/neu receptor inhibitors, and farnesyl transferase inhibitors, as described in U.S. Pat. No. 8,008,281 by Prendergast et al., incorporated herein by this reference.

Repair inhibition agents are described in N. M. Martin, “DNA Repair Inhibition and Cancer Therapy,” J. Photochem. Photobiol. B 63: 162-170 (2001), incorporated herein by this reference.

When the improvement is made by biotherapeutic enhancement, the biotherapeutic enhancement can be performed by use in combination as sensitizers/potentiators with a therapeutic agent or technique that can be, but is not limited to, a therapeutic agent or technique selected from the group consisting of:
(a) biological response modifiers;
(b) cytokines;
(c) lymphokines;
(d) therapeutic antibodies;
(e) antisense therapies;
(f) gene therapies;
(g) ribozymes;
(h) RNA interference;
(i) vaccines (cellular and non-cellular); and
(j) stem cells.

Biological response modifiers are described in T. E. G. K. Murthy et al., “Biological Response Modifiers,’ Int. J. Pharmtech Res. 2: 2152-2160 (2010), incorporated herein by this reference.

Antisense therapies are described, for example, in B. Weiss et al., “Antisense RNA Gene Therapy for Studying and Modulating Biological Processes,” Cell. Mol. Life Sci. 55: 334-358 (1999), incorporated herein by this reference.

Ribozymes are described, for example, in S. Pascolo, “RNA-Based Therapies” in Drug Discovery Handbook (S. C. Gad, ed., Wiley-Interscience, Hoboken, N. J., 2005), ch. 27, pp. 1273-1278, incorporated herein by this reference.

RNA interference is described, for example, in S. Pascolo, “RNA-Based Therapies” in Drug Discovery Handbook (S. C. Gad, ed., Wiley-Interscience, Hoboken, N. J., 2005), ch. 27, pp. 1278-1283, incorporated herein by this reference.

When the biotherapeutic enhancement is use in combination as sensitizers/potentiators with a therapeutic antibody, the therapeutic antibody can be, but is not limited to, a therapeutic antibody selected from the group consisting of bevacizumab (Avastin), rituximab (Rituxan), trastuzumab (Herceptin), and cetuximab (Erbitux).

Cancer vaccines are being developed. Typically, cancer vaccines are based on an immune response to a protein or proteins occurring in cancer cells that does not occur in normal cells. Cancer vaccines include Provenge for metastatic hormone-refractory prostate cancer, Oncophage for kidney cancer, CimaVax-EGF for lung cancer, MOBILAN, Neuvenge for Her2/neu expressing cancers such as breast cancer, colon cancer, bladder cancer, and ovarian cancer, Stimuvax for breast cancer, and others. Cancer vaccines are described in S. Pejawar-Gaddy & O. Finn, “Cancer Vaccines: Accomplishments and Challenges,” Crit. Rev. Oncol. Hematol. 67: 93-102 (2008), incorporated herein by this reference.

Therapeutic applications of the use of stem cells for the treatment of malignancies are also being developed. One avenue for the use of stem cells in the treatment of malignancies involves the administration of stem cells to initiate immunoreconstruction following high dose chemotherapy or radiation. Typically, in this alternative, the stem cells used are hemopoietic stem cells (HSCs). This use of stem cells is described in J. Sagar et al., “Role of Stem Cells in Cancer Therapy and Cancer Stem Cells: A Review,” Cancer Cell Internat. 7:9 (2007), incorporated herein by this reference. This may be particularly useful for malignancies affecting the immune system, such as lymphomas. Another use of stem cells in cancer therapy is by targeting malignant cells directly with stem cells. Stem cells have tumoritropic migratory properties, and can be modified by the insertion of transgenes with antitumor effects. Transgene effects can include direct tumor-cell killing, promotion of local immune responses, oncolytic virus production, and prodrug activation schemes. This use of stem cells in cancer therapy is described in M. F. Corsten & K. Shah, “Therapeutic Stem-Cells for Cancer Treatment: Hopes and Hurdles in Tactical Warfare,” Lancet Oncol. 9: 376-384 (2008), incorporated herein by this reference.

When the improvement is made by use of biotherapeutic resistance modulation, the biotherapeutic resistance modulation can be, but is not limited to, use against tumors resistant to a therapeutic agent or technique selected from the group consisting of:
(a) biological response modifiers;
(b) cytokines;
(c) lymphokines;
(d) therapeutic antibodies;
(e) antisense therapies;
(f) gene therapies;
(g) ribozymes; and
(h) RNA interference.

When the biotherapeutic resistance modulation is use against tumors resistant to therapeutic antibodies, the therapeutic antibody can be, but is not limited to, a therapeutic antibody selected from the group consisting of bevacizumab (Avastin), rituximab (Rituxan), trastuzumab (Herceptin), and cetuximab (Erbitux).

When the improvement is made by radiation therapy enhancement, the radiation therapy enhancement can be, but is not limited to, a radiation therapy enhancement agent or technique selected from the group consisting of:
(a) use with hypoxic cell sensitizers;
(b) use with radiation sensitizers/protectors;
(c) use with photosensitizers;
(d) use with radiation repair inhibitors;
(e) use with thiol depletion;
(f) use with vaso-targeted agents;
(g) use with radioactive seeds;
(h) use with radionuclides;
(i) use with radiolabeled antibodies; and
(j) use with brachytherapy; and
(k) use with bioreductive alkylating agents.

Hypoxic cell sensitizers are described in C. C. Ling et al., “The Effect of Hypoxic Cell Sensitizers at Different Irradiation Dose Rates,” Radiation Res. 109: 396-406 (1987), incorporated herein by this reference. Radiation sensitizers are described in T. S. Lawrence, “Radiation Sensitizers and Targeted Therapies,” Oncology 17 (Suppl. 13) 23-28 (2003), incorporated herein by this reference. Radiation protectors are described in S. B. Vuyyuri et al., “Evaluation of D-Methionine as a Novel Oral Radiation Protector for Prevention of Mucositis,” Clin. Cancer Res. 14: 2161-2170 (2008), incorporated herein by this reference. Photosensitizers are described in R. R. Allison & C. H. Sibata, “Oncologic Photodynamic Therapy Photosensitizers: A Clinical Review,” Photodiagnosis Photodynamic Ther. 7: 61-75 (2010), incorporated herein by this reference. Radiation repair inhibitors and DNA repair inhibitors are described in M. Hingorani et al., “Evaluation of Repair of Radiation-Induced DNA Damage Enhances Expression from Replication-Defective Adenoviral Vectors,” Cancer Res. 68: 9771-9778 (2008), incorporated herein by this reference. Thiol depleters are described in K. D. Held et al., “Postirradiation Sensitization of Mammalian Cells by the Thiol-Depleting Agent Dimethyl Fumarate,” Radiation Res. 127: 75-80 (1991), incorporated herein by this reference. Vaso-targeted agents are described in A. L. Seynhaeve et al., “Tumor Necrosis Factor α Mediates Homogeneous Distribution of Liposomes in Murine Melanoma that Contributes to a Better Tumor Response,” Cancer Res. 67: 9455-9462 (2007), incorporated herein by this reference. Bioreductive alkylating agents include tirapazamine, described in W. A. Denny, “Prospects for Hypoxia-Activated Anticancer Drugs,” Curr. Med. Chem. 4: 395-399 (2004), incorporated herein by this reference. Bioreductive alkylating agents also include nitroimidazoles, such as metronidazole, tinidazole, and nimorazole, and other substituted nitroheterocycles, described in A. Mital, “Synthetic Nitroimidazoles: Biological Activities and Mutagenicity Relationships,” Sci. Pharm. 77: 497-520 (2009) and in M. R. Juchau, “Bioactivation in Chemical Teratogenesis,” Annu. Rev. Pharmacol. Toxicol. 29: 165-187 (1989).

When the improvement is made by use of a novel mechanism of action, the novel mechanism of action can be, but is not limited to, a novel mechanism of action that is a therapeutic interaction with a target or mechanism selected from the group consisting of:
(a) inhibitors of poly-ADP ribose polymerase;
(b) agents that affect vasculature;
(c) agents that promote vasodilation;
(d) oncogenic targeted agents;
(e) signal transduction inhibitors;
(f) agents inducing EGFR inhibition;
(g) agents inducing Protein Kinase C inhibition;
(h) agents inducing Phospholipase C downregulation;
(i) agents including jun downregulation;
(j) agents modulating expression of histone genes;
(k) agents modulating expression of VEGF;
(l) agents modulating expression of ornithine decarboxylase;
(m) agents modulating expression of jun D;
(n) agents modulating expression of v-jun;
(o) agents modulating expression of GPCRs;
(p) agents modulating expression of protein kinase A;
(q) agents modulating expression of protein kinases other than protein kinase A;
(r) agents modulating expression of telomerase;
(s) agents modulating expression of prostate specific genes;
(t) agents modulating expression of histone deacetylase; and
(u) agents modulating expression of CHK2 checkpoint kinase.

Inhibitors of poly ADP-ribose polymerase include veliparib (ABT-888), AG014699, iniparib (BSI-201), carboplatin, gemcitabine, INO-1001, MK4827, nicotinamide, olaparib, paclitaxel, temozolomide, and topotecan, and are described in E. A. Comen & M. Robson, “Inhibition of Poly(ADP)-Ribose Polymerase as a Therapeutic Strategy for Breast Cancer,” Oncology 24: 55-62 (2010), incorporated herein by this reference. Agents promoting vasodilation include levosimendan, described in W. G. Toiler et al., “Levosimendan, a New Inotropic and Vasodilator Agent,” Anesthesiology 104: 556-569 (2006), incorporated herein by this reference. EGFR inhibition is described in G. Giaccone & J. A. Rodriguez, “EGFR Inhibitors: What Have We Learned from the Treatment of Lung Cancer,” Nat. Clin. Pract. Oncol. 11: 554-561 (2005), incorporated herein by this reference. Protein kinase C inhibition is described in H. C. Swannie & S. B. Kaye, “Protein Kinase C Inhibitors,” Curr. Oncol. Rep. 4: 37-46 (2002), incorporated herein by this reference. Phospholipase C downregulation is described in A. M. Martelli et al., “Phosphoinositide Signaling in Nuclei of Friend Cells: Phospholipase C β Downregulation Is Related to Cell Differentiation,” Cancer Res. 54: 2536-2540 (1994), incorporated herein by this reference. Downregulation of Jun (specifically, c-Jun) is described in A. A. P. Zada et al., “Downregulation of c-Jun Expression and Cell Cycle Regulatory Molecules in Acute Myeloid Leukemia Cells Upon CD44 Ligation,” Oncogene 22: 2296-2308 (2003), incorporated herein by this reference. The role of histone genes as a target for therapeutic intervention is described in B. Calabretta et al., “Altered Expression of G1-Specific Genes in Human Malignant Myeloid Cells,” Proc. Natl. Acad. Sci. USA 83: 1495-1498 (1986), incorporated herein by this reference. The role of VEGF as a target for therapeutic intervention is described in A. Zielke et al., “VEGF-Mediated Angiogenesis of Human Pheochromocytomas Is Associated to Malignancy and Inhibited by anti-VEGF Antibodies in Experimental Tumors,” Surgery 132: 1056-1063 (2002), incorporated herein by this reference. The role of ornithine decarboxylase as a target for therapeutic intervention is described in J. A. Nilsson et al., “Targeting Ornithine Decarboxylase in Myc-Induced Lymphomagenesis Prevents Tumor Formation,” Cancer Cell 7: 433-444 (2005), incorporated herein by this reference. The role of ubiquitin C as a target for therapeutic intervention is described in C. Aghajanian et al., “A Phase I Trial of the Novel Proteasome Inhibitor PS341 in Advanced Solid Tumor Malignancies,” Clin. Cancer Res. 8: 2505-2511 (2002), incorporated herein by this reference. The role of Jun D as a target for therapeutic intervention is described in M. M. Caffarel et al., “JunD Is Involved in the Antiproliferative Effect of Δ⁹-Tetrahydrocannibinol on Human Breast Cancer Cells,” Oncogene 27: 5033-5044 (2008), incorporated herein by this reference. The role of v-Jun as a target for therapeutic intervention is described in M. Gao et al., “Differential and Antagonistic Effects of v-Jun and c-Jun,” Cancer Res. 56: 4229-4235 (1996), incorporated herein by this reference. The role of protein kinase A as a target for therapeutic intervention is described in P. C. Gordge et al., “Elevation of Protein Kinase A and Protein Kinase C in Malignant as Compared With Normal Breast Tissue,” Eur. J. Cancer 12: 2120-2126 (1996), incorporated herein by this reference. The role of telomerase as a target for therapeutic intervention is described in E. K. Parkinson et al., “Telomerase as a Novel and Potentially Selective Target for Cancer Chemotherapy,” Ann. Med. 35: 466-475 (2003), incorporated herein by this reference. The role of histone deacetylase as a target for therapeutic intervention is described in A. Melnick & J. D. Licht, “Histone Deacetylases as Therapeutic Targets in Hematologic Malignancies,” Curr. Opin. Hematol. 9: 322-332 (2002), incorporated herein by this reference.

CHK2 checkpoint kinase is a serine/threonine protein kinase which is required for checkpoint-mediated cell cycle arrest, activation of DNA repair and apoptosis in response to the presence of DNA double-strand breaks. CHK2 checkpoint kinase may also negatively regulate cell cycle progression during unperturbed cell cycles. Following activation, CHK2 checkpoint kinase phosphorylates numerous effectors preferentially at the consensus sequence L-X—R—X-S/T (SEQ ID NO: 1) CHK2 checkpoint kinase regulates cell cycle checkpoint arrest through phosphorylation of CDC25A, CDC25B and CDC25C, inhibiting their activity. The inhibition of CDC25 phosphatase activity leads to increased inhibitory tyrosine phosphorylation of CDK-cyclin complexes and blocks cell cycle progression. CHK2 checkpoint kinase may also phosphorylate NEK6 which is involved in G2/M cell cycle arrest. CHK2 checkpoint kinase also regulates also phosphorylate NEK6 which is involved in G2/M cell cycle arrest. CHK2 checkpoint kinase also phosphorylates NEK6 which is involved in G2/M cell cycle arrest. Additionally, CHK2 checkpoint kinase stimulates the transcription of genes involved in DNA repair (including BRCA2) through the phosphorylation and activation of the transcription factor FOXM1. CHK2 checkpoint kinase also regulates apoptosis through the phosphorylation of p53/TP53, MDM4 and PML; phosphorylation of p53/TP53 at Ser20 by CHK2 may alleviate inhibition by MDM2, leading to accumulation of active p53/TP53. Phosphorylation of MDM4 may also reduce degradation of p53/TP53. CHK2 checkpoint kinase also controls the transcription of pro-apoptotic genes through phosphorylation of the transcription factor. It is also believed to act as a tumor suppressor. It may also have a DNA damage-independent function in mitotic spindle assembly by phosphorylating BRCA1. Its absence may be a cause of the chromosomal instability observed in some cancer cells. A deletion mutation at position 1100 of CHEK2, which encodes the CHK2 checkpoint kinase, is associated with an increased risk of breast cancer, particularly in the European population (H. Meijers-Heijboer et al., “Low-Penetrance Susceptibility to Breast Cancer Due to CHEK2(*)1100delC in Noncarriers of BRCA1 or BRCA2 Mutations,” Nat. Genet. 31: 55-59 (2002), incorporated herein by this reference). The activity of CHK2 checkpoint kinase is further described in J. Li et al., “Structural and Functional Versatility of the FHA Domain in DNA-Damage Signaling by the Tumor Supressor Chk2,” Mol. Cell 9: 1045-1054 (2002), incorporated herein by this reference. Inhibitors and modulators of the activity of CHK2 checkpoint kinases are known in the art, and are described, for example, in U.S. Pat. No. 8,334,309 to Klein et al., U.S. Pat. No. 8,329,709 to Banka et al., U.S. Pat. No. 8,329,701 to Mitchell et al., U.S. Pat. No. 8,318,740 to Wu, U.S. Pat. No. 8,318,735 to Shipps, Jr. et al., U.S. Pat. No. 8,252,795 to Fink et al., U.S. Pat. No. 8,227,605 to Shipps, Jr., et al., U.S. Pat. No. 8,211,054 to Guzi et al., U.S. Pat. No. 8,202,876 to Albaugh et al., and U.S. Pat. No. 8,168,651 to Chua et al., all of which are incorporated herein by this reference.

When the improvement is made by use of selective target cell population therapeutics, the use of selective target cell population therapeutics can be, but is not limited to, a use selected from the group consisting of:
(a) use against radiation sensitive cells;
(b) use against radiation resistant cells;
(c) use against energy depleted cells; and
(d) use against endothelial cells.

When the improvement is made by use with an agent to enhance the activity of a substituted naphthalimide such as amonafide or a derivative or analog of amonafide, the agent to enhance the activity of the substituted naphthalimide can be, but is not limited to, an agent selected from the group consisting of:
(a) nicotinamide;
(b) caffeine;
(c) tetandrine; and
(d) berberine.

Another aspect of the present invention is a composition to improve the efficacy and/or reduce the side effects of suboptimally administered drug therapy comprising an alternative selected from the group consisting of:

(i) a therapeutically effective quantity of a modified therapeutic agent or a derivative, analog, or prodrug of a therapeutic agent or modified therapeutic agent, wherein the modified therapeutic agent or the derivative, analog or prodrug of the therapeutic agent or modified therapeutic agent possesses increased therapeutic efficacy or reduced side effects as compared with an unmodified therapeutic agent;

(ii) a composition comprising:
(a) a therapeutically effective quantity of a therapeutic agent, a modified therapeutic agent or a derivative, analog, or prodrug of a therapeutic agent or modified therapeutic agent; and
(b) at least one additional therapeutic agent, therapeutic agent subject to chemosensitization, therapeutic agent subject to chemopotentiation, diluent, excipient, solvent system, drug delivery system, or agent for enhancing the activity or efficacy of the therapeutic agent, the modified therapeutic agent or the derivative, analog, or prodrug of a therapeutic agent or modified therapeutic agent of (a), wherein the composition possesses increased therapeutic efficacy or reduced side effects as compared with an unmodified therapeutic agent;

(iii) a therapeutically effective quantity of a therapeutic agent, a modified therapeutic agent, or a derivative, analog, or prodrug of a therapeutic agent or modified therapeutic agent that is incorporated into a dosage form, wherein the therapeutic agent, the modified therapeutic agent, or the derivative, analog, or prodrug of a therapeutic agent or modified therapeutic agent incorporated into the dosage form possesses increased therapeutic efficacy or reduced side effects as compared with an unmodified therapeutic agent;

(iv) a therapeutically effective quantity of a therapeutic agent, a modified therapeutic agent, or a derivative, analog, or prodrug of a therapeutic agent or modified therapeutic agent that is incorporated into a dosage kit and packaging, wherein the therapeutic agent, the modified therapeutic agent, or the derivative, analog, or prodrug of a therapeutic agent or modified therapeutic agent incorporated into the dosage kit and packaging possesses increased therapeutic efficacy or reduced side effects as compared with an unmodified therapeutic agent; and

(v) a therapeutically effective quantity of a therapeutic agent, a modified therapeutic agent, or a derivative, analog, or prodrug of a therapeutic agent or modified therapeutic agent that is subjected to a bulk drug product improvement, wherein the therapeutic agent, the modified therapeutic agent, or the derivative, analog, or prodrug of a therapeutic agent or modified therapeutic agent subject to the bulk drug product improvement possesses increased therapeutic efficacy or reduced side effects as compared with an unmodified therapeutic agent.

Typically, the composition possesses increased efficacy or reduced side effects for cancer therapy. Typically, the unmodified therapeutic agent is amonafide or a derivative or analog of amonafide, as described above, the modified therapeutic agent is a modification of amonafide or a derivative or analog of amonafide, and the derivative, analog, or prodrug is a derivative, analog, or prodrug of amonafide or of a derivative or analog of amonafide.

In one alternative, the composition comprises a drug combination comprising:

(i) amonafide or a derivative or analog of amonafide; and

(ii) an additional therapeutic agent selected from the group consisting of:
(a) fraudulent nucleosides;
(b) fraudulent nucleotides;
(c) thymidylate synthetase inhibitors;
(d) signal transduction inhibitors;
(e) cisplatin or platinum analogs;
(f) alkylating agents;
(g) anti-tubulin agents;
(h) antimetabolites;
(i) berberine;
(j) apigenin;
(k) colchicine or an analog thereof;
(l) genistein;
(m) etoposide;
(n) cytarabine;
(o) camptothecins;
(p) vinca alkaloids;
(q) topoisomerase inhibitors;
(r) 5-fluorouracil;
(s) curcumin;
(t) NF-κB inhibitors;
(u) rosmarinic acid;
(v) mitoguazone;
(w) meisoindigo;
(x) imatinib;
(y) dasatinib;
(z) nilotinib;
(aa) epigenetic modulators;
(ab) transcription factor inhibitors;
(ac) taxol;
(ad) homoharringtonine;
(ae) pyridoxal;
(af) spirogermanium;
(ag) caffeine;
(ah) nicotinamide;
(ai) methylglyoxalbisguanylhydrazone;
(aj) PARP inhibitors;
(ak) EGFR inhibitors;
(al) Bruton's tyrosine kinase (BTK) inhibitors;
(am) c-Myc inhibitors;
(an) PTEN inhibitors;
(ao) IDH inhibitors;
(ap) polyamine analogs;
(aq) thalidomide and analogs;
(ar) homoharringtonine and analogs;
(as) bruceantin and analogs;
(at) bisantrene, amsacrine, or analogs of bisantrene or amsacrine;
(au) mitoxantrone;
(av) vosaroxin;
(aw) dianhydrogalactitol or dibromodulcitol;
(ax) 5-azacytidine;
(ay) decitabine;
(az) anti-VEGF agents;
(ba) anti-CD20 agents;
(bb) anti-EGFR vaccines;
(bc) T-cell stimulants;
(bd) dendritic cell vaccines; and
(be) PD inhibitors.

Typically, in this composition, the amonafide or a derivative or analog of amonafide is amonafide.

In another alternative, the composition comprises:

(i) amonafide or a derivative or analog of amonafide; and

(ii) a therapeutic agent subject to chemosensitization selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(i) berberine;
(k) apigenin;
(l) colchicine or an analog of colchicine;
(m) genistein;
(n) etoposide;
(o) cytarabine;
(p) camptothecin;
(q) vinca alkaloids;
(r) 5-fluorouracil;
(s) curcumin;
(t) NF-κB inhibitors;
(u) rosmarinic acid; and
(v) mitoguazone.

Typically, in this composition, the amonafide or a derivative or analog of amonafide is amonafide.

In yet another alternative, the composition comprises:

(i) amonafide or a derivative or analog of amonafide; and

(ii) a therapeutic agent subject to chemopotentiation selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(i) berberine;
(k) apigenin;
(l) colchicine or an analog of colchicine;
(m) genistein;
(n) etoposide;
(o) cytarabine;
(p) camptothecin;
(q) vinca alkaloids;
(r) 5-fluorouracil;
(s) curcumin;
(t) NF-κB inhibitors;
(u) rosmarinic acid; and
(v) mitoguazone.

Typically, in this composition, the amonafide or derivative or analog of amonafide is amonafide.

In yet another alternative, the therapeutic agent is amonafide or a derivative or analog of amonafide, and the amonafide or derivative or analog of amonafide is subjected to a bulk drug product improvement, wherein the bulk drug product improvement is selected from the group consisting of:
(a) preparation as a free base form;
(b) salt formation;
(c) preparation as a homogeneous crystalline structure;
(d) amorphous structure;
(e) preparation as a pure isomer;
(f) increased purity;
(g) preparation with lower residual solvent content; and
(h) preparation with lower residual heavy metal content.

Typically, in this composition, the amonafide or derivative or analog of amonafide is amonafide.

In still another alternative, the therapeutic agent is amonafide or a derivative or analog of amonafide and the composition comprises a diluent, wherein the diluent is selected from the group consisting of:
(a) an emulsion;
(b) dimethylsulfoxide (DMSO);
(c) N-methylformamide (NMF)
(d) dimethylformamide (DMF)
(e) dimethylacetamide (DMA);
(f) ethanol;
(g) benzyl alcohol;
(h) dextrose-containing water for injection;
(i) Cremophor;
(j) cyclodextrins; and
(k) PEG.

Typically, in this composition, the amonafide or derivative or analog of amonafide is amonafide.

In still another alternative, the therapeutic agent is amonafide or a derivative or analog of amonafide and the composition comprises a solvent system, wherein the solvent system is selected from the group consisting of: of:
(a) an emulsion;
(b) DMSO;
(c) NMF;
(d) DMF;
(e) DMA;
(f) ethanol;
(g) benzyl alcohol;
(h) dextrose-containing water for injection;
(i) Cremophor;
(j) PEG; and
(k) salt systems.

Typically, in this composition, the amonafide or derivative or analog of amonafide is amonafide.

In yet another alternative, the therapeutic agent is amonafide or a derivative or analog of amonafide and the composition comprises an excipient, wherein the excipient is selected from the group consisting of:
(a) mannitol;
(b) albumin;
(c) EDTA;
(d) sodium bisulfite;
(e) benzyl alcohol;
(f) carbonate buffers;
(g) phosphate buffers;
(h) PEG;
(i) vitamin A;
(j) vitamin D;
(k) vitamin E;
(l) esterase inhibitors;
(m) cytochrome P450 inhibitors;
(n) multi-drug resistance (MDR) inhibitors;
(o) organic resins; and
(p) detergents.

Typically, in this composition, the amonafide or derivative or analog of amonafide is amonafide.

In yet another alternative, the therapeutic agent is amonafide or a derivative or analog of amonafide, and the amonafide or derivative or analog of amonafide is incorporated into a dosage form selected from the group consisting of:
(a) tablets;
(b) capsules;
(c) topical gels;
(d) topical creams;
(e) patches;
(f) suppositories;
(g) lyophilized dosage fills;
(h) immediate-release formulations;
(i) slow-release formulations;
(j) controlled-release formulations; and
(k) liquid in capsules.

Typically, in this composition, the amonafide or derivative or analog of amonafide is amonafide.

In yet another alternative, the therapeutic agent is amonafide or a derivative or analog of amonafide and the amonafide or derivative or analog of amonafide is incorporated into a dosage kit and packaging selected from the group consisting of amber vials to protect from light and stoppers with specialized coatings to improve shelf-life stability.

Typically, in this composition, the amonafide or derivative or analog of amonafide is amonafide.

In still another alternative, the therapeutic agent is amonafide or a derivative or analog of amonafide and the composition comprises a drug delivery system selected from the group consisting of:
(a) oral dosage forms;
(b) nanocrystals;
(c) nanoparticles;
(d) cosolvents;
(e) slurries;
(f) syrups;
(g) bioerodible polymers;
(h) liposomes;
(i) slow-release injectable gels; and
(j) microspheres.

Typically, in this composition, the amonafide or derivative or analog of amonafide is amonafide.

In yet another alternative, the therapeutic agent is amonafide or a derivative or analog of amonafide and the amonafide or derivative or analog of amonafide is present in the composition in a drug conjugate form selected from the group consisting of:
(a) a polymer system;
(b) polylactides;
(c) polyglycolides;
(d) amino acids;
(e) peptides; and
(f) multivalent linkers.

Typically, in this composition, the amonafide or derivative or analog of amonafide is amonafide.

In yet another alternative, the therapeutic agent is a modified amonafide or a modified derivative or analog of amonafide and the modification is selected from the group consisting of:
(a) alteration of side chains to increase or decrease lipophilicity;
(b) addition of an additional chemical functionality to alter a property selected from the group consisting of reactivity, electron affinity, and binding capacity; and
(c) alteration of salt form.

Typically, in this composition, the modified amonafide or modified derivative or analog of amonafide is a modified amonafide.

In still another alternative of a composition according to the present invention, the therapeutic agent is amonafide or a derivative or analog of amonafide and the amonafide or derivative or analog of amonafide is in the form of a prodrug system, wherein the prodrug system is selected from the group consisting of:
(a) enzyme sensitive esters;
(b) dimers;
(c) Schiff bases;
(d) pyridoxal complexes;
(e) caffeine complexes;
(f) plasmin-activated prodrugs; and
(g) drug targeting complexes comprising a targeting carrier molecule that is selectively distributed to a specific cell type or tissue containing the specific cell type; a linker which is acted upon by a molecule that is present at an effective concentration in the environs of the specific cell type; and a therapeutically active agent to be delivered to the specific cell type.

Typically, in this composition, the amonafide or modified derivative or analog of amonafide is amonafide.

In yet another alternative, the therapeutic agent is amonafide or a derivative or analog of amonafide and the composition further comprises at least one additional therapeutic agent to form a multiple drug system, wherein the at least one additional therapeutic agent is selected from the group consisting of:
(a) inhibitors of multi-drug resistance;
(b) specific drug resistance inhibitors;
(c) specific inhibitors of selective enzymes;
(d) signal transduction inhibitors;
(e) meisoindigo;
(f) imatinib;
(g) hydroxyurea;
(h) dasatinib;
(i) capecitabine;
(j) nilotinib;
(k) repair inhibition agents;
(l) topoisomerase inhibitors with non-overlapping side effects; and
(m) PARP inhibitors.

Typically, in this composition, the amonafide or modified derivative or analog of amonafide is amonafide.

In still another alternative, the therapeutic agent is amonafide or a derivative or analog of amonafide and the composition further comprises at least one agent for enhancing the activity or efficacy of the amonafide or derivative or analog of amonafide, wherein the at least one agent for enhancing the activity or efficacy of the amonafide or derivative or analog of amonafide is selected from the group consisting of:
(i) nicotinamide;
(ii) caffeine;
(iii) tetandrine; and
(iv) berberine.

Typically, in this composition, the amonafide or modified derivative or analog of amonafide is amonafide.

When a pharmaceutical composition according to the present invention includes a prodrug, prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992), all incorporated herein by this reference.

When the pharmacologically active compound in a pharmaceutical composition according to the present invention possesses a sufficiently acidic, a sufficiently basic, or both a sufficiently acidic and a sufficiently basic functional group, these group or groups can accordingly react with any of a number of inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methyl benzoates, dinitrobenzoates, hydroxybenzoates, methoxybenzoates, phthalates, sulfonates, xylenesulfonates, phenylacetates, phenyl propionates, phenyl butyrates, citrates, lactates, 6-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

The amount of a given pharmacologically active agent that is included in a unit dose of a pharmaceutical composition according to the present invention will vary depending upon factors such as the particular compound, disease condition and its severity, the identity (e.g., weight) of the subject in need of treatment, but can nevertheless be routinely determined by one skilled in the art. Typically, such pharmaceutical compositions include a therapeutically effective quantity of the pharmacologically active agent and an inert pharmaceutically acceptable carrier or diluent. Typically, these compositions are prepared in unit dosage form appropriate for the chosen route of administration, such as oral administration or parenteral administration. A pharmacologically active agent as described above can be administered in conventional dosage form prepared by combining a therapeutically effective amount of such a pharmacologically active agent as an active ingredient with appropriate pharmaceutical carriers or diluents according to conventional procedures. These procedures may involve mixing, granulating and compressing or dissolving the ingredients as appropriate to the desired preparation. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a pharmacologically active agent as described above is dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease and/or condition being treated. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of factors affecting pharmacokinetics, such as liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Optimal dosages for a given set of conditions can be ascertained by those skilled in the art using conventional dosage-determination tests in view of the experimental data for an agent. For oral administration, an exemplary daily dose generally employed is from about 0.001 to about 3000 mg/kg of body weight, with courses of treatment repeated at appropriate intervals. In some embodiments, the daily dose is from about 1 to 3000 mg/kg of body weight.

Methods and compositions according to the present invention are suitable for use in treating diseases and conditions of both humans and non-humans, including treatment of socially and economically important animals such as dogs, cats, cows, horses, sheep, pigs, goats, and other species. Unless specified, methods and compositions according to the present invention are not limited to treatment of humans. The effectiveness of methods and compositions according to the present invention can be monitored by conventional methods and evaluated in terms of such factors as reduction in pain, improvement in mobility or quality of life, improvement of Karnofsky Performance Score, reduction of tumor burden, reduction of metastases, improvement of effectiveness of other concurrently administered agents, or other factors known in the art. The use of the term “treatment” herein does not imply a cure for any disease or condition for which amonafide or a derivative or analog thereof or a pharmaceutical composition comprising amonafide or a derivative or analog thereof is administered.

Typical daily doses in a patient may be anywhere between about 500 mg to about 3000 mg, given once or twice daily, e.g., 3000 mg can be given twice daily for a total dose of 6000 mg. In one embodiment, the dose is between about 1000 to about 3000 mg. In another embodiment, the dose is between about 1500 to about 2800 mg. In other embodiments, the dose is between about 2000 to about 3000 mg. In particular, for amonafide or derivatives or analogs thereof, suitable doses typically are from about 50 m g/m² to about 500 mg/m² or from about 0.1 mg/kg to about 10 mg/kg. These doses are particularly suitable for amonafide.

Plasma concentrations in the subjects may be between about 1 μM to about 1000 μM. In some embodiments, the plasma concentration may be between about 200 μM to about 800 μM. In other embodiments, the concentration is about 300 μM to about 600 μM. In still other embodiments the plasma concentration may be between about 400 to about 800 μM. In one typical alternative, dosages of amonafide or a derivative or analog of amonafide are from about 1 mg/m²/day to about 600 mg/m²/day. Administration of prodrugs is typically dosed at weight levels which are chemically equivalent to the weight levels of the fully active form.

The compositions of the invention may be manufactured using techniques generally known for preparing pharmaceutical compositions, e.g., by conventional techniques such as mixing, dissolving, granulating, dragee-making, levitating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, solutions, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the composition to allow for the preparation of highly concentrated solutions, or can contain suspending or dispersing agents. Pharmaceutical preparations for oral use can be obtained by combining the pharmacologically active agent with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-α-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

For administration intranasally or by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

A pharmaceutical composition can be administered by a variety of methods known in the art. The routes and/or modes of administration vary depending upon the desired results. Depending on the route of administration, the pharmacologically active agent may be coated in a material to protect the targeting composition or other therapeutic agent from the action of acids and other compounds that may inactivate the agent. Conventional pharmaceutical practice can be employed to provide suitable formulations or compositions for the administration of such pharmaceutical compositions to subjects. Any appropriate route of administration can be employed, for example, but not limited to, intravenous, parenteral, intraperitoneal, intravenous, transcutaneous, subcutaneous, intramuscular, intraurethral, or oral administration. Depending on the severity of the malignancy or other disease, disorder, or condition to be treated, as well as other conditions affecting the subject to be treated, either systemic or localized delivery of the pharmaceutical composition can be used in the course of treatment. The pharmaceutical composition as described above can be administered together with additional therapeutic agents intended to treat a particular disease or condition, which may be the same disease or condition that the pharmaceutical composition is intended to treat, which may be a related disease or condition, or which even may be an unrelated disease or condition.

Pharmaceutical compositions according to the present invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, and implantable infusion systems. Formulations for inhalation may contain excipients, for example, lactose, or may be aqueous solutions containing, e.g., polyoxyethylene-9-lauryl ether, glycocholate and deoxycholate, or can be oily solutions for administration or gels.

Pharmaceutical compositions according to the present invention are usually administered to the subjects on multiple occasions. Intervals between single dosages can be weekly, monthly or yearly. Intervals can also be irregular as indicated by therapeutic response or other parameters well known in the art. Alternatively, the pharmaceutical composition can be administered as a sustained release formulation, in which case less frequent administration is required. Dosage and frequency vary depending on the half-life in the subject of the pharmacologically active agent included in a pharmaceutical composition. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

For the purposes of the present application, treatment can be monitored by observing one or more of the improving symptoms associated with the disease, disorder, or condition being treated, or by observing one or more of the improving clinical parameters associated with the disease, disorder, or condition being treated, as described above.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising a pharmacologically active agent according to the present invention by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the pharmacologically active agent, such as aqueous solubility, partition coefficient, molecular size, stability, and nonspecific binding to proteins and other biological macromolecules. This process of preparation also takes into account biological factors, such as absorption, distribution, metabolism, duration of action, the possible existence of side effects, and margin of safety, for the pharmacologically active agent. Accordingly, one of ordinary skill in the art could modify the formulations into a formulation having the desirable properties described above for a particular application.

U.S. Pat. No. 6,573,292 by Nardella, U.S. Pat. No. 6,921,722 by Nardella, U.S. Pat. No. 7,314,886 to Chao et al., and U.S. Pat. No. 7,446,122 by Chao et al., which disclose methods of use of various pharmacologically active agents and pharmaceutical compositions in treating a number of diseases and conditions, including cancer, and methods of determining the therapeutic effectiveness of such pharmacologically active agents and pharmaceutical compositions, are all incorporated herein by this reference.

The invention is illustrated by the following Example. This Example is included for illustrative purposes and is not intended to limit the invention.

### Example 1

### Use of Amonafide in Combination with Other Anti-Neoplastic Drugs

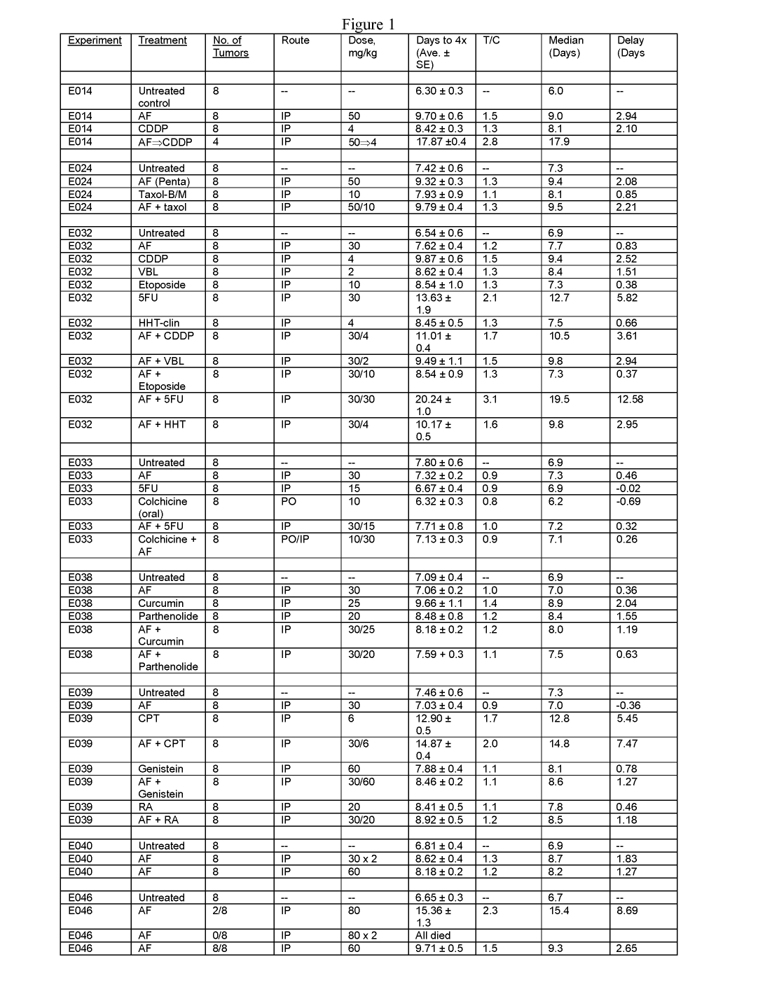

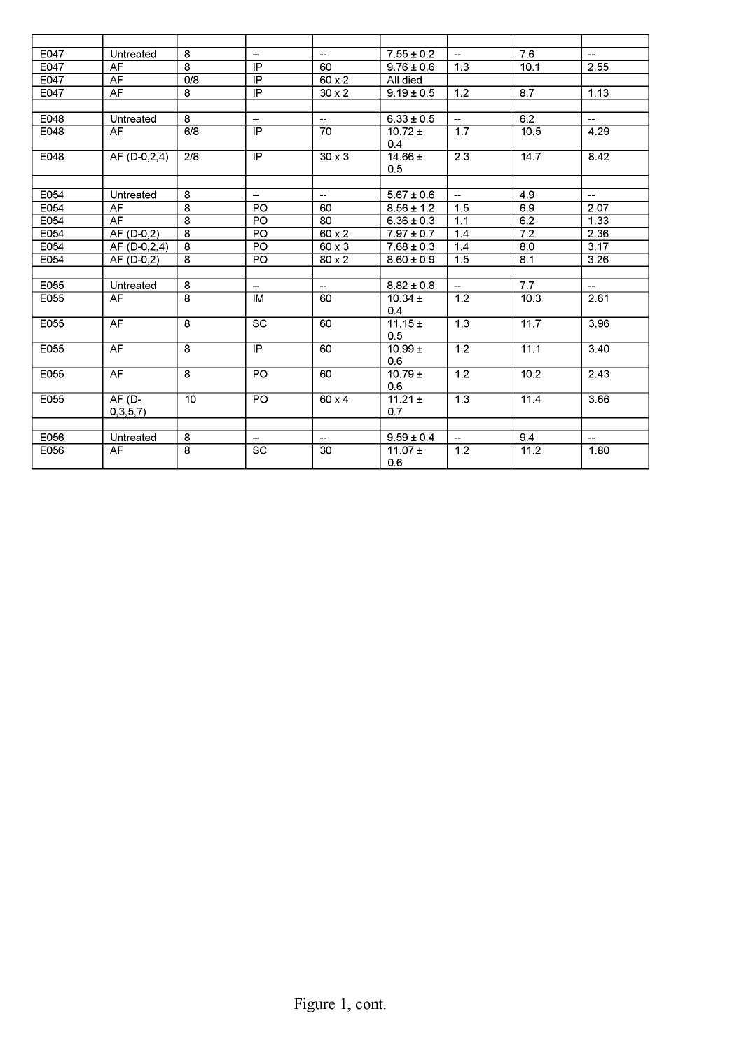

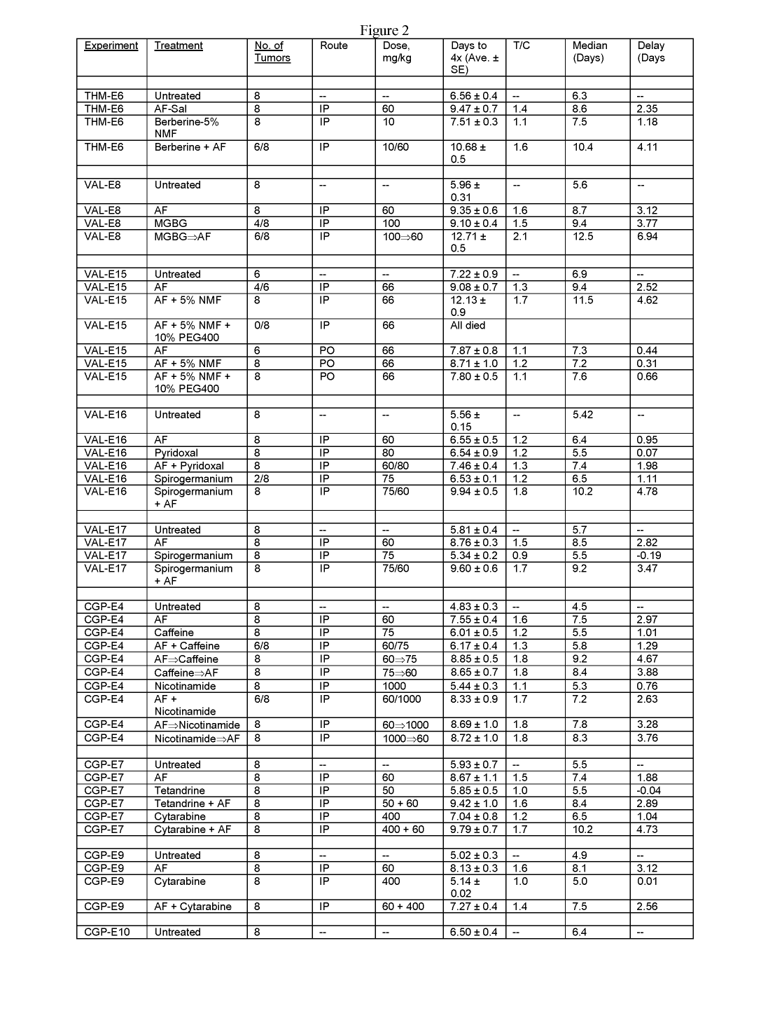

Tables 1 and 2, shown in FIGS. 1 and 2, respectively, show the use of amonafide, alone or in combination with another anti-neoplastic drug, in a tumor model in mice as described below

Female C3H mice (Charles River Laboratories, Hollister, Calif.), approximately 3 months old, were used for the study. The average body weight was approximately 25 g. Animals were maintained in isolator cages on a 12-hour light-and-dark cycle. Food and water were available ad libitum. The RIF-1 murine fibrosarcoma cell line was maintained in in vitro culture (Waymouth medium supplemented with 20% fetal bovine serum) at 37° C. in a humidified 5% CO₂ incubator. Log-phase RIF-1 cells were trypsinized and harvested from cell culture flasks to yield a concentration of 4×10⁶ cells/mL, then injected intradermally in a volume of 50 μL (equivalent to 2×10⁵ cells per injection) into both flanks of each mouse. Nine days later, when tumors reached approximately 100 mm³ in size, the animals were randomized to different treatment groups. The number of animals per treatment group is as shown in Tables 1 and 2. The intraperitoneal injection volume was 100 μL. The oral administration volume was 100 μL.

For evaluation of tumor growth delay, tumors were measured three times weekly for up to 22 days with Vernier calipers. Tumor volume (cubic millimeters, mm³) was calculated according to the following formula:

V=π/6×D₁×D₂×D₃,

where D₁, D₂, and D₃ are perpendicular diameters measured in milliliters. Tumor volume quadrupling time (TVQT), defined as the time required for a tumor to grow to four times (4×) its initial volume (at the time of treatment), was used as a study endpoint. The TVQT was determined for each treatment group and expressed in days as the mean±standard error (SE).

In these tables, “CDDP” is cisplatin, “VBL” is vinblastine, “HHT” is homoharringtonine, “5FU” is 5-fluorouracil, “CPT” is camptothecin, “RA” is rosmarinic acid, and “MGBG” is methylglyoxalbisguanylhydrazone. For solvents and excipients, “Sal” is saline, “NMF” is N-methylformamide, and “PEG400” is polyethylene glycol 400. For routes of administration, “IP” is intraperitoneal, “PO” is oral, “IM” is intramuscular, and “SC” is subcutaneous. In these tables, the use of the “+” symbol means that both drugs were administered at the same time, while the use of the “ [symbol] ” symbol means that the drug to the left of the symbol was administered first, followed by the drug to the right of the symbol; the second drug was administered 30 minutes after the first drug. In the headings for these tables, “Days to 4×” is the number of days it takes the tumors to grow to four times their original size. “T/C” represents the average number of days that it takes the tumors to grow to four times their original size for the specified treatment divided by the number of days that it takes the tumors to grow to four times their original size for the control.

This data shows that amonafide has a significant effect on tumor growth, either alone or in combination with another anti-neoplastic drug. The results when amonafide is administered together with another anti-neoplastic drug are evidence of synergism.

The data presented demonstrates that amonafide is an active antiproliferative agent delivered by direct systemic administration (e.g., by intraperitoneal administration) or by the oral route. Moreover, amonafide was active after a single administration or given as multiple doses orally. As a chemosensitizer or chemopotentiatior, amonafide enhanced the antitumor effects of cisplatin, 5-fluorouracil, homoharringtonine, vinblastine, camptothecin, methylglyoxalbisguanylhydrazone, and cytarabine.

In addition, amonafide could be administered with alternative diluents such as N-methylformamide.

The activity of amonafide could be enhanced by the use of agents to sensitize or potentiate its activity. For example, nicotinamide, caffeine, tetandrine, or berberine enhances amonafide activity.

### Advantages of the Invention

The present invention provides more effective and efficient methods of using therapeutic drugs that have previously been evaluated for treatment of a number of diseases and conditions, especially hyperproliferative disorders, but whose evaluations resulted in a premature conclusion of lack of sufficient efficacy or of occurrence of side effects sufficient to prevent the use of the therapeutic drug. Such more effective and efficient methods of therapeutic drugs will improve efficacy, prevent or reduce the occurrence of significant side effects, and will identify categories of patients and situations in which such drugs can be effectively employed. Such drugs particularly include amonafide and derivatives and analogs thereof.

Methods according to the present invention possess industrial applicability for the preparation of a medicament for the treatment of a number of diseases and conditions, especially hyperproliferative diseases, and compositions according to the present invention possess industrial applicability as pharmaceutical compositions.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1. A method to improve the efficacy and/or reduce the side effects of suboptimally administered drug therapy comprising the steps of:
(a) identifying at least one factor or parameter associated with the efficacy and/or occurrence of side effects of the drug therapy; and
(b) modifying the factor or parameter to improve the efficacy and/or reduce the side effects of the drug therapy;
wherein the drug therapy comprises administration of amonafide or a derivative or analog thereof.

2. The method of claim 1 wherein the drug therapy comprises administration of amonafide.

3. The method of claim 1 wherein the drug therapy comprises administration of a derivative or analog of amonafide.

4. The method of claim 3 wherein the derivative or analog of amonafide is selected from the group consisting of:
(a) a derivative of amonafide wherein the amino group attached to one of the six-membered aromatic rings has one or both of the hydrogens replaced with C₁-C₃ lower alkyl;
(b) a derivative of amonafide wherein the nitrogen connected to one of the six-membered rings through an ethylene linkage has one or both of the methyl groups bound thereto replaced with C₂-C₃ lower alkyl;
(c) a derivative of amonafide wherein the ethylene linkage is replaced with a propylene (C₃) or a butylene (C₄) linkage;
(d) a derivative of amonfide of Formula (II) 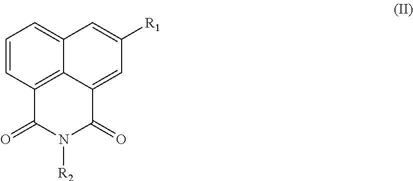
wherein: R₁ is selected from the group consisting of C₁-C₅ alkyl, amino, nitro, cyano, C₁-C₅ alkoxy, and hydrogen; and wherein R₂ is C₁-C₅ alkyl;
(e) a derivative of amonfide of Formula (III) 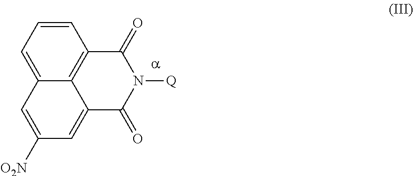
wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s) 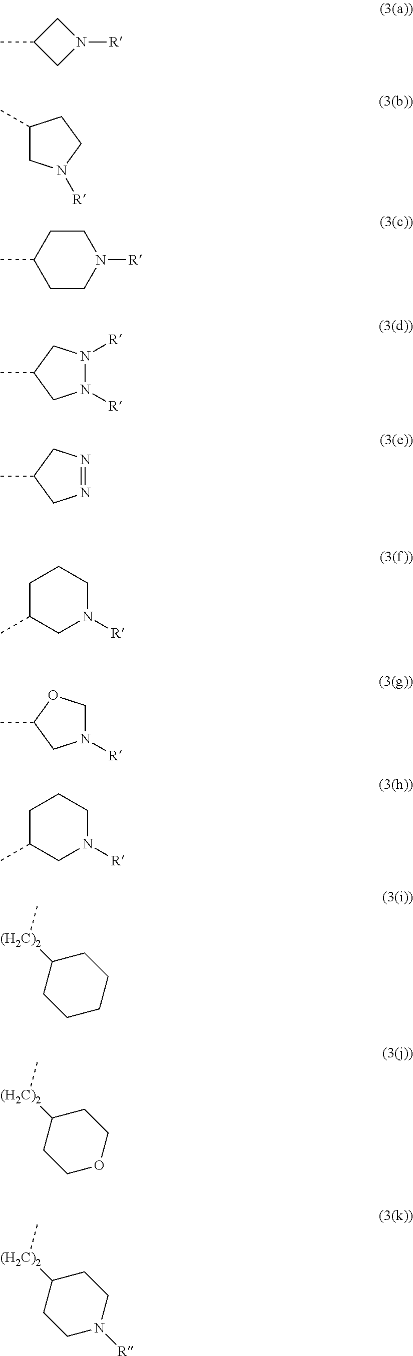 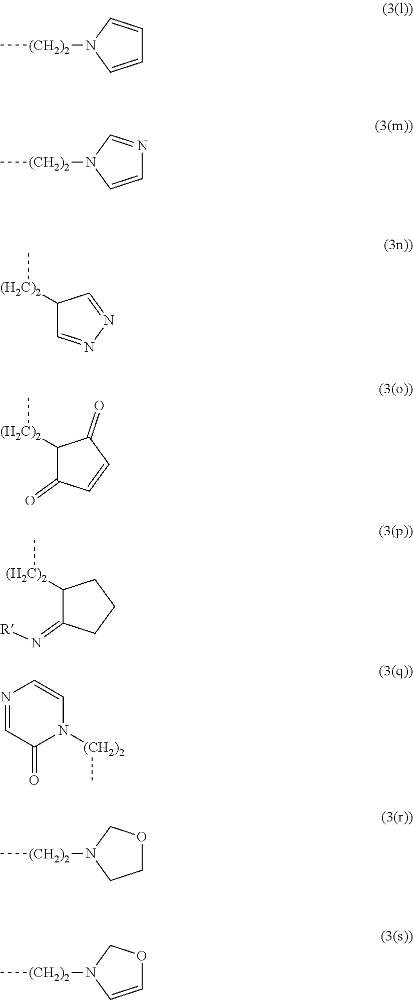
(f) a derivative of amonafide of Formula (III) wherein Q is selected from the group consisting of 1-R′-azetid-3-yl, 1-R′-pyrrolid-3-yl, 1-R′-piperid-4-yl, 1,2-diR′-1,2-diazolid-4-yl, 1,2-diazol-1-en-4-yl, 1-R′-piperid-4-yl, or 3-R′-oxazolid-5-yl, wherein R′ is selected from the group consisting of alkyl, alkenyl, acyl, alkoxy, aryl, amino, substituted amino, sulfo, sulfamoyl, carboxyl, carbamyl, and cyano;
(g) a derivative of amonafide of Formula (III) that is a naphthalimide wherein Q is —(CH₂)₂NR₂, where R is lower alkyl;
(h) a derivative of amonafide of Formula (III) that is a naphthalimide wherein Q is —(CH₂)₂NR₂, wherein NR₂ forms a heterocyclic group;
(i) a derivative of amonafide of Formula (III) that is a naphthalimide wherein Q is —(CH₂)₂NR₂ and wherein R₂ is —(CH₂)ₙ— or —(CH₂)ₘ—X—(CH₂)ₙ—, wherein m or n can be 0 to 5 and wherein X is NR″; wherein R″ is hydrogen, alkyl, alkenyl, acyl, alkoxy, aryl, amino, substituted amino, sulfo, sulfamoyl, carboxyl, carbamyl, cyano, or is not present; O; or S;
(j) a derivative of amonafide of Formula (III) wherein the tricyclic framework is derivatized so that it has one or more unsaturated bonds therein;
(k) a derivative of amonafide of Formula (III) wherein the tricyclic framework is derivativized so that it has at least one substituent selected from the group consisting of alkyl, aryl, and heteroaryl;
(l) a derivative of amonafide of Formula (III) wherein Q is selected from the group consisting of 1-pyrrolidyl, 3-R′-piperidyl, morpholino, 1-R′-piperazin-4-yl, 1-pyrrolyl, 1-imidazolyl, 1,3,5-triazol-1-yl, N-maleimido, 2-(R′-imino)pyrrolidyl, pyrazin-2-on-1-yl, 3-oxazolidyl, 3-oxazolyl, 2-pyrrolyl, 3-chloro-1-pyrrolidyl, 2-nitro-1-imidazolyl, 4-methoxy-1-imidazolyl, and 3-methyl-1-imidazolyl;
(m) a derivative of amonafide of Formula (III) wherein Q is selected from the group consisting of Subformulas 3(h), 3(i), 3(j), 3(k), 3(1), 3(m), 3(n), 3(o), 3(p), 3(q), 3(r), and 3(s), wherein R′ is selected from the group consisting of alkyl, alkenyl, acyl, alkoxy, aryl, amino, substituted amino, sulfo, sulfamoyl, carboxyl, carbamyl, and cyano;
(n) a derivative of amonafide of Formula (III) wherein the naphthalimide ring is modified to include one or more amino groups at positions other than position 3 of the naphthalimide ring;
(o) a derivative of amonafide of Formula (III) wherein the amino group at position 3 is replaced with an alternative substituent group selected from the group consisting of alkyl, aryl, nitro, amino, substituted amino, sulfamoyl, halo, carboxyl, carbamyl, and cyano;
(p) a derivative of amonafide of Formula (III) wherein an additional group is attached to the naphthalimide ring also comprising an amino group at position 3, the additional group being selected from the group consisting of alkyl, aryl, nitro, substituted amino, sulfamoyl, halo, carboxyl, carbamyl, and cyano;
(q) an analog of amonafide wherein the naphthalene ring is replaced with one bearing one or more nitrogen atoms in either or both rings;
(r) an analog of amonafide that is an isoquinoline analog of Formula (IV) 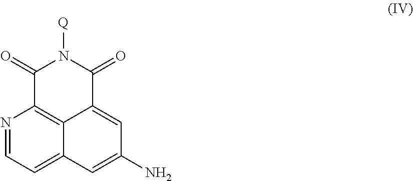
wherein Q is selected from the group consisting of Subformulas 3(a), 3(b), 3(c), 3(d), 3(e), 3(f), 3(g), 3(h), 3(i), 3(j), 3(k), 3(1), 3(m), 3(n), 3(o), 3(p), 3(q), 3(r), and 3(s);
(s) an analog of amonafide that is an isoquinoline analog of Formula (IV) wherein Q is —(CH₂)ₙ—N(CH₃)₂, wherein n is 1-12; and
(t) a derivative or analog of amonafide or of alternatives (a)-(s) including one or more optional substituents, provided that the optionally substituted amonafide derivative or analog possesses substantially equivalent pharmacological activity to amonafide as defined in terms of either or both topoisomerase II inhibition and DNA intercalation.

5. The method of claim 3 wherein the derivative or analog of amonafide is selected from the group consisting of derivatives of amonafide, derivatives of azonafide, derivatives of mitonafide, and derivatives of elinafide.

6. The method of claim 3 wherein the derivative or analog of amonafide is selected from the group consisting of heterocyclic-substituted bis-1,8-naphthalimide compounds, 1,8 naphthalimide imidazo{4,5,1-de}acridones, 2-substituted-1,2-dihydro-3H-dibenz[de,h]isoquinoline-1,3-diones, amino-substituted-[2′-(dimethylamino)ethyl]1,2-dihydro-3H-dibenz[de,h]isoquinoline-1,3-diones, tetrahydroazonafides, phenanthrene analogs of azonafide, and azaphenanthrenes.

7. The method of claim 1 wherein the factor or parameter is selected from the group consisting of:
(a) dose modification;
(b) route of administration;
(c) schedule of administration;
(d) indications for use;
(e) selection of disease stage;
(f) other indications;
(g) patient selection;
(h) patient/disease phenotype;
(i) patient/disease genotype;
(j) pre/post-treatment preparation
(k) toxicity management;
(l) pharmacokinetic/pharmacodynamic monitoring;
(m) drug combinations;
(n) chemosensitization;
(o) chemopotentiation;
(p) post-treatment patient management;
(q) alternative medicine/therapeutic support;
(r) bulk drug product improvements;
(s) diluent systems;
(t) solvent systems;
(u) excipients;
(v) dosage forms;
(w) drug delivery systems;
(x) drug conjugate forms;
(y) prodrugs;
(z) multiple drug systems;
(aa) biotherapeutic enhancement;
(ab) biotherapeutic resistance modulation;
(ac) radiation therapy enhancement;
(ad) novel mechanisms of action;
(ae) selective target cell population therapeutics; and
(af) use with an agent to enhance its activity.

8. The method of claim 1 wherein the drug therapy is administered to treat a hyperproliferative disease.

9. The method of claim 8 wherein the hyperproliferative disease is cancer.

10. The method of claim 9 wherein the cancer is a form of cancer selected from the group consisting of: (1) melanoma; (2) colon cancer; (3) chronic lymphocytic leukemia; (4) skin cancer; (5) lung cancer, including small-cell lung cancer and non-small-cell lung cancer; (6) throat cancer; (7) stomach cancer; (8) salivary gland cancer; (9) breast cancer, including triple-negative breast cancer and breast cancer characterized by overexpression of Her-2/neu; (10) prostate cancer, including androgen-resistant prostate cancer; (11) pancreatic cancer; (12) ovarian cancer; (13) uterine cancer; (14) endometrial cancer; (15) other leukemias; (16) renal cell carcinoma; (17) multiple myeloma; (18) liver cancer; (19) pituitary gland cancer; (20) acute myeloid leukemia; (21) oophoroma; (22) glioma; (23) head and neck cancer; (23) colorectal cancer; (24) bladder cancer; (25) HPV-induced papilloma; (26) lymphoma, including both non-Hodgkin's lymphoma and Hodgkin's lymphoma; (27) myelodysplastic syndrome; (28) chronic myelocytic leukemia, including treatment of chronic myelocytic leukemia subsequent to the administration of homoharringtonine; (29) malignancies with overexpressed or mutated EGFR; (30) malignancies with overexpressed or mutated Her2/neu; (31) malignancies with overexpressed or mutated Braf; (32) malignancies with overexpressed or mutated BTK; (33) malignancies with overexpressed or mutated KRAS; (34) malignancies with overexpressed or mutated c-Myc; and (3S) malignancies with overexpressed or mutated p53.

11. The method of claim 9 wherein the cancer is a form of cancer selected from the group consisting of: (1) triple-negative breast cancer; (2) acute leukemia; (3) myelodysplastic syndrome; (4) chronic myelocytic leukemia, subsequent to or in combination with the administration of tyrosine kinase inhibitors or homoharringtonine; (5) chronic lymphocytic leukemia; (6) Hodgkin's lymphoma; (7) non-Hodgkin's lymphoma; (8) mycosis fungoides; (9) prostate cancer; (10) lung small cell carcinoma, subsequent to or in combination with an EGFR inhibitor, wherein the lung small cell carcinoma is characterized by either wild-type or mutated EGFR; (11) lung non-small cell carcinoma, subsequent to or in combination with an EGFR inhibitors, wherein the lung non-small cell carcinoma is characterized by either wild-type or mutated EGFR; (12) breast cancer characterized by overexpressed Her-2-neu; (13) glioblastoma that is resistant to one or both of the following therapeutic agents: temozolomide (Temodar) or bevacizumab (Avastin), or is characterized by EGFR variant III, either alone or in combination with other therapeutic agents; and (14) malignancies characterized by overexpressed topoisomerase II.

12. The method of claim 11 wherein the cancer is acute leukemia and the acute leukemia is selected from the group consisting of acute myeloid leukemia, acute erythroid leukemia, and acute lymphoblastic leukemia.

13. The method of claim 11 wherein the cancer is prostate cancer and wherein the prostate-cancer is androgen-resistant prostate cancer.

14. The method of claim 8 wherein the hyperproliferative disease is a non-malignant proliferative disease selected from the group consisting of psoriasis and HSV-induced shingles.

15. The method of claim 1 wherein the improvement is made by dose modification.

16. The method of claim 15 wherein the suboptimally administered drug therapy comprises administration of amonafide.

17. The method of claim 15 wherein the suboptimally administered drug therapy comprises administration of a derivative or analog of amonafide.

18. The method of claim 15 wherein the dose modification is a modification selected from the group consisting of:
(a) continuous i.v. infusion for hours to days;
(b) biweekly administration;
(c) doses greater than 5 mg/m²/day;
(d) progressive escalation of dosing from 1 mg/m²/day based on patient tolerance;
(e) doses less than 1 mg/m² for greater than 14 days;
(f) use of caffeine to modulate metabolism;
(g) use of isoniazid to modulate metabolism;
(h) selected and intermittent boost dose administrations;
(i) bolus single and multiple doses of 1-5 mg/m²;
(j) oral dosing including multiple daily dosing;
(k) micro-dosing;
(l) immediate release dosing;
(m) slow release dosing; and
(n) controlled release dosing.

19.-213. (canceled)
